SEQUENCE LISTING (522 sequences)

SEQ ID NO: 1 - protein, 8 aa, Artificial Sequence
  2-B16-B Heavy Chain  CDR1
GYTFTSYG

SEQ ID NO: 2 - protein, 8 aa, Artificial Sequence
  2-B16-B Heavy Chain CDR2
IYIGNGYT

SEQ ID NO: 3 - protein, 11 aa, Artificial Sequence
  2-B16-B Heavy Chain CDR3
ARDGTRDAMDY

SEQ ID NO: 4 - protein, 6 aa, Artificial Sequence
  2-B16-B Light Chain CDR1
ENIYSN

SEQ ID NO: 5 - DNA, 3 bp, Artificial Sequence
  2-B16-B Light Chain CDR2
aat

SEQ ID NO: 6 - protein, 9 aa, Artificial Sequence
  2-B16-B Light Chain CDR3
QHFYSSPWT

SEQ ID NO: 7 - protein, 118 aa, Artificial Sequence
  2-B16-B Heavy Chain
EVQLQQSGAELVRPGSSVKMSCKTSGYTFTSYGINWVKQRPGQGLEWIGYIYIGNGYTAYNEKFKGKATLTSDTSSSTAYMQLSSLTSEDSAIYFCARDGTRDAMDYWGQGTSVTVSS

SEQ ID NO: 8 - DNA, 354 bp, Artificial Sequence
  2-B16-B Heavy Chain
gaggtccagcttcagcagtctggagctgagctggtgaggcctgggtcctcagtgaaaatgtcctgcaagacttctggatatacattcacaagctacggtataaactgggtgaagcagaggcctggacagggcctggaatggattggatatatttatattggaaatggttatactgcgtacaatgagaagttcaagggcaaggccacactgacttcagacacatcctccagcacagcctacatgcagctcagcagtctgacatctgaggactctgcaatctatttctgtgcaagagacggtactagggatgctatggactactggggtcaaggaacctcagtcaccgtctcctca

SEQ ID NO: 9 - protein, 107 aa, Artificial Sequence
  2-B16-B Light Chain
DIQMTQSPASLSASVGETVTITCRASENIYSNLAWYQQKQGKSPQLLVYAATNLADGVPSRFSGSGSGTQFSLKINSLQPEDFGSYYCQHFYSSPWTFGGGTKLEIK

SEQ ID NO: 10 - DNA, 321 bp, Artificial Sequence
  2-B16-B Light Chain
gacatccaaatgactcagtctccagcctccctatctgcatctgtgggagaaactgtcaccatcacatgtcgagcaagtgaaaatatttacagtaatttagcatggtatcagcagaaacagggaaaatctcctcaactcctggtctatgctgcaacaaatttagcagatggtgtgccatcaaggttcagtggcagtggatcaggcacacagttttctctgaagatcaacagcctgcagcctgaagattttgggagttattactgtcaacatttttatagtagtccgtggacgttcggtggaggcaccaagctggaaatcaaa

SEQ ID NO: 11 - protein, 8 aa, Artificial Sequence
  2-B20-A Heavy Chain CDR1
GYTFTTYG

SEQ ID NO: 12 - protein, 8 aa, Artificial Sequence
  2-B20-A Heavy Chain CDR2
INTFSGLP

SEQ ID NO: 13 - protein, 17 aa, Artificial Sequence
  2-B20-A Heavy Chain CDR3
ARVKVHDYDGDFYGMDY

SEQ ID NO: 14 - protein, 11 aa, Artificial Sequence
  2-B20-A Light Chain CDR1
KSLLNSDGFTY

SEQ ID NO: 15 - protein, 3 aa, Artificial Sequence
  2-B20-A Light Chain CDR2
LVS

SEQ ID NO: 16 - protein, 9 aa, Artificial Sequence
  2-B20-A Light Chain CDR3
FQSNYLPLT

SEQ ID NO: 17 - protein, 124 aa, Artificial Sequence
  2-B20-A Heavy Chain
QIQLVQSGPELKKPGETVKISCKASGYTFTTYGMSWVKQAPGKGLKWMGWINTFSGLPTYADDFNGRFAFSLETSASTAYLQINNLKDEDTATYFCARVKVHDYDGDFYGMDYWGQGTSVTVSS

SEQ ID NO: 18 - DNA, 372 bp, Artificial Sequence
  2-B20-A Heavy Chain
cagatccagttggtacagtctggacctgagctgaagaagcctggagagacagtcaaaatctcctgcaaggcttctggatataccttcacaacctatggaatgagctgggtgaaacaggcaccaggaaagggtttaaagtggatgggctggataaacaccttctctggactgccaacatatgctgatgacttcaacggacggtttgccttctctttggaaacctctgccagcactgcctatttgcagatcaacaacctcaaagatgaggacacggcgacatatttctgtgcaagagtgaaggttcatgattacgacggggatttctatggtatggactactggggtcaaggaacctcagtcaccgtctcctca

SEQ ID NO: 19 - protein, 112 aa, Artificial Sequence
  2-B20-A Light Chain
DVVLTQTPLSLPVNIGDQASISCKSTKSLLNSDGFTYLDWYLQKPGQSPQLLIYLVSNRFSGVPDRFSGSGSGTDFTLKISRVEAEDLGVYYCFQSNYLPLTFGAGTKLELK

SEQ ID NO: 20 - DNA, 336 bp, Artificial Sequence
  2-B20-A Light Chain
gatgttgttctgacccaaactccactctctctgcctgtcaatattggagatcaagcctctatctcttgcaagtctactaagagtcttctgaatagtgatggattcacttatttggactggtacctgcagaagccaggccagtctccacagctcctaatatatttggtttctaatcgattttctggagttccagacaggttcagtggcagtgggtcaggaacagatttcacactcaagatcagcagagtggaggctgaggatttgggagtttattattgcttccagagtaactatcttcctctcacgttcggtgctgggaccaagctggagctgaaa

SEQ ID NO: 21-30 - skipped (10 entries)

SEQ ID NO: 31 - protein, 8 aa, Artificial Sequence
  2-O15-A Heavy Chain CDR1
GYTFTDCE

SEQ ID NO: 32 - protein, 8 aa, Artificial Sequence
  2-O15-A Heavy Chain CDR2
IDPETGDT

SEQ ID NO: 33 - protein, 15 aa, Artificial Sequence
  2-O15-A Heavy Chain CDR3
TRRRDYGNPYWYFDV

SEQ ID NO: 34 - protein, 7 aa, Artificial Sequence
  2-O15-A Light Chain CDR1
SSISSSN

SEQ ID NO: 35 - protein, 3 aa, Artificial Sequence
  2-O15-A Light Chain CDR2
GTS

SEQ ID NO: 36 - protein, 9 aa, Artificial Sequence
  2-O15-A Light Chain CDR3
QQWTSYPYT

SEQ ID NO: 37 - protein, 122 aa, Artificial Sequence
  2-O15-A Heavy Chain
QVQLQQSGAELVRPGASVKLSCKASGYTFTDCEMHWVKQTPVHGLEWIGVIDPETGDTAYNQKFKGKATLTADKSSSTAYMELRSLTSEDSAVYYCTRRRDYGNPYWYFDVWGAGTTVTVSS

SEQ ID NO: 38 - DNA, 366 bp, Artificial Sequence
  2-O15-A Heavy Chain
caggttcaactgcagcagtctggggctgagctggtgaggcctggggcttcagtgaagctgtcctgcaaggcttcgggctacacatttactgactgtgaaatgcactgggtgaagcagacccctgtgcatggcctggaatggattggagttattgatcctgaaactggtgatactgcctacaatcagaagttcaagggcaaggccacactgactgcagacaaatcctccagcacagcctacatggagctccgcagcctgacatctgaggactctgccgtctattactgtacaagacgcagggactatggtaacccctactggtacttcgatgtctggggcgcagggaccacggtcaccgtctcctca

SEQ ID NO: 39 - protein, 108 aa, Artificial Sequence
  2-O15-A Light Chain
EIVLTQSPALMAASPGEKVTITCSVSSSISSSNLHWYQQKSGTSPKPWIYGTSNLASGVPVRFSGSGSGTSYSLTISSMEAEDAATYYCQQWTSYPYTFGSGTKLEIK

SEQ ID NO: 40 - DNA, 324 bp, Artificial Sequence
  2-O15-A Light Chain
gaaattgtgctcacccagtctccagcactcatggctgcatctccaggggagaaggtcaccatcacctgcagtgtcagctcaagtataagttccagcaacttacactggtaccagcagaagtcaggaacctcccccaaaccctggatttatggcacatccaaccttgcttctggagtccctgttcgcttcagtggcagtggatctgggacctcttattctctcacaatcagcagcatggaggctgaagatgctgccacttattactgtcaacagtggactagttacccatatacgttcggatcggggaccaagctggaaataaaa

SEQ ID NO: 41 - protein, 8 aa, Artificial Sequence
  3-K11-A Heavy Chain CDR1
GYTFTSYV

SEQ ID NO: 42 - protein, 8 aa, Artificial Sequence
  3-K11-A Heavy Chain CDR2
INPYNDGT

SEQ ID NO: 43 - protein, 10 aa, Artificial Sequence
  3-K11-A Heavy Chain CDR3
AREAYRYFDV

SEQ ID NO: 44 - protein, 6 aa, Artificial Sequence
  3-K11-A Light Chain CDR1
ENIYSN

SEQ ID NO: 45 - protein, 3 aa, Artificial Sequence
  3-K11-A Light Chain CDR2
GAT

SEQ ID NO: 46 - protein, 9 aa, Artificial Sequence
  3-K11-A Light Chain CDR3
QHFWGIPWT

SEQ ID NO: 47 - protein, 117 aa, Artificial Sequence
  3-K11-A Heavy Chain
EVQLQQSGPELVKPGASVKMSCKASGYTFTSYVMHWVKQKPGQGLEWIGYINPYNDGTEYNEKFKGKATLTSDKSSSTAYMELSSLTSEDSAVYHCAREAYRYFDVWGTGTTVTVSS

SEQ ID NO: 48 - DNA, 351 bp, Artificial Sequence
  3-K11-A Heavy Chain
gaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactgagtacaatgagaaattcaaaggcaaggccacactgacttcagacaaatcctccagcacagcctacatggagctcagcagcctgacctctgaggactctgcggtctatcactgtgcaagagaggcctaccggtacttcgatgtctggggcacagggaccacggtcaccgtctcctca

SEQ ID NO: 49 - protein, 107 aa, Artificial Sequence
  3-K11-A Light Chain
DIQMTQSPASLSVSVGETVTITCRASENIYSNLAWYQQKQGKYPQLLVYGATNLADGVPSRFSGSGSGTQYFLKINSLQSEDFGSYYCQHFWGIPWTFGGGTKLEIK

SEQ ID NO: 50 - DNA, 321 bp, Artificial Sequence
  3-K11-A Light Chain
gacatccagatgactcagtctccagcctccctatctgtatctgtgggagaaactgtcaccatcacatgtcgagcaagtgagaatatttacagtaatttagcatggtatcagcagaaacagggaaaatatcctcagctcctggtctatggtgcaacaaacttagcagatggtgtgccatcaaggttcagtggcagtggatcaggcacacagtatttcctcaagatcaacagcctgcagtctgaagattttgggagttattactgtcaacatttttggggtattccgtggacgttcggtggaggcaccaagctggaaatcaaa

SEQ ID NO: 51 - protein, 8 aa, Artificial Sequence
  3-P17-A Heavy Chain CDR1
GFSLTSYG

SEQ ID NO: 52 - protein, 7 aa, Artificial Sequence
  3-P17-A Heavy Chain CDR2
IWSGGIT

SEQ ID NO: 53 - protein, 13 aa, Artificial Sequence
  3-P17-A Heavy Chain CDR3
ARIITTVLDQFAD

SEQ ID NO: 54 - protein, 10 aa, Artificial Sequence
  3-P17-A Light Chain CDR1
QSVSTSTYSY

SEQ ID NO: 55 - protein, 3 aa, Artificial Sequence
  3-P17-A Light Chain CDR2
YAS

SEQ ID NO: 56 - protein, 9 aa, Artificial Sequence
  3-P17-A Light Chain CDR3
QHSWEIPPT

SEQ ID NO: 57 - protein, 119 aa, Artificial Sequence
  3-P17-A Heavy Chain
QVQLKQSGPGLVQPSQSLSITCTVSGFSLTSYGVHWVRQSPGKGLEWLGVIWSGGITDYNAGFISRLSISKDNSKSQVFFTMNSLQADDTAIYYCARIITTVLDQFADWGQGTLVTVSA

SEQ ID NO: 58 - DNA, 357 bp, Artificial Sequence
  3-P17-A Heavy Chain
caggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaatcacagactataatgcaggtttcatatccagactgagcatcagcaaggacaattccaagagccaagttttctttacaatgaacagtctgcaagctgatgacacagccatatattactgtgccaggattattactacggttctagatcagtttgctgactggggccaagggactctggtcactgtctctgca

SEQ ID NO: 59 - protein, 111 aa, Artificial Sequence
  3-P17-A Light Chain
DIVLTQSPASLAVSLGQRATISCRASQSVSTSTYSYIHWYQQKPGQPPKLLIKYASNLESGVPARFSGSGSGTDFTLNIHPVEEEDTATYYCQHSWEIPPTFGAGTKLELK

SEQ ID NO: 60 - DNA, 333 bp, Artificial Sequence
  3-P17-A Light Chain
gacattgtgctgacacagtctcctgcttccttagctgtatctctggggcagagggccaccatctcatgcagggccagccaaagtgtcagtacatctacctatagttatattcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattcctcccacgttcggtgctgggaccaagctggagctgaaa

SEQ ID NO: 61 - protein, 8 aa, Artificial Sequence
  4-A22-A Heavy Chain CDR1
GYTFTSYV

SEQ ID NO: 62 - protein, 8 aa, Artificial Sequence
  4-A22-A Heavy Chain CDR2
INPYNDGT

SEQ ID NO: 63 - protein, 10 aa, Artificial Sequence
  4-A22-A Heavy Chain CDR3
AREAYRYFDV

SEQ ID NO: 64 - protein, 6 aa, Artificial Sequence
  4-A22-A Light Chain CDR1
ENIYSN

SEQ ID NO: 65 - protein, 3 aa, Artificial Sequence
  4-A22-A Light Chain CDR2
AAT

SEQ ID NO: 66 - protein, 9 aa, Artificial Sequence
  4-A22-A Light Chain CDR3
QHFWGTPWT

SEQ ID NO: 67 - protein, 117 aa, Artificial Sequence
  4-A22-A Heavy Chain
EVQLQQSGPELVKPGASVKMSCKASGYTFTSYVMHWVKQKPGQGLEWIGYINPYNDGTEYNEKFKGKATLTSDKSSSTAYMELSSLTSEDSAVYYCAREAYRYFDVWGTGTTVTVSS

SEQ ID NO: 68 - DNA, 351 bp, Artificial Sequence
  4-A22-A Heavy Chain
gaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactgagtacaatgagaagttcaaaggcaaggccacactgacttcagacaaatcctccagcacagcctacatggagctcagcagcctgacctctgaggactctgcggtctattactgtgcaagagaggcctaccggtacttcgatgtctggggcacagggaccacggtcaccgtctcctca

SEQ ID NO: 69 - protein, 107 aa, Artificial Sequence
  4-A22-A Light Chain
DIQMTQSPASLSVSVGETVTITCRASENIYSNLAWYQQKQGKSPQLLVYAATNLAEGVPSRFSGSGSGTQYSLKINSLQSEDFGSYYCQHFWGTPWTFGGGTKLEIK

SEQ ID NO: 70 - DNA, 321 bp, Artificial Sequence
  4-A22-A Light Chain
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagaaggtgtgccatcaaggttcagtggcagtggatcaggcacacagtattccctcaagatcaacagcctgcagtctgaagattttgggagttattactgtcaacatttttggggtactccgtggacgttcggtggaggcaccaaactggaaatcaaa

SEQ ID NO: 71 - protein, 8 aa, Artificial Sequence
  4-C22-A Heavy Chain CDR1
GFTFSSFG

SEQ ID NO: 72 - protein, 8 aa, Artificial Sequence
  4-C22-A Heavy Chain CDR2
ISSGGGFT

SEQ ID NO: 73 - protein, 12 aa, Artificial Sequence
  4-C22-A Heavy Chain CDR3
ARRLYDYDWFAY

SEQ ID NO: 74 - protein, 6 aa, Artificial Sequence
  4-C22-A Light Chain CDR1
QDVVTA

SEQ ID NO: 75 - protein, 3 aa, Artificial Sequence
  4-C22-A Light Chain CDR2
WAS

SEQ ID NO: 76 - protein, 9 aa, Artificial Sequence
  4-C22-A Light Chain CDR3
QQYSSYPLT

SEQ ID NO: 77 - protein, 119 aa, Artificial Sequence
  4-C22-A Heavy Chain
EVQLVESGGDLVKPGGSLKLSCAASGFTFSSFGMSWVRQTPDKRLEWVATISSGGGFTYYPDSVEGRFTISRDNAKNTLYLQMSSLKSEDTAMYYCARRLYDYDWFAYWGQGTLVTVSA

SEQ ID NO: 78 - DNA, 357 bp, Artificial Sequence
  4-C22-A Heavy Chain
gaggtgcagctggtggagtctgggggagacttagtgaagcctggagggtccctgaaactctcctgtgcagcctctggattcactttcagtagctttggcatgtcttgggttcgccagactccagacaagaggctggagtgggtcgcaaccattagtagtggtggtggtttcacctactatccagacagtgtggaggggcgattcaccatctccagagacaatgccaagaacaccctgtacctgcaaatgagcagtctgaagtctgaggacacagccatgtattactgtgcaagacggctctatgattacgactggtttgcttactggggccaagggactctggtcactgtctctgca

SEQ ID NO: 79 - protein, 107 aa, Artificial Sequence
  4-C22-A Light Chain
DIVMTQSHKIMSTSVGDRVSITCKASQDVVTAVAWYQKKPGQSPKLLIHWASTRHTGVPDRFTGSGSGTDFTLTISNVQSEDLADYFCQQYSSYPLTFGAGTKLELK

SEQ ID NO: 80 - DNA, 321 bp, Artificial Sequence
  4-C22-A Light Chain
gacattgtgatgacccagtctcacaaaatcatgtccacatcagtaggagacagggtcagcatcacctgcaaggccagtcaggatgtggttactgctgtagcctggtatcaaaagaaaccagggcaatctcctaaactactgattcattgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaa

SEQ ID NO: 81 - protein, 8 aa, Artificial Sequence
  4-O14-B Heavy Chain CDR1
GYAFISHW

SEQ ID NO: 82 - protein, 8 aa, Artificial Sequence
  4-O14-B Heavy Chain CDR2
IDPNSSGS

SEQ ID NO: 83 - protein, 15 aa, Artificial Sequence
  4-O14-B Heavy Chain CDR3
ARSPYYSNPYWYFDV

SEQ ID NO: 84 - protein, 7 aa, Artificial Sequence
  4-O14-B Light Chain CDR1
SSISSSN

SEQ ID NO: 85 - protein, 3 aa, Artificial Sequence
  4-O14-B Light Chain CDR2
GTS

SEQ ID NO: 86 - protein, 9 aa, Artificial Sequence
  4-O14-B Light Chain CDR3
QQWSSYPFT

SEQ ID NO: 87 - protein, 122 aa, Artificial Sequence
  4-O14-B Heavy Chain
QVQLQQPGAELVKPGASVKLSCKASGYAFISHWMHWVRQRPGRGLEWIGRIDPNSSGSKDNEKFRSKATLTVDKPSSTAYMQLSSLTSEDSAVYYCARSPYYSNPYWYFDVWGAGTTVTVSS

SEQ ID NO: 88 - DNA, 366 bp, Artificial Sequence
  4-O14-B Heavy Chain
caggtccaactgcagcagcctggggctgagcttgtgaagcctggggcttcagtgaagctgtcctgcaaggcttctggctacgccttcattagccactggatgcactgggtgaggcagaggcctggacgaggccttgagtggattggaaggattgatcctaatagtagtggttctaaagataatgagaagttcaggagcaaggccacactgactgtagacaaaccctccagcacagcctacatgcagctcagcagcctgacatctgaggactctgcggtctattattgtgcaagatccccctactatagtaacccttactggtacttcgatgtctggggcgcagggaccacggtcaccgtctcctca

SEQ ID NO: 89 - protein, 108 aa, Artificial Sequence
  4-O14-B Light Chain
EIVLTQSPALMAASPGEKVTITCSVSSSISSSNLHWYQQKSETSPKPWIYGTSNLASGVPVRFSGSGSGTSYSLTITNMEAEDAATYYCQQWSSYPFTFGSGTKLEIK

SEQ ID NO: 90 - DNA, 324 bp, Artificial Sequence
  4-O14-B Light Chain
gaaattgtgctcacccagtctccagcactcatggctgcatctccaggggagaaggtcaccatcacctgcagtgtcagttcaagtataagttccagcaacttgcactggtaccagcagaagtcagaaacctcccccaaaccctggatttatggcacatccaacctggcttctggagtcccggttcgcttcagtggcagtggatctgggacctcttattctctcacaatcaccaacatggaggctgaagatgctgccacttattactgtcaacagtggagtagttatccattcacgttcggctcggggacaaagttggaaataaaa

SEQ ID NO: 91 - protein, 8 aa, Artificial Sequence
  6-C19-A Heavy Chain CDR1
GNTFTTAG

SEQ ID NO: 92 - protein, 8 aa, Artificial Sequence
  6-C19-A Heavy Chain CDR2
INTHSGEP

SEQ ID NO: 93 - protein, 11 aa, Artificial Sequence
  6-C19-A Heavy Chain CDR3
ARYGDYYYFDC

SEQ ID NO: 94 - protein, 6 aa, Artificial Sequence
  6-C19-A Light Chain CDR1
GNIHNY

SEQ ID NO: 95 - protein, 3 aa, Artificial Sequence
  6-C19-A Light Chain CDR2
NAK

SEQ ID NO: 96 - protein, 9 aa, Artificial Sequence
  6-C19-A Light Chain CDR3
QHFWTTPWS

SEQ ID NO: 97 - protein, 118 aa, Artificial Sequence
  6-C19-A Heavy Chain
QIQLVQSGPELKKPGETVKISCKASGNTFTTAGMQWVQKMPGKGFKWIGWINTHSGEPKYAEDFKGRFAFSLETSASTAYLQISNLKNEDTATYFCARYGDYYYFDCWGQGTTLTVSS

SEQ ID NO: 98 - DNA, 354 bp, Artificial Sequence
  6-C19-A Heavy Chain
cagatccagttggtgcagtctggacctgagctgaagaagcctggagagacagtcaagatctcctgcaaggcttctgggaataccttcacaactgctggaatgcagtgggtgcaaaagatgccaggaaagggttttaagtggattggctggataaacacccactctggagagccaaaatatgcagaagacttcaagggacggtttgccttctctttggaaacctctgccagcactgcctatttacagataagcaacctcaaaaatgaggacacggctacgtatttctgtgcgagatatggtgattactactactttgactgctggggccaaggcaccactctcacagtctcctca

SEQ ID NO: 99 - protein, 107 aa, Artificial Sequence
  6-C19-A Light Chain
DIQMTQSPASLSASVGETVTITCRASGNIHNYLAWYQQKQGKSPQLLVYNAKTLADGVPSRFSGSGSGTQYSLKINSLQPEDFGSFYCQHFWTTPWSFGGGTKLEIK

SEQ ID NO: 100 - DNA, 321 bp, Artificial Sequence
  6-C19-A Light Chain
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagtttttactgtcaacatttttggacgactccgtggtcgttcggtggaggcaccaagctggaaatcaaa

SEQ ID NO: 101 - protein, 8 aa, Artificial Sequence
  6-N1-A Heavy Chain CDR1
GYTFTDYY

SEQ ID NO: 102 - protein, 8 aa, Artificial Sequence
  6-N1-A Heavy Chain CDR2
INPNNGGI

SEQ ID NO: 103 - protein, 17 aa, Artificial Sequence
  6-N1-A Heavy Chain CDR3
ARRQVFYDGYYDYAMDY

SEQ ID NO: 104 - protein, 10 aa, Artificial Sequence
  6-N1-A Light Chain CDR1
QSVSTSSYSY

SEQ ID NO: 105 - protein, 3 aa, Artificial Sequence
  6-N1-A Light Chain CDR2
YAS

SEQ ID NO: 106 - protein, 9 aa, Artificial Sequence
  6-N1-A Light Chain CDR3
QHNWEIPPT

SEQ ID NO: 107 - protein, 124 aa, Artificial Sequence
  6-N1-A Heavy Chain
EVQLQQSGPELVKPGASVKISCKASGYTFTDYVMNWVQQSHGKSLEWIGDINPNNGGIRYNQNFKGKATLTVEKSSSTAYMELRSLTSEDSAVYYCARRQVFYDGYVDYAMDYWGQGTSVTVSS

SEQ ID NO: 108 - DNA, 372 bp, Artificial Sequence
  6-N1-A Heavy Chain
gaggtccagctgcaacaatctggacctgagctggtgaagcctggggcttcagtgaagatatcctgtaaggcttctggatacacgttcactgactactacatgaactgggtgcagcagagccatggaaagagccttgagtggattggtgatattaatcctaacaatggtggtattcgctacaaccagaacttcaagggcaaggccacattgactgtagagaagtcctccagcacagcctacatggagctccgcagcctgacatctgaggactctgcagtctattactgtgcaagaaggcaggtattctatgatggttactacgactatgctatggactattggggtcaaggaacctcagtcaccgtctcctca

SEQ ID NO: 109 - protein, 111 aa, Artificial Sequence
  6-N1-A Light Chain
DIVLTQSPASLAVSLGQRATISCRASQSVSTSSYSYMHWYQQKPGQPPKLLIKYASNLESGVPARFSGSGSGTDFTLNIHPVEEEDTATYFCQHNWEIPPTFGGGTKLEIK

SEQ ID NO: 110 - DNA, 333 bp, Artificial Sequence
  6-N1-A Light Chain
gacattgtgctgacacagtctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaagatactgcaacatatttctgtcagcacaattgggagattcctcccacgttcggaggggggaccaagctggaaataaaa

SEQ ID NO: 111-130 - skipped (20 entries)

SEQ ID NO: 131 - protein, 8 aa, Artificial Sequence
  9-G24-A Heavy Chain CDR1
GYTFTDYE

SEQ ID NO: 132 - protein, 8 aa, Artificial Sequence
  9-G24-A Heavy Chain CDR2
IDPETGDT

SEQ ID NO: 133 - protein, 15 aa, Artificial Sequence
  9-G24-A Heavy Chain CDR3
ARRRDYGNPYWYFDV

SEQ ID NO: 134 - protein, 7 aa, Artificial Sequence
  9-G24-A Light Chain CDR1
SSISSSN

SEQ ID NO: 135 - protein, 3 aa, Artificial Sequence
  9-G24-A Light Chain CDR2
GTS

SEQ ID NO: 136 - protein, 9 aa, Artificial Sequence
  9-G24-A Light Chain CDR3
QQWSNYPYT

SEQ ID NO: 137 - protein, 122 aa, Artificial Sequence
  9-G24-A Heavy Chain
QVQLQQSGAELVRPGASVKLSCKASGYTFTDYEMHWVKQTPVHGLEWIGVIDPETGDTAYNQKFKDKATLTADKSSSTAYMELRSLTSEDSAVYYCARRRDYGNPYWYFDVWGAGTAVTVSS

SEQ ID NO: 138 - DNA, 366 bp, Artificial Sequence
  9-G24-A Heavy Chain
caggttcaactgcagcagtctggggctgagctggtgaggcctggggcttcagtgaagctgtcctgcaaggcttcgggctacacatttactgactatgaaatgcactgggtgaagcagacacctgtgcatggcctggaatggattggagttattgatcctgaaactggtgatactgcctacaatcagaagttcaaggacaaggccacactgactgcagacaaatcctccagcacagcctacatggagctccgcagcctgacatctgaggactctgccgtctattactgtgcaagacgcagggactatggtaacccctactggtacttcgatgtctggggcgcagggaccgcggtcaccgtctcctca

SEQ ID NO: 139 - protein, 108 aa, Artificial Sequence
  9-G24-A Light Chain
EIVLTQSPALMAASPGEKVTITCSASSSISSSNLHWYQQKSGTSPKPWIYGTSNLASGVPVRFSGSGSGTSYSLTISSMEAEDAATYVCQQWSNYPYTFGSGTKLEIK

SEQ ID NO: 140 - DNA, 324 bp, Artificial Sequence
  9-G24-A Light Chain
gaaattgtgctcacccagtctccagcactcatggctgcatctccaggggagaaggtcaccatcacctgcagtgccagctcaagtataagttccagcaacttacactggtaccagcagaagtcaggaacctcccccaaaccctggatttatggcacatccaaccttgcttctggagtccctgttcgcttcagtggcagtggatctgggacctcttattctctcacaatcagcagcatggaggctgaagatgctgccacttattactgtcaacagtggagtaattacccatatacgttcggatcggggaccaagttggagattaaa

SEQ ID NO: 141 - protein, 8 aa, Artificial Sequence
  9-M12-A Heavy Chain CDR1
GFTFSSYG

SEQ ID NO: 142 - protein, 8 aa, Artificial Sequence
  9-M12-A Heavy Chain CDR2
ISSGGGYT

SEQ ID NO: 143 - protein, 14 aa, Artificial Sequence
  9-M12-A Heavy Chain CDR3
ARPLIPTMVEAFDY

SEQ ID NO: 144 - protein, 6 aa, Artificial Sequence
  9-M12-A Light Chain CDR1
QSVGNN

SEQ ID NO: 145 - protein, 3 aa, Artificial Sequence
  9-M12-A Light Chain CDR2
YAS

SEQ ID NO: 146 - protein, 9 aa, Artificial Sequence
  9-M12-A Light Chain CDR3
QQHYSSPFT

SEQ ID NO: 147 - protein, 121 aa, Artificial Sequence
  9-M12-A Heavy Chain
EVQLVESGGDLVKPGGSLKLSCTASGFTFSSYGMSWVRQTPDKRLEWVTTISSGGGYTYVPDSVKGRFTISRDNAKNTLYLHMSSLKSEDTAMYFCARPLIPTMVEAFDYWGQGTSLTVSS

SEQ ID NO: 148 - DNA, 363 bp, Artificial Sequence
  9-M12-A Heavy Chain
gaggtgcagctggtggagtctgggggagacttagtgaagcctggagggtccctgaaactctcctgtacagcctctggattcactttcagtagctatggcatgtcttgggttcgccagactccagacaagaggctggagtgggtcacaaccattagtagtggtggtggttacacctactatccagacagtgtgaaggggcgattcaccatctccagagacaatgccaagaacaccctgtacctgcacatgagcagtctgaagtctgaggacacagccatgtatttctgtgcaagaccccttattcctacgatggtagaagcatttgactactggggccaaggcacctctctcacagtctcctca

SEQ ID NO: 149 - protein, 107 aa, Artificial Sequence
  9-M12-A Light Chain
SIVMTQTPKFLPVSAGDRVTMTCKASQSVGNNVAWYQQKPGQSPKLLIYYASNRYTGVPDRFTGSGSGADFTFTISSVQVEDLAVYFCQQHYSSPFTFGTGTKLEIK

SEQ ID NO: 150 - DNA, 321 bp, Artificial Sequence
  9-M12-A Light Chain
agtattgtgatgacccagactcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggcagtggatctggggcagatttcactttcaccatcagcagtgtgcaggttgaagacctggcagtttatttctgtcagcagcattatagctctccattcacgttcggcacggggacaaaattggaaataaaa

SEQ ID NO: 151 - protein, 8 aa, Artificial Sequence
  10-J24-A Heavy Chain CDR1
GFTFSSYA

SEQ ID NO: 152 - protein, 8 aa, Artificial Sequence
  10-J24-A Heavy Chain CDR2
ISGGGGFT

SEQ ID NO: 153 - protein, 13 aa, Artificial Sequence
  10-J24-A Heavy Chain CDR3
ARDEDGKHDYFDY

SEQ ID NO: 154 - protein, 6 aa, Artificial Sequence
  10-J24-A Light Chain CDR1
QDVGTA

SEQ ID NO: 155 - protein, 3 aa, Artificial Sequence
  10-J24-A Light Chain CDR2
WAS

SEQ ID NO: 156 - protein, 9 aa, Artificial Sequence
  10-J24-A Light Chain CDR3
QQYASYPLT

SEQ ID NO: 157 - protein, 104 aa, Artificial Sequence
  10-J24-A Heavy Chain
EVQLVESGGGLVKPGGSLKLSCAASGFTFSSYAMSWVRQNPAKRLEWVATISGGGGFTYYPDSVKGRFTISRDNAKNTLYLQMNSLRSEDTAMYYCARDEDGKH

SEQ ID NO: 158 - DNA, 360 bp, Artificial Sequence
  10-J24-A Heavy Chain
gaagtgcagctggtggagtctgggggaggcttagtgaagcctggagggtccctgaaactctcctgtgcagcctctggattcactttcagtagctatgccatgtcttgggttcgccagaatccggcgaagaggctggagtgggtcgcaaccattagcggtggtggtggtttcacctactatccagacagtgtgaagggccgattcaccatctccagagacaatgccaagaacaccctatacctgcaaatgaacagtctgaggtctgaggacacagccatgtattactgtgcaagggatgaagatggtaaacacgactactttgactactggggccaaggcaccactctcacagtctcctca

SEQ ID NO: 159 - protein, 107 aa, Artificial Sequence
  10-J24-A Light Chain
DIVMTQSHKFMSTSVGDRVSITCKASQDVGTAVAWYQQKPGQSPKLLLYWASTRHTGVPDRFTGSGSGTDFTLTISNVQSEDLADYFCQQYASYPLTFGGGTKLVIK

SEQ ID NO: 160 - DNA, 321 bp, Artificial Sequence
  10-J24-A Light Chain
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccathagcaatgtgcagtctgaagacttggcagattatttctgtcagcaatatgctagctatcctctgacgttcggtggaggcaccaagctggtaatcaaa

SEQ ID NO: 161 - protein, 8 aa, Artificial Sequence
  1-B11-A Heavy Chain CDR1
GYTFTDHY

SEQ ID NO: 162 - protein, 8 aa, Artificial Sequence
  1-B11-A Heavy Chain CDR2
IFPGSGST

SEQ ID NO: 163 - protein, 12 aa, Artificial Sequence
  1-B11-A Heavy Chain CDR3
ARSGGLRLGFAY

SEQ ID NO: 164 - protein, 6 aa, Artificial Sequence
  1-B11-A Light Chain CDR1
QDISNY

SEQ ID NO: 165 - protein, 3 aa, Artificial Sequence
  1-B11-A Light Chain CDR2
YTS

SEQ ID NO: 166 - protein, 9 aa, Artificial Sequence
  1-B11-A Light Chain CDR3
QQVNTLPYT

SEQ ID NO: 167 - protein, 119 aa, Artificial Sequence
  1-B11-A Heavy Chain
QVQLQQSGPELVKPGASVKISCKASGYTFTDHYINWVKQRPGQGLEWIGWIFPGSGSTYYNEQFKGKATLTVDKSSNTAYMLLSSLTSEDSAVYFCARSGGLRLGFAYWGQGTLVTVSA

SEQ ID NO: 168 - DNA, 357 bp, Artificial Sequence
  1-B11-A Heavy Chain
caggtccagctacagcagtctggacctgaactggtgaagcctggggcttcagtgaagatatcctgcaaggcttctggctacaccttcactgaccactatataaattgggtgaagcagaggcctggacagggacttgagtggattggatggatttttcctggaagtggtagtacttactacaatgagcagttcaagggcaaggccacgcttactgtagacaaatcctccaacacagcctacatgttgctcagcagcctgacctctgaggactctgcggtctatttctgtgcaagatcagggggattacgactggggtttgcttactggggccaagggactctggtcactgtctctgca

SEQ ID NO: 169 - protein, 107 aa, Artificial Sequence
  1-B11-A Light Chain
DIQMTQTTSSLSASLGDRVTISCRASQDISNYLNWYQQKPDGTVKLLIYYTSRLHSGVPSRFSGSGSGTDYSLTISNLEQEDISTYFCQQVNTLPYTFGGGTKLEIK

SEQ ID NO: 170 - DNA, 321 bp, Artificial Sequence
  1-B11-A Light Chain
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatatttccacttacttttgccaacaggttaatacgcttccgtacacgttcggaggggggaccaagctggaaataaaa

SEQ ID NO: 171 - protein, 8 aa, Artificial Sequence
  1-L10-A Heavy Chain CDR1
GFTFNNYW

SEQ ID NO: 172 - protein, 10 aa, Artificial Sequence
  1-L10-A Heavy Chain CDR2
MRLKSDNYAT

SEQ ID NO: 173 - protein, 7 aa, Artificial Sequence
  1-L10-A Heavy Chain CDR3
IEGYWTY

SEQ ID NO: 174 - protein, 7 aa, Artificial Sequence
  1-L10-A Light Chain CDR1
SSVSSSY

SEQ ID NO: 175 - protein, 3 aa, Artificial Sequence
  1-L10-A Light Chain CDR2
STS

SEQ ID NO: 176 - protein, 10 aa, Artificial Sequence
  1-L10-A Light Chain CDR3
LQYHRSPPYT

SEQ ID NO: 177 - protein, 116 aa, Artificial Sequence
  1-L10-A Heavy Chain
EVKLEESGGGLVQPGGSMKLSCVASGFTFNNYWMNWVRQSPEKGLEWVAQMRLKSDNYATHYAESVKGRFTISRDDSKSSVYLQMNNLRAEDTGIYYCIEGYWTYWGQGTLVTVSA

SEQ ID NO: 178 - DNA, 348 bp, Artificial Sequence
  1-L10-A Heavy Chain
gaagtgaagcttgaggagtctggaggaggcttggtgcaacctggaggatccatgaaactctcctgtgttgcctctggattcactttcaataactactggatgaactgggtccgccagtctccagagaaggggcttgagtgggttgctcaaatgagattgaaatctgataattatgcaacacattatgcggagtctgtgaaagggaggttcaccatctcaagagatgattccaaaagtagtgtctatctgcaaatgaacaacttaagggctgaagacactggaatttattactgtattgaaggttactggacttactggggccaagggactctggtcactgtctctgca

SEQ ID NO: 179 - protein, 109 aa, Artificial Sequence
  1-L10-A Light Chain
QIVLTQSPAIMSASLGERVTMTCTASSSVSSSYLHWYQQKPGSSPKLWIYSTSNLASGVPARFSGNGSGTSYSLTISSMEAEDAATYYCLQYHRSPPYTFGGGTKLEIK

SEQ ID NO: 180 - DNA, 327 bp, Artificial Sequence
  Light Chain Nucleotide Sequence of 1-L10-A
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcaatgggtctgggacctcttactctctcacaatcagcagcatggaggctgaagatgctgccacttattactgcctccagtatcatcgctccccaccgtacacgttcggaggggggaccaagctggaaataaaa

SEQ ID NO: 181 - protein, 8 aa, Artificial Sequence
  2-H7-A Heavy Chain CDR1
GYTFTGYT

SEQ ID NO: 182 - protein, 8 aa, Artificial Sequence
  2-H7-A Heavy Chain CDR2
IYPENGDT

SEQ ID NO: 183 - protein, 13 aa, Artificial Sequence
  2-H7-A Heavy Chain CDR3
ARDGYDVLYGMDY

SEQ ID NO: 184 - protein, 10 aa, Artificial Sequence
  2-H7-A Light Chain CDR1
KSVSTSGYSY

SEQ ID NO: 185 - protein, 3 aa, Artificial Sequence
  2-H7-A Light Chain CDR2
LAS

SEQ ID NO: 186 - protein, 9 aa, Artificial Sequence
  2-H7-A Light Chain CDR3
QHSRELPWT

SEQ ID NO: 187 - protein, 120 aa, Artificial Sequence
  2-H7-A Heavy Chain
QAYLQQSGAELVRPGASVKMSCKASGYTFTGYTMHWVKQTPRQGLEWIGAIYPENGDTSYNQKFKGKATLTVDKSSSTAYMQLSSLTSEDSAVYFCARDGYDVLYGMDYWGQGTSVTVSS

SEQ ID NO: 188 - DNA, 360 bp, Artificial Sequence
  2-H7-A Heavy Chain
caggcttatctacagcagtctggggctgagctggtgaggcctggggcctcagtgaagatgtcctgcaaggcttctggctacacatttaccggttacactatgcactgggtaaagcagacacctagacagggcctggaatggattggagctatttatccagaaaatggtgatacttcctacaatcagaagttcaagggcaaggccacactgactgtcgacaaatcctccagcacagcctacatgcagctcagcagcctgacatctgaagactctgcggtctatttctgtgcaagagatggttacgacgttctctatggtatggactactggggtcaaggaacctcagtcaccgtctcctca

SEQ ID NO: 189 - protein, 111 aa, Artificial Sequence
  2-H7-A Light Chain
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPWTFGGGTKLDIK

SEQ ID NO: 190 - DNA, 333 bp, Artificial Sequence
  2-H7-A Light Chain
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccaagctggatatcaaa

SEQ ID NO: 191 - protein, 8 aa, Artificial Sequence
  2-J9-A Heavy Chain CDR1
GYTFTDYN

SEQ ID NO: 192 - protein, 8 aa, Artificial Sequence
  2-J9-A Heavy Chain CDR2
INPNNGGT

SEQ ID NO: 193 - protein, 17 aa, Artificial Sequence
  2-J9-A Heavy Chain CDR3
ARGGFYGNYGAYYGMDF

SEQ ID NO: 194 - protein, 6 aa, Artificial Sequence
  2-J9-A Light Chain CDR1
ENIYSH

SEQ ID NO: 195 - protein, 3 aa, Artificial Sequence
  2-J9-A Light Chain CDR2
NTK

SEQ ID NO: 196 - protein, 9 aa, Artificial Sequence
  2-J9-A Light Chain CDR3
QHHYGIPPE

SEQ ID NO: 197 - protein, 124 aa, Artificial Sequence
  2-J9-A Heavy Chain
EVQLQQSGPELVKPGASVKMSCKASGYTFTDYNIHWVKQSHGKSLEWIGYINPNNGGTDYNQKFQGKATLTVNKSSSTAYMELRSLTSEDSAVYYCARGGFYGNYGAYYGMDFWGQGTSVTVSS

SEQ ID NO: 198 - DNA, 372 bp, Artificial Sequence
  2-J9-A Heavy Chain
gaggtccagctgcaacagtctggacctgagttggtgaagcctggggcttcagtgaagatgtcctgcaaggcttctggatacacattcactgactacaacatacactgggtgaagcagagccatggaaagagccttgagtggattggatatattaaccctaacaatggtggtactgactacaaccagaagttccagggcaaggccacattgactgtaaacaagtcctccagcacagcctacatggagctccgcagcctgacatcggaagattctgcagtctattactgtgcaagaggggggttttatggcaattacggggcttactatggtatggacttctggggtcaaggaacctcagtcaccgtctcctca

SEQ ID NO: 199 - protein, 107 aa, Artificial Sequence
  2-J9-A Light Chain
DIQMTQSPASLSASVGETVTITCRTSENIYSHLAWYQQTQGKSPQLLVYNTKTLAEGVPSRFTGSGSGTQFSLKINSLQPEDFGSYYCQHHYGIPPEFGGGTKLEIK

SEQ ID NO: 200 - DNA, 321 bp, Artificial Sequence
  2-J9-A Light Chain
gacatccagatgactcagtctccagcctccctatctgcatctgtgggagaaactgtcaccatcacatgtcgaacaagtgagaatatttatagtcatttagcatggtatcagcagacacagggaaaatctcctcagctcctggtctataatacaaaaaccttagcagaaggtgtgccatcaaggttcactggcagtggatcaggcacacagttttctctgaagatcaacagcctgcagcctgaagattttgggagttattactgtcaacatcattatggtattcctccggagttcggtggaggcaccaagctggaaatcaaa

SEQ ID NO: 201 - protein, 8 aa, Artificial Sequence
  2-O12-A Heavy Chain CDR1
GYTFTDYY

SEQ ID NO: 202 - protein, 8 aa, Artificial Sequence
  2-O12-A Heavy Chain CDR2
INSNNGGT

SEQ ID NO: 203 - protein, 13 aa, Artificial Sequence
  2-O12-A Heavy Chain CDR3
ARRGDGHFGGFTY

SEQ ID NO: 204 - protein, 10 aa, Artificial Sequence
  2-O12-A Light Chain CDR1
ESVDNSGISF

SEQ ID NO: 205 - protein, 3 aa, Artificial Sequence
  2-O12-A Light Chain CDR2
AAS

SEQ ID NO: 206 - protein, 9 aa, Artificial Sequence
  2-O12-A Light Chain CDR3
QQSKEVPWT

SEQ ID NO: 207 - protein, 120 aa, Artificial Sequence
  2-O12-A Heavy Chain
EVQLQQSGPELVKPGASVKISCKASGYTFTDYYMNWVKQSHGKSLEWIGDINSNNGGTTYDQKFKGKATLTVDRSSSTAYMELRSLTSEDSAVYYCARRGDGHFGGFTYWGQGTLVTVSA

SEQ ID NO: 208 - DNA, 360 bp, Artificial Sequence
  2-O12-A Heavy Chain
gaggtccagctgcaacaatctggacctgaactggtgaagcctggggcctcagtgaagatatcctgtaaggcttctggatacacgttcactgactactacatgaactgggtgaagcagagccatggaaagagccttgagtggattggagatattaattctaacaatggtggtactacctacgaccagaagttcaagggcaaggccactttgactgtagacaggtcctccagcacagcctacatggagctccgcagcctgacatctgaggactctgcagtctattactgtgcaaggagaggggatggtcactttggggggtttacttattggggccaagggactctggtcactgtctctgca

SEQ ID NO: 209 - protein, 111 aa, Artificial Sequence
  2-O12-A Light Chain
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 210 - DNA, 333 bp, Artificial Sequence
  2-O12-A Light Chain
gacattgtgctgacccaatctccagcttctttggctgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 211 - protein, 8 aa, Artificial Sequence
  2-P2-A Heavy Chain CDR1
GYTFTSYG

SEQ ID NO: 212 - protein, 8 aa, Artificial Sequence
  2-P2-A Heavy Chain CDR2
IYIGNGYT

SEQ ID NO: 213 - protein, 7 aa, Artificial Sequence
  2-P2-A Heavy Chain CDR3
ARGGVDY

SEQ ID NO: 214 - protein, 11 aa, Artificial Sequence
  2-P2-A Light Chain CDR1
QSIVHSNGNTY

SEQ ID NO: 215 - protein, 3 aa, Artificial Sequence
  2-P2-A Light Chain CDR2
KVS

SEQ ID NO: 216 - protein, 9 aa, Artificial Sequence
  2-P2-A Light Chain CDR3
FQGSHVPYT

SEQ ID NO: 217 - protein, 114 aa, Artificial Sequence
  2-P2-A Heavy Chain
EVQLQQSGAELVRPGSSVKMSCKTSGYTFTSYGINWVKQRPGQGLEWIGYIYIGNGYTEYNEKFKGKATLTSDTSSSTAYMQLSSLTSEDSAIYFCARGGVDYWGQGTTLTVSS

SEQ ID NO: 218 - DNA, 342 bp, Artificial Sequence
  2-P2-A Heavy Chain
gaggtccagcttcagcagtctggagctgagctggtgaggcctgggtcctcagtgaagatgtcctgcaagacttctggatatacattcacaagctacggtataaactgggtgaagcagaggcctggacagggcctggaatggattggatatatttatattggaaatggttatactgaatacaatgagaagttcaagggtaaggccacactgacttcagacacatcctccagcacagcctacatgcagctcagcagcctgacatctgaggactctgcaatctatttctgtgcaagaggaggggttgactactggggccaaggcaccactctcacagtctcctca

SEQ ID NO: 219 - protein, 112 aa, Artificial Sequence
  2-P2-A Light Chain
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 220 - DNA, 336 bp, Artificial Sequence
  2-P2-A Light Chain
gatgttttgatgacccaaactccactctccctgcctgtcagtcttggagatcaagcctccatctcttgcagatctagtcagagtattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 221 - protein, 8 aa, Artificial Sequence
  3-E13-A Heavy Chain CDR1
GYTFTDYN

SEQ ID NO: 222 - protein, 8 aa, Artificial Sequence
  3-E13-A Heavy Chain CDR2
INPNNGGT

SEQ ID NO: 223 - protein, 17 aa, Artificial Sequence
  3-E13-A Heavy Chain CDR3
ARGGFYGNYGAYYGMDF

SEQ ID NO: 224 - protein, 6 aa, Artificial Sequence
  3-E13-A Light Chain CDR1
ENIYSH

SEQ ID NO: 225 - protein, 3 aa, Artificial Sequence
  3-E13-A Light Chain CDR2
NAK

SEQ ID NO: 226 - protein, 9 aa, Artificial Sequence
  3-E13-A Light Chain CDR3
QHHYGIPPE

SEQ ID NO: 227 - protein, 124 aa, Artificial Sequence
  3-E13-A Heavy Chain
EVQLQQSGPELVKPGASVKMSCKASGYTFTDYNIHWVKQSHGKSLEWIGYINPNNGGTDYNQKFQGKATLTVNKSSSTAYMELRSLTSEDFAVYYCARGGFYGNYGAYYGMDFWGQGTSVTVSS

SEQ ID NO: 228 - DNA, 372 bp, Artificial Sequence
  3-E13-A Heavy Chain
gaggtccagctgcaacagtctggacctgagctggtgaagcctggggcttcagtgaagatgtcctgcaaggcttctggatacacattcactgactacaacatacactgggtgaaacagagccatggaaagagccttgagtggattggatatattaaccctaacaatggtggtactgactacaaccagaagttccagggcaaggccacattgactgtaaacaagtcctccagcacagcctacatggagctccgcagcctgacatcggaagattttgcagtctattactgtgcaagaggggggttttatggcaattacggggcttactatggtatggacttctggggtcaaggaacctcagtcaccgtctcctca

SEQ ID NO: 229 - protein, 107 aa, Artificial Sequence
  3-E13-A Light Chain
DIQMTQSPASLSASVGETVTITCRTSENIYSHLAWYQQTQGKSPQLLVYNAKTLAEGVPSRFSGSGSGTHFSLKINSLQPEDFGSYYCQHHYGIPPEFGGGTKLEIK

SEQ ID NO: 230 - DNA, 321 bp, Artificial Sequence
  3-E13-A Light Chain
gacatccagatgactcagtctccagcctccctatctgcatctgtgggagaaactgtcaccatcacatgtcgaacaagtgagaatatttatagtcatttagcatggtatcagcagacacagggaaaatctcctcagctcctggtctataatgcaaaaaccttagcagaaggtgtgccatcaaggttcagtggcagtggatcaggcacacatttttctctgaagatcaacagcctgcagcctgaagattttgggagttattactgtcaacatcattatggtattcctccggagttcggtggaggcaccaagctggaaatcaaa

SEQ ID NO: 231 - protein, 8 aa, Artificial Sequence
  4-A15-A Heavy Chain CDR1
GNAFSSYW

SEQ ID NO: 232 - protein, 8 aa, Artificial Sequence
  4-A15-A Heavy Chain CDR2
IYPGDGDT

SEQ ID NO: 233 - protein, 15 aa, Artificial Sequence
  4-A15-A Heavy Chain CDR3
ASGGYGSSYERDFDY

SEQ ID NO: 234 - protein, 6 aa, Artificial Sequence
  4-A15-A Light Chain CDR1
QNINVW

SEQ ID NO: 235 - protein, 3 aa, Artificial Sequence
  4-A15-A Light Chain CDR2
KAS

SEQ ID NO: 236 - protein, 9 aa, Artificial Sequence
  4-A15-A Light Chain CDR3
QQGHSYPLT

SEQ ID NO: 237 - protein, 122 aa, Artificial Sequence
  4-A15-A Heavy Chain
QVQLQQSGAELVKPGASVKISCKASGNAFSSYWMNWVKQRPGKGLEWIGQIYPGDGDTNFNGRFKGKATLTADISSTTAYMLLSSLTSEDSAVYFCASGGYGSSYERDFDYWGQGTTLTVSS

SEQ ID NO: 238 - DNA, 366 bp, Artificial Sequence
  4-A15-A Heavy Chain
caggttcagctgcagcagtctggggctgagctggtgaagcctggggcctcagtgaagatttcctgcaaagcttcaggcaacgcattcagtagttactggatgaactgggtgaagcagaggcctggaaagggtcttgagtggattggacagatttatcctggagatggtgatactaacttcaacggaaggttcaagggcaaggccacactgactgcagacatatcctccaccacagcctacatgctgctcagcagcctgacctctgaggactctgcggtctatttctgtgcaagcgggggctacggtagtagctacgagagggactttgactactggggccaaggcaccactctcacagtctcctca

SEQ ID NO: 239 - protein, 107 aa, Artificial Sequence
  4-A15-A Light Chain
DIQMNQSPSSLSASLGDTITITCHASQNINVWLSWYQQKPGNIPKLLIYKASNLHTGVPSRFSGSGSGTGFTLTISSLQPEDIATYYCQQGHSYPLTFGGGTKLEIK

SEQ ID NO: 240 - DNA, 321 bp, Artificial Sequence
  4-A15-A Light Chain
gacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcacagttatcctctcacgttcggaggggggaccaagctggaaataaaa

SEQ ID NO: 241 - protein, 8 aa, Artificial Sequence
  4-C3-A Heavy Chain CDR1
GYSFTGYY

SEQ ID NO: 242 - protein, 8 aa, Artificial Sequence
  4-C3-A Heavy Chain CDR2
IYPYNGVS

SEQ ID NO: 243 - protein, 16 aa, Artificial Sequence
  4-C3-A Heavy Chain CDR3
ARAYGSSYDYYYGLDY

SEQ ID NO: 244 - protein, 10 aa, Artificial Sequence
  4-C3-A Light Chain CDR1
QSVDYDGAGY

SEQ ID NO: 245 - protein, 3 aa, Artificial Sequence
  4-C3-A Light Chain CDR2
SAS

SEQ ID NO: 246 - protein, 9 aa, Artificial Sequence
  4-C3-A Light Chain CDR3
QQSNDDPYT

SEQ ID NO: 247 - protein, 123 aa, Artificial Sequence
  4-C3-A Heavy Chain
EVQLQQSGPELVKPGASVKISCKASGYSFTGYYMHWVKQSHGNILDWIGYIYPYNGVSSYNQKFKGKATLTVDKSSSTAYMELRSLTSEASAVYYCARAYGSSYDYYYGLDYWGQGTSVTVSS

SEQ ID NO: 248 - DNA, 369 bp, Artificial Sequence
  4-C3-A Heavy Chain
gaggttcagctgcagcagtctggacctgagctggtgaagcctggggcttcagtgaagatatcctgcaaggcctctggttactcattcactggctactacatgcactgggtgaagcagagccatggaaatatcctcgattggattggatatatttatccttacaatggtgtttctagctacaaccagaaattcaagggcaaggccacattgactgtagacaagtcctctagcacagcctacatggagctccgcagcctgacatctgaggcctctgcagtctattactgtgcaagagcctacggcagtagctacgattattactatggtttggactactggggtcaaggaacctcagtcaccgtctcctca

SEQ ID NO: 249 - protein, 111 aa, Artificial Sequence
  4-C3-A Light Chain
DIVLTQSPASLAVSLGQRATISCKASQSVDYDGAGYMNWYQQKPGQPPKLLISSASNLQSGIPARFSGSGSGTDFTLNIHPVEEEDAATYYCQQSNDDPYTFGGGTKLEIK

SEQ ID NO: 250 - DNA, 333 bp, Artificial Sequence
  4-C3-A Light Chain
gacattgtactgacccaatctccagcttctttggctgtgtctctggggcagagggccaccatctcctgcaaggccagccaaagtgttgattatgatggtgctggttatatgaactggtaccaacagaaaccaggacagccacccaaactcctcatctcttctgcatccaatctacaatctgggatcccagccaggtttagtggcagtgggtctgggacagacttcaccctcaacatccatcctgtggaggaggaggatgctgcaacctattactgtcagcaaagtaatgacgatccgtacacgttcggcggggggaccaagctggaaataaaa

SEQ ID NO: 251 - protein, 8 aa, Artificial Sequence
  4-K13-A Heavy Chain CDR1
GYTFTSYT

SEQ ID NO: 252 - protein, 8 aa, Artificial Sequence
  4-K13-A Heavy Chain CDR2
IYPGNVDT

SEQ ID NO: 253 - protein, 10 aa, Artificial Sequence
  4-K13-A Heavy Chain CDR3
ARDGYYAMDY

SEQ ID NO: 254 - protein, 12 aa, Artificial Sequence
  4-K13-A Light Chain CDR1
QSLLYSSNQKNY

SEQ ID NO: 255 - protein, 3 aa, Artificial Sequence
  4-K13-A Light Chain CDR2
WAS

SEQ ID NO: 256 - protein, 9 aa, Artificial Sequence
  4-K13-A Light Chain CDR3
QQYYSYPWT

SEQ ID NO: 257 - protein, 117 aa, Artificial Sequence
  4-K13-A Heavy Chain
QAYLQQSGAELVRPGASVKMSCKASGYTFTSYTVHWVKQTPRQGLEWIGAIYPGNVDTSYNQNFKGKATLTVDKSSSTAYMQLSSLTSEDSAVYFCARDGYYAMDYWGQGTSVTVSS

SEQ ID NO: 258 - DNA, 351 bp, Artificial Sequence
  4-K13-A Heavy Chain
caggcttatctacagcagtctggggctgagctggtgaggcctggggcctcagtgaagatgtcctgcaaggcttctggctacacatttaccagttacactgtgcactgggtaaagcagacacctagacagggcctggaatggattggagctatttatccaggaaatgttgatacttcctacaatcagaatttcaagggcaaggccacactgactgtagacaaatcctccagcacagcctacatgcagctcagcagcctgacatctgaagactctgcggtctatttctgtgcaagagatggttactatgctatggactactggggtcaaggaacctcagtcaccgtctcctca

SEQ ID NO: 259 - protein, 113 aa, Artificial Sequence
  4-K13-A Light Chain
DIVLSQSPSSLPVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEIK

SEQ ID NO: 260 - DNA, 339 bp, Artificial Sequence
  4-K13-A Light Chain
gacattgtgttgtcacagtctccatcctccctacctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgtggacgttcggtggaggcaccaagctggaaatcaaa

SEQ ID NO: 261 - protein, 8 aa, Artificial Sequence
  4-L4-A Heavy Chain CDR1
GYTFTSYT

SEQ ID NO: 262 - protein, 8 aa, Artificial Sequence
  4-L4-A Heavy Chain CDR2
IYPGNVDT

SEQ ID NO: 263 - protein, 10 aa, Artificial Sequence
  4-L4-A Heavy Chain CDR3
ARDGYYAMDY

SEQ ID NO: 264 - protein, 12 aa, Artificial Sequence
  4-L4-A Light Chain CDR1
QSLLYSSNQKNY

SEQ ID NO: 265 - protein, 3 aa, Artificial Sequence
  4-L4-A Light Chain CDR2
WAS

SEQ ID NO: 266 - protein, 9 aa, Artificial Sequence
  4-L4-A Light Chain CDR3
QQYYSYPWT

SEQ ID NO: 267 - protein, 117 aa, Artificial Sequence
  4-L4-A Heavy Chain
QAYLQQSGAELVRPGASVKMSCKASGYTFTSYTVHWVKQTPRQGLEWIGGIYPGNVDTSYNQNFKGKATLTVDKSSSTAYMQLSSLTSEDSAVYFCARDGYYAMDYWGQGTSVTVSS

SEQ ID NO: 268 - DNA, 351 bp, Artificial Sequence
  4-L4-A Heavy Chain
caggcttatctacagcagtctggggctgagctggtgaggcctggggcctcagtgaagatgtcctgcaaggcttctggctacacatttaccagttacactgtgcactgggtaaagcagacacctagacagggcctggaatggattggaggtatttatccaggaaatgttgatacttcctacaatcagaatttcaagggcaaggccacactgactgtagacaaatcctccagcacagcctacatgcagctcagcagcctgacatctgaagactctgcggtctatttctgtgcaagagatggttactatgctatggactactggggtcaaggaacctcagtcaccgtctcctca

SEQ ID NO: 269 - protein, 113 aa, Artificial Sequence
  4-L4-A Light Chain
DIVLSQSPSSLPVSVGEKVTMSCKSSQSLLYSSNQKNYLGWYQQKPGQSPKLLIYWASTRESGVPDRFTGSGSGTDFTLTISSVKAEDLAVYYCQQYYSYPWTFGGGTKLEIK

SEQ ID NO: 270 - DNA, 339 bp, Artificial Sequence
  4-L4-A Light Chain
gacattgtgttgtcacagtctccatcctccctacctgtgtcagttggagagaaggttactatgagctgcaagtccagtcagagccttttatatagtagcaatcaaaagaactacttgggctggtaccagcagaaaccagggcagtctcctaaactgctgatttactgggcatccactagggaatctggggtccctgatcgcttcacaggcagtggatctgggacagattttactctcaccatcagtagtgtgaaggctgaagacctggcagtttattactgtcagcaatattatagctatccgtggacgttcggtggaggcaccaagctggaaatcaaa

SEQ ID NO: 271 - protein, 8 aa, Artificial Sequence
  5-H22-A Heavy Chain CDR1
GYTFTDYE

SEQ ID NO: 272 - protein, 8 aa, Artificial Sequence
  5-H22-A Heavy Chain CDR2
IDLETGGT

SEQ ID NO: 273 - protein, 16 aa, Artificial Sequence
  5-H22-A Heavy Chain CDR3
TREFPHYYGSRYGFPY

SEQ ID NO: 274 - protein, 5 aa, Artificial Sequence
  5-H22-A Light Chain CDR1
SSVSY

SEQ ID NO: 275 - protein, 3 aa, Artificial Sequence
  5-H22-A Light Chain CDR2
STS

SEQ ID NO: 276 - protein, 9 aa, Artificial Sequence
  5-H22-A Light Chain CDR3
QQRSSYPYT

SEQ ID NO: 277 - protein, 123 aa, Artificial Sequence
  5-H22-A Heavy Chain
QVQLQQSGAELVRPGASVTLSCKASGYTFTDYEMHWMKQTPVHGLEWIGAIDLETGGTVYNQKFKGKAILTADKSSNTAYMALRSLTSEDSAVYYCTREFPHYYGSRYGFPYWGQGTLVTVSA

SEQ ID NO: 278 - DNA, 369 bp, Artificial Sequence
  5-H22-A Heavy Chain
caggttcaactgcagcagtctggggctgagctggtgaggcctggggcttcagtgacgctgtcctgcaaggcttcgggctacacatttactgactatgaaatgcactggatgaaacagacacctgtgcatggcctggaatggattggagctattgatcttgaaactggtggtactgtctacaatcagaagttcaagggcaaggccatactgactgcagacaaatcctccaacacagcctacatggcgctccgcagtctgacatctgaggactctgccgtctattactgtacaagagagttcccccattactacggtagtaggtacggttttccttactggggccaggggactctggtcactgtctctgca

SEQ ID NO: 279 - protein, 106 aa, Artificial Sequence
  5-H22-A Light Chain
QIVLTQSPAIMSASPGEKVTITCSASSSVSYMHWLQQKPGTSPKLWIYSTSNLASGVPARFSGSGSGTSYSLTISRMEAEDAATYYCQQRSSYPYTFGGGTKLEIK

SEQ ID NO: 280 - DNA, 318 bp, Artificial Sequence
  5-H22-A Light Chain
caaattgttctcacccagtctccagcaatcatgtctgcatctccaggggagaaggtcaccataacctgtagtgccagctcaagtgtaagttacatgcactggttacagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccgtacacgttcggaggggggaccaagctggaaataaaa

SEQ ID NO: 281 - protein, 8 aa, Artificial Sequence
  6-O12-A Heavy Chain CDR1
GYTFTSYW

SEQ ID NO: 282 - protein, 8 aa, Artificial Sequence
  6-O12-A Heavy Chain CDR2
INPSNGGT

SEQ ID NO: 283 - protein, 11 aa, Artificial Sequence
  6-O12-A Heavy Chain CDR3
ARLGWFLPLGS

SEQ ID NO: 284 - protein, 5 aa, Artificial Sequence
  6-O12-A Light Chain CDR1
SSVSY

SEQ ID NO: 285 - protein, 3 aa, Artificial Sequence
  6-O12-A Light Chain CDR2
STS

SEQ ID NO: 286 - protein, 9 aa, Artificial Sequence
  6-O12-A Light Chain CDR3
HQWSGYMYT

SEQ ID NO: 287 - protein, 118 aa, Artificial Sequence
  6-O12-A Heavy Chain
QVQLQQPGTELVKPGASVKLSCKASGYTFTSYWMHWVKQRPGQGLEWIGNINPSNGGTNYNEKFRSKATLTVDKSSSTAYMQLSSLTSEDSAVYYCARLGWFLPLGSWGQGTTLTVSS

SEQ ID NO: 288 - DNA, 354 bp, Artificial Sequence
  6-O12-A Heavy Chain
caggtccaactgcagcagcctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaggagcaaggccacactgactgtagacaaatcctccagcacagcctacatgcagctcagcagcctgacatctgaggactctgcggtctattattgtgcaagactgggatggtttctaccccttgggtcctggggccaaggcaccactctcacagtctcctca

SEQ ID NO: 289 - protein, 106 aa, Artificial Sequence
  6-O12-A Light Chain
QIVLTQSPAIMSASLGEVITLTCSASSSVSYMHWYQQKSGTSPKLLIYSTSNLASGVPSRFSGSGSGTFYSLTISSVEAEDAADYYCHQWSGYMYTFGGGTKLEIK

SEQ ID NO: 290 - DNA, 318 bp, Artificial Sequence
  6-O12-A Light Chain
caaattgttctcacccagtctccagcaatcatgtctgcatctctaggggaggtgatcaccctaacctgcagtgccagctcgagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtggttatatgtacacattcggaggggggaccaaactggaaataaaa

SEQ ID NO: 291 - protein, 8 aa, Artificial Sequence
  8-N24-A Heavy Chain CDR1
GYTFTSYW

SEQ ID NO: 292 - protein, 8 aa, Artificial Sequence
  8-N24-A Heavy Chain CDR2
IHPSDSDT

SEQ ID NO: 293 - protein, 18 aa, Artificial Sequence
  8-N24-A Heavy Chain CDR3
AIPLYSYDNSGGYYPMDY

SEQ ID NO: 294 - protein, 6 aa, Artificial Sequence
  8-N24-A Light Chain CDR1
QNVRTA

SEQ ID NO: 295 - protein, 3 aa, Artificial Sequence
  8-N24-A Light Chain CDR2
LAS

SEQ ID NO: 296 - protein, 9 aa, Artificial Sequence
  8-N24-A Light Chain CDR3
LQHWNYPFT

SEQ ID NO: 297 - protein, 125 aa, Artificial Sequence
  8-N24-A Heavy Chain
QVQLQQPGAELVKPGASVKVSCKASGYTFTSYWMHWVKQRPGQGLEWIGRIHPSDSDTNYYQKFKGKATLSVDKSSSTAYMQLSSLTSEDSAVYYCAIPLYSYDNSGGYYPMDYWGQGTSVTVSS

SEQ ID NO: 298 - DNA, 375 bp, Artificial Sequence
  8-N24-A Heavy Chain
caggtccaactgcagcagcctggggctgaactggtgaagcctggggcttcagtgaaggtgtcctgcaaggcttctggctacaccttcaccagctactggatgcactgggtgaagcagaggcctggccaaggccttgagtggattggaaggattcatccttctgatagtgatactaactactatcaaaagttcaagggcaaggccacattgtctgtagacaaatcctccagcacagcctacatgcagctcagcagcctgacatctgaggactctgcggtctattactgtgcaattcccctttattcctacgataatagcggaggttactatcctatggactactggggtcaaggaacctcagtcaccgtctcctca

SEQ ID NO: 299 - protein, 107 aa, Artificial Sequence
  8-N24-A Light Chain
DIVMTQSQKFMSTSVGDRVTITCKASQNVRTAVAWYQQKPGQSPKKLIYLASNRHTGVPDRFTGSGSGTDFTLAISNVQSEDLADYFCLQHWNYPFTFGSGTRLEIK

SEQ ID NO: 300 - DNA, 321 bp, Artificial Sequence
  8-N24-A Light Chain
gacattgtgatgacccagtctcaaaaattcatgtccacatcagtaggagacagggtcaccatcacctgcaaggccagtcagaatgtccgtactgctgtagcctggtatcaacagaaaccagggcagtctcctaaaaaactgatttacttggcatccaaccggcacactggagtccctgatcgcttcacaggcagtggatctgggacagatttcactctcgccattagcaatgtgcaatctgaagacctggcagattatttctgtctgcaacattggaattatccattcacgttcggctcggggacaaggttggaaataaaa

SEQ ID NO: 301 - protein, 8 aa, Artificial Sequence
  9-J11-A Heavy Chain CDR1
GYTFTTYP

SEQ ID NO: 302 - protein, 8 aa, Artificial Sequence
  9-J11-A Heavy Chain CDR2
FHPYNDDS

SEQ ID NO: 303 - protein, 13 aa, Artificial Sequence
  9-J11-A Heavy Chain CDR3
ARGSYGPYWYFDV

SEQ ID NO: 304 - protein, 6 aa, Artificial Sequence
  9-J11-A Light Chain CDR1
GDIHNY

SEQ ID NO: 305 - protein, 3 aa, Artificial Sequence
  9-J11-A Light Chain CDR2
NAK

SEQ ID NO: 306 - protein, 9 aa, Artificial Sequence
  9-J11-A Light Chain CDR3
QHFWSIPYT

SEQ ID NO: 307 - protein, 120 aa, Artificial Sequence
  9-J11-A Heavy Chain
QVQLQQSGAELVKPGASVKMSCMASGYTFTTYPIEWMKQIHGKSLEWIGNFHPYNDDSKYNEKFKGKATLSVEKSSSTVYLELSRSTSDDSAVYYCARGSYGPYWYFDVWGTGTTVTVSS

SEQ ID NO: 308 - DNA, 360 bp, Artificial Sequence
  9-J11-A Heavy Chain
caggttcagctgcagcagtctggggctgaactagtgaagcctggagcctcagtgaagatgtcctgcatggcttctggctacaccttcactacctatcctatagagtggatgaagcagattcatggaaagagcctagagtggattggaaattttcatccttacaatgatgattctaagtacaatgaaaagttcaagggcaaggccacattgagtgtagaaaaatcctctagcacagtctacttggagctcagccgatcaacatctgatgactctgctgtttattactgtgcaagggggtcttatggcccctactggtacttcgatgtctggggcacagggaccacggtcaccgtctcctca

SEQ ID NO: 309 - protein, 107 aa, Artificial Sequence
  9-J11-A Light Chain
DIQMTQSPASLSASVGETVTITCRASGDIHNYLVWYQQKQGKSPQLLVYNAKALADGVPSRFSGSGSGTQYSLKINSLQPDDFGSYYCQHFWSIPYTFGGGTKLEIK

SEQ ID NO: 310 - DNA, 321 bp, Artificial Sequence
  9-J11-A Light Chain
gacatccagatgactcagtctccagcctccctatctgcatctgtgggagaaactgtcaccatcacatgtcgagcaagtggggatattcacaattatttagtatggtatcagcagaaacagggaaaatctcctcagctcctggtctataatgcaaaagccttagcagatggtgtgccatcaaggttcagtggcagtggatcaggaacacaatattctctcaagatcaacagcctgcagcctgatgattttgggagttattactgtcaacatttttggagtattccgtacacgttcggaggggggaccaagctggaaataaaa

SEQ ID NO: 311 - protein, 8 aa, Artificial Sequence
  9-L13-A Heavy Chain CDR1
GFSLTSYA

SEQ ID NO: 312 - protein, 7 aa, Artificial Sequence
  9-L13-A Heavy Chain CDR2
IWTGGGT

SEQ ID NO: 313 - protein, 17 aa, Artificial Sequence
  9-L13-A Heavy Chain CDR3
ARGPPLIYYGDLYYFDY

SEQ ID NO: 314 - protein, 6 aa, Artificial Sequence
  9-L13-A Light Chain CDR1
QDINKY

SEQ ID NO: 315 - protein, 3 aa, Artificial Sequence
  9-L13-A Light Chain CDR2
YTS

SEQ ID NO: 316 - protein, 8 aa, Artificial Sequence
  9-L13-A Light Chain CDR3
LQYDNLWT

SEQ ID NO: 317 - protein, 123 aa, Artificial Sequence
  9-L13-A Heavy Chain
QVQLKESGPGLVAPSQSLSITCTVSGFSLTSYAISWVRQPPGKGLEWLGLIWTGGGTNYNSALKSRLSISKDNSKSQIFLKMNSLQTDDTARYYCARGPPLIYYGDLYYFDYWGQGTTLTVSS

SEQ ID NO: 318 - DNA, 369 bp, Artificial Sequence
  9-L13-A Heavy Chain
caggtgcagctgaaggagtcaggacctggcctggtggcgccctcacagagcctgtccatcacatgcactgtctctgggttctcattaaccagctatgctataagctgggttcgccagccaccagggaagggtctggagtggcttggactaatatggactggtggaggcacaaattataattcagctctcaaatccagactgagcatcagcaaagacaactccaagagtcaaattttcttaaaaatgaacagtctgcaaactgatgacacagccaggtactactgtgccagagggcctcctctcatctattatggtgacttgtactactttgactactggggccaaggcaccactctcacagtctcctca

SEQ ID NO: 319 - protein, 106 aa, Artificial Sequence
  9-L13-A Light Chain
DIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKLEIK

SEQ ID NO: 320 - DNA, 318 bp, Artificial Sequence
  9-L13-A Light Chain
gacatccagatgacacagtctccatcctcactgtctgcatctctgggaggcaaagtcaccatcacttgcaaggcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatctgtggacgttcggtggaggcaccaagctggaaatcaaa

SEQ ID NO: 321 - protein, 8 aa, Artificial Sequence
  9-P9-A Heavy Chain CDR1
GFSFSNFG

SEQ ID NO: 322 - protein, 8 aa, Artificial Sequence
  9-P9-A Heavy Chain CDR2
ISSGGDYI

SEQ ID NO: 323 - protein, 7 aa, Artificial Sequence
  9-P9-A Heavy Chain CDR3
TRGYFDY

SEQ ID NO: 324 - protein, 11 aa, Artificial Sequence
  9-P9-A Light Chain CDR1
QSIVHSNGNTY

SEQ ID NO: 325 - protein, 3 aa, Artificial Sequence
  9-P9-A Light Chain CDR2
TVS

SEQ ID NO: 326 - protein, 9 aa, Artificial Sequence
  9-P9-A Light Chain CDR3
FQGSHVPYT

SEQ ID NO: 327 - protein, 114 aa, Artificial Sequence
  9-P9-A Heavy Chain
DVKLVESGEGLVKPGGSLKLSCAASGFSFSNFGMSWVRQTPEKRLEWVAYISSGGDYIYYADTVKGRFTISRDNARNTLYLQMSSLKSEDTAIYYCTRGYFDYWGQGTTLTVSS

SEQ ID NO: 328 - DNA, 342 bp, Artificial Sequence
  9-P9-A Heavy Chain
gacgtgaagctggtggagtctggggaaggcttagtgaagcctggagggtccctgaaactctcctgtgcagcctccggattcagtttcagtaactttggcatgtcttgggttcgccagactccagagaagaggctggagtgggtcgcatacattagtagtggtggtgattacatctactatgcagacactgtgaagggccgattcaccatctccagagacaatgccaggaacaccctgtacctgcaaatgagcagtctgaagtctgaggacacagccatatattactgtacaaggggatactttgactactggggccaaggcaccactctcacagtctcctca

SEQ ID NO: 329 - protein, 112 aa, Artificial Sequence
  9-P9-A Light Chain
DVLMTQTPLSLPVSLGDQASISCRSSQSIVHSNGNTYLEWYLQKPGQSPKLLIYTVSNRFSGVPDRFSGSGSGTDFTLKISRVEAEDLGVYYCFQGSHVPYTFGGGTKLEMR

SEQ ID NO: 330 - DNA, 336 bp, Artificial Sequence
  9-P9-A Light Chain
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacacagtttccaaccgattttctggggtcccagacaggttcagtggcagtggatcggggacagatttcacactcaagatcagcagagtggaggctgaggatctgggagtttattactgctttcaaggttcacatgttccgtacacgttcggaggggggaccaagctggaaatgaga

SEQ ID NO: 331 - protein, 8 aa, Artificial Sequence
  10-B11-A Heavy Chain CDR1
GYTFTSYW

SEQ ID NO: 332 - protein, 8 aa, Artificial Sequence
  10-B11-A Heavy Chain CDR2
IHPSDSDT

SEQ ID NO: 333 - protein, 18 aa, Artificial Sequence
  10-B11-A Heavy Chain CDR3
AIPLYSYDNSGGYYPMDY

SEQ ID NO: 334 - protein, 6 aa, Artificial Sequence
  10-B11-A Light Chain CDR1
QNVRTA

SEQ ID NO: 335 - protein, 3 aa, Artificial Sequence
  10-B11-A Light Chain CDR2
LAS

SEQ ID NO: 336 - protein, 9 aa, Artificial Sequence
  10-B11-A Light Chain CDR3
LQHWNYPFT

SEQ ID NO: 337 - protein, 125 aa, Artificial Sequence; identical to SEQ ID NO: 297
  10-B11-A Heavy Chain

SEQ ID NO: 338 - DNA, 375 bp, Artificial Sequence; identical to SEQ ID NO: 298
  10-B11-A Heavy Chain

SEQ ID NO: 339 - protein, 107 aa, Artificial Sequence; identical to SEQ ID NO: 299
  10-B11-A Light Chain

SEQ ID NO: 340 - DNA, 321 bp, Artificial Sequence; identical to SEQ ID NO: 300
  10-B11-A Light Chain

SEQ ID NO: 341 - protein, 8 aa, Artificial Sequence
  10-L24-A Heavy Chain CDR1
GYTFTDYY

SEQ ID NO: 342 - protein, 8 aa, Artificial Sequence
  10-L24-A Heavy Chain CDR2
INSNNGGT

SEQ ID NO: 343 - protein, 13 aa, Artificial Sequence
  10-L24-A Heavy Chain CDR3
ARRGDGHFGGFTY

SEQ ID NO: 344 - protein, 10 aa, Artificial Sequence
  10-L24-A Light Chain CDR1
ESVDNSGISF

SEQ ID NO: 345 - protein, 3 aa, Artificial Sequence
  10-L24-A Light Chain CDR2
AAS

SEQ ID NO: 346 - protein, 9 aa, Artificial Sequence
  10-L24-A Light Chain CDR3
QQSKEVPWT

SEQ ID NO: 347 - protein, 120 aa, Artificial Sequence; identical to SEQ ID NO: 207
  10-L24-A Heavy Chain

SEQ ID NO: 348 - DNA, 360 bp, Artificial Sequence
  10-L24-A Heavy Chain
gaggtccagctgcaacaatctggacctgaactggtgaagcctggggcctcagtgaagatatcctgtaaggcttctggatacacgttcactgactactacatgaactgggtgaagcagagccatggaaagagccttgagtggattggagatattaattctaacaatggtggtactacctacgaccagaagttcaagggcaaggccactttgactgtagacaggtcctccagtacagcctacatggagctccgcagcctgacatctgaggactctgcagtctattactgtgcaaggagaggggatggtcactttggggggtttacttattggggccaagggactctggtcactgtctctgca

SEQ ID NO: 349 - protein, 111 aa, Artificial Sequence; identical to SEQ ID NO: 209
  10-L24-A Light Chain

SEQ ID NO: 350 - DNA, 333 bp, Artificial Sequence; identical to SEQ ID NO: 210
  10-L24-A Light Chain

SEQ ID NO: 351 - protein, 8 aa, Artificial Sequence
  10-O3-A Heavy Chain CDR1
GFSLTNYA

SEQ ID NO: 352 - protein, 7 aa, Artificial Sequence
  10-O3-A Heavy Chain CDR2
IWSDGRT

SEQ ID NO: 353 - protein, 15 aa, Artificial Sequence
  10-O3-A Heavy Chain CDR3
VRIEGGSYVGWYFDV

SEQ ID NO: 354 - protein, 6 aa, Artificial Sequence
  10-O3-A Light Chain CDR1
QDINKF

SEQ ID NO: 355 - protein, 3 aa, Artificial Sequence
  10-O3-A Light Chain CDR2
YTS

SEQ ID NO: 356 - protein, 8 aa, Artificial Sequence
  10-O3-A Light Chain CDR3
LQYDNLLT

SEQ ID NO: 357 - protein, 121 aa, Artificial Sequence
  10-O3-A Heavy Chain
QVQLKESGPGLVAPSQSLSITCTVSGFSLTNYAVHWVRQSPGKGLEWLGVIWSDGRTDYNAAFISRLSISKDNSKSQVFFKMNSLQADDTAIYYCVRIEGGSYVGWYFDVWGAGTTVTVSS

SEQ ID NO: 358 - DNA, 363 bp, Artificial Sequence
  10-O3-A Heavy Chain
caggtgcagctgaaggagtcaggacctggcctggtggcgccctcacagagcctgtccatcacctgcacagtctctggtttctcattaaccaactatgctgtacactgggttcgccagtctccaggaaagggtctggagtggctgggagtgatatggagtgatggaaggacagactataatgcagctttcatatctagactgagcatcagcaaggacaactccaagagccaagttttctttaaaatgaacagtctgcaagctgatgacacagccatatactactgtgtcagaatcgagggtggtagctacgttggctggtacttcgatgtctggggcgcagggaccacggtcaccgtctcctca

SEQ ID NO: 359 - protein, 106 aa, Artificial Sequence
  10-O3-A Light Chain
DIQMTQSPSSLSASLGGTVTITCKASQDINKFIAWYQHKPGKGPRLLIHYTSTLQPGIPSRFSGSGSGRDYSFSISNLEPEDIATYYCLQYDNLLTFGAGTKLELK

SEQ ID NO: 360 - DNA, 318 bp, Artificial Sequence
  10-O3-A Light Chain
gacatccagatgacacagtctccatcctcactgtctgcatctctgggaggcacagtcaccatcacttgcaaggcaagccaagacattaacaagtttatagcttggtaccaacacaagcctggaaaaggtcctaggctgctcattcattacacatctacattacagccaggcatcccatcaaggttcagtggaagtgggtctgggagagattattccttcagcatcagcaacctggagcctgaagatattgcaacttattattgtctacagtatgataatctgctcacgttcggtgctgggaccaagctggagctgaaa

SEQ ID NO: 361 - protein, 8 aa, Artificial Sequence
  4-M3-A Heavy Chain CDR1
GYSFTGYF

SEQ ID NO: 362 - protein, 8 aa, Artificial Sequence
  4-M3-A Heavy Chain CDR2
INPNNGET

SEQ ID NO: 363 - protein, 8 aa, Artificial Sequence
  4-M3-A Heavy Chain CDR3
ARGGTGTG

SEQ ID NO: 364 - protein, 6 aa, Artificial Sequence
  4-M3-A Light Chain CDR1
QGISSN

SEQ ID NO: 365 - protein, 3 aa, Artificial Sequence
  4-M3-A Light Chain CDR2
YGT

SEQ ID NO: 366 - protein, 9 aa, Artificial Sequence
  4-M3-A Light Chain CDR3
IQYAQFPYT

SEQ ID NO: 367 - protein, 115 aa, Artificial Sequence
  4-M3-A Heavy Chain
EVQLQQSGPELVKPGDSVKISCKASGYSFTGYFMNWVMQSHGKSLEWIGRINPNNGETFYNQKFKGKATLTVDKSSDTAHMELRSLTSEDSAVYYCARGGTGTGWGQGTTLTVSS

SEQ ID NO: 368 - DNA, 345 bp, Artificial Sequence
  4-M3-A Heavy Chain
gaggttcagctgcagcagtctggacctgagctggtgaagcctggggattcagtgaagatatcctgcaaggcttctggttactcatttactggctactttatgaactgggtgatgcagagccatggaaagagccttgagtggattggacgtattaatcctaacaatggtgaaactttctacaaccagaagttcaagggcaaggccacattgactgtagacaaatcctctgacacagcccacatggagctccggagcctgacatctgaggactctgcagtctattattgtgcaagaggggggactgggacggggtggggccaaggcaccactctcacagtctcctca

SEQ ID NO: 369 - protein, 107 aa, Artificial Sequence
  4-M3-A Light Chain
DILMTQSPSSMSVSLGDTVSITCHASQGISSNIGWLQQKPGKSFKGLIYYGTNLEDGVPSRFSGSGSGADYSLTISSLESEDLADYYCIQYAQFPYTFGGGTKLEIK

SEQ ID NO: 370 - DNA, 321 bp, Artificial Sequence
  4-M3-A Light Chain
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctattatggaaccaacttggaagatggagttccatcaaggttcagtggcagtggatctggagcagattattctctcaccatcagcagcctggaatctgaagatcttgcagactattactgtatacagtatgctcagtttccgtacacgttcggaggggggaccaaactggaaataaaa

SEQ ID NO: 371 - protein, 8 aa, Artificial Sequence
  4-N22-A Heavy Chain CDR1
GYTFTNYW

SEQ ID NO: 372 - protein, 8 aa, Artificial Sequence
  4-N22-A Heavy Chain CDR2
INPSNGGT

SEQ ID NO: 373 - protein, 7 aa, Artificial Sequence
  4-N22-A Heavy Chain CDR3
ARETGNY

SEQ ID NO: 374 - protein, 6 aa, Artificial Sequence
  4-N22-A Light Chain CDR1
QGISSN

SEQ ID NO: 375 - protein, 3 aa, Artificial Sequence
  4-N22-A Light Chain CDR2
HGT

SEQ ID NO: 376 - protein, 9 aa, Artificial Sequence
  4-N22-A Light Chain CDR3
VQYAQFPYT

SEQ ID NO: 377 - protein, 114 aa, Artificial Sequence
  4-N22-A Heavy Chain
QVQLQQPGTELVKPGASVKLSCKASGYTFTNYWMYWVKQRPGQGLDWIGNINPSNGGTNYNEKFKSKATLTVDKSSSTAYMQLSSLTSEDSAVYYCARETGNYWGQGTTLTVSS

SEQ ID NO: 378 - DNA, 342 bp, Artificial Sequence
  4-N22-A Heavy Chain
caggtccaactgcagcagcctgggactgaactggtgaagcctggggcttcagtgaagctgtcctgcaaggcttctggctacaccttcaccaattactggatgtactgggtgaagcagaggcctggacaaggccttgattggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagagagactgggaactactggggccaaggcaccactctcacagtctcctca

SEQ ID NO: 379 - protein, 107 aa, Artificial Sequence
  4-N22-A Light Chain
DILMTQSPSSMSVSLGDTVSISCHASQGISSNIGWLQQKPGKSFKGLIYHGTNLEDGVPSRFSGSGSGADYSLTISSLESEDFADYYCVQYAQFPYTFGGGTKLEIK

SEQ ID NO: 380 - DNA, 321 bp, Artificial Sequence
  4-N22-A Light Chain
gacatcctgatgacccaatctccatcctccatgtctgtatctctgggagacacagtcagcatctcttgccatgcgagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgctcagtttccgtacacgttcggaggggggaccaagctggaaataaaa

SEQ ID NO: 381 - protein, 8 aa, Artificial Sequence
  7-B10-A Heavy Chain CDR1
GFTFNTYA

SEQ ID NO: 382 - protein, 10 aa, Artificial Sequence
  7-B10-A Heavy Chain CDR2
IRSKSSNYAR

SEQ ID NO: 383 - protein, 15 aa, Artificial Sequence
  7-B10-A Heavy Chain CDR3
VRDHDYYGSSYWFAY

SEQ ID NO: 384 - protein, 11 aa, Artificial Sequence
  7-B10-A Light Chain CDR1
QSLVHNNGNTY

SEQ ID NO: 385 - protein, 3 aa, Artificial Sequence
  7-B10-A Light Chain CDR2
KVS

SEQ ID NO: 386 - protein, 9 aa, Artificial Sequence
  7-B10-A Light Chain CDR3
SQSTHVPWT

SEQ ID NO: 387 - protein, 124 aa, Artificial Sequence
  7-B10-A Heavy Chain
EVQLVESGGGLVQPKGSLKLSCAASGFTFNTYAMHWVRQAPGKGLEWVARIRSKSSNYARYYADSVKDRFTISRDESQSMLYLQMNNLKTEDTAMYYCVRDHDYYGSSYWFAYWGQGTLVTVSA

SEQ ID NO: 388 - DNA, 372 bp, Artificial Sequence
  7-B10-A Heavy Chain
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaagatattatgccgattcagtgaaagacagattcaccatttccagagatgagtcacaaagcatgctctatctgcaaatgaacaacctgaaaactgaggacacagccatgtattactgtgtgagagatcacgattactacggtagtagttactggtttgcttactggggccaagggactctggtcactgtctctgca

SEQ ID NO: 389 - protein, 112 aa, Artificial Sequence
  7-B10-A Light Chain
DVVMTQTPLSLPVSLGDQASISCSSSQSLVHNNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 390 - DNA, 336 bp, Artificial Sequence
  7-B10-A Light Chain
gatgttgtgatgacccaaactccactctccctgcctgtcagtcttggagatcaagcctccatctcttgcagctctagtcagagccttgtacacaataatggaaacacctatttacattggtacctgcagaagccgggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 391 - protein, 10 aa, Artificial Sequence
  8-H5-A Heavy Chain CDR1
GFSLSTSGMG

SEQ ID NO: 392 - protein, 7 aa, Artificial Sequence
  8-H5-A Heavy Chain CDR2
IYWDDDK

SEQ ID NO: 393 - protein, 14 aa, Artificial Sequence
  8-H5-A Heavy Chain CDR3
ARRDLHDYDGWFAY

SEQ ID NO: 394 - protein, 6 aa, Artificial Sequence
  8-H5-A Light Chain CDR1
QNVGTN

SEQ ID NO: 395 - protein, 3 aa, Artificial Sequence
  8-H5-A Light Chain CDR2
SAS

SEQ ID NO: 396 - protein, 9 aa, Artificial Sequence
  8-H5-A Light Chain CDR3
QQYNSYPLT

SEQ ID NO: 397 - protein, 122 aa, Artificial Sequence
  8-H5-A Heavy Chain
QVTLKESGPGILQSSQTLSLTCSLSGFSLSTSGMGVSWVRQPSGKGLEWLAHIYWDDDKRYTPSLKSRLTISTDSSRKQVFLKITSVDTADTATYYCARRDLHDYDGWFAYWGQGTLVTVSA

SEQ ID NO: 398 - DNA, 366 bp, Artificial Sequence
  8-H5-A Heavy Chain
caggttactctgaaagagtctggccctgggatattgcagtcctcccagaccctcagtctgacttgttctttgtctgggttttcactgagcacttctggtatgggtgtgagctgggttcgtcagccttcaggaaagggtctggagtggctggcacacatttactgggatgatgacaagcgctataccccttccctgaagagccggctcacaatctccacggattcctccagaaagcaggtattcctcaagatcaccagtgtggacactgcagatactgccacatactactgtgctcgaagagatcttcatgattacgacggctggtttgcttactggggccaagggactctggtcactgtctctgca

SEQ ID NO: 399 - protein, 107 aa, Artificial Sequence
  8-H5-A Light Chain
DIVMTQSQKFMSTAVGDRVSVTCKASQNVGTNVAWYQQKPGQSPKALIYSASFRYSGVPDRFSGSGSGTDFTLTISNVQSEDLAEYFCQQYNSYPLTFGSGTKLEIK

SEQ ID NO: 400 - DNA, 321 bp, Artificial Sequence
  8-H5-A Light Chain
gacattgtgatgacccagtctcaaaaattcatgtccacagcagtaggagacagggtcagcgtcacctgcaaggccagtcaaaatgtgggtactaatgtcgcctggtatcaacagaaaccagggcaatctcctaaagcactgatttactcggcatccttccgctacagtggagtccctgatcgcttctcaggcagtggatctgggacagatttcactctcaccatcagcaatgtgcagtctgaagacttggcagagtatttctgtcagcaatataacagctatcctctcacgttcggctcggggacaaagttggaaataaaa

SEQ ID NO: 401 - protein, 8 aa, Artificial Sequence
  2-G20-A Heavy Chain CDR1
GYTFPSYV

SEQ ID NO: 402 - protein, 8 aa, Artificial Sequence
  2-G20-A Heavy Chain CDR2
IYPYNDGT

SEQ ID NO: 403 - protein, 13 aa, Artificial Sequence
  2-G20-A Heavy Chain CDR3
ARGDYGDSAWFPY

SEQ ID NO: 404 - protein, 10 aa, Artificial Sequence
  2-G20-A Light Chain CDR1
KSVSTSGYSY

SEQ ID NO: 405 - protein, 3 aa, Artificial Sequence
  2-G20-A Light Chain CDR2
LAS

SEQ ID NO: 406 - protein, 9 aa, Artificial Sequence
  2-G20-A Light Chain CDR3
QLSRELPWT

SEQ ID NO: 407 - protein, 120 aa, Artificial Sequence
  2-G20-A Heavy Chain
EVQLQQSGPELVKPGASVKMSCKASGYTFPSYVLHWVKQKPGQGLEWIGYIYPYNDGTNYNEKFKGKATLTSDKSSSTAYMELSSLTSEDSAVYYCARGDYGDSAWFPYWGQGTLVTVSA

SEQ ID NO: 408 - DNA, 360 bp, Artificial Sequence
  2-G20-A Heavy Chain
gaggtccagctgcagcagtctggacctgagctggtaaagcctggggcttcagtgaagatgtcctgcaaggcttctggatacacattccctagttatgttttgcactgggtgaagcagaagcctgggcagggccttgagtggattggatatatttatccttacaatgatggtactaactacaatgagaagttcaaaggcaaggccacactgacttcagacaaatcctccagcacagcctacatggagctcagcagcctgacctctgaggactctgcggtctattactgtgcaaggggagactatggtgactccgcctggtttccttactggggccaagggactctggtcactgtctctgca

SEQ ID NO: 409 - protein, 111 aa, Artificial Sequence
  2-G20-A Light Chain
DIVLTQSPASLAVSLGQRATISCRASKSVSTSGYSYMHWYQQKPGQPPKLLIYLASNLKSGVPARFSGGGSGTDFTLNIHPVEEEDAATYYCQLSRELPWTFGGGTKLEIK

SEQ ID NO: 410 - DNA, 333 bp, Artificial Sequence
  2-G20-A Light Chain
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctaaaatctggggtccctgccaggttcagtggcggtgggtctgggacagacttcaccctcaacatccatcctgtggaggaggaggatgctgcaacctattactgtcagctcagtagggagcttccgtggacgttcggtggaggcaccaagctggaaatcaaa

SEQ ID NO: 411 - protein, 8 aa, Artificial Sequence
  3-E2-A Heavy Chain CDR1
GYTFTDYY

SEQ ID NO: 412 - protein, 8 aa, Artificial Sequence
  3-E2-A Heavy Chain CDR2
INPNNGGT

SEQ ID NO: 413 - protein, 13 aa, Artificial Sequence
  3-E2-A Heavy Chain CDR3
ARRGDGHYGGFTY

SEQ ID NO: 414 - protein, 10 aa, Artificial Sequence
  3-E2-A Light Chain CDR1
ESVDNSGISF

SEQ ID NO: 415 - protein, 3 aa, Artificial Sequence
  3-E2-A Light Chain CDR2
AAS

SEQ ID NO: 416 - protein, 9 aa, Artificial Sequence
  3-E2-A Light Chain CDR3
QQSKEVPWT

SEQ ID NO: 417 - protein, 120 aa, Artificial Sequence
  3-E2-A Heavy Chain
EVQLQQSGPELVKPGASVKISCKASGYTFTDYYMNWVKQSHGKSLEWIGDINPNNGGTSYTQKFKGKATLTVDKSSSKAYMELRSLTSEDSAVYYCARRGDGHYGGFTYWGQGTLVTVSA

SEQ ID NO: 418 - DNA, 360 bp, Artificial Sequence
  3-E2-A Heavy Chain
gaggtccagctgcaacaatctggacctgaactggtgaagcctggggcttcagtgaagatatcctgtaaggcttctggatacacgttcactgactactacatgaactgggtgaagcagagccatggaaagagccttgagtggattggagatattaatcctaacaatggtggtactagctacacccagaagttcaagggcaaggccacattgactgtagacaagtcctccagcaaagcctacatggagctccgcagcctgacatctgaggactctgcagtctattactgtgcaagaagaggggatggtcactacgggggatttacttactggggccaagggactctggtcactgtctctgca

SEQ ID NO: 419 - protein, 111 aa, Artificial Sequence
  3-E2-A Light Chain
DIVLTQSPASLVVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 420 - DNA, 333 bp, Artificial Sequence
  3-E2-A Light Chain
gacattgtgctgacccaatctccagcttctttggttgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 421 - protein, 8 aa, Artificial Sequence
  4-K16-A Heavy Chain CDR1
GYTFTTYG

SEQ ID NO: 422 - protein, 8 aa, Artificial Sequence
  4-K16-A Heavy Chain CDR2
MSTYSGVP

SEQ ID NO: 423 - protein, 7 aa, Artificial Sequence
  4-K16-A Heavy Chain CDR3
ASGDVGY

SEQ ID NO: 424 - protein, 6 aa, Artificial Sequence
  4-K16-A Light Chain CDR1
QNVGTN

SEQ ID NO: 425 - protein, 3 aa, Artificial Sequence
  4-K16-A Light Chain CDR2
SAS

SEQ ID NO: 426 - protein, 9 aa, Artificial Sequence
  4-K16-A Light Chain CDR3
QQYNIYPST

SEQ ID NO: 427 - protein, 114 aa, Artificial Sequence
  4-K16-A Heavy Chain
QIQLVQSGPELKKPGETVKISCKASGYTFTTYGMSWVKQAPGKGLKWMGWMSTYSGVPTYADDFKGRFAFSLETSASTAYLQINNLKNEDTATYFCASGDVGYWGQGTTLTVSS

SEQ ID NO: 428 - DNA, 342 bp, Artificial Sequence
  4-K16-A Heavy Chain
cagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggatgagcacctactctggagtgccaacatatgctgatgacttcaagggacggtttgccttctctttggaaacctctgccagcactgcctatttgcagatcaacaacctcaaaaatgaggacacggctacatatttctgtgcaagcggggacgtcggctactggggccaaggcaccactctcacagtctcctca

SEQ ID NO: 429 - protein, 107 aa, Artificial Sequence
  4-K16-A Light Chain
DIVMTQSQKFMSTSVGDRVSVTCKASQNVGTNVAWYQQKPGQSPKALIYSASYRYSGVPDRFTGSGSGTDFTLTISTVQSEDLAEYFCQQYNIYPSTFGGGTKLEIK

SEQ ID NO: 430 - DNA, 321 bp, Artificial Sequence
  4-K16-A Light Chain
gacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcactgtgcagtctgaagacttggcagagtatttctgtcagcaatataacatctatccgtccacgttcggaggggggaccaagctggaaataaaa

SEQ ID NO: 431 - protein, 8 aa, Artificial Sequence
  6-C19-A-WT Heavy Chain CDR1
GYTFTDYN

SEQ ID NO: 432 - protein, 8 aa, Artificial Sequence
  6-C19-A-WT Heavy Chain CDR2
INPNNGGT

SEQ ID NO: 433 - protein, 17 aa, Artificial Sequence
  6-C19-A-WT Heavy Chain CDR3
ARGGFYGNYGSYYGMDF

SEQ ID NO: 434 - protein, 6 aa, Artificial Sequence
  6-C19-A-WT Light Chain CDR1
ENIYSH

SEQ ID NO: 435 - protein, 3 aa, Artificial Sequence
  6-C19-A-WT Light Chain CDR2
NAK

SEQ ID NO: 436 - protein, 9 aa, Artificial Sequence
  6-C19-A-WT Light Chain CDR3
QHHYGIPPE

SEQ ID NO: 437 - protein, 124 aa, Artificial Sequence
  6-C19-A-WT Heavy Chain
EVQLQQSGPELVKPGASVKMSCKASGYTFTDYNIHWVKQSHGKSLEWIGYINPNNGGTDYNQKFQGKATLTVDKSSSTAYMELRSLTSEDSAVYYCARGGFYGNYGSYYGMDFWGQGTSVTVSS

SEQ ID NO: 438 - DNA, 372 bp, Artificial Sequence
  6-C19-A-WT Heavy Chain
gaggtccagctgcaacagtctggacctgagctggtgaagcctggggcttcagtgaagatgtcctgcaaggcttctggatacacattcactgactacaacatacactgggtgaagcagagccatggaaagagccttgagtggattggatatattaaccctaacaatggtggtactgactacaaccagaagttccagggcaaggccacattgactgtagacaagtcctccagcacagcctacatggagctccgcagcctgacatcggaagattctgcagtctattactgtgcaagaggggggttttatggcaattacgggtcttactatggtatggacttctggggtcaaggaacctcagtcaccgtctcctca

SEQ ID NO: 439 - protein, 107 aa, Artificial Sequence
  6-C19-A-WT Light Chain
DIQMTQSPASLSASVGETVTITCRTSENIYSHLAWYQQTQGKSPHLLVYNAKTLAEGVPSRFSGSGSGTQFSLKINSLQPEDFGSYFCQHHYGIPPEFGGGTKLEIK

SEQ ID NO: 440 - DNA, 321 bp, Artificial Sequence
  6-C19-A-WT Light Chain
gacatccagatgactcagtctccagcctccctatctgcatctgtgggagaaactgtcaccatcacatgtcgaacaagtgagaatatttatagtcatttagcatggtatcagcagacacagggaaaatctcctcacctcctggtctataatgcaaaaaccttagcagaaggtgtgccatcaaggttcagtggcagtggatcaggcacacagttttctctgaagatcaacagcctgcagcctgaagattttgggagttatttctgtcaacatcattatggtattcctccggagttcggtggaggcaccaagctggaaatcaaa

SEQ ID NO: 441 - protein, 8 aa, Artificial Sequence
  6-L8-A Heavy Chain CDR1
GFNIKNTY

SEQ ID NO: 442 - protein, 8 aa, Artificial Sequence
  6-L8-A Heavy Chain CDR2
INPANDDT

SEQ ID NO: 443 - protein, 10 aa, Artificial Sequence
  6-L8-A Heavy Chain CDR3
VYDVRWYFDV

SEQ ID NO: 444 - protein, 11 aa, Artificial Sequence
  6-L8-A Light Chain CDR1
QGLVHSNGNTY

SEQ ID NO: 445 - protein, 3 aa, Artificial Sequence
  6-L8-A Light Chain CDR2
KVS

SEQ ID NO: 446 - protein, 9 aa, Artificial Sequence
  6-L8-A Light Chain CDR3
SQSTHVPPT

SEQ ID NO: 447 - protein, 117 aa, Artificial Sequence
  6-L8-A Heavy Chain
EVQLQQSVAELVRPGASVKLSCTPSGFNIKNTYMHWMKQRPEQGLEWIGRINPANDDTKYAPKFQDKATITADTSSNTAYLQLSSLTSEDTAIYYCVYDVRWYFDVWGTGTTVTVSS

SEQ ID NO: 448 - DNA, 351 bp, Artificial Sequence
  6-L8-A Heavy Chain
gaggttcagctgcagcagtctgtggcagagcttgtgaggccaggggcctcagtcaagttgtcctgcacaccttctggcttcaacattaaaaacacctatatgcactggatgaagcagaggcctgaacagggcctggagtggattggaaggattaatcctgcgaatgatgatactaaatatgccccgaagttccaggacaaggccactataactgcagacacatcctccaacacagcctacctgcagctcagcagcctgacatctgaggacactgccatctattactgtgtctacgacgtacggtggtatttcgatgtctggggcacagggaccacggtcaccgtctcctca

SEQ ID NO: 449 - protein, 112 aa, Artificial Sequence
  6-L8-A Light Chain
DVVMTQTPLSLPVSLGDQASISCRSSQGLVHSNGNTYLHWYLQKPGQSPKLLIYKVSNRFSGVPDRFSGSGSGTDFTLKISRVEAEDLGVYFCSQSTHVPPTFGAGTKLELK

SEQ ID NO: 450 - DNA, 336 bp, Artificial Sequence
  6-L8-A Light Chain
gatgttgtgatgacccaaactccactctccctgcctgtcagtcttggagatcaagcctccatctcttgcagatctagtcagggccttgtacacagtaatgggaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctcctacgttcggtgctgggaccaagctggagctgaaa

SEQ ID NO: 451 - protein, 8 aa, Artificial Sequence
  7-D7-A Heavy Chain CDR1
GFSLTSYA

SEQ ID NO: 452 - protein, 7 aa, Artificial Sequence
  7-D7-A Heavy Chain CDR2
IWPGGGT

SEQ ID NO: 453 - protein, 9 aa, Artificial Sequence
  7-D7-A Heavy Chain CDR3
ARTGYYFDY

SEQ ID NO: 454 - protein, 5 aa, Artificial Sequence
  7-D7-A Light Chain CDR1
LSVNY

SEQ ID NO: 455 - protein, 3 aa, Artificial Sequence
  7-D7-A Light Chain CDR2
DTS

SEQ ID NO: 456 - protein, 11 aa, Artificial Sequence
  7-D7-A Light Chain CDR3
QQWSSNPPMYT

SEQ ID NO: 457 - protein, 115 aa, Artificial Sequence
  7-D7-A Heavy Chain
QVQLKESGPGLVAPSQSLSITCTVSGFSLTSYAISWVRQPPGKGLEWLGVIWPGGGTNYNSALKSRLSISKDNSKSQVFLKMNSLQTDDTAKYYCARTGYYFDYWGQGTTLTVSS

SEQ ID NO: 458 - DNA, 345 bp, Artificial Sequence
  7-D7-A Heavy Chain
caggtgcagctgaaggagtcaggacctggcctggtggcgccctcacagagcctgtccatcacatgcactgtctctgggttctcattaaccagctatgctataagctgggttcgccagccaccaggaaagggtctggagtggcttggagtaatatggcctggtggaggcacaaattataattcagctctcaaatccagactgagcatcagcaaagacaactccaagagtcaagttttcttaaaaatgaacagtctgcaaactgatgacacagccaaatactactgtgccagaactgggtactactttgactactggggccaaggcaccactctcacagtctcctca

SEQ ID NO: 459 - protein, 108 aa, Artificial Sequence
  7-D7-A Light Chain
QLILTQSPAIMSASPGEKVTMTCSASLSVNYMYWFQQKPGTSPKRWIHDTSKLASGVPARFSGSGSGTSYSLTISSMEAEDAATYYCQQWSSNPPMYTFGGGTKLEIK

SEQ ID NO: 460 - DNA, 324 bp, Artificial Sequence
  7-D7-A Light Chain
caacttattctcacccagtctccagcaatcatgtctgcatctccaggggagaaggtcaccatgacctgcagtgccagcttaagtgtaaattacatgtactggttccagcagaagccaggcacctcccccaaaagatggattcatgacacatccaaactggcttctggagtccctgctcgcttcagtggcagtgggtctgggacctcttactctctcacaatcagcagcatggaggctgaagatgctgccacttattactgccagcagtggagtagtaacccacccatgtacacgttcggaggggggaccaagctggaaataaaa

SEQ ID NO: 461 - protein, 8 aa, Artificial Sequence
  7-N20-A Heavy Chain CDR1
GFTFSNYR

SEQ ID NO: 462 - protein, 10 aa, Artificial Sequence
  7-N20-A Heavy Chain CDR2
ITDKSDNYGA

SEQ ID NO: 463 - protein, 11 aa, Artificial Sequence
  7-N20-A Heavy Chain CDR3
SRDGYDWYFDL

SEQ ID NO: 464 - protein, 6 aa, Artificial Sequence
  7-N20-A Light Chain CDR1
QDVGIV

SEQ ID NO: 465 - protein, 3 aa, Artificial Sequence
  7-N20-A Light Chain CDR2
WAS

SEQ ID NO: 466 - protein, 8 aa, Artificial Sequence
  7-N20-A Light Chain CDR3
QQYSSYPT

SEQ ID NO: 467 - protein, 120 aa, Artificial Sequence
  7-N20-A Heavy Chain
QVQLVETGGGLVRPGNSLKLSCVTSGFTFSNYRMHWLRQPPGKRLEWIGIITDKSDNYGANYAESVKGRFAISRDDSKSSVYLEMKRLREEDTATYFCSRDGYDWYFDLWGTGTTVTVSS

SEQ ID NO: 468 - DNA, 360 bp, Artificial Sequence
  7-N20-A Heavy Chain
caggtgcagcttgtagagaccgggggaggcttggtgaggcctggaaattctctgaaactctcctgtgttacgtcgggattcactttcagtaactaccggatgcactggcttcgccagcctccagggaagaggctggagtggattggtataattacagacaaatctgataattatggagcaaattatgcagagtctgtgaaaggcagattcgccatttcaagagatgattcaaaaagtagtgtctatctagagatgaagagattaagagaggaagacactgccacttatttttgtagtagagatggttacgactggtacttcgatctctggggcacagggaccacggtcaccgtctcctca

SEQ ID NO: 469 - protein, 106 aa, Artificial Sequence
  7-N20-A Light Chain
DIVMTQSHKFMSTSVGDRVRITCKASQDVGIVVAWYQQKPGQSPKLLIYWASTRHTGVPDRFTGSGSGTDFTLTISNVQSEDLADYFCQQYSSYPTFGGGTKLEIK

SEQ ID NO: 470 - DNA, 318 bp, Artificial Sequence
  7-N20-A Light Chain
gacattgtgatgacccagtctcacaaattcatgtccacatcagtaggagacagggtcagaatcacctgtaaggccagtcaggatgtgggaattgttgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcccacgttcggaggggggaccaagctggaaataaaa

SEQ ID NO: 471 - protein, 8 aa, Artificial Sequence
  8-A17-A Heavy Chain CDR1
GFSLTSYA

SEQ ID NO: 472 - protein, 7 aa, Artificial Sequence
  8-A17-A Heavy Chain CDR2
VWPGGGT

SEQ ID NO: 473 - protein, 9 aa, Artificial Sequence
  8-A17-A Heavy Chain CDR3
ARTGYHFDY

SEQ ID NO: 474 - protein, 5 aa, Artificial Sequence
  8-A17-A Light Chain CDR1
SSVSY

SEQ ID NO: 475 - protein, 3 aa, Artificial Sequence
  8-A17-A Light Chain CDR2
DTS

SEQ ID NO: 476 - protein, 11 aa, Artificial Sequence
  8-A17-A Light Chain CDR3
QQWSSNPPMYT

SEQ ID NO: 477 - protein, 115 aa, Artificial Sequence
  8-A17-A Heavy Chain
QVQLKESGPGLVAPSQSLSITCTVSGFSLTSYAISWVRQPPGKGLEWLGVVWPGGGTNYNSALKSRLSINKDNSKSQVFLKMNSLQTDDTGRYYCARTGYHFDYWGQGTTLTVSS

SEQ ID NO: 478 - DNA, 345 bp, Artificial Sequence
  8-A17-A Heavy Chain
caggtgcagctgaaggagtcaggacctggcctggtggcgccctcacagagcctgtccatcacatgcactgtctctgggttctcattaaccagctatgctataagctgggttcgccagccaccaggaaagggtctggagtggcttggagtagtatggcctggtggaggcacaaattataattcagctctcaaatccagactgagcatcaacaaagacaactccaagagtcaagttttcttaaagatgaacagtctgcaaactgatgacacaggcaggtactactgtgccagaactgggtaccattttgactactggggccaaggcaccactctcacagtctcctca

SEQ ID NO: 479 - protein, 108 aa, Artificial Sequence
  8-A17-A Light Chain
QIVLTQSPAIMSASPGEKVTMTCSVSSSVSYMHWYQQKSGTSPKRWIYDTSKLASGVPARFSGSGSGTSYSLTISSMEAEDAATYYCQQWSSNPPMYTFGGGTKLEIK

SEQ ID NO: 480 - DNA, 324 bp, Artificial Sequence
  8-A17-A Light Chain
caaattgttctcacccagtctccagcaatcatgtctgcatctccaggggagaaggtcaccatgacctgcagtgtcagctcaagtgtaagttacatgcactggtaccagcagaagtcaggcacctcccccaaaagatggatttatgacacatccaaactggcttctggagtccctgctcgtttcagtggcagtgggtctgggacctcttactctctcacaatcagcagcatggaggctgaagatgctgccacttattactgccagcagtggagtagtaacccacccatgtacacgttcggaggggggaccaagctggaaataaaa

SEQ ID NO: 481 - protein, 8 aa, Artificial Sequence
  8-H3-A Heavy Chain CDR1
GFTFNTYA

SEQ ID NO: 482 - protein, 10 aa, Artificial Sequence
  8-H3-A Heavy Chain CDR2
IRSKSSNYAT

SEQ ID NO: 483 - protein, 15 aa, Artificial Sequence
  8-H3-A Heavy Chain CDR3
VRDHDYYGSSYWFTY

SEQ ID NO: 484 - protein, 11 aa, Artificial Sequence
  8-H3-A Light Chain CDR1
QSLVHDNGNTY

SEQ ID NO: 485 - protein, 3 aa, Artificial Sequence
  8-H3-A Light Chain CDR2
KVS

SEQ ID NO: 486 - protein, 9 aa, Artificial Sequence
  8-H3-A Light Chain CDR3
SQSTHVPWT

SEQ ID NO: 487 - protein, 124 aa, Artificial Sequence
  8-H3-A Heavy Chain
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVRDHDYYGSSYWFTYWGQGTLVTVSA

SEQ ID NO: 488 - DNA, 372 bp, Artificial Sequence
  8-H3-A Heavy Chain
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaacatattatgccgattcagtgaaagacagattcaccatctccagagatgattcacaaagcatgctctatctgcaaatgaacaacctgaaaactgaggacacggccatgtattactgtgtgagagatcacgattactacggtagtagctactggtttacttactggggccaagggactctggtcactgtctctgca

SEQ ID NO: 489 - protein, 112 aa, Artificial Sequence
  8-H3-A Light Chain
DVVMTQTPLSLPVSLGDQASISCRSSQSLVHDNGNTYLHWFLQKPGQSPKLLIYKVSNRFSGVPDRFSGSGSGTDFTLKISRVETEDLGVYFCSQSTHVPWTFGGGTKLEIK

SEQ ID NO: 490 - DNA, 336 bp, Artificial Sequence
  8-H3-A Light Chain
gatgttgtgatgacccaaactccactctccctgcctgtcagtcttggagatcaagcctccatctcttgcagatctagtcagagccttgtacacgataatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggagactgaggatctgggagtttatttctgctctcaaagtacacatgttccgtggacgttcggtggaggcaccaagctggaaatcaaa

SEQ ID NO: 491 - protein, 8 aa, Artificial Sequence
  8-L17-A Heavy Chain CDR1
GFTFNTYA

SEQ ID NO: 492 - protein, 10 aa, Artificial Sequence
  8-L17-A Heavy Chain CDR2
IRSKSSNYAR

SEQ ID NO: 493 - protein, 15 aa, Artificial Sequence
  8-L17-A Heavy Chain CDR3
VRDHDYYGSSYWFAY

SEQ ID NO: 494 - protein, 11 aa, Artificial Sequence
  8-L17-A Light Chain CDR1
QSLVHNNGNTY

SEQ ID NO: 495 - protein, 3 aa, Artificial Sequence
  8-L17-A Light Chain CDR2
KVS

SEQ ID NO: 496 - protein, 9 aa, Artificial Sequence
  8-L17-A Light Chain CDR3
SQSTHVPWT

SEQ ID NO: 497 - protein, 124 aa, Artificial Sequence
  8-L17-A Heavy Chain
EVQLVESGGGLVQPKGSLKLSCAASGFTFNTYAMHWVRQAPGKGLEWVARIRSKSSNYARYYADSVKDRFTISRDDSQSMLYLQMNNLKTEDTAMYYCVRDHDYYGSSYWFAYWGQGTLVTVSA

SEQ ID NO: 498 - DNA, 372 bp, Artificial Sequence
  8-L17-A Heavy Chain
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaagatattatgccgattcagtgaaagacagattcaccatctccagagatgattcacaaagcatgctctatctgcaaatgaacaacctgaaaactgaggacacagccatgtattactgtgtgagagatcacgattactacggtagtagttactggtttgcttactggggccaagggactctggtcactgtctctgca

SEQ ID NO: 499 - protein, 112 aa, Artificial Sequence
  8-L17-A Light Chain
DVVMTQTPLSLPVSLGDQASISCRSSQSLVHNNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 500 - DNA, 336 bp, Artificial Sequence
  8-L17-A Light Chain
gatgttgtgatgacccaaactccactctccctgcctgtcagtcttggagatcaagcctccatctcttgcagatctagtcagagccttgtacacaataatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 501 - protein, 8 aa, Artificial Sequence
  9-F6-A Heavy Chain CDR1
GYTFTSYI

SEQ ID NO: 502 - protein, 8 aa, Artificial Sequence
  9-F6-A Heavy Chain CDR2
ILPYNDGT

SEQ ID NO: 503 - protein, 11 aa, Artificial Sequence
  9-F6-A Heavy Chain CDR3
ARWGYSNFLDS

SEQ ID NO: 504 - protein, 6 aa, Artificial Sequence
  9-F6-A Light Chain CDR1
QDINNY

SEQ ID NO: 505 - protein, 3 aa, Artificial Sequence
  9-F6-A Light Chain CDR2
YTS

SEQ ID NO: 506 - protein, 9 aa, Artificial Sequence
  9-F6-A Light Chain CDR3
QQGNTLPPT

SEQ ID NO: 507 - protein, 118 aa, Artificial Sequence
  9-F6-A Heavy Chain
EVQLQQSGPELVKPGASVKMSCKASGYTFTSYIMHWVKQKPGQGLEWIGYILPYNDGTKYNEKFKGKATLTSDKSSSTAYMELRSLTSEDSAVYYCARWGYSNFLDSWGQGTTLTVSS

SEQ ID NO: 508 - DNA, 354 bp, Artificial Sequence
  9-F6-A Heavy Chain
gaggtccagctgcagcagtctggacctgagctggtaaagcctggggcttcagtgaagatgtcctgcaaggcttctggatacacattcactagctatattatgcactgggtgaagcagaagcctggacagggccttgagtggattggatatattcttccttacaatgatggtactaagtacaatgagaagttcaaaggcaaggccacactgacttcagacaaatcctccagcacagcctatatggagctcagaagcctgacctctgaggactctgcggtctattactgtgcaagatggggctatagtaactttcttgactcctggggccaaggcaccactctcacagtctcctca

SEQ ID NO: 509 - protein, 107 aa, Artificial Sequence
  9-F6-A Light Chain
DIQMTQTTSSLSASLGDRVTISCRASQDINNYLNWYQQKPDGTVKLLIYYTSRLHSGVPSRFSGSGSGTDYSLTIRNLEQEDIATYFCQQGNTLPPTFGGGTKLEIK

SEQ ID NO: 510 - DNA, 321 bp, Artificial Sequence
  9-F6-A Light Chain
gatatccagatgacacagactacatcctccctgtctgcctctctgggagacagagtcaccatcagttgcagggcaagtcaggacattaacaattatttaaactggtatcagcagaaaccagatggaactgttaaactcctgatctactacacatcaagattacactcaggagtcccatcaaggttcagtggcagtgggtctggaacagattattctctcaccattaggaacctggagcaagaagacattgccacttacttttgccaacagggtaatacgcttcctccgacgttcggtggaggcaccaagctggaaatcaaa

SEQ ID NO: 511 - protein, 8 aa, Artificial Sequence
  10-I12-A Heavy Chain CDR1
GYTFTDSY

SEQ ID NO: 512 - protein, 8 aa, Artificial Sequence
  10-I12-A Heavy Chain CDR2
IFPGNDNT

SEQ ID NO: 513 - protein, 12 aa, Artificial Sequence
  10-I12-A Heavy Chain CDR3
ARSGGLRLGFAY

SEQ ID NO: 514 - protein, 10 aa, Artificial Sequence
  10-I12-A Light Chain CDR1
ESVDSYGHSF

SEQ ID NO: 515 - protein, 3 aa, Artificial Sequence
  10-I12-A Light Chain CDR2
LAS

SEQ ID NO: 516 - protein, 9 aa, Artificial Sequence
  10-I12-A Light Chain CDR3
QQNNEDPWT

SEQ ID NO: 517 - protein, 119 aa, Artificial Sequence
  10-I12-A Heavy Chain
RVQLQQSGPELVRPGTSVKISCKASGYTFTDSYINWVTQRPGQGLDWIGWIFPGNDNTYYNEKFEGKATLTLDKSSSTAYMLLSSLTSEDSAVYFCARSGGLRLGFAYWGQGTLVTVSA

SEQ ID NO: 518 - DNA, 357 bp, Artificial Sequence
  10-I12-A Heavy Chain
cgggtccagctacagcagtctggacctgagctggtgaggcctgggacttcagtgaagatatcctgcaaggcttctggctacaccttcactgactcctatataaactgggtgacgcagaggcctggacagggacttgattggattggatggatttttcctggaaatgataatacttactacaatgagaagttcgagggcaaggccacacttactcttgacaaatcttccagtacagcctacatgttgctcagcagcctgacctctgaggactctgcggtctatttctgtgcaagatcagggggattacgactggggtttgcttactggggccaagggactctggtcactgtctctgca

SEQ ID NO: 519 - protein, 111 aa, Artificial Sequence
  10-I12-A Light Chain
NIVLTQSPASLAVSRGQRATISCRASESVDSYGHSFIHWYQQKPGQPPKILIYLASKLESGVPARFSGSGSRTDFTLTIDPVEADDAATYYCQQNNEDPWTFGGGTKLEIK

SEQ ID NO: 520 - DNA, 333 bp, Artificial Sequence
  10-I12-A Light Chain
aacattgtgctgacccaatctccagcttctttggctgtgtctcgagggcagagggccaccatatcctgcagagccagtgaaagtgttgatagttatggccatagttttattcactggtaccagcagaaaccaggacagccacccaaaatcctcatctatcttgcatccaagttagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 521 - protein, 1262 aa, Artificial Sequence
  SARS-CoV-2 Spike Protein-T4 Fibritin-HRV3C-His8
MEWSWVFLFFLSVTTGVHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLLEVLFQGPHHHHHHHH

SEQ ID NO: 522 - protein, 1259 aa, Artificial Sequence
  SARS-CoV-2 Beta Variant Spike Protein- T4 Fibritin-HRV3C-His8
MEWSWVFLFFLSVTTGVHSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLLEVLFQGPHHHHHHHH

(19) United States
(12) Patent Application Publication
(10) Pub. No.: US 2023/0115257 A1
(43) Pub. Date: Apr. 13, 2023

(54) SARS-COV-2 SPIKE PROTEIN ANTIBODIES

(71) Applicant: CURIA IP HOLDINGS, LLC, Albany, NY (US)

(72) Inventors: Brian A. ZABEL, Redwood City, CA (US); Dan LUO, Newark, CA (US); Ling ZHANG, Menlo Park, CA (US); Vydehi KANNEGANTI, Hayward, CA (US); Joyce YU, Menlo Park, CA (US); Sophie YANG, Mountain View, CA (US); Lequn ZHAO, c/o Curia IP Holdings, LLC, CA (US); Hua TU, Flower Mound, TX (US); Xiaomei GE, Foster City, CA (US)

(21) Appl. No.: 17/746,859

(22) Filed: May 17, 2022

Related U.S. Application Data
(60) Provisional application No. 63/216,406, filed on Jun. 29, 2021.
(60) Provisional application No. 63/317,441, filed on Mar. 7, 2022.
(60) Provisional application No. 63/189,635, filed on May 17, 2021.

(51) Int. Cl. C07K 16/10; G01N 33/569

(52) U.S. Cl. CPC C07K 16/10; G01N 33/56983; C07K 2317/24; C07K 2317/76; C07K 2317/92; G01N 2333/165; G01N 2800/26

(57) ABSTRACT

Embodiments include monoclonal antibodies (mAbs) that recognize SARS-Cov-2 spike protein. The mAbs are capable of distinguishing among variants of the virus. The present disclosure also provides a composition and methods of making and using such a composition for treating, preventing, and/or detecting SARS-CoV-2 infection.

This application claims the priority benefit of U.S. Provisional Patent Application Serial Nos. 63/189,635, filed May 17, 2021; 63/216,406, filed Jun. 29, 2021; and 63/317,441, filed Mar. 7, 2022. Each of the above-listed priority applications is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 9, 2022, is named 147471_002100.txt and is 245,631 bytes in size.

### FIELD OF THE INVENTION

The invention relates to therapeutic antibodies, and more specifically, it relates to antibodies for treating, preventing, and/or detecting SARS-CoV-2 infection.

### BACKGROUND OF THE INVENTION

Coronaviruses (CoV) are a group of related RNA viruses that cause diseases in mammals and birds. More specifically, they cause respiratory tract infections that can range from mild to lethal. A CoV was described in 2003 that caused severe acute respiratory syndrome coronavirus (SARS-CoV). It was characterized by severe respiratory distress leading to mortality in 9.6% of individuals infected. By July of 2003, SARS-CoV was responsible for more than 774 deaths and 8,096 cases worldwide involving 29 countries. Since the conclusion of the SARS outbreak, several reports of confirmed cases of SARS of unknown origin indicate that the environmental threat of SARS-CoV still exists. SARS-CoV-like virus can be isolated from horseshoe bats in China, and researchers postulate that this is the natural reservoir for the virus (see, e.g., Li, W., et al. 2005. Bats are the natural reservoirs of SARS-like coronaviruses. Science. 310: 676-679). A virus similar to SARS was discovered in late 2019. This virus, named severe acute respiratory syndrome coronavirus 2 (SARS-CoV-2), is the causative pathogen of COVID-19, the propagation of which started the COVID-19 pandemic.

In late December 2019, SARS-CoV-2 emerged from Wuhan, China, and resulted in significant outbreaks in 216 countries, with over 159M reported cases and over 3.3 million deaths. The disease was officially named Coronavirus Disease—2019 (COVID-19, by WHO). COVID-19 is a potential zoonotic disease with a mortality rate estimated from 2%-5%. Currently, there is no effective treatment for COVID-19 although treatments are being investigated in clinical trials. Efforts have focused on contact tracing and mass vaccinations.

Several COVID-19 vaccines have been used in various countries with limited success. Vaccine hesitancy and breakthrough cases present the need for new therapeutics. Moreover, those awaiting vaccines and immunocompromised subjects may rely on therapeutics. Monoclonal antibodies (mAbs) have demonstrated success in patients under certain circumstances.

Monoclonal antibodies (mAbs) are laboratory-produced molecules that act as substitute antibodies that can restore, enhance or mimic the immune system's attack on cells. Monoclonal antibodies for COVID-19 may block the virus that causes COVID-19 from attaching to human cells, making it more difficult for the virus to reproduce and cause harm. Monoclonal antibodies may also neutralize a virus.

Currently, three anti-SARS-CoV-2 mAb products have received Emergency Use Authorizations (EUAs) from the Food and Drug Administration (FDA) for the treatment of mild to moderate COVID-19 in non-hospitalized patients with laboratory-confirmed SARS-CoV-2 infection who are at high risk for progressing to severe disease and/or hospitalization. These products include (1) Bamlanivimab plus etesevimab, (2) Casirivimab plus imdevima and (3) Sotrovimab. In 2021, the monoclonal antibody therapies bamlanivimab/etesevimab and casirivimab/imdevimab were found to reduce the number of hospitalizations, emergency room visits and deaths. Both combination drugs have emergency use authorization by the US Food and Drug Administration (FDA).

As with other viruses, SARS-CoV-2 changes through mutation which inevitably leads to new variants of the virus. Some of the new variants have been termed variants of concern (VOC) by the World Health Organization (WHO) and the Centers for Disease Control and Prevention (CDC) because they show increased transmissibility, increased virulence, or decreased effectiveness of vaccines and therapeutics. These include the Alpha (B.1.1.7), Beta (B.1.351), Gamma (P.1), Delta (B.1.617.2) and Epsilon (B.1.427/B.1.429) SARS-CoV-2 variants (formerly, the United Kingdom, South Africa, Brazil, India, and California variants, respectively). The Omicron (B.1.1.529) variant has also been labeled as a variant of concern. In addition to increased transmissibility, the B.1.351, P.1 and B.1.427/B.1.429 variants have demonstrated reduced susceptibility to a combination of two therapeutic monoclonal antibodies, bamlanivimab (LY-CoV555) and etesevimab (LY-CoV016).

As the COVID-19 pandemic progresses, various resistant strains have begun to spread through the population. In laboratory studies, some SARS-CoV-2 variants that h arbor certain mutations have markedly reduced susceptibility to anti-SARS-CoV-2 mAbs. The clinical relevance of reduced in vitro susceptibility of select variants to anti-SARS-CoV-2 mAbs is under investigation. As of December 2021, in vitro neutralization tests indicate that all available monoclonal antibody therapies (with the exception of sotrovimab and Tixagevimab/cilgavimab) are likely not active against the Omicron variant. Accordingly, new antibodies will be needed to effectively provide prophylactic and/or therapeutic value to patients. Even as more monoclonal therapeutic antibodies are authorized for emergency use, the number of SARS-CoV-2 variants that harbor mutations in the viral spike protein are increasing in incidence throughout the world, raising questions on the long-term efficacy of current vaccines and therapeutic antibodies.

With the rapid spread of SARS-CoV-2 variants, including those that are resistant to antibodies, it is apparent that new antibodies will be needed to effectively protect patients against more severe disease. The difference between the murine and human antibody repertoire may allow for the isolation of murine monoclonal antibodies that recognize a different or broader range of SARS-CoV-2 variants than the human antibodies that have been characterized so far.

Accordingly, there is a need for new antibodies to diagnose, prevent and/or treat coronaviruses infections. Embodiments of the invention include novel antibodies to fulfill these needs.

### SUMMARY OF THE INVENTION

In various embodiments, the present invention meets this need by providing new anti-CoV-S antibodies that can prevent, treat and/or detect SARS-CoV-2 infection. Embodiments also include methods of making and administering these antibodies to subjects in need thereof.

Embodiments include monoclonal antibodies obtained from mice using the PENTAMICE® platform, that recognize RBD in neutralization assays against wild-type SARS-CoV-2 virus and SARS-CoV-2 pseudovirus variants. The antibodies demonstrate excellent neutralizing potency against wild-type SARS-CoV-2 and several tested variants. Because of their broad specificity against new variants of SARS-CoV-2 virus, the antibodies are promising candidates for diagnostics and therapy.

In some embodiments, the present invention provides antigen binding domains, including antibodies, which bind to CoV-S, comprising the vhCDR1, vhCDR2, vhCDR3, vICDR1, vICDR2 and vICDR3 sequences from an antibody selected from the group consisting of clone IDs: 1-B11-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B111-A, 10-L24-A, 10-O3-A, 4-M3-A, 4-N22-A, 7-B10-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A (as provided in the SEQ ID's herein).

In some embodiments, the present invention provides anti-CoV-S antigen binding domains (including antibodies) comprising the variable heavy domain (VH) and variable light domain (VL) from an antibody selected from the group consisting of clone IDs: 1-B111-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B111-A, 10-L24-A, 10-O3-A, 4-M3-A, 4-N22-A, 7-B10-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A.

In some embodiments, the present invention provides anti-CoV-S antigen binding domains (including antibodies) selected from the group consisting of clone IDs: 1-B11-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B11-A, 10-L24-A, 10-O3-A, 4-M3-A, 4-N22-A, 7-B110-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A.

In some embodiments, the present invention provides an antigen binding domain (including antibodies) that competes with the antibodies or antigen-binding domains referenced above or herein for binding to CoV-S.

In some embodiments, the present invention provides a pharmaceutical composition and formulation comprising an isolated antibody, as discussed above or herein, and a pharmaceutically acceptable carrier or diluent.

In some embodiments, the present invention provides nucleic acid compositions comprising: a) a first nucleic acid encoding the heavy chain variable domain comprising the vhCDR1, vhCDR2 and vhCDR3 from an antibody; and b) a second nucleic acid encoding a light chain variable domain comprising vICDR1, vICDR2 and vICDR3 from an antibody selected from the group consisting of clone IDs: 1-B11-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B11-A, 10-L24-A, 10-O3-A, 4-M3-A, 4-N22-A, 7-B10-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A.

In some embodiments, the present invention provides nucleic acid compositions comprising: a) a first nucleic acid encoding the heavy chain variable domain (VH); and b) a second nucleic acid encoding a light chain variable domain (VL), wherein the heavy and light chain variable domains are from an antibody selected from the group consisting of clone IDs: 1-B11-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B111-A, 10-L24-A, 10-O3-A, 4-M3-A, 4-N22-A, 7-B110-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A.

In some embodiments, the present invention provides expression vectors comprising the first and/or second nucleic acids as outlined herein and above.

In some embodiments, the present invention provides host cells comprising the expression vector compositions, either as single expression vectors or two expression vectors.

In some embodiments, the present invention provides methods of making an anti-CoV-S antibody comprising a) culturing a host cell of the invention with expression vector(s) under conditions wherein the antibody is produced; and b) recovering the antibody.

In some embodiments, the present invention provides methods for treating SARS-CoV-2 infection comprising administering an antibody as provided herein to a patient in need.

In some embodiments, the present invention provides methods for preventing SARS-CoV-2 infection comprising administering an antibody as provided herein to a patient in need.

In some embodiments, the present invention provides methods for detecting SARS-CoV-2 in a human sample.

In some embodiments, the method for detecting includes contacting a human sample with an antibody provided herein, and detecting binding of the antibody to SARS-CoV-2 spike protein (CoV-S) as an indication of presence of SARS-CoV-2 in the sample.

### BRIEF DESCRIPTION OF THE DRAWINGS

The accompanying drawings illustrate aspects of the present invention. In such drawings:

FIG. 1 illustrates the primary amino acid sequence (SEQ ID NO:521) of a SARS-CoV-2 prefusion stabilized trimer protein immunogen that was derived from the SARS-CoV-2 of WIV02 isolate (see Genbank Reference No. MN996527.1, which is hereby incorporated by reference in its entirety). The fusion polypeptide includes an N-terminal signal sequence, SARS-CoV-2 spike protein bearing five substitutions (R691G, R692S, R694A, K995P, V996P), a T4 fibritin trimerization domain, followed by an HRV3C cleavage site, and a C-terminal His8 tag.

FIGS. 2A-2D are graphs showing EC₅₀ ELISA binding curves for selected SARS-CoV-2 spike-binding mAbs against spike trimer (FIG. 2A), S2 domain (FIG. 2B), RBD domain (FIG. 2C), and S1 domain (FIG. 2D).

FIGS. 3A-3D are graphs showing EC₅₀ ELISA binding curves for selected SARS-CoV-2 spike-binding mAbs against spike trimers from SARS-CoV-1 (FIG. 3A), HKU1 (FIG. 3B), HCOV—OC43 (FIG. 3C), and MERS (FIG. 3D).

FIG. 4 is a graph depicting IC₅₀ ELISA neutralization curves for selected SARS-CoV-2 spike-binding mAbs inhibiting the binding of SARS-CoV-2 spike trimer to huACE2.

FIG. 5A shows neutralization IC₅₀ titration of 3-E2-A in SARS-CoV-2 pseudovirus.

FIG. 5B shows neutralization IC₅₀ titration of 8-H3-A in SARS-CoV-2 pseudovirus.

FIG. 6 shows the binding kinetics for selected SARS-CoV-2 spike-binding mAbs against RBD, including 5-H22-A, SinoBio-40592-MM57, and 10-B11-A.

FIG. 7 is an illustrative summary of binding and function of multiple SARS-CoV-2 spike binding mAbs.

FIG. 8 is a SARS-CoV-2 spike binding mAb dendrogram.

FIG. 9A is a graph depicting EC₅₀ ELISA binding curves for selected SARS-CoV-2 spike-binding mAbs against SARS-CoV-2 Delta Spike.

FIG. 9B is a summary of the SARS-CoV-2 Delta Spike binding EC₅₀ values.

FIG. 10A is a graph depicting IC₅₀ ELISA neutralization curves for selected SARS-CoV-2 Delta spike-binding mAbs inhibiting the binding of SARS-CoV-2 spike trimer to huACE2.

FIG. 10B is a summary of the SARS-CoV-2 Delta Spike/huACE2 ELISA binding Neutralization IC₅₀ values.

FIG. 11A is a graph depicting the SARS-CoV-2 Delta Spike Pseudovirus Neutralization IC₅₀ of eight mAbs.

FIG. 11B is a summary of the SARS-CoV-2 Delta Spike Pseudovirus Neutralization IC₅₀ of eight mAbs.

FIG. 12 is a graph depicting authentic SARS-CoV-2 Delta variant neutralization IC₅₀ of two mAbs, 4-N22-A and 10-L24-A.

FIG. 13 illustrates the primary amino acid sequence (SEQ ID NO:522) of the SARS-CoV-2 Beta (R, South Africa, B.1.351) variant in the form of a prefusion stabilized trimer protein immunogen, which was used for generating spike-specific mAbs. The fusion polypeptide includes an N-terminal signal sequence, SARS-CoV-2 Beta spike protein bearing the indicated substitutions to prevent furin cleavage and to stabilize the prefusion confirmation (similar to WT spike in FIG. 1), a T4 fibritin trimerization domain, followed by an HRV3C cleavage site, and a C-terminal His8 tag.

FIG. 14 is a graphical depiction of a primary screen of 74 β spike-selective antibodies for binding with WT and Beta spike proteins.

FIG. 15 is a graphical depiction of a tertiary screen of 65 β spike-selective antibodies for binding with WT, Alpha, Beta, and Gamma spike proteins.

FIG. 16A is a graph depicting EC₅₀ binding of purified mAbs to R-spike for thirteen antibodies.

FIG. 16B is a summary of EC₅₀ binding of the thirteen antibodies.

FIG. 17 is a chart of assessed isotype of clonal hybridomas.

FIGS. 18A-18E is a summary of SARS-CoV-2 spike binding monoclonal antibodies. In FIGS. 18A-18E, the HC-CDR3 and LC-CDR3 amino acid sequences of RBD− binding antibodies are shown as follows: 9-L13-A (SEQ ID NOS: 313 and 316, respectively); 10-L24-A (SEQ ID NOS: 343 and 346, respectively); 2-O12-A (SEQ ID NOS: 213 and 216, respectively); 3-E2-A (SEQ ID NOS: 413 and 416, respectively); 4-K13-A (SEQ ID NOS: 253 and 256, respectively); 4-L4-A (SEQ ID NOS: 263 and 266, respectively); 5-H22-A (SEQ ID NOS: 273 and 276, respectively); 2-H7-A (SEQ ID NOS: 183 and 186, respectively); 8-H3-A (SEQ ID NOS: 483 and 486, respectively); 8-L17-A (SEQ ID NOS: 493 and 496, respectively); 7-B10-A (SEQ ID NOS: 383 and 386, respectively); 4-N22-A (SEQ ID NOS: 373 and 376, respectively); 4-M3-A (SEQ ID NOS: 363 and 366, respectively); 8-H5-A (SEQ ID NOS: 393 and 396, respectively); and 8-N24 (SEQ ID NOS: 293 and 296, respectively). In FIGS. 18A-18E, the HC-CDR3 and LC-CDR3 amino acid sequences of S1 non-RBD-binding antibodies are shown as follows: 10-O3-A (SEQ ID NOS: 353 and 356, respectively); 4-A15-A (SEQ ID NOS: 233 and 2, respectively); 4-K16-A (SEQ ID NOS: 423 and 426, respectively); 4-C3-A (SEQ ID NOS: 243 and 246, respectively); and 6-L8-A (SEQ ID NOS: 443 and 446, respectively). In FIGS. 18A-18E, the HC-CDR3 and LC-CDR3 amino acid sequences of S2-binding antibodies are shown as follows: 10-B11-A (SEQ ID NOS: 333 and 336, respectively); 2-P2-A (SEQ ID NOS: 213 and 216, respectively); 3-E13-A (SEQ ID NOS: 223 and 226, respectively); 6-C19-A-WT (SEQ ID NOS: 433 and 436, respectively); 2-J9-A (SEQ ID NOS: 193 and 196, respectively); 9-P9-A (SEQ ID NOS: 323 and 326, respectively); 1-B11-A (SEQ ID NOS: 163 and 166, respectively); and 10-I12-A (SEQ ID NOS: 513 and 516, respectively). In FIGS. 18A-18E, the HC-CDR3 and LC-CDR3 amino acid sequences of non-RBD, non-S1, and non-S2 binding antibodies are shown as follows: 6-O12-A (SEQ ID NOS: 283 and 286, respectively); 1-L10-A (SEQ ID NOS: 173 and 176, respectively); 2-G20-A (SEQ ID NOS: 413 and 416, respectively); 7-D7-A (SEQ ID NOS: 453 and 456, respectively); 8-A17-A (SEQ ID NOS: 473 and 476, respectively); and 9-F6-A (SEQ ID NOS: 503 and 506, respectively). In FIGA. 18A-18E, the HC-CDR3 and LC-CDR3 amino acid sequences of SARS-CoV-2 spike-selective antibodies are shown as follows: 7-N20-A (SEQ ID NOS: 463 and 466, respectively) and 9-J11-A (SEQ ID NOS: 303 and 306, respectively). FIGS. 18A-18E provide a summary of SARS-CoV-2 spike binding monoclonal antibody binding to SARS-CoV-2 Domains (FIG. 18A), binding to coronavirus spike proteins (FIG. 18B), virus neutralization (FIG. 18C), spike variant binding (FIG. 18D), and spike variant ELISA neutralization (FIG. 18E).

FIG. 19A is a graph depicting dose-dependent mAb binding to Omicron B.1.1.529 (8 point dose response). The binding domain is also identified (if known).

FIG. 19B shows EC₅₀ ELISA binding potency values in μg/ml and nM for Omicron B.1.1.529-binding mAbs (DNS: did not saturate, EC50 could not be calculated for these mAbs).

FIG. 20A is a graph depicting dose-dependent spike mAb binding to Omicron BA.2 (8 point dose response). The binding domain is also identified (if known).

FIG. 20B shows EC₅₀ ELISA binding potency values in g/ml and nM for Omicron BA.2-binding mAbs (NB: No binding).

FIG. 21A is a graph depicting IC₅₀ ELISA neutralization curves for Omicron-binding mAbs 4-N22-A, 8-H5-A, and 10-L24-A4-N22-A inhibiting the binding of SARS-CoV-2 Omicron B.1.1.529 spike trimer to huACE2. IC₅₀ potency values are listed in nanomolar (NI: no inhibition).

FIG. 21B shows IC₅₀ ELISA neutralizations curve for Omicron-binding mAbs 4-N22-A, 8-H5-A, and 10-L24-A4-N22-A inhibiting the binding of SARS-CoV-2 Omicron BA.2 spike trimer to huACE2. IC₅₀ potency values are listed in nanomolar.

### DETAILED DESCRIPTION OF THE INVENTION

### Definitions

In describing the present invention, the following terms will be employed, and are intended to be defined as indicated below. In order that the invention may be more completely understood, several definitions are set forth below. Such definitions are meant to encompass grammatical equivalents. Unless explained otherwise, all technical and scientific terms used herein have the same meaning as commonly understood by one of ordinary skill in the art to which this disclosure belongs.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, second ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “coronavirus” or “CoV” refers to any virus of the coronavirus family, including but not limited to SARS-CoV-2, MERS-CoV, and SARS-CoV-1. SARS-CoV-2 refers to the newly emerged coronavirus which was identified as the cause of a serious outbreak starting in Wuhan, China, and which has spread throughout the globe. SARS-CoV-2 has also been known as 2019-nCoV and Wuhan coronavirus. It binds via the viral spike protein to human host cell receptor angiotensin-converting enzyme 2 (ACE2). The spike protein also binds to and is cleaved by TMPRSS2, which activates the spike protein for membrane fusion of the virus.

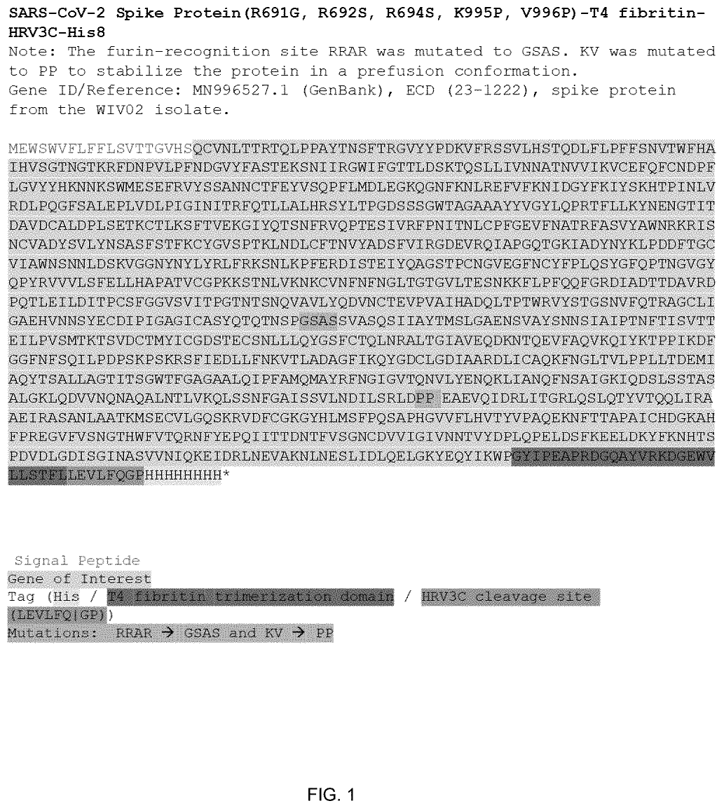

The term “CoV-S”, also called “S” or “S protein”, refers to the spike protein of SARS-CoV-2. The SARS-CoV-2-Spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 domain. The amino acid sequence of SARS-CoV-2 spike protein used in the present invention is exemplified by the amino acid sequence provided in FIG. 1. The term “CoV-S” includes protein variants of SARS-CoV-2 spike protein isolated from different CoV isolates, whether identified herein or arising later, as well as recombinant CoV spike protein or a fragment thereof. The term also encompasses CoV spike protein or a fragment thereof coupled to, for example, a histidine tag, T4 fibritin trimerization domain, mouse or human Fc, or a signal sequence.

The term “coronavirus infection”, “SARS-CoV-2 infection”, or “CoV infection” refers to infection with a coronavirus such as SARS-CoV-2. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

The term “monoclonal antibody therapy” refers to a form of immunotherapy that uses monoclonal antibodies (mAbs) to bind monospecifically to certain cells or proteins. The objective is that this treatment will stimulate the patient's immune system to attack those cells. Human monoclonal antibodies (suffix -umab) can be produced using transgenic mice or phage display libraries by transferring human immunoglobulin genes into the murine genome and vaccinating the transgenic mouse against the desired antigen, leading to the production of appropriate monoclonal antibodies. Murine antibodies in vitro are thereby transformed into fully human antibodies. The heavy and light chains of human IgG proteins are expressed in structural polymorphic (allotypic) forms. Human IgG allotype is one of the many factors that can contribute to immunogenicity.

The term “polypeptide” or “protein” encompasses native or artificial proteins, protein fragments and polypeptide analogs of a protein sequence. A polypeptide may be monomeric or polymeric.

The term “peptide” refers to a polymer in which the monomers are amino acids and are joined together through amide bonds, alternatively referred to as a peptide. Additionally, unnatural amino acids, for example, β-alanine, phenylglycine and homoarginine are also included. Amino acids that are not nucleic acid-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer thereof. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally-associated components by isolation, using protein purification techniques well known in the art. The lower end of the range of purity for the isolated polypeptides is about 60%, about 70% or about 80% and the upper end of the range of purity is about 70%, about 80%, about 90% or more than about 90%.

When the polypeptides are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity. An exemplary “isolated” polypeptide is a polypeptide that is at least about 95%, 98%, 99% or 99.5% pure.

Purity can be determined by any art-recognized method of analysis (e.g., band intensity on a silver-stained gel, polyacrylamide gel electrophoresis, HPLC, or a similar means).

The term “immunoglobulin (Ig)” refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (β2M), CD3, CD4 and CD8. Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, β, γ, and μ. The type of heavy chain present defines the class of antibody, i.e., these chains are found in IgA, IgD, IgE, IgG, and IgM antibodies, respectively, each performing different roles, and directing the appropriate immune response against different types of antigens. Distinct heavy chains differ in size and composition; α and γ and comprise approximately 450 amino acids, while β has approximately 550 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (about 66%), followed by IgG2 (about 23%), IgG3 (about 7%) and IgG4 (about 4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437).

Antibodies are not only found as monomers but are also known to form dimers of two Ig units (e.g., IgA), tetramers of four Ig units (e.g., IgM of teleost fish), or pentamers of five Ig units (e.g., mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel β-strands arranged in two β-sheets. Typically, the “heavy chain” of an antibody comprises four Ig domains with three of them being constant (CH domains: CH1, CH2, CH3) domains and one of them being a variable domain (V), with the exception of IgM and IgE which contain one variable (VH) and four constant regions (CH1, CH2, CH3, CH4). The additional domain (CH2: Cμ2, C∈2) in the heavy chains of IgM and IgE molecules connects the two heavy chains instead of the hinge region contained in other Ig molecules (Perkins et al., (1991) J Mol Biol. 221(4):1345-66; Beavil et al., (1995) Biochem 34(44):14449-61; Wan et al., (2002) Nat Immunol. 3(7):681-6). The “light chain” typically comprises one constant Ig domain (CL) and one variable Ig domain (VL). Exemplified, the human IgM heavy chain is composed of four Ig domains linked from N- to C-terminus in the order VH—CH1-CH2-CH3-CH4 (also referred to as VH—Cμ1-Cμ2-Cμ3-Cμ4), whereas the human IgM light chain is composed of two immunoglobulin domains linked from N- to C-terminus in the order VL-CL, being either of the kappa or lambda type (Vκ-Cκ or Vλ-Cλ).

Exemplified, the constant chain of human IgM comprises 452 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5th ed. U.S. Department of Health and Human Service, National Institutes of Health, Bethesda, Md. The “EU index as in Kabat” refers to the residue numbering of the human IgM EU antibody. Accordingly, CH domains in the context of IgM are as follows: “CH1” refers to amino acid positions 118-215 according to the EU index as in Kabat; “CH2” refers to amino acid positions 231-340 according to the EU index as in Kabat; “CH3” refers to amino acid positions 341-446 according to the EU index as in Kabat. “CH4” refers to amino acid positions 447-558 according to the OU index as in Kabat.

Whilst in human IgA, IgG, and IgD molecules two heavy chains are connected via their hinge region, IgE and IgM antibodies do not comprise such hinge region. Instead, IgE and IgM antibodies possess an additional Ig domain, their CH2 domain, which functions as dimerization domain between two heavy chains. In contrast to rather flexible and linear hinge regions of other antibodies, the CH2 domain of IgE and IgM are composed of two beta sheets stabilized by an intradomain disulfide bond forming a c-type immunoglobulin fold (Bork et al., (1994) J Mol Biol. 242(4):309-20; Wan et al., (2002) Nat Immunol. 3(7):681-6). Furthermore, the MHD2 and EHD2 domains contain one N-glycosylation site.

The “IgM heavy chain domain 2” (“MHD2”) consists of 111 amino acid residues 12.2 kDa) forming a homodimer covalently held together by a disulfide bond formed between cysteine residue 337 of two domains (Davis et al., (1989) EMBO J 8(9):2519-26; Davis & Shulman, (1989) Immunol Today. 10(4):118-22; 127-8). The domain is further stabilized by an intradomain disulfide bond formed between Cys261 and Cys321. Typically, two MHD2 domains are covalently linked by an interdomain disulfide bond between Cys337. The MHD2 contains an N-glycosylation site at Asn333.

“Fc” or “Fc region” or “Fc domain” as used herein refers to the polypeptide comprising the constant region of an antibody excluding the first constant region immunoglobulin domain and, in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may or may not include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, as is more fully described below, amino acid modifications are made to the Fc region, for example to alter binding to one or more FcγR receptors or to the FcRn receptor.

As used herein, the term “human antibody” means any antibody in which the variable and constant domain sequences are human sequences. The term encompasses antibodies acquired from and/or enriched from a human sourced starting material, e.g., plasma from a recovered donor infected with SARS-CoV-2.

A “neutralizing antibody”, an antibody with “neutralizing activity”, “antagonistic antibody”, or “inhibitory antibody”, as used herein, means an antibody capable of preventing, retarding or diminishing replication of the viral target of the antibody. In some embodiments, neutralizing antibodies are effective at antibody concentrations of <0.2 μg/mL. In some embodiments, neutralizing antibodies are effective at antibody concentrations of <0.1 μg/mL. An exemplary neutralizing antibody “neutralizes” a virus (e.g., SARS-CoV-2) if it partly or fully impedes the virus' ability to infect a cell that, absent the antibody, it would otherwise infect, or if it prevents viral replication within an infected cell. An exemplary neutralizing antibody is one that neutralizes 200 times the tissue culture infectious dose required to infect 50% of cells (200×TCID50) in the presence of the SARS-CoV-2. In some embodiments, neutralizing antibodies are effective at antibody concentrations of <12.5 μg/mL, <3.125 μg/mL, or <0.8 μg/mL. One measure for assessing the neutralization capacity of an antibody (or antigen-binding portion thereof) for inhibiting the ability of a pseudovirus or virus to infect cells involves a dose-response evaluation, which allows for the determination of the concentration of antibody (or antigen-binding portion thereof) required to neutralize 50% of infection (IC₅₀). IC₅₀ values can be calculated using the methods described in the accompanying Examples.

The term “TCID50” refers to the amount of virus necessary to infect 50% of cells in tissue culture. 100× and 200× refer to 100 or 200 times the concentration of virus compared to the TCID50. In a TCID50 assay, serial dilutions of a virus are added onto monolayers of cells, and left until a cytopathic effect can be seen. From the resulting dose-response curve, it is possible to determine the accurate TC₅₀ values.

The term “KD” refers to the equilibrium dissociation constant of a particular protein-ligand interaction. K_{D} values can be calculated using the methods described in the accompanying Examples.

The term “enzyme-linked immunosorbent assay” or “ELISA” refers to an assay that uses a solid-phase type of enzyme immunoassay (EIA) to detect the presence of a ligand (commonly a protein) in a liquid sample using antibodies directed against the protein to be measured. For example, antigens from a sample to be tested are attached to a surface of a substrate. Then, a matching antibody is applied over the surface so it can bind the antigen. This antibody is linked to an enzyme and then any unbound antibodies are removed. In the final step, a substance containing the enzyme's substrate is added. If there was binding, the subsequent reaction produces a detectable signal, typically a color change. Because the ELISA can be performed to evaluate either the presence of antigen or the presence of antibody in a sample, it is a useful tool for determining serum antibody concentrations. There are variations of ELISA tests based on how the analytes and antibodies are bonded and used which include Direct ELISA, Sandwich ELISA, Competitive ELISA and Reverse ELISA.

The term “epitope” includes any protein determinant capable of specific binding to an immunoglobulin or T-cell receptor or otherwise interacting with a molecule. Epitopic determinants generally consist of chemically-active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. An antibody is said to specifically bind an antigen when the dissociation constant is ≤1 mM, preferably ≤100 nM and most preferably ≤10 nM. In certain embodiments, the KD is from about 1 pM to about 500 pM. In some embodiments, the KD is from about 500 pM to about 1 μM. In some embodiments, the KD is from about 1 μM to about 100 nM. In some embodiments, the KD is from about 100 mM to about 10 nM. It is possible to competitively screen antibodies for binding to the same epitope. An approach to achieve this is to conduct cross-competition studies to find antibodies that competitively bind with one another, e.g., the antibodies compete for binding to the antigen. A high throughput process for “binding” antibodies based upon their cross-competition is described in International Patent Application No. WO 03/48731.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. In general terms, the hydrogen/deuterium exchange method involves deuterium-labeling the protein of interest, followed by binding the antigen-binding protein, e.g., antibody or fragment or polypeptide, to the deuterium-labeled protein. Next, the CoV-S protein/antigen-binding protein complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antigen-binding protein interface may retain deuterium and therefore exhibit relatively higher mass compared to amino acids not included in the interface. After dissociation of the antigen-binding protein (e.g., antibody or fragment or polypeptide), the target protein is subjected to protease cleavage and mass spectrometry analysis, thereby revealing the deuterium-labeled residues which correspond to the specific amino acids with which the antigen-binding protein interacts. See, e.g., Ehring (1999) Analytical Biochemistry 267: 252-259; Engen and Smith (2001) Anal. Chem. 73: 256A-265A.

The term “PENTAMICE® platform” refers to an antibody generation platform that uses a set of mice comprising five wild type (WT) strains that cover nine distinct MHC haplotypes. A total of ten mice (i.e., two mice of each strain) are included in each set to achieve maximum plasma titers, thus boosting the opportunity to generate high-quality antibodies in vivo. Conventional immunization approaches typically utilized in hybridoma-based antibody discovery campaigns use one or two common wildtype (WT) mouse strains (e.g., Balb/c or C57Bl/6). The PENTAMICE© platform achieves maximum plasma titers, thus boosting the opportunity to generate high-quality antibodies in vivo.

As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments thereof. A binding molecule, e.g., an antibody or antibody-like molecule, comprising two or more, e.g., two, five, or six binding units, is referred to herein as “multimeric.”

The term “fusion protein” or “fused protein”, as used interchangeably herein, refers to a protein coded by a single gene and the single gene is made up of coding sequences that originally coded for at least two or more separate proteins. A fusion protein may retain the one or more functional domains of the two or more separate proteins. Part of the coding sequence for a fusion protein may code for an epitope tag. In certain embodiments, antibodies, or antigen binding portions thereof, may be present within a fusion protein.

A “disease” is a state of health of a subject wherein the subject cannot maintain homeostasis, and wherein if the disease is not ameliorated then the subject's health continues to deteriorate. An exemplary disease is infection by SARS-CoV-2 (COVID) or a symptom caused by such infection.

The term “pharmaceutically acceptable carrier” includes solvents, dispersion media, coatings, antibacterial and antifungal agents, isotonic and absorption delayin g agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, amino acids (e.g., glycine, proline, etc.), or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Compositions comprising such carriers are formulated by well-known conventional methods. Exemplary formulations of the invention include one, two, or more, different amino acids. In an exemplary embodiment, the presence of the amino acid(s) improves the stability of the antibodies, even at high concentrations at which the antibody is typically not stable in formulations absent the amino acid(s). In various embodiments, the carrier is selected to provide a “stable pharmaceutical formulation.”

The term “stable formulation” such as “stable pharmaceutical formulation” as used in connection with the formulations described herein denotes, without limitation, a formulation, which preserves its physical stability/identity/integrity and/or chemical stability/identity/integrity and/or biological activity/identity/integrity during manufacturing, storage and administration. Various analytical techniques for evaluating protein stability are available in the art and reviewed in Reubsaet, et al. (1998) J Pharm Biomed Anal 17(6-7): 955-78 and Wang, W. (1999) Int J Pharm 185(2): 129-88. Stability can be evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. The stability may be determined by, for example, at least one of the methods selected from the group consisting of visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, and kappa/lambda ELISA, without limitation. Exemplary characteristics of use with visual inspection include turbidity and aggregate formation.

In an embodiment, a formulation is considered stable when the protein in the formulation (1) retains its physical stability, (2) retains its chemical stability and/or (3) retains its biological activity.

A protein may be said to “retain its physical stability” in a formulation if, for example, without limitation, it shows no signs of aggregation, precipitation and/or denaturation upon visual examination of color and/or clarity, or as measured by UV light scattering or by size exclusion chromatography (SEC) or electrophoresis, such as with reference to turbidity or aggregate formation.

A protein may be said to “retain its chemical stability” in a formulation, if, for example, without limitation, the chemical stability at a given time is such that there is no significant modification of the protein by bond formation or cleavage resulting in a new chemical entity. In a further embodiment, chemical stability can be assessed by detecting and quantifying chemically altered forms of the protein. Chemical alteration may involve, example, without limitation, size modification (e.g., clipping) which can be evaluated using size exclusion chromatography, SDS-PAGE and/or matrix-assisted laser desorption ionization/time-of-flight mass spectrometry (MALDI/TOF MS). Other types of chemical alteration include, for example, without limitation, charge alteration (e.g. occurring as a result of deamidation), which can be evaluated by ion-exchange chromatography, for example. Oxidation is another commonly seen chemical modification.

In an embodiment, a protein may be said to “retain its biological activity” relative to native unmodified protein in a pharmaceutical formulation, if, for example, without limitation, the biological activity of the protein, at a given time is from about 50% to about 200%, or alternatively from about 60% to about 170%, or alternatively from about 70% to about 150%, or alternatively from about 80% to about 125%, or alternatively from about 90% to about 110%, of the biological activity exhibited at the time the formulation was prepared as determined, e.g., in an antigen binding assay or virus neutralization assay. In a further embodiment, a protein may be said to “retain its biological activity” in a pharmaceutical formulation, if, for example, without limitation, the biological activity of the protein, at a given time is at least 1%, 5%, 10%, 15%, 20%, 25%, 30%, 35%, 40%, 45%, 50%, 55%, 60%, 65%, 70%, 75%, 80%, 85%, 90%, 95% or 100%.

In one embodiment, a stable pharmaceutical formulation contains one or more proteins and at least one amino acid selected based on the amino acid's ability to increase the stability of the protein and/or reduce solution viscosity. In one embodiment, the amino acid contains a positively charged side chain, such as R, H, and K. In some embodiments, the amino acid contains a negatively charged side chain, such as D and E. In some embodiments, the amino acid contains a hydrophobic side chain, such as A, F, I, L, M, V, W, and Y. In some embodiments, the amino acid contains a polar uncharged side chain, such as S, T, N, and Q. In some embodiments, the amino acid does not have a side chain, i.e., G.

In one embodiment, the amino acid is any one of A, N, D, Q, E, I, L, K, F, P, S, T, W, Y, or V.

As used herein, the term “amino acid” refers to either natural and/or unnatural or synthetic amino acids.

“Linker”, or grammatical equivalents thereof, as used herein, means a linker joining two or more amino acids, or two or more peptides together. As is more fully described below, generally, there are a number of suitable linkers that can be used, including traditional peptides, produced by chemical synthetic methods or generated by recombinant techniques.

“Modified” or “modification”, as used herein, means an amino acid substitution, insertion, and/or deletion in a polypeptide sequence or an alteration to a moiety chemically linked to a polypeptide. For example, a modification may be an altered carbohydrate or PEG structure attached to a polypeptide. For clarity, unless otherwise noted, the amino acid modification is always applied to an amino acid coded by DNA, e.g., the 20 amino acids that have codons in DNA and RNA.

“Conservative substitutions” will produce molecules having functional and chemical characteristics similar to those of the molecule from which such modifications are made. For example, a “conservative amino acid substitution” may involve a substitution of an amino acid residue with another residue such that there is little or no effect on the polarity or charge of the amino acid residue at that position. Desired amino acid substitutions (whether conservative or non-conservative) can be determined by those skilled in the art. For example, amino acid substitutions can be used to identify important residues of the molecule sequence, or to increase or decrease the affinity of the molecules described herein. Variants comprising one or more conservative amino acid substitutions can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

“Amino acid insertion” or “insertion”, as used herein, means the addition of an amino acid sequence at a particular position in a parent polypeptide sequence.

“Amino acid deletion” or “deletion”, as used herein, means the removal of an amino acid sequence at a particular position in a parent polypeptide sequence.

“Fused”, as used herein, means the components (e.g., a polypeptide and a tag) are linked by covalent bonds, either directly or indirectly via linkers.

The polypeptides of the present invention are generally recombinant. “Recombinant” means the polypeptides are generated using recombinant nucleic acid techniques in exogenous host cells.

“Specific binding” or “specifically binds to”, as used herein, means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

The term “immunoassay” refers to a biochemical test that measures the presence or concentration of a substance in a sample, such as a biological sample. It is common to use the reaction of an antibody to its cognate antigen, for example the specific binding of an antibody to a protein. Both the presence of antigen and the amount of antigen present can be measured. The presence and amount (i.e., abundance) of the protein can determined or measured. Measuring the quantity of antigen (such as a biomarker) can be achieved by a variety of methods. A common method is to label either the antigen or antibody with a detectable label (e.g., a fluorescent tag, enzymatic linkage or radioactive isotope).

The term “lateral flow assay,” “lateral flow immunoassay” or “LFA” refers to a diagnostic device used to confirm the presence or absence of a target analyte. LFA-based tests often use a paper-based platform for the detection and quantification of analytes, where the sample is placed on a test device and the results are displayed within 5-30 minutes. LFA-based tests are widely used in hospitals and clinical laboratories for the qualitative and quantitative detection of specific antigens and antibodies, as well as products of gene amplification. The principle behind the LFA is relatively simple. A liquid sample (or its extract) containing the analyte of interest moves via capillary action (i.e., without the assistance of external forces) through various zones of polymeric strips, on which molecules that can interact with the analyte are attached. A typical lateral flow test strip has overlapping membranes that are mounted on a backing card.

The term “target analyte” or “analyte” refers to a molecule, compound or particle to be detected. Target analytes bind to binding ligands (both capture and soluble binding ligands). In some embodiments, the target analyte is a virus, such as SARS-CoV-2 as described herein.

The term “substrate” or “solid support” refers to a material that can be modified to contain discrete individual sites appropriate for the attachment or association of capture ligands. Suitable substrates include metal surfaces such as gold, electrodes, glass (including modified or functionalized glass), fiberglass, resins, silica or silica-based materials, carbon, metals, inorganic glasses and other polymers.

The term “up-converting phosphor” or “upconverting materials” refers to compounds that emit light at a wavelength that is shorter than the wavelength of light they have been photoexcited which give them applications in biomedical imaging. The so-called anti-Stokes shift in these materials limits the autofluorescence of nearby molecules within a sample. Compared with gold nanoparticles, a lateral flow test using upconverting phosphor nanoparticles (UCNPs) is more sensitive (approximately tenfold) and robust, due to the unique feature of using the lower energy 980 nm infrared light (excitation light) to generate higher energy visual light (emission light). This light process is called “upconversion,” which does not occur in nature. Thus, UCNPs as a reporter label do not generate background fluorescence (autofluorescence) compared with conventional fluorescent labels, such as fluorescently labeled nanoparticles and quantum dots. Moreover, UCNPs do not fade, allowing the lateral flow strips based on UCNPs to be stored in the long term.

In some embodiments, reduced expression of the target polynucleotide sequence is observed. The terms “decrease,” “reduced,” “reduction,” and “decrease” are all used herein generally to mean a decrease by a statistically significant amount. However, for avoidance of doubt, “decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease from about 10-100% as compared to a reference level.

The terms “increased”, “increase” or “enhance” or “activate” are all used herein to generally mean an increase by a statically significant amount; for the avoidance of any doubt, the term “increased”, “increase” or “enhance” or “activate” means an increase of at least 10% as compared to a reference level, for example an increase of at least about 10%, or at least about 20%, or at least about 30%, or at least about 40%, or at least about 50%, or at least about 60%, or at least about 70%, or at least about 80%, or at least about 90% or up to and including a 100% increase or any increase from about 10-100% as compared to a reference level, or at least about a 2-fold, or at least about a 3-fold, or at least about a 4-fold, or at least about a 5-fold or at least about a 10-fold increase, or any increase from about 2-fold to about 10-fold or greater as compared to a reference level.

The terms “inactivate” and “inactivation” are used herein to generally mean that the expression of a gene of interest is reduced as compared to a reference level or not expressed in a functional or active protein form. The terms “partially inactivate” and “partial inactivation” refer to an expression of the gene of interest that is reduced but not eliminated as compared to a reference level, or that a percentage of the proteins expressed by the gene still retain their activity and function. The terms “fully inactivate” and “full inactivation” as used herein mean that the gene of interest does not express any protein, or all of the expressed proteins encoded by the gene of interest are inactive and nonfunctional.

The terms “inhibitors,” “activators,” and “modulators” refer to agents that affect a function or expression of a biologically-relevant molecule. The term “modulator” includes both inhibitors and activators. They may be identified using in vitro and in vivo assays for expression or activity of a target molecule. In some cases, “inhibitors” are agents that, e.g., inhibit expression or bind to target molecules or proteins. They may partially or totally block stimulation or have protease inhibitor activity. They may reduce, decrease, prevent, or delay activation, including inactivation, desensitization, or down regulation of the activity of the described target protein. Modulators may be antagonists or agonists of the target molecule or protein. In some cases, “activators” are agents that, e.g., induce or activate the function or expression of a target molecule or protein. They may bind to, stimulate, increase, open, activate, or facilitate the target molecule activity. Activators may be agonists of the target molecule or protein.

The terms “subject”, “host”, and “individual” are used interchangeably herein, and refer to an animal, for example, a human from whom cells can be obtained and/or to whom treatment, including prophylactic treatment, is provided. For treatment of those infections, conditions or disease states which are specific for a specific animal such as a human subject, the term subject refers to that specific animal. The terms “non-human animals” and “non-human mammals” as used interchangeably herein, include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

“Percent (%) amino acid sequence identity” or “amino acid sequence with percent (%) identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. The degree of identity between an amino acid sequence of the present invention (“invention sequence”) and the parental amino acid sequence is calculated as the number of exact matches in an alignment of the two sequences, divided by the length of the “invention sequence”, or the length of the parental sequence, whichever is the shortest. The result is expressed in percent identity.

The term “vaccination” or “vaccinate” means administration of a vaccine that can elicit an immune response or confer immunity from a disease.

A “protein tag” or “tag” refers to an amino acid sequence within a recombinant protein that provides new characteristics to the recombinant protein that assist in protein purification, identification, or activity based on the tag's characteristics and affinity. A protein tag may provide a novel enzymatic property to the recombinant protein such as a biotin tag, or a tag may provide a means of protein identification such as with fluorescence tags encoding for green fluorescent protein or red fluorescent protein. Protein tags may be added onto the N- or C-terminus of a protein. A common protein tag used in protein purification is a poly-His tag where a series of approximately six histidine amino acid residues are added which enables the protein to bind to protein purification matrices chelated to metal ions such as nickel or cobalt. Other tags commonly used in protein purification include chitin binding protein, maltose binding protein, glutathione-S-transferase, Myc tag, and FLAG-tag. Tags such as “epitope tags” may also confer the protein to have an affinity towards an antibody. Common antibody epitope tags include the V5-tag, Myc-tag, and HA-tag.

The term “J-chain” refers to an acidic 15-kDa polypeptide, which is associated with pentameric IgM and dimeric IgA via disulfide bonds involving the penultimate cysteine residue in the 18-amino acid secretory tail-piece (tp) at the C-terminus of the IgM β or IgA a heavy chain. The three disulfide bridges are formed between Cys 12 and 100, Cys 71 and 91, and Cys 108 and 133, respectively. See, e.g. Frutiger et al. 1992, Biochemistry 31, 12643-12647. Structural requirements for incorporation of the J-chain into human IgM and IgA and for polymeric immunoglobulin assembly and association with the J-chain are reported by Sorensen et al. 2000, Int. Immunol. 12(1): 19-27 and Yoo et al. 1999, 1 Biol. Chem. 274(47):33771-33777, respectively. Recombinant production of soluble J-chain in E coli is reported by Redwan et al. 2006, Human Antibodies 15:95-102.

The term “adjuvant” refers to agents that augment, stimulate, activate, potentiate, or modulate the immune response to the active ingredient of the composition at either the cellular or humoral level, e.g. immunologic adjuvants stimulate the response of the immune system to the actual antigen, but have no immunological effect themselves. Adjuvants are used to accomplish three objectives: (1) they slow the release of antigens from the injection site; (2) they stimulate the immune system; and (3) the addition of an adjuvant may permit the use of a smaller dose of antigen to stimulate a similar immune response, thereby reducing the production cost of the vaccine. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-1p, IL-2, IL-7, IL-12, IL-18, GM-CFS, and INF-y) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g. polyarginine or polylysine).

The term “Cytotoxic T lymphocyte” (CTL) refers to a T lymphocyte that expresses CD8 on the surface thereof (i.e., a CD8+ T cell). In some embodiments such cells are preferably “memory” T cells (TM cells) that are antigen-experienced.

The term “Central memory” T cell (or “TCM”) refers to an antigen experienced CTL that expresses CD62L or CCR7 and CD45RO on the surface thereof, and does not express or has decreased expression of CD45RA as compared to naive cells. In embodiments, central memory cells are positive for expression of CD62L, CCR7, CD28, CD127, CD45RO, and CD95, and have decreased expression of CD54RA as compared to naive cells.

The term “Effector memory” T cell (or “TEM”) refers to an antigen experienced T cell that does not express or has decreased expression of CD62L on the surface thereof as compared to central memory cells, and does not express or has decreased expression of CD45RA as compared to naive cells. In some embodiments, effector memory cells are negative for expression of CD62L and CCR7, compared to naive cells or central memory cells, and have variable expression of CD28 and CD45RA.

The term “Naive” T cell refers to a non antigen experienced T lymphocyte that expresses CD62L and CD45RA, and does not express CD45RO— as compared to central or effector memory cells. In some embodiments, naive CD8+T lymphocytes are characterized by the expression of phenotypic markers of naive T cells including CD62L, CCR7, CD28, CD127, and CD45RA.

The term “Effector” or “TE” T cells refers to antigen experienced cytotoxic T lymphocyte cells that do not express or have decreased expression of CD62L, CCR7, CD28, and are positive for granzyme B and perforin as compared to central memory or naive T cells.

The term “administering” or “administered” means, intravenous, intranasal, intraperitoneal, intramuscular, intralesional, or subcutaneous administration, intrathecal administration, or instillation into a surgically created pouch or surgically placed catheter or device to the subject.

The term “prevent,” “preventing,” or “prevention” refers to a prophylactic treatment of a subject who is not and was not previously infected with a disease but is at risk of developing the disease or who was previously infected with a disease, and is at risk of regression of the disease. In certain embodiments, the subject is at a higher risk of developing the disease or at a higher risk of regression of the disease than an average healthy member of a population of subjects. Alternatively, when prevention is not possible, therapeutic intervention for inhibiting progression of the disease state (COVID) is contemplated (see “treating” infra).

The terms “condition,” “disease,” and “disorder” are used interchangeably.

The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The unit dosage forms may be administered once or multiple unit dosages may be administered, for example, throughout an organ, or solid tumor.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “reducing the likelihood” of a human subject becoming symptomatic of a SARS-CoV-2 infection includes, without limitation, reducing such likelihood by at least 10%, at least 20%, at least 30%, at least 40%, at least 50%, at least 60%, at least 70%, at least 80%, at least 90%, or at least 95%. In various embodiments, these percentages are relevant to the likelihood of infection in a similar subject having had or likely to have similar exposure as the subject to whom the prophylactically effective amount of a pharmaceutical formulation of the invention is administered. Preferably, reducing the likelihood of a human subject becoming symptomatic of a SARS-CoV-2 infection means preventing the subject from becoming symptomatic of a SARS-CoV-2 infection.

In an exemplary embodiment, the subject administered a prophylactically effective amount of the pharmaceutical formulation of the invention is at risk of being exposed to SARS-CoV-2. As used herein, an event wherein a subject is “at risk of being exposed” to SARS-CoV-2 includes, without limitation, an event wherein the subject may come into close contact with aerosols derived from tissue or secretions (e.g., the mucous membrane secretions) of infected animals, including infected human subjects.

In an exemplary embodiment, the subject has or may have recently been exposed to SARS-CoV-2. As used herein, a subject who “has or may have recently been exposed to” SARS-CoV-2 includes, for example, a subject who experienced a high risk event (e.g., one in which he/she may have come into close contact with tissue or aerosols derived from the tissue of infected animals, including infected human subjects) within the past month, three weeks, two weeks, one week, five days, four days, three days, two days or 24 hours.

As used herein, a human subject is “symptomatic” of a SARS-CoV-2 infection if the subject shows one or more symptoms known to appear in a SARS-CoV-2-infected human subject after a suitable incubation period. Such symptoms include, without limitation, detectable SARS-CoV-2 in the subject, and those symptoms shown by patients afflicted with SARS-CoV-2. SARS-CoV-2-related symptoms include, without limitation, respiratory distress, hypoxia, difficulty breathing (dyspnea), cardiovascular collapse, arrhythmia (e.g., atrial fibrillation, tachycardia, bradycardia), fatigue, altered mental status (including confusion), cough, fever, chills, abnormal blood coagulation events, myalgia, loss of smell and/or taste, loss of appetite, nausea, red/watery eyes, dizziness, stomach-ache, rash, sneezing, sputum/phlegm, and runny nose.

As used herein, “treating” a subject infected with SARS-CoV-2 and symptomatic of that infection includes, (i) slowing, stopping or reversing the progression of one or more of the symptoms, (ii) slowing, stopping or reversing the progression of illness underlying such symptoms, (iii) reducing or eliminating the likelihood of the symptom's recurrence, and/or (iv) slowing the progression of, lowering or eliminating the infection. In one exemplary embodiment, treating a subject infected with SARS-CoV-2 and symptomatic of that infection includes (i) reversing the progression of one or more of the symptoms, (ii) reversing the progression of illness underlying such symptoms, (iii) preventing the recurrence of a symptom or symptoms, and/or (iv) eliminating the infection. The progress of treating a subject infected with SARS-CoV-2 and symptomatic of that infection can be measured according to a number of clinical endpoints. These include lower or negative viral titer (also known as viral load) and the amelioration or elimination of one or more SARS-CoV-2 symptoms. In addition, “treating” may result in regression or elimination or inhibiting the need for supplemental oxygen, the need for mechanical breathing assistance, or any other COVID-19 symptom that requires the patient to be hospitalized. Symptoms that may require hospitalization include a number of more severe SARS-CoV-2-related symptoms defined above. In various embodiments, the invention provides for treatment of subjects who are infected with SARS-CoV-2 and have no limiting symptoms from this infection.

In an exemplary embodiment, treating reduces the risk of mortality of the subject. In some embodiments, treatment results in shortened time of recovery.

In one embodiment, the progress of treating a subject infected with SARS-CoV-2 and symptomatic of that infection can be measured by using RNA PCR to test for lower or negative viral titer in total lung tissue and/or sputum.

In exemplary embodiments, treatment results in one or more desirable clinical results including reduction of risk of mortality, and/or shortened time to recovery from an active SARS-CoV-2 infection.

The term “nucleic acid” includes RNA or DNA molecules having more than one nucleotide in any form including single-stranded, double-stranded, oligonucleotide or polynucleotide.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. The term “vector” also includes both viral and nonviral means for introducing a nucleic acid molecule into a cell in vitro, in vivo, or ex vivo. Vectors may be introduced into the desired host cells by well-known methods, including, but not limited to, transfection, transduction, cell fusion, and lipofection. Vectors can comprise various regulatory elements including promoters.

Reference will now be made in detail to implementation of exemplary embodiments of the present disclosure. Those of ordinary skill in the art will understand that the following detailed description is illustrative only and it is not intended to be in any way limiting. The embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming but would nevertheless be a routine undertaking of engineering for those of ordinary skill in the art having the benefit of this disclosure.

Many modifications and variations of the exemplary embodiments set forth in this disclosure are made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

### Virus

The present invention includes methods for treating or preventing a viral infection in a subject. The term “virus” includes any virus whose infection in the body of a subject is treatable or preventable by administration of an anti-CoV-S antibody or antigen-binding fragment thereof (e.g., wherein infectivity of the virus is at least partially dependent on CoV-S). In an embodiment of the invention, a “virus” is any virus that expresses spike protein (e.g., CoV-S). The term “virus” also includes a CoV-S-dependent respiratory virus which is a virus that infects the respiratory tissue of a subject (e.g., upper and/or lower respiratory tract, trachea, bronchi, lungs) and is treatable or preventable by administration of an anti-CoV-S antibody or antigen-binding fragment thereof. For example, in an embodiment of the invention, virus includes coronavirus, SARS-CoV-2 (severe acute respiratory syndrome coronavirus 2), SARS-CoV-1 (severe acute respiratory syndrome coronavirus 1), and MERS-CoV (Middle East respiratory syndrome (MERS) coronavirus). Coronaviruses can include the genera of alphacoronaviruses, betacoronaviruses, gammacoronaviruses, and deltacoronaviruses. In some embodiments, the antibodies or antigen-binding fragments provided herein can bind to and/or neutralize an alphacoronavirus, a betacoronavirus, a gammacoronavirus, and/or a deltacoronavirus. In certain embodiments, this binding and/or neutralization can be specific for a particular genus of coronavirus or for a particular subgroup of a genus. “Viral infection” refers to the invasion and multiplication of a virus in the body of a subject.

Coronavirus virions are spherical with diameters of approximately 125 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. SARS-CoV-2, MERS-CoV, and SARS-CoV-1 belong to the coronavirus family. The initial attachment of the virion to the host cell is initiated by interactions between the S protein and its receptor. The sites of receptor binding domains (RBD) within the S1 domain of a coronavirus S protein vary depending on the virus, with some having the RBD at the C-terminus of S1. The S-protein/receptor interaction is the primary determinant for a coronavirus to infect a host species and also governs the tissue tropism of the virus. Many coronaviruses utilize peptidases as their cellular receptor. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes.

### Antibody

Accordingly, the invention provides a pharmaceutical composition comprising an anti-CoV-S antibody. The antibodies of the invention are specific for the spike protein of SARS-CoV-2 as more fully outlined herein and below.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to CoV-S. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

Traditional full-length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein are based on IgG2 heavy constant regions, the anti-CoV-S antibodies of the invention include those using IgG1, IgG3 and IgG4 sequences, or combinations thereof. For example, as is known in the art, different IgG isotypes have different effector functions which may or may not be desirable. Accordingly, the antibodies of the invention can also swap out the IgG2 constant domains for IgG1, IgG3 or IgG4 constant domains.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35 (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vIFR1-vICDR1-vIFR2-vICDR2-vIFR3-vICDR3-vIFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g, Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to CoV-S antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V—HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-CoV-S antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. Alternatively, such recombinant human antibodies have variable regions in which the framework are derived from human germline immunoglobulin sequences and CDR sequences can be any of those described herein (see Tables 1-49). In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see Kabat et al., SEQUENCES OF PROTEINS OF IMMUNOLOGICAL INTEREST, National Institutes of Health Publication No. 91-3242 (1992); Chothia et al., “Canonical Structures For The Hypervariable Regions of Immunoglobulins,” J. Mol. Biol. 196:901-917 (1987), which are hereby incorporated by reference in their entirety), by molecular modeling and/or empirically, or as described in Gonzales et al., “SDR Grafting of a Murine Antibody Using Multiple Human Germline Templates to Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

### Antibody Engineering

The antibodies of the invention can be modified, or engineered, to alter the amino acid sequences by amino acid substitutions. The term “amino acid substitution” or “substitution” refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for CoV-S including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRic; FcγRII (CD32), including isoforms FcγRIIa (including allotypes H131 and R131), FcγRIIb (including FcγRIIb-1 and FcγRIIb-2), and FcγRIIc; and FcγRIIl (CD16), including isoforms FcγRIIla (including allotypes V158 and F158) and FcγRIIlb (including allotypes FcγRIIlb-NA1 and FcγRIIlb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIIl-1 (CD16), and FcγRIIl-2 (CD16-2), as well as any undiscovered mouse FcγRs or FcγR isoforms or allotypes.

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In some embodiments, the antibodies of the invention are modified to increase its biological half-life. Various approaches are used. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half-life are disclosed in U.S. Pat. Nos. 8,883,973, 6,737,056 and 7,371,826, and include 428L, 434A, 434S, and 428L/434S.

In some embodiments, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α(1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the VISG1 antibodies of the invention. In general, only 1 or 2 or 3-amino acids are substituted in any single CDR, and generally no more than from 1, 2, 3, 4, 5, 6, 7, 8, 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the SARS-CoV-2 spike antigen by at least about 10% to 50-100-150% or more, or from 1 to 5 fold as compared to the “parent” antibody. Exemplary affinity matured antibodies will have nanomolar or even picomolar affinities for the SARS-CoV-2 spike antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### DETAILED DESCRIPTION

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Embodiments include antibodies that recognize the spike protein of SARS-CoV-2. Strains of wild type (WT) mice in the PENTAMICE© platform were immunized with adjuvanted spike protein and monoclonal antibodies were obtained using an optimized hybridoma-based antibody recovery workflow as described herein. Based on screening assays, the antibodies displayed a wide range of binding specificities and functional properties. The spike antibodies can be grouped according to reactivity profiles based on binding to the receptor binding domain (RBD) and/or S1 or S2 domains; blocking spike protein binding to the human ACE2 receptor; neutralizing SARS-CoV-2 pseudovirus or SARS-CoV-2 infection of ACE2+ target cells; cross-reactivity with spike proteins from other coronaviruses (SARS-CoV-1, MERS, HKU1, HCoV-NL63, HCoV-229E, HCoV—OC43); and/or binding/neutralization of spike proteins from SARS-CoV-2 variants of concern (B.1.1.7, B.1.351, P.1, B.1.617.2, B.1.1.529). Indeed, it is contemplated that the antibodies and CoV-S binding fragments thereof, as described herein, can be used to bind to SARS-CoV-2 variants that are now known as well as those that arise in the future, either for purposes of detection or neutralization (i.e., treatment or prevention of infection). Exemplary SARS-CoV-2 variants include, without limitation, Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), and Omicron (B.1.1.529, BA.1, BA.1.1, BA.2, BA.3, BA.4 and BA.5 lineages). The profiles demonstrate applications in therapeutics, prevention of SARS-CoV-2 infection and selective SARS-CoV-2 detection diagnostics.

### SARS-CoV-2 Antibody and Antigen-Binding Fragments

The present invention provides a pharmaceutical composition comprising an anti-CoV-S antibody or antigen-binding fragment thereof. (For convenience, “anti-CoV-S antibodies” and “CoV-S antibodies” are used interchangeably). The anti-CoV-S antibodies of the invention specifically bind CoV-S, and particularly the extra cellular domain (ECD) of the spike protein CoV-S.

In some embodiments, in order to generate anti-CoV-S antibodies of the present invention, one or more mutations are introduced to the wild type CoV-S sequence. In some embodiments, one or more mutations introduced to CoV-S comprise R691G, R692S, R694S, K995P, V996P, or any combination thereof. In some embodiments, the CoV-S protein of the present invention comprises R691G, R692S, R694S, K995P, and V996P. In some embodiments, the CoV-S protein of the present invention is fused to the T4 fibritin trimerization domain.

In some embodiments, the present invention provides CoV-S antibodies that bind to the receptor binding domain (RBD) within the S1 domain. In some embodiments, the present invention provides CoV-S antibodies that bind to a portion of the S1 domain outside the RBD (i.e., non-RBD S1 domains). In some embodiments, the present invention provides CoV-S antibodies that bind to the S2 domain. In some embodiments, the present invention provides CoV-S antibodies that bind to neither of the S1 (including the RBD) and S2 domains. In some embodiments, the present invention provides CoV-S antibodies that are SARS-CoV-2 spike selective.

In some embodiments, the CoV-S antibodies provided herein can be grouped according to reactivity profiles based on binding to the receptor binding domain (RBD) and/or S1 or S2 domains; blocking spike protein binding to the human ACE2 receptor; neutralizing SARS-CoV-2 pseudovirus or SARS-CoV-2 infection of ACE2+ target cells; cross-reactivity with spike proteins from other coronaviruses (e.g., SARS-CoV-1, MERS, HKU1, HCoV-NL63, HCoV-229E, HCoV—OC43); and binding/neutralization of spike proteins from SARS-CoV-2 variants of concern (e.g., B.1.1.7, B.1.351, P.1, B.1.617.2, B.1.1.529).

Specific binding for CoV-S or epitope can be exhibited, for example, by an antibody having a KD of at least about 10⁻⁴ M, at least about 10⁻⁵ M, at least about 10⁻⁶ M, at least about 10⁻⁷ M, at least about 10⁻⁸ M, at least about 10⁻⁹ M, or alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the CoV-S antigen or epitope.

Also, specific binding for a particular antigen or an epitope can be exhibited, for example, by an antibody having a KA or Ka for CoV-S of at least 20-, 50-, 100-, 500-, 1000-, 5,000-, 10,000- or more times greater for the epitope relative to a control, where KA or Ka refers to an association rate of a particular antibody-antigen interaction.

In some embodiments, the anti-CoV-S antibodies of the invention bind to CoV-S with a KD of 100 nM or less, 50 nM or less, 10 nM or less, or 1 nM or less (that is, higher binding affinity), or 1 pM or less, wherein KD is determined by known methods, e.g. surface plasmon resonance (SPR, e.g. Biacore assays), ELISA, KINEXA, and most typically SPR at 25 or 37° C.

In some embodiments, the antigen-binding portions and variants of the above-identified antibodies retain binding activity that is essentially the same as the binding activity of the whole antibody from which it is derived. By “essentially the same”, it is intended that the antigen-binding portions and variants retain at least 80% (such as at least 85%, or at least 90%, or at least 95%) of the binding affinity (K_{D}) for Cov-S or neutralizing capacity (IC₅₀) for SARS-CoV-2 variants as compared to the parent antibody. In some other embodiments, the antigen-binding portions and variants of the above-identified antibodies retain at least 50% (such as at least 60%, at least 65%, at least 70%, or at least 75%) of the binding activity of the whole antibody (e.g., binding affinity (K_{D}) for Cov-S or neutralizing capacity (IC₅₀) for SARS-CoV-2 variants) from which it is derived.

A. Specific Anti-CoV-S Antibodies

The invention provides antigen binding domains, including full length antibodies, which contain a number of specific, enumerated sets of 6 CDRs. Embodiments include antibodies with different specificities for SARS-CoV-2 spike proteins from wild type (WT) and variants (i.e., α, β, γ, Δ, ο) such that distinct binding profiles can differentiate between SARS-CoV-2 variants.

The distinct binding pattern of the antibodies in Table A (listed with their mAb clone IDs) against the spike proteins in each of the five columns enables an Ab-based method for unambiguously distinguishing among the five SARS-CoV-2 variants. In addition, Applicants discovered a pan-coronavirus binding mAb (1-B11-A) that binds to the S2 domain of the spike protein from all seven coronaviruses known to infect people, including SARS-CoV-2 and its α, β, γ, Δ, ο variants. These antibodies are particularly useful in diagnostics. For example, they can be used to develop a Rapid Lateral Flow Assay (LFA) where the pan-coronavirus binding mAb captures virus from a biosample (e.g., swab or plasma), and the panel of antibodies described below can be used to detect the specific SARS-CoV-2 variant present in the sample.

The antibodies and/or panel of antibodies disclosed herein can also be used in a characterization/release assay in vaccine development. For example, the antibodies and/or panel of antibodies described herein can be used to verify that the spike protein of a specific SARS-CoV-2 variant or multiple different variants (e.g., WT, α, β, γ, Δ, ο) are present in a candidate vaccine. In order to verify the composition of a delta-specific variant vaccine, for example, antibodies are needed that can distinguish delta from other variants. This is particularly useful for a multi-variant vaccine comprising a mixture of multiple variant spike proteins; commercially available antibodies cannot distinguish between variants and thus it is not possible to confirm that a putative multi-variant vaccine product contains spike protein from each variant. This also applies to mRNA vaccines. To verify that a candidate mRNA vaccine encodes the specific targeted SARS-CoV-2 variant(s), test cells or animals can be treated with the mRNA vaccine. The protein expressed following introduction of the mRNA can be assayed by a panel of antibodies to verify expression of the specific targeted spike protein variant, confirming generation of a variant(s)-specific mRNA vaccine.

TABLE A
Antibodies with Variant Specificities

| | mAb | | | | | | | |
|---|---|---|---|---|---|---|---|---|
| mAb | clone ID | WT | alpha | beta | gamma | delta | omicron | BA.2 |
| 1 | 10-O3-A | + | − | − | + | − | − | − |
| 2 | 8-H3-A | + | + | − | − | + | − | − |
| 3 | 9-M12-A, | − | − | + | − | − | − | − |
| | 3-P17-A, | | | | | | | |
| | 9-G24-A | | | | | | | |
| 4 | 7-N20-A | + | + | − | + | − | − | − |
| 5 | 3-K11-A | − | − | + | − | − | + | − |
| 6 | (pan) | + | + | + | + | + | + | + |
| | 1-B11-A | | | | | | | |

The invention further provides CDRs, variable heavy and light domains as well as full length heavy and light chains including those identified in mAbs 1-B11-A, 1-L10-A, 2-H7-A, 2-J9-A, 2-O12-A, 2-P2-A, 3-E13-A, 4-A15-A, 4-C3-A, 4-K13-A, 4-L4- A, 5-H22-A, 6-O12-A, 8-N24-A, 9-J11-A, 9-L13-A, 9-P9-A, 10-B111-A, 10-L24-A, 10-O3-A, 4-M3-A, 4-N22-A, 7-B10-A, 8-H5-A, 2-G20-A, 3-E2-A, 4-K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A.

As discussed herein, the invention further provides variants of the above components, including variants in the CDRs, as outlined above. In addition, variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein, and/or contain from 1, 2, 3, 4, 5, 6, 7, 8, 9, 10 amino acid changes, or more, when Fc variants are used. Similarly, heavy and light chains are provided that are 80%, 90%, 95%, 98% or 99% identical to the “HC” and “LC” sequences herein, and/or contain from 1, 2, 3, 4, 5, 6, 7, 8, 9, 10 amino acid changes, or more, when Fc variants are used.

Accordingly, the antibodies of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Additionally, included in the definition of CoV-S antibodies are antibodies that share identity to the CoV-S antibodies enumerated herein. That is, in certain embodiments, an anti-CoV-S antibody according to the invention comprises heavy and light chain variable regions comprising amino acid sequences that are homologous to isolated anti-CoV-S amino acid sequences of exemplary anti-CoV-S immune molecules, respectively, wherein the antibodies retain the desired functional properties of the parent anti-CoV-S antibodies. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The antibodies of the invention include those antibodies having the identical CDRs but differing in the variable domain (or entire heavy or light chain). For example, antibodies include those with CDRs identical to those shown in Tables 1-49 but whose identity along the variable region can be lower, for example 95 or 98% percent identical.

The present invention provides not only the enumerated antibodies but additional antibodies that compete with the enumerated antibodies to specifically bind to CoV-S. Additional antibodies that compete with the enumerated antibodies are generated, as is known in the art and generally outlined below. Competitive binding studies can be done as is known in the art, generally using SPR/Biacore© binding assays, as well as ELISA and cell-based assays.

Methods of generating antibodies as well as subsequent screening assays are well-known in the art, such as those outlined in the examples. In some embodiments, anti-CoV-S antibodies are generated by traditional methods such as immunizing mice (sometimes using DNA immunization), followed by screening against CoV-S and hybridoma generation, with antibody purification and recovery.

B. Formulations of Anti-CoV-S Antibodies

The therapeutic compositions used in the practice of the present invention can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibodies of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Exemplary ones are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

Administration of the pharmaceutical composition comprising antibodies of the present invention, for example in the form of a sterile aqueous solution, may be done in a variety of ways, including, but not limited to subcutaneously, intravenously, and intranasally. Subcutaneous administration may be done in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO04091658). Fc polypeptides of the present invention may be more amenable to subcutaneous administration due to, for example, increased potency, improved serum half-life, or enhanced solubility.

As is known in the art, protein therapeutics are often delivered by IV infusion or bolus. The antibodies of the present invention may also be delivered using such methods. For example, administration may be by intravenous infusion with 0.9% sodium chloride as an infusion vehicle.

In addition, any of a number of delivery systems are known in the art and may be used to administer the Fc variants of the present invention. Examples include, but are not limited to, encapsulation in liposomes, microparticles, microspheres (eg. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT©, and poly-D-(−)-3-hydroxyburyric acid. The antibodies disclosed herein may also be formulated as immunoliposomes. A liposome is a small vesicle comprising various types of lipids, phospholipids and/or surfactant that is useful for delivery of a therapeutic agent to a mammal. Liposomes containing the antibody are prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The antibodies may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the antibody in the formulation may vary from about 0.1 to 100 weight %. In some embodiments, the concentration of the Fc variant is in the range of 0.003 to 1.0 molar. In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from about 0.0001 to 100 mg/kg of body weight or greater, for example about 0.1, 1, 10, or 50 mg/kg of body weight, and in an exemplary embodiment, from about 1 to 10 mg/kg.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

C. Nucleic Acids, Expression Vectors, Host Cells

In some embodiments, the present invention provides nucleic acids encoding the antibodies or antigen-binding domains as described herein. As will be appreciated by those in the art, the protein sequences depicted herein can be encoded by any number of possible nucleic acid sequences, due to the degeneracy of the genetic code. In some embodiments, the nucleic acid molecules are DNA. In some embodiments, the nucleic acid molecules are RNA.

The nucleic acid compositions that encode the CoV-S antibodies will depend on the format of the antibody. In exemplary embodiments, tetrameric antibodies containing two heavy chains and two light chains are encoded by two different nucleic acids, one encoding the heavy chain and one encoding the light chain. These can be put into a single expression vector or two expression vectors, as is known in the art, transformed into host cells, where they are expressed to form the antibodies of the invention. In some embodiments, for example when scFv constructs are used, a single nucleic acid encoding the variable heavy chain-linker-variable light chain is generally used, which can be inserted into an expression vector for transformation into host cells. The nucleic acids can be put into expression vectors that contain the appropriate transcriptional and translational control sequences, including, but not limited to, signal and secretion sequences, regulatory sequences, promoters, origins of replication, selection genes, etc.

Exemplary mammalian host cells for expressing the recombinant antibodies according to at least some embodiments of the invention include Chinese Hamster Ovary (CHO cells), PER.C6, HEK293 and others as is known in the art.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

To create a scFv gene, the VH— and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

D. Therapeutic Application of the Antibodies or Antigen-Binding Fragments

The present invention provides methods for treating or preventing viral infection (e.g., coronavirus infection) by administering a therapeutically effective amount of anti-CoV-S spike antigen-binding protein, e.g., antibody or antigen-binding fragment, (e.g., of TABLES 1-52) to a subject (e.g., a human) in need of such treatment or prevention.

Coronavirus infection may be treated or prevented, in a subject, by administering an antibody or antigen-binding fragment of the present invention to a subject.

An effective or therapeutically effective dose of anti-CoV-S antigen-binding protein, e.g., antibody or antigen-binding fragment (e.g., of TABLES 1-49), for treating or preventing a viral infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the invention, an effective or therapeutically effective dose of antibody or antigen-binding fragment thereof of the present invention, for treating or preventing viral infection, e.g., in an adult human subject, is about 0.01 to about 200 mg/kg, e.g., up to about 150 mg/kg. In an embodiment of the invention, the dosage is up to about 10.8 or 11 grams (e.g., about 1, 2, 3, 4, 5, 6, 7, 8, 9, 10 or 11 grams). Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antibody or antigen-binding fragment thereof in an amount that can be approximately the same, more or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks, or more.

In some embodiments, the method of preventing viral infection provided herein comprises prophylactically administering an antibody or antigen-binding fragment of the present invention (e.g., of TABLES 1-49), to a subject who is at risk of viral infection so as to prevent such infection. Passive antibody-based immunoprophylaxis has proven an effective strategy for preventing viral infection. See e.g., Berry et al., Passive broad-spectrum influenza immunoprophylaxis. Influenza Res Treat. 2014; 2014:267594. Epub 2014 Sep. 22; and Jianqiang et al., Passive immune neutralization strategies for prevention and control of influenza A infections, Immunotherapy. 2012 February; 4(2): 175-186; Prabhu et al., Antivir Ther. 2009; 14(7):911-21, Prophylactic and therapeutic efficacy of a chimeric monoclonal antibody specific for H5 hemagglutinin against lethal H5N1 influenza. “Prevent” or “preventing” means to administer an antibody or antigen-binding fragment of the present invention (e.g., of TABLES 1-49), to a subject to inhibit the manifestation of a disease or infection (e.g., viral infection) in the body of a subject, for which the antigen-binding protein is effective when administered to the subject at an effective or therapeutically effective amount or dose (as discussed herein).

In an embodiment of the invention, a sign or symptom of a viral infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection are discussed herein.

As noted above, in some embodiments the subject may be a non-human animal, and the antigen-binding proteins (e.g., antibodies and antigen-binding fragments) discussed herein may be used in a veterinary context to treat and/or prevent disease in the non-human animals (e.g., cats, dogs, pigs, cows, horses, goats, rabbits, sheep, and the like).

In some embodiments, the present invention provides a method for treating or preventing viral infection (e.g., coronavirus infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of an antibody or antigen-binding fragment (e.g., of Tables 1-52) to the subject, for example, by injection of the protein into the body of the subject.

E. Diagnostic Application of the Antibodies or Antigen-Binding Fragments

In some embodiments, the antibody or antigen-binding fragment thereof of the present invention (e.g., of TABLES 1-49), may be used to detect and/or measure SARS-Cov-2 in a sample. Exemplary assays for CoV-S may include, e.g., contacting a sample with an SARS-CoV-2 antibody of the invention, wherein the antibody is labeled with a detectable label or reporter molecule or used as a capture ligand to selectively isolate CoV-S from samples. The presence of a CoV-S antibody complexed with CoV-S indicates the presence of the SARS-Cov-2 virus in the sample. Alternatively, an unlabeled SARS-CoV-2 antibody can be used in combination with a secondary antibody which is itself detectably labeled. The detectable label or reporter molecule can be a radioisotope, such as 3H, 14C, 32P 35S, or 1251; a fluorescent or chemiluminescent moiety such as fluorescein isothiocyanate, or rhodamine; or an enzyme such as alkaline phosphatase, R-galactosidase, horseradish peroxidase, or luciferase. Specific exemplary assays that can be used to detect or measure CoV-S in a sample include neutralization assays, enzyme-linked immunosorbent assay (ELISA), radioimmunoassay (RIA), and fluorescence-activated cell sorting (FACS). Thus, the present invention includes a method for detecting the presence of spike protein or polypeptide in a sample comprising contacting the sample with a CoV-S antibody and detecting the presence of the antibody wherein the presence of the complex (CoV-S antibody complexed with CoV-S) indicates the presence of the SARS-CoV-2 virus in the sample.

Any of a variety of suitable biological (patient) samples can be used for diagnostic detection of SARS-Cov-2 in a sample. Exemplary biological samples include, without limitation, bronchoalveolar fluid, nasopharyngeal swabs, sputum, blood, feces and anal swabs, and urine.

In some embodiments, the CoV-S antibodies of the invention (e.g., of TABLES 1-49) may be used in a Western blot or immune-protein blot procedure for detecting the presence of SARS-CoV-2 in a sample.

In some embodiments, the CoV-S antibodies disclosed herein may also be used for immunohistochemistry.

### EXAMPLES

The following examples are provided to illustrate certain embodiments of the invention. The invention is not limited to these examples and the full scope of the invention is reflected in the claims appended hereto.

### Example 1: Mouse Antibodies with Activities Against SARS-CoV-2 Immunizations and Antibody Recovery

FIG. 1 depicts the sequence of the SARS-CoV-2 prefusion stabilized trimer protein immunogen. The SARS-CoV-2 spike protein extracellular domain (Gene ID/Reference: MN996527.1 (GenBank), ECD (23-1222), WIV02 isolate) was expressed using the TunaCHO™ manufacturing platform. The furin-recognition site RRAR was mutated to GSAS. KV was mutated to PP to stabilize the protein in a prefusion conformation. A T4 fibritin trimerization domain was added to the carboxyl-terminal domain to facilitate trimerization.

Strains of wild type (WT) mice in the PENTAMICE© platform were immunized with adjuvanted spike protein. PENTAMICE© mice are a proprietary set of WT mice generated via in-house breeding that comprise five strains of F1 and outbred WT mice and cover nine distinct major histocompatibility complex (MHC) class II (I-A, I-E) haplotypes (b, d, g7, k, q, s, u, v, and mixed). When in-life plasma titers indicated that a strong anti-spike protein humoral immune response was achieved, the animals were euthanized and lymphocytes harvested and fused with a myeloma partner via electrofusion to generate hybridomas. The hybridomas were plated into ten 384-well plates and supernatants were screened for reactivity against SARS-CoV-2 spike protein by ELISA. Candidate parental hybridomas were subjected to limiting dilution cloning to generate monoclonal hybridomas. Variable heavy and light chain sequences were determined for various monoclonal antibodies. Purified antibodies were generated and assessed for various binding and functional characteristics. Select monoclonal antibodies (mAbs) were reformatted and expressed as human Fc IgG2 chimeras (e.g., 6-O12-A). Other mAbs (e.g., 4-C3-A and 2-J9-A) were expressed recombinantly as mouse Fc IgG2b antibodies. Further studies demonstrated that the recombinantly expressed mAbs retained their binding properties.

### ELISA

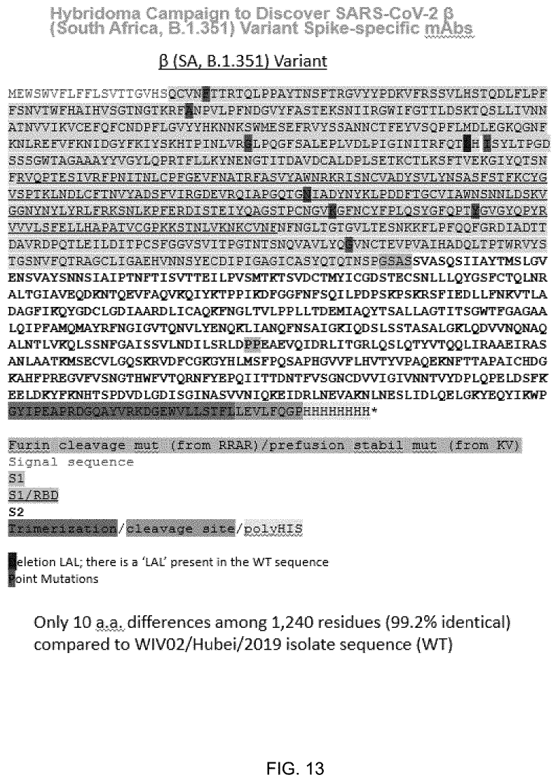

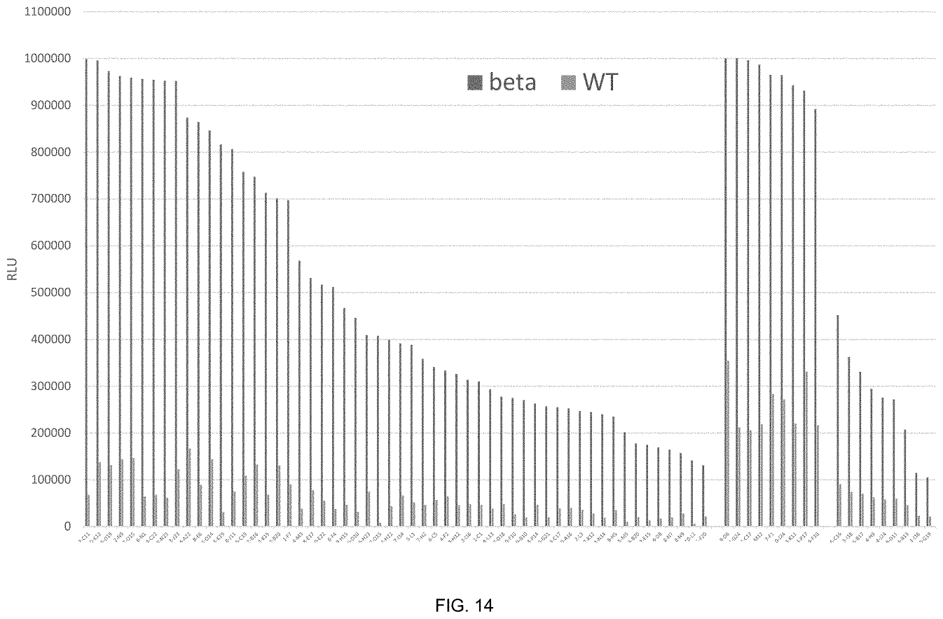

mAb binding reactivity was assessed by ELISA against the following antigens: SARS-CoV-2 (WIV02 isolate) spike protein (see FIG. 1); SARS-CoV-2 S1 domain (sequence contained within FIG. 1); SARS-CoV-2 S2 domain (sequence contained within FIG. 1); SARS-CoV-2 receptor binding domain (RBD, sequence contained within FIG. 1); SARS-CoV-1 spike protein; MERS spike protein; HKU1 spike protein; HCoV-NL63 spike protein; HCoV229E spike protein; HCoV—OC43 spike protein; SARS-CoV-2 B.1.1.7 spike protein; SARS-CoV-2 B.1.351 spike protein; SARS-CoV-2 P.1 spike protein; BVP (baculovirus particles, non-specific binding); ICOS-His (irrelevant His-tagged negative control protein). The MERS spike protein corresponds to the sequence reported at GenBank AFY13307.1, UniProtKB K9N5Q8, which are hereby incorporated by reference in their entirety; the SARS-CoV-1 spike protein corresponds to the sequence reported at GenBank AAP13441.1, UniProtKB P59594, which are hereby incorporated by reference in their entirety); the HKU1 spike protein corresponds to the sequence reported at Genbank ADN03339.1, UniProtKB EOYJ44, which are hereby incorporated by reference in their entirety); the HCoV-NL63 spike protein corresponds to the sequence reported at UniProtKB Q6Q1S2 (residues 24-1294), which is hereby incorporated by reference in its entirety; the HCoV229E spike protein corresponds to the sequence reported at UniProtKB P15423 (residues 17-1103), which is hereby incorporated by reference in its entirety; and the HCoV—OC43 spike protein corresponds to the sequence reported at UniProtKB Q696P8, GenBank: AAT84354.1 (residues 1-1263), which are hereby incorporated by reference in their entirety. The SARS-CoV-2 B.1.1.7 spike protein, SARS-CoV-2 B.1.351 spike protein (see FIG. 13), and SARS-CoV-2 P.1 spike protein variants were formed by mutating the sequence corresponding to the sequence reported at GenBank MN996527.1/UniProtKB J2778 with the mutations identified on the CDC website for those spike protein variants. ELISA plates were coated with antigen (1-10 ug/mL) and blocked with 3% bovine serum albumin (BSA). Various dilutions of antibodies are added to the coated blocked plates and incubated 1 hour at room temperature and then washed. Anti-mouse IgG-horse radish peroxidase (HRP) in blocking buffer is added to the wells and incubated 1 hour at room temperature and washed. Pre-mixed SuperSignal ELISA Pico substrate (Thermo) solution is added to each well and bound protein is detected using Molecular Devices SpectraMax M3 luminometer and Softmax Pro Version 6.2 within 15 minutes of adding substrate.

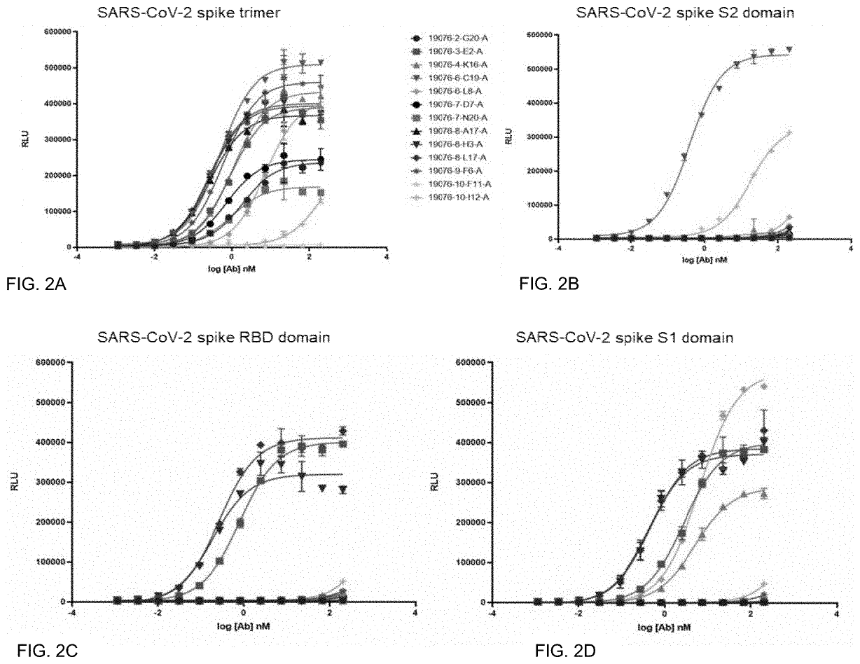

FIGS. 2A-2D illustrate EC₅₀ ELISA binding curves for selected SARS-CoV-2 spike-binding mAbs against spike trimer (FIG. 2A), S2 domain (FIG. 2B), RBD domain (FIG. 2C), and S1 domain (FIG. 2D). 10-F11-A is included as a negative control mAb that does not bind to SARS-CoV-2 spike protein.

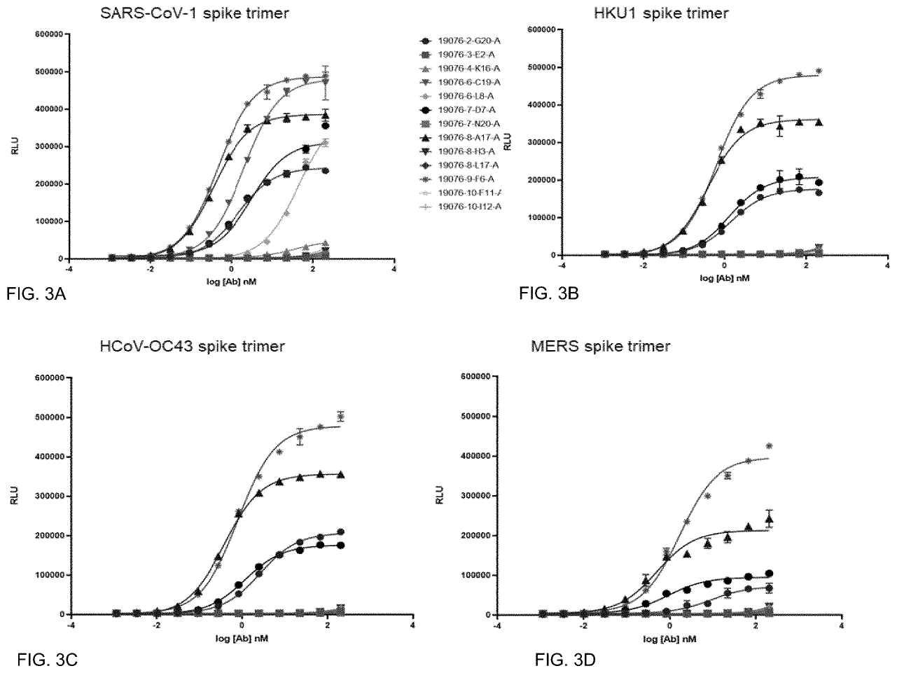

FIGS. 3A-3D illustrate EC₅₀ ELISA binding curves for selected SARS-CoV-2 spike-binding mAbs against spike trimers from SARS-CoV-1 (FIG. 3A), HKU1 (FIG. 3B), HCOV—OC43 (FIG. 3C) and MERS spike trimer (FIG. 3D). As in the above study, 10-F11-A is included as a negative control mAb that does not bind to SARS-CoV-2 spike protein.

### ELISA Neutralization Assay

Human angiotensin-converting enzyme 2 (ACE2) is an entry receptor for SARS-CoV-2 and SARS-CoV-1 via binding to the RBD domain of the viral spike protein. An ELISA was developed to evaluate the ability of spike-binding mAbs to neutralize the interaction of the SARS-CoV-2 S protein RBD with the ACE2 receptor. The neutralizing antibody assay is similar to a COVID-19 Spike-ACE2 binding assay kit II for COVID-19 drug and antibody screening (Ray Biotech, Inc., Peachtree Corners, Ga.) and is described in the literature (Byrnes et al. 2020; Tai et al. 2020). In this assay, a 384-well ELISA plate is coated with recombinant huACE2 protein-human fragment crystallizable region (Fc); (5 ug/mL), blocked with 3% BSA for 1 hour at room temperature. Various dilutions of antibodies are pre-mixed with histidine-tagged spike proteins (either SARS-CoV-2 WT WIV02 spike trimer; SARS-CoV-2 B.1.1.7 spike trimer variant; SARS-CoV-2 B.1.351 spike trimer variant; SARS-CoV-2 P.1 spike trimer variant; or SARS-CoV-1 spike trimer; all 1 ug/mL) for at least 15 minutes at room temperature and then added to the 384-well plate and incubated at room temperature for 1 hour. After incubation, plates are washed 4 times, rotated 180 degrees, and washed an additional 4 times. Bound protein is detected following incubation with anti-His-HRP antibody for 1 hour at room temperature. Pre-mixed SuperSignal ELISA Pico substrate (Thermo) solution is added to each well and bound protein is detected using Molecular Devices SpectraMax M3 luminometer and Softmax Pro Version 6.2 within 15 minutes of adding substrate.

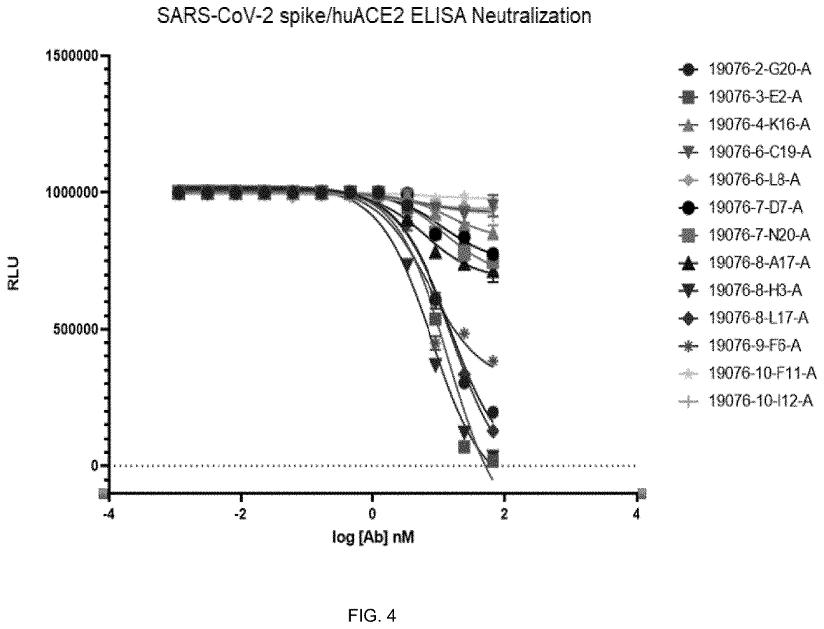

FIG. 4 illustrates IC₅₀ ELISA neutralization curves for selected SARS-CoV-2 spike-binding mAbs inhibiting the binding of SARS-CoV-2 spike trimer to huACE2. 10-F11-A is included as a negative control mAb that does not bind/neutralize SARS-CoV-2 spike protein.

### SARS-CoV-2 and SARS-CoV-1 Pseudovirus Infection of ACE2+ TMPRSS2+ Target Cells

Targeted 293T cells were transfected with pcDNA3.1(+)-ACE2 and pCSDest-TMPRSS2 for 6 h. The cells were then trypsinized and seeded 1×10⁵ cells/well in DMEM complete into 96-well plates (100 μL/well) then incubated for 16 hours at 37° C. and 5% CO₂. The antibodies were 3-fold serially diluted in a pseudovirus/buffer mixture. Based on the antibody concentration, 1 M HEPES buffer was used to dilute the pseudovirus to the correct percent buffer concentration in all wells except the first. Virions were incubated with the test samples at room temperature for one hour, and then added to the target cells in 96-well plates. Plates were incubated for 48 hours and degree of viral infection was determined by luminescence using the neolite reporter gene assay system (PerkinElmer). All error bars represent S.D. from three replicates.

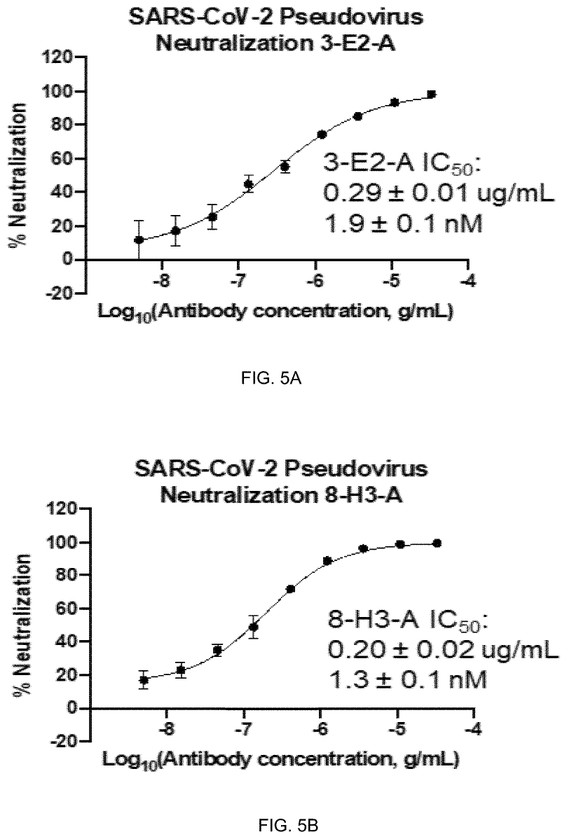

FIGS. 5A-5B show IC₅₀ titration of 3-E2-A, and 8-H3-A in SARS-CoV-2 pseudovirus ACE2+ TMPRSS2+ target cell infection assay. IC₅₀ values were determined by fitting the dose-response curves with four-parameter logistic regression in Prism GraphPad (version 8.1.2). All data was normalized to pseudovirus alone. All error bars represent S.D. from three replicates.

### Binding Kinetics Via Surface Plasma Resonance (SPR)

Binding experiments were performed on Carterra LSA. Candidate antibodies (ligands) were diluted to 10 μg/mL in 10 mM NaOAc pH 4.5 containing 0.01% Tween-20 and coupled to a HC30M chip via sulpho-NHS/EDC coupling chemistry and blocked with ethanolamine. Buffer exchange of antigen SARS-CoV-2 Spike Protein RBD were performed using Zeba column prior to Carterra analysis. SARS-CoV-2 (2019-nCoV) Spike RBD-His Recombinant Protein, Lot TP31549F S Protein RBD (319-591)-His10. Original formulation: 50 mM Tris pH 7.5, 150 mM NaCl, 0.05% NaN3. Serial dilutions (1000 nM start, 1:3 dilution, 8 points) of RBD were injected for kinetic constant determination. At the end of each cycle, the chip was regenerated with 10 mM Glycine pH 2.0 to remove bound antigen. Kinetics analysis was performed using Carterra kinetics software.

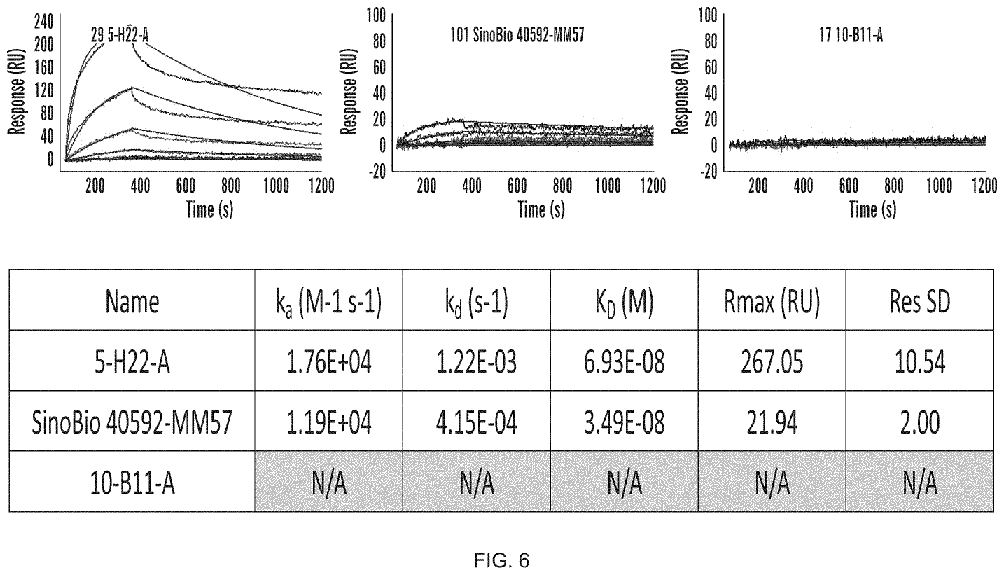

FIG. 6 shows the binding kinetics for selected SARS-CoV-2 spike-binding antibodies against RBD. Carterra LSA was used to determine on/off rates and binding affinities (KD). Candidate antibodies (ligands) were coupled to a HC30M chip and blocked. Serial dilutions (1000 nM start, 1:3 dilution, 8 points) of RBD were injected for kinetic constant determination. At the end of each cycle, the chip was regenerated to remove bound antigen. Kinetics analysis was performed using Carterra kinetics software. 10-B11-A and 1-L10-A do not bind the RBD; spike-binding mAb SinoBio 40592-MM57 was included as a positive control.

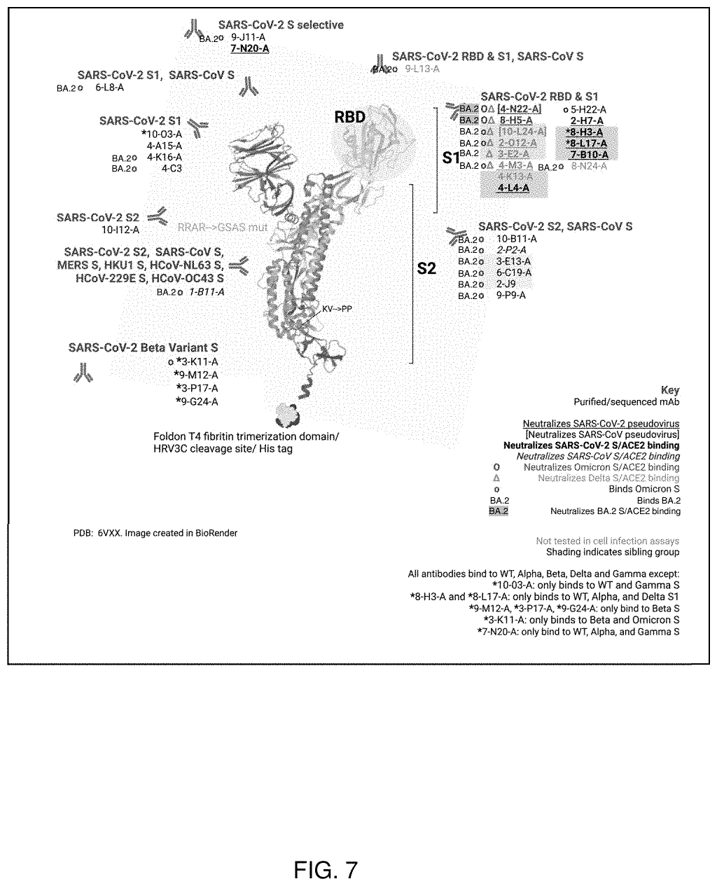

FIG. 7 shows binding and functional summary of SARS-CoV-2 spike-binding antibodies. A wide variety of antibodies with a range of binding and functional activities are summarized. Certain antibodies are specific for SARS-CoV-2 RBD, cross-react with SARS-CoV-1, bind to three CDC variants of concern, and neutralize both SARS-CoV-2 and SARS-CoV-1 in in vitro infection models. Certain antibodies can be produced recombinantly with a mouse IgG2b Fc and are specific to non-RBD domains of the S1 domain (e.g., 4-C3-A). Certain antibodies are specific for the S2 domain of SARS-CoV2 (e.g., 10-I12-A); some also cross-react with SARS-CoV-1 (e.g., 10-B11-A); some also cross-react with all of the coronavirus spike proteins known to infect humans (e.g., 1-B11-A). Certain neutralizing antibodies are selective for SARS-CoV-2 spike trimer and do not seem to bind to recombinantly-expressed subdomains (e.g., 7-N20-A). All antibodies bind to B.1.1.7, B.1.351, and P.1 spike variants of concern except 10-O3-A, which only binds to P.1; and 8-H3-A and 8-L17-A, which only bind to B.1.1.7.

### Dendrogram

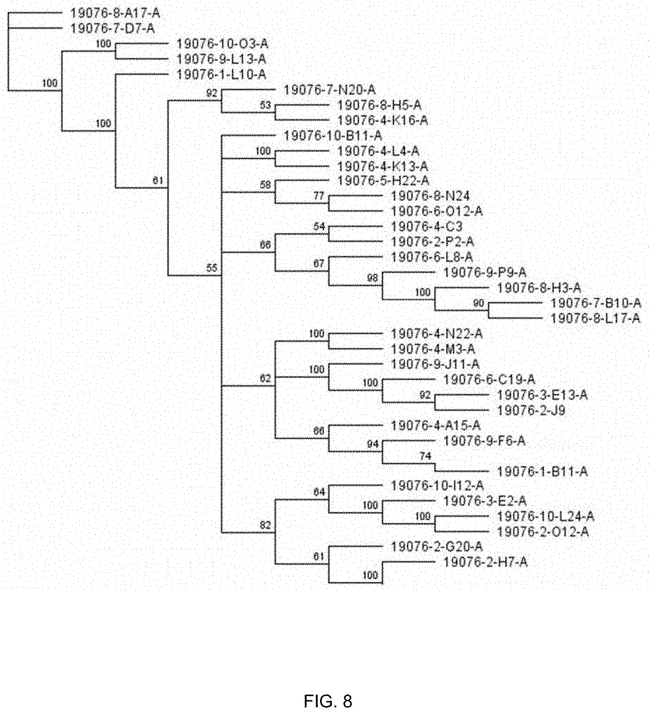

FIG. 8 is a SARS-CoV-2 spike binding mAb dendrogram. A phylogenetic dendrogram for 42 spike-binding mAb protein sequences was built by MUSCLE alignment and Neighbor-joining using Geneious software. The heavy chain and light chain sequences for each mAb were concatenated into one sequence (separated by a 4×GGGS linker). The confidence (%) after resampling against the consensus tree is displayed at each node. The resample method is bootstrap. The number of resampling replicates is 100.

### Example 2: Mouse Antibodies with Activities Against the SARS-CoV-2 Delta Variant B.1.617.2

The antibodies produced according to Example 1 were tested for activity against the SARS-CoV-2 Delta variant.

### ELISA

mAb binding reactivity was assessed by ELISA against SARS-CoV-2 B.1.617.2 spike trimer variant. ELISA plates were coated with antigen (1-10 ug/mL) and blocked with about 3% bovine serum albumin (BSA). Various dilutions of antibodies were added to the coated blocked plates and incubated about 1 hour at room temperature and then washed. Anti-mouse IgG-horse radish peroxidase (HRP) in blocking buffer was added to the wells and incubated about one hour at room temperature and washed. Pre-mixed SuperSignal ELISA Pico substrate (Thermo) solution was added to each well and bound protein was detected using Molecular Devices SpectraMax M3 luminometer and Softmax Pro Version 6.2 within about 15 minutes of adding substrate.

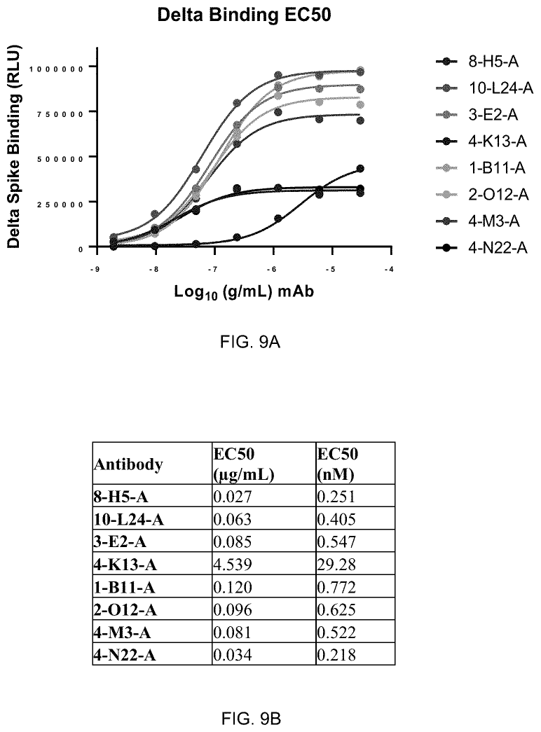

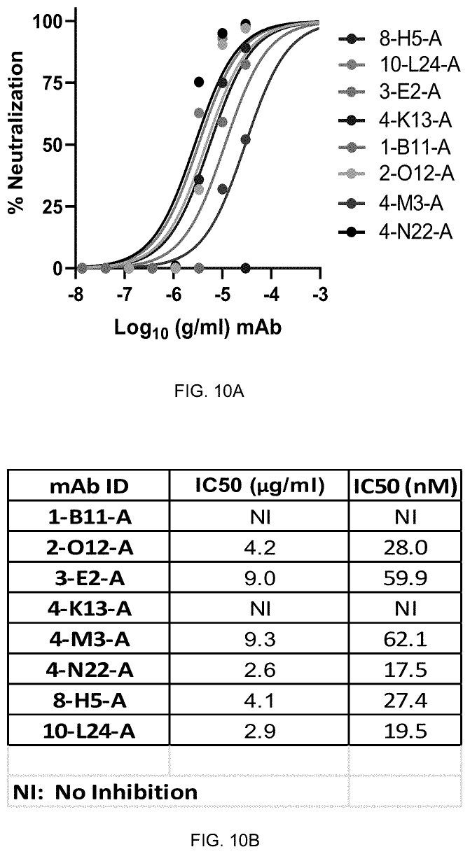

FIG. 9A illustrates EC₅₀ ELISA binding curves for numerous SARS-CoV-2 spike-binding mAbs against SARS-CoV-2 B.1.617.2 spike trimer variant. Eight potent Delta spike binding mAbs were identified (8-H5-A, 10-L24-A, 3-E2-A, 4-K13-A, 1-B11-A, 2-O12-A, 4-M3-A and 4-N22-A). FIG. 9B is a table of the EC₅₀ values for the mAbs and indicates that the values range between 171 pM and 29 nM.

### ELISA Neutralization Assay

The ELISA neutralization assay described in Example 1 was used to evaluate the neutralization activity of the following Delta spike binding mAbs: 8-H5-A, 10-L24-A, 3-E2-A, 4-K13-A, 1-B11-A, 2-O12-A, 4-M3-A and 4-N22-A. In this assay, a 384 well ELISA plate was coated with recombinant huACE2 protein-human fragment crystallizable region (Fc); (5 ug/mL), blocked with 3% BSA for 1 hour at room temperature. Various dilutions of antibodies were pre-mixed with histidine-tagged SARS-CoV-2 B.1.617.2 spike trimer variant at about 1 ug/mL for at least about 15 minutes at room temperature and then added to the 384-well plate and incubated at room temperature for about 1 hour. After incubation, plates were washed 4 times, rotated 180 degrees, and washed an additional 4 times. Bound protein was detected following incubation with anti-His-HRP antibody for about 1 hour at room temperature. Pre-mixed SuperSignal ELISA Pico substrate (Thermo) solution was added to each well and bound protein was detected using Molecular Devices SpectraMax M3 luminometer and Softmax Pro Version 6.2 within about 15 minutes of adding substrate.

FIGS. 10A and 10B show the results of an ELISA Neutralization BioFunction assay. Delta SARS-CoV-2 Spike/ACE2 Neutralization IC₅₀ of these eight monoclonal antibodies is provided. As shown, the top Delta spike neutralizers in the ELISA Biofunction assay had an IC₅₀ between about 18 and 62 nM.

### SARS-CoV-2 Delta/HIV Pseudovirus Infection Inhibition: ACE2+ and HeLa Targets

Targeted HeLa-hACE2 (stable human Angiotensin Converting Enzyme—2 HeLa clone) cells were seeded 5×10⁴ cells/well in DMEM complete into 96-well plates (100 μL/well) then incubated for 16 hours at 37C and 5% CO₂. The antibodies/plasma samples were 3-fold serially diluted in a pseudovirus/buffer mixture. Based on the sample concentration, 100 mM HEPES buffer was used to dilute the pseudovirus to the correct percent buffer concentration in all wells except the first. Virions were incubated with the test samples at room temperature for one hour, and then added to the target cells in 96-well plates. Plates were incubated for 48 hours, and degree of viral infection was determined by luminescence using the neolite reporter gene assay system (PerkinElmer). All error bars represent SD from three replicates.

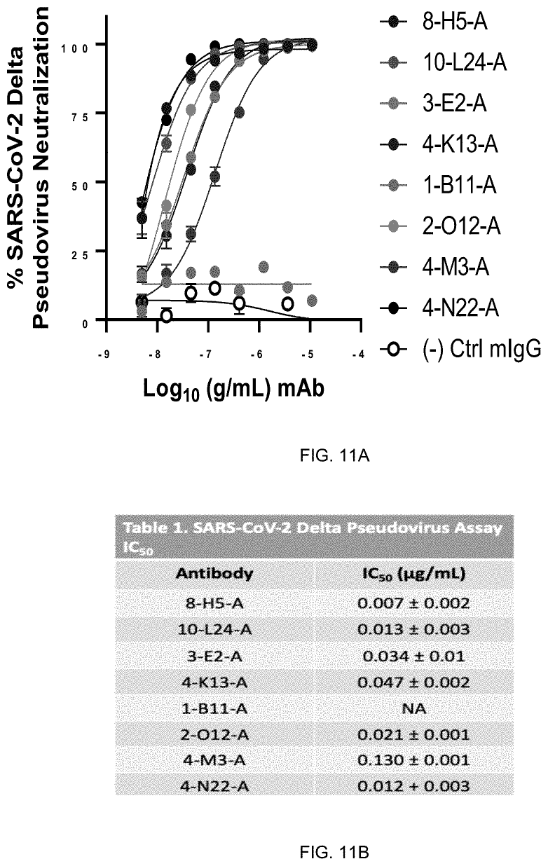

Nine purified monoclonal antibody samples were sent for pseudovirus neutralization testing in SARS-CoV-2 Delta/HIV pseudovirus. All samples were serially diluted (3-fold) starting at a dilution of 33 μg/mL. FIGS. 11A and 11B show SARS-CoV-2 Delta/HIV pseudovirus infection inhibition and provide a summary of the IC₅₀ values of the mAbs. IC₅₀ values were determined by fitting the dose-response curves with four-parameter logistic regression in Prism GraphPad (version 8.1.2). All data was normalized to pseudovirus alone. All error is the SD from three replicates. (NA=no activity). Seven of the antibodies showed dose-dependent neutralization.

### Authentic SARS-CoV-2 Delta Variant (BSL3) Neutralization: Vero E6 Targets

Plaque assay was performed on Vero E6 cells expressing ACE2 protein by plating them in 12-well plate at 90-95% confluency. PRNT assay was performed according the recent protocol “Quantification of SARS-CoV-2 neutralizing antibody by wild-type plaque reduction neutralization . . . ” by Bewley et. al. (Nat Protoc 16, 3114-3140 (2021)). 100 μl of the antibody, for each dilution, at 2× concentration in the PRNT assay diluent medium (MEM with 1% FBS) were mixed with 100 μl of the virus in the assay diluent medium to obtain 200 μl of the virus-antibody mix, which was incubated at 37° C. for 1 h before adding onto the cells for plaque assay. Starting concentration: 33 μg/ml at ⅓-dilutions (9-points). Only virus ‘without the antibody’ and ‘no virus’ were used as controls.

SARS-CoV-2, Isolate hCoV-19/USA/MD-HP05647/2021 (Delta Variant) (NR-55672, BEI Resources) was used for the plaque reduction assay. Cell monolayers were stained for the determination of plaques at 6-days post infection. Plaques were counted and % reduction in the plaque by antibodies were determined based on the number of plaques in the virus only (no antibody control) wells. Standard deviations were calculated based on the 3-replicates. B.1.617.2 variant (Delta) of SARS-CoV-2 formed very small plaques as compared to the Wuhan-Hu1 SARS-CoV-2. Two novel mouse mAbs were identified (4-N22-A and 10-L24-A) that are potent in neutralizing authentic SARS-CoV-2 Delta variant infection of Vero E6 cells (IC50: 0.56 nM [4-N22-A] and 1.10 nM [10-L24-A]).

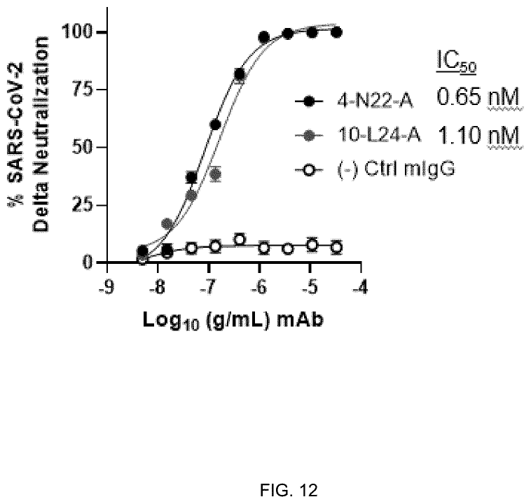

FIG. 12 shows authentic SARS-CoV-2 Delta variant neutralization (inhibition of infection of ACE2+ Vero E6 cells) by 4-N22-A and 10-L24-A.

### Example 3: SARS-CoV-2 β-Variant Spike-Specific mAbs

PENTAMICE® mice were immunized with beta variant SARS-CoV-2 spike B.1.351 protein (FIG. 13). When in-life plasma titers indicated that a strong anti-spike protein humoral immune response was achieved, the animals were euthanized and lymphocytes harvested and fused with a myeloma partner via electrofusion to generate hybridomas. The top parental hybridomas with neutralizing activity were selected for single cell cloning to generate monoclonal hybridomas, and for further analysis.

FIGS. 1 and 13 show the sequences of wild type (WT) and Beta (p, South Africa, B.1.351) variants of SARS-CoV-2, respectively, used for generating spike-specific monoclonal antibodies (mAbs). Notable differences are highlighted/shaded (in blue and red in color version). Specifically, there is a deletion of LAL that is present in the WT sequence. Point mutations are indicated in red in color versions. As described herein, Applicants generated antibodies specific to each variant.

As described in the above examples, the antibodies were screened for reactivity against SARS-CoV-2 β-Variant spike protein by ELISA. FIG. 14 is a graphical depiction of a primary screen identifying 74 β spike-selective antibodies. The wild type (WT) is shown in orange; the Beta variant is shown in blue. Approximately 80% of the primary hits were confirmed in a secondary screen.

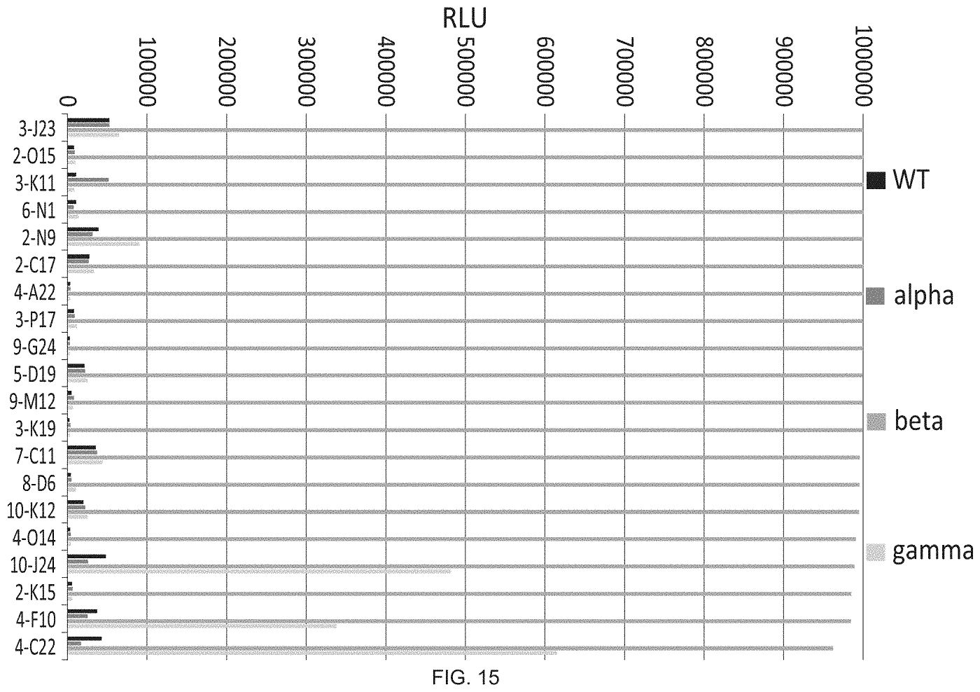

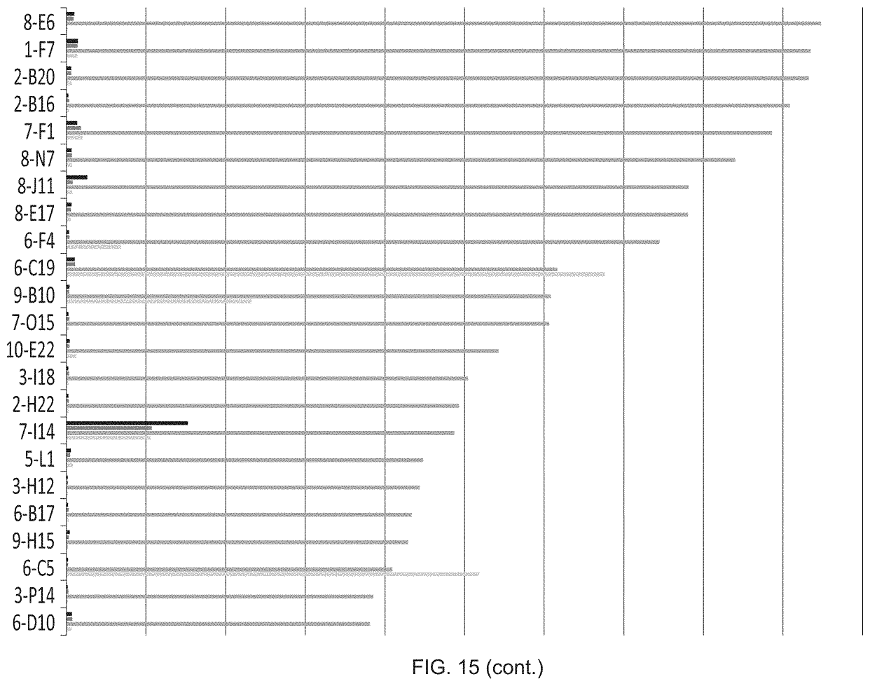

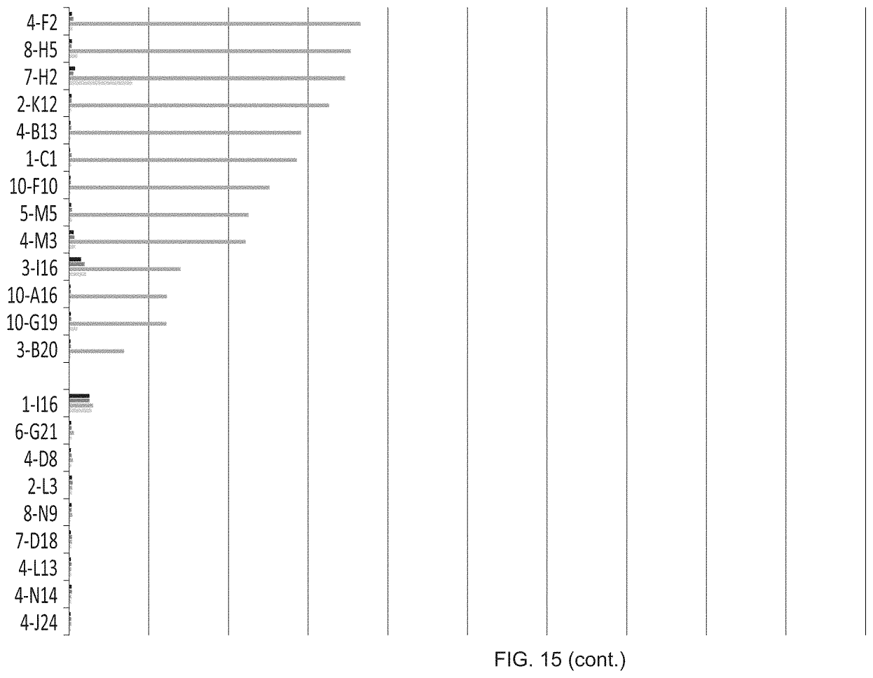

Despite sharing 99.2% identity with other SARS-CoV-2 spike proteins, Applicants discovered 49 antibodies selective for p. Further, Applicants discovered six antibodies that selectively bind both β and γ. FIG. 15 is a graphical depiction of a tertiary screen of β and β/γ spike-selective antibodies.

The top 13 hybridomas were selected for single cell cloning, VH/VL mAb sequencing, and EC₅₀ Binding. R-selective mAbs included 2-B16, 2-B20, 2-015, 3-K11, 3-P17, 4-A22, 4-O14, 6-N1, 9-G24 and 9-M12. β/γ selective mAbs included 4-C22, 6-C19 and 10-J24.

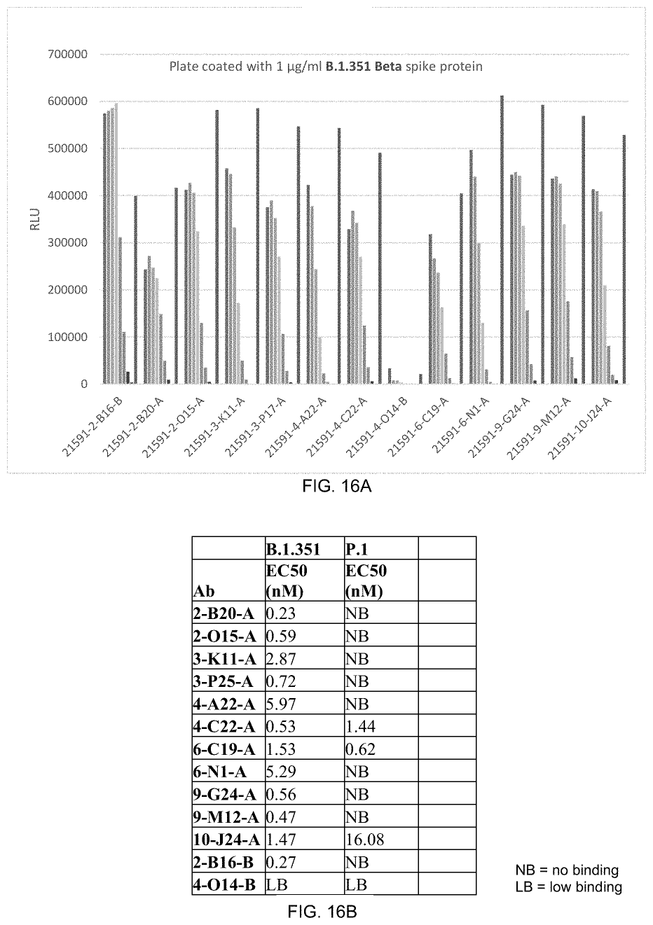

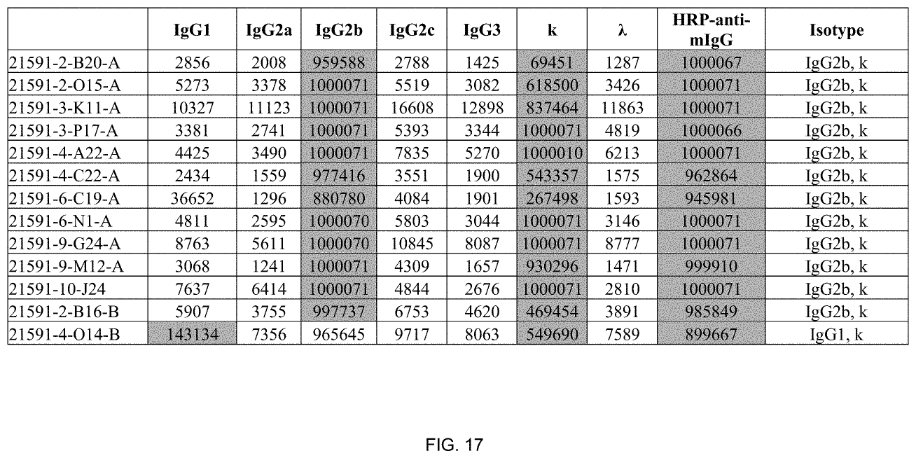

FIGS. 16A and 16B show EC₅₀ binding of purified mAbs to R-spike for thirteen antibodies (2-B20-A, 2-O15-A, 3-K11-A, 3-P17-A, 4-A22-A, 4-C22-A, 6-C19-A, 6-N1-A, 9-G24-A, 9-M12-A, 10-J24-A, 2-B16-B and 4-O14-B). FIG. 17 is a chart of the assessed isotypes of the clonal hybridomas.

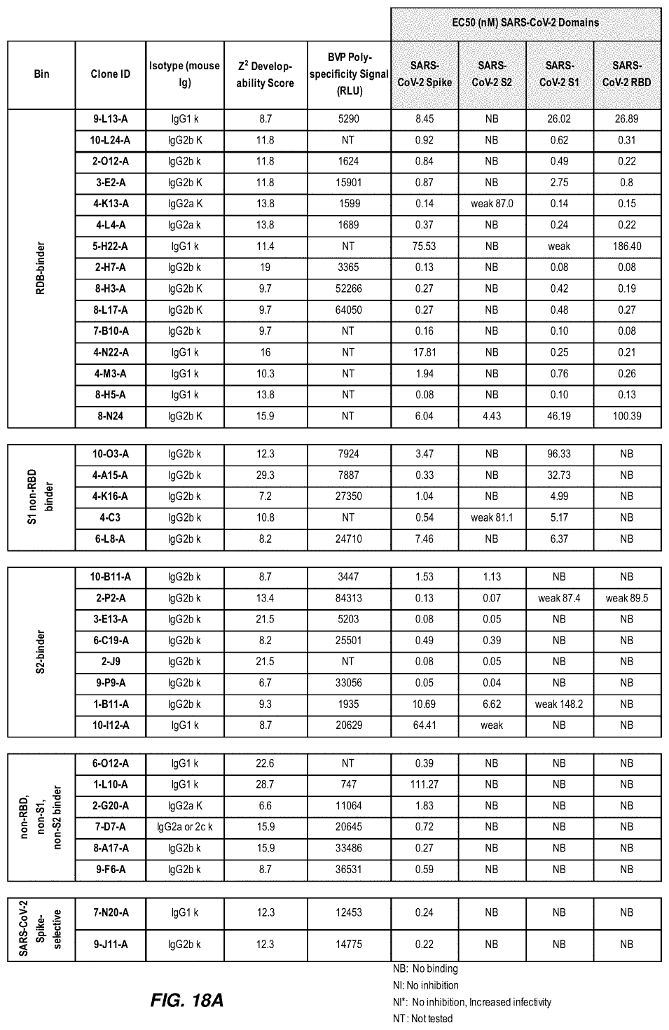

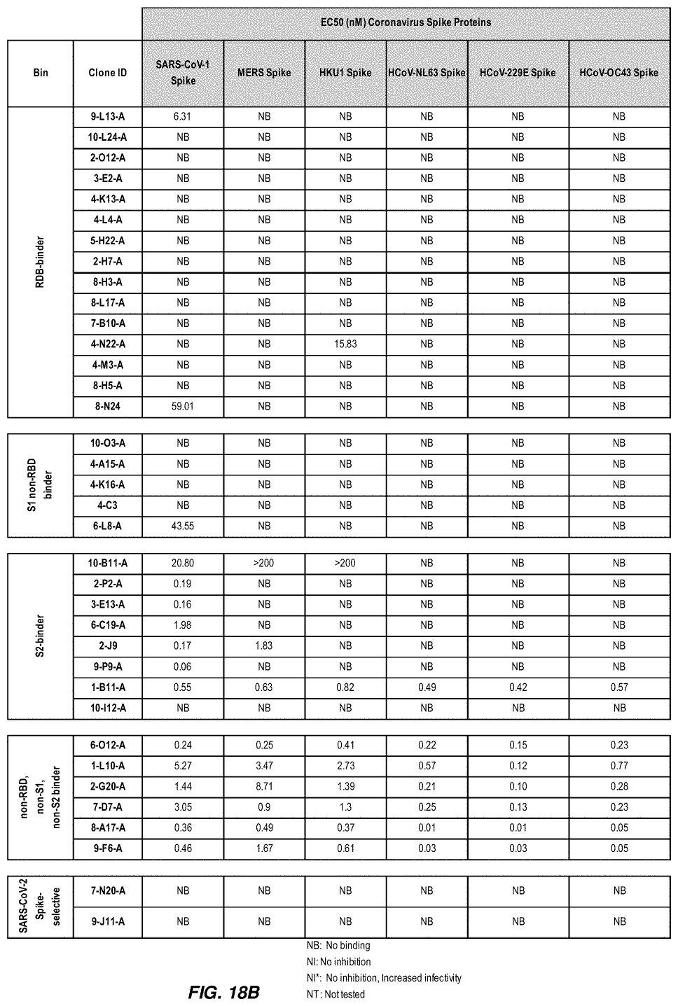

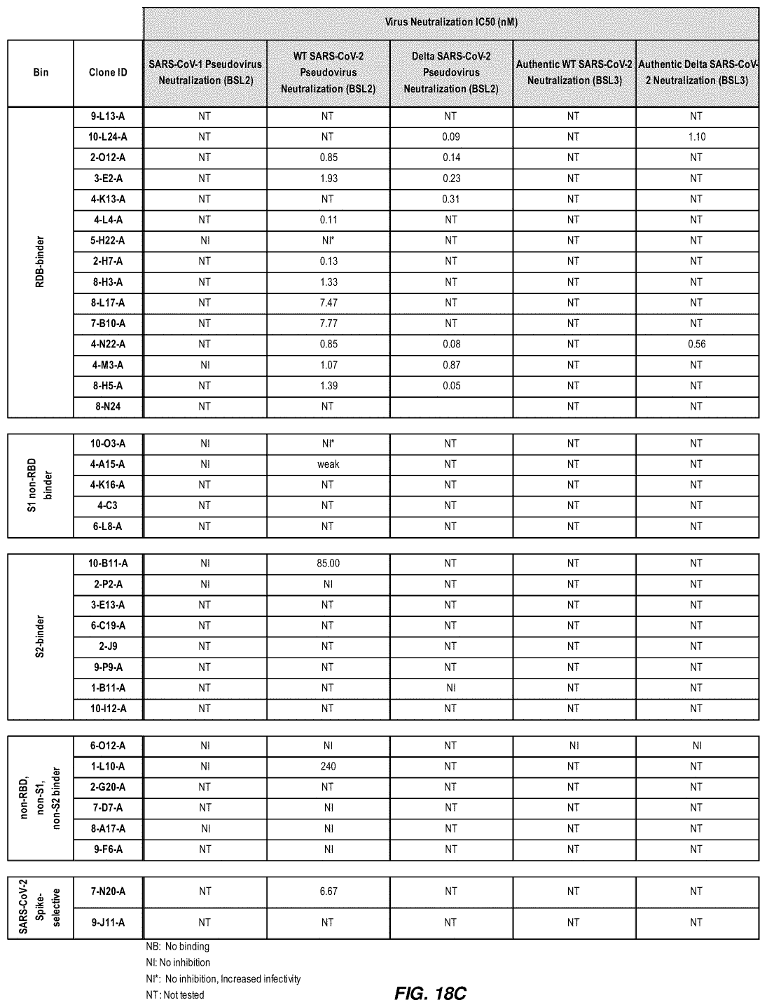

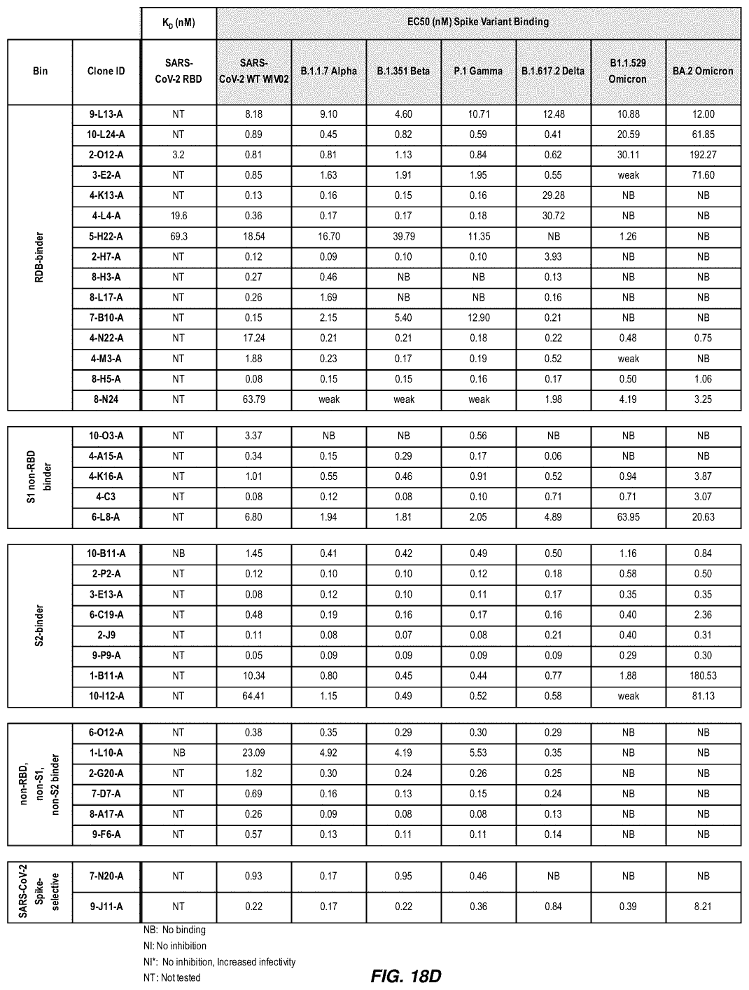

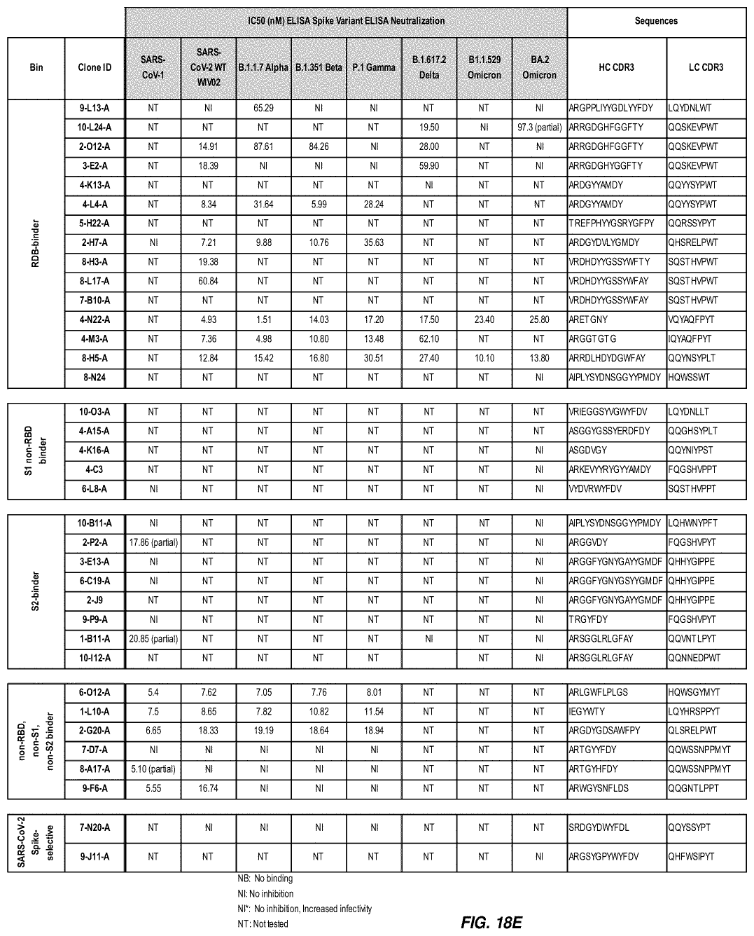

FIGS. 18A-18E provide a summary of SARS-CoV-2 spike binding monoclonal antibodies.

### Example 4: Lateral Flow Immunoassay for Detecting COVID-19 and Distinguishing Variants

The antibodies described herein can be identified and levels determined using antibody-based methods, such as, an enzyme-linked immunosorbant assay (ELISA), a radioimmunoassay (RIA) or a lateral flow immunoassay (LFA). In one embodiment, the method uses a lateral flow assay that can detect protein biomarkers at low levels. The levels of protein can also be determined by analyzing the amount of an indicator of fluorescence.

Immunoassays are frequently used to identify infectious agents, among other uses. Lateral flow assays (LFAs) are well known in the art and have played a critical role in COVID-19 testing as they have the benefit of delivering a result in a relatively short time (e.g., 5-30 minutes). One embodiment is a method of detecting SARS-CoV-2 with a lateral flow test strip that uses a sandwich-antibody capture technique.

In this example, a biological sample is applied to a portion of the strip (i.e., sample pad) of the lateral flow assay. This leads to the accumulation of a labelled binding reagent, in an analyte concentration-dependent at a detection zone on the test strip. Most LFAs are intended to operate on a purely qualitative basis. However, it is possible to measure the intensity of the test line to determine the quantity of analyte in the sample. In this regard, the test strip can then be analyzed to detect/quantify the amount of target analytes by assay reading components. A microprocessor (e.g., ASIC) can analyze and interpret the readings and display the assay results to a user and/or health care provider.

One or more of the antibodies described herein can be used in a LFA to detect SARS-CoV-2 spike protein (e.g., COVID-19) and/or distinguish among variants. In one example, an antibody specific to each variant (e.g., wild type, alpha, beta, gamma, delta and omicron) are used. This allows one to both detect the presence of COVID-19 and to distinguish among variants. Common techniques can be used to improve the sensitivity of the LFA including tags such as colloidal gold or upconverting phosphor nanoparticles.

TABLE A
Antibodies with Variant Specificities

| | mAb | | | | | | | |
|---|---|---|---|---|---|---|---|---|
| mAb | clone ID | WT | alpha | beta | gamma | delta | omicron | BA.2 |
| 1 | 10-O3-A | + | − | − | + | − | − | − |
| 2 | 8-H3-A | + | + | − | − | + | − | − |
| 3 | 9-M12-A, | − | − | + | − | − | − | − |
| | 3-P17-A, | | | | | | | |
| | 9-G24-A | | | | | | | |
| 4 | 7-N20-A | + | + | − | + | − | − | − |
| 5 | 3-K11-A | − | − | + | − | − | + | − |
| 6 | (pan) | + | + | + | + | + | + | + |
| | 1-B11-A | | | | | | | |

### Example 5: Mouse Antibodies with Activities Against the SARS-CoV-2 Omicron Variant B.1.1.529

The antibodies produced according to the Examples were tested for activity against the SARS-CoV-2 Omicron variant.

### ELISA

mAb binding reactivity was assessed by ELISA against SARS-CoV-2 B.1.1.529 spike trimer variant. ELISA plates were coated with antigen (1-10 ug/mL) and blocked with about 3% bovine serum albumin (BSA). Various dilutions of antibodies were added to the coated blocked plates and incubated about 1 hour at room temperature and then washed. Anti-mouse IgG-horse radish peroxidase (HRP) in blocking buffer was added to the wells and incubated about one hour at room temperature and washed. Pre-mixed SuperSignal ELISA Pico substrate (Thermo) solution was added to each well and bound protein was detected using Molecular Devices SpectraMax M3 luminometer and Softmax Pro Version 6.2 within about 15 minutes of adding substrate.

A panel of mAbs was screened by ELISA against SARS-CoV-2 B.1.1.529 (Omicron) spike trimer variant.

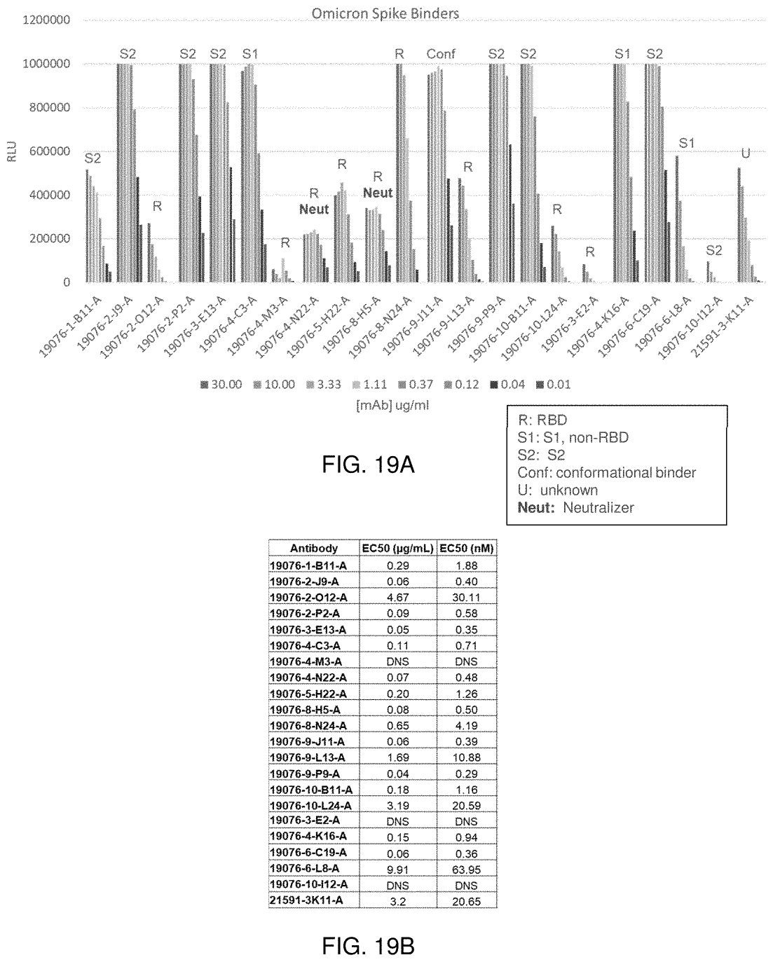

FIG. 19A is a graph depicting dose-dependent mAb binding to Omicron B.1.1.529 (8 point dose response). The binding domain is also identified (if known).

FIG. 19B shows EC₅₀ ELISA binding potency values in ug/ml and nM for Omicron B.1.1.529-binding mAbs (DNS: did not saturate, EC50 could not be calculated for these mAbs).

### Example 6: Mouse Antibodies with Activities Against the SARS-CoV-2 Omicron Variant BA.2

The antibodies produced according to the Examples were tested for activity against the SARS-CoV-2 Omicron variant BA.2.

### ELISA

mAb binding reactivity was assessed by ELISA against SARS-CoV-2 BA.2 spike trimer variant. ELISA plates were coated with antigen (1-10 ug/mL) and blocked with about 3% bovine serum albumin (BSA). Various dilutions of antibodies were added to the coated blocked plates and incubated about 1 hour at room temperature and then washed. Anti-mouse IgG-horse radish peroxidase (HRP) in blocking buffer was added to the wells and incubated about one hour at room temperature and washed. Pre-mixed SuperSignal ELISA Pico substrate (Thermo) solution was added to each well and bound protein was detected using Molecular Devices SpectraMax M3 luminometer and Softmax Pro Version 6.2 within about 15 minutes of adding substrate.

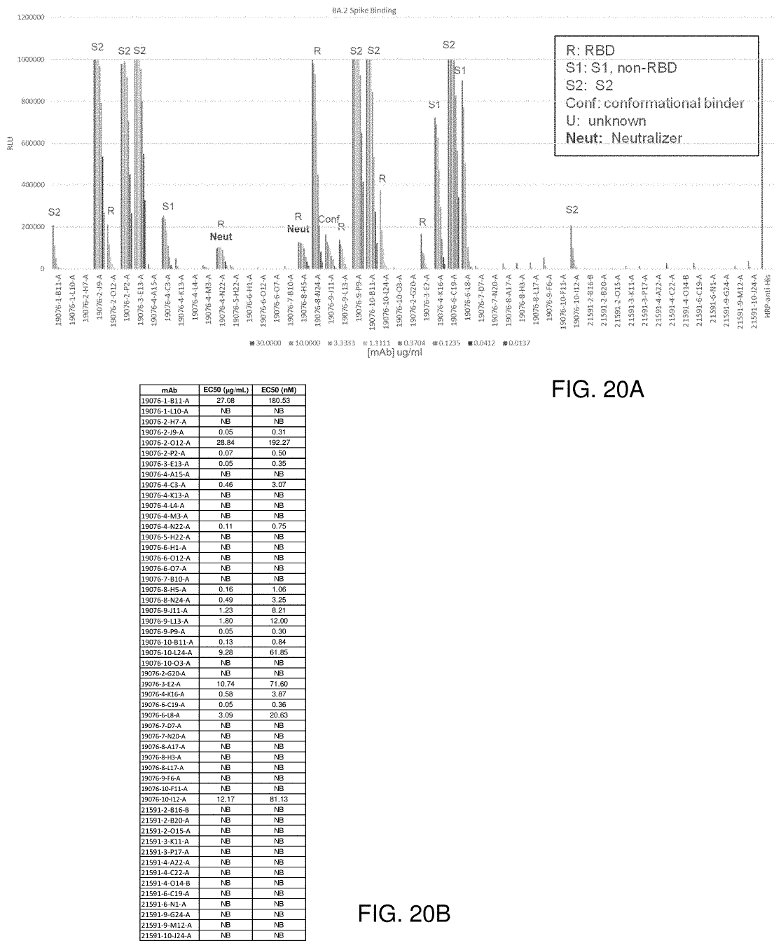

FIG. 20A is a graph depicting dose-dependent spike mAb binding to Omicron BA.2 (8 point dose response). The binding domain is also identified (if known). Omicron B.1.1.529 binders 5-H22-A and 3-K11-A do not bind to BA.2.

FIG. 20B shows EC₅₀ ELISA binding potency values in ug/ml and nM for Omicron BA.2-binding mAbs (NB: No binding).

### Example 7: Mouse Antibodies with Activities Against the SARS-CoV-2 Omicron Variants B.1.1.529 and BA.2

### ELISA Neutralization Assay

The ELISA neutralization assay described in Example 1 was used to evaluate the neutralization activity of the following mAbs: 4-N22-A, 10-L24-A, and 1-B11-A. In this assay, a 384 well ELISA plate was coated with recombinant huACE2 protein-human fragment crystallizable region (Fc); (5 ug/mL), blocked with 3% BSA for 1 hour at room temperature. Various dilutions of antibodies were pre-mixed with histidine-tagged SARS-CoV-2 B.1.1.529 spike trimer variant or SARS-CoV-2 BA.2 spike trimer variant at about 1 ug/mL for at least about 15 minutes at room temperature and then added to the 384-well plate and incubated at room temperature for about 1 hour. After incubation, plates were washed 4 times, rotated 180 degrees, and washed an additional 4 times. Bound protein was detected following incubation with anti-His-HRP antibody for about 1 hour at room temperature. Pre-mixed SuperSignal ELISA Pico substrate (Thermo) solution was added to each well and bound protein was detected using Molecular Devices SpectraMax M3 luminometer and Softmax Pro Version 6.2 within about 15 minutes of adding substrate.

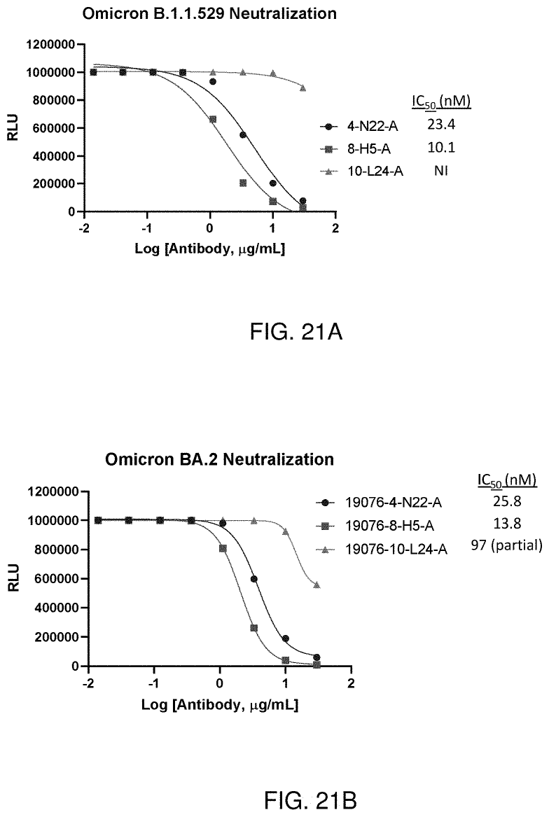

FIG. 21A is a graph depicting IC₅₀ ELISA neutralization curves for Omicron-binding mAbs 4-N22-A, 8-H5-A, and 10-L24-A. 4-N22-A inhibits the binding of SARS-CoV-2 Omicron B.1.1.529 spike trimer to huACE2. IC₅₀ potency values are listed in nanomolar (NI: no inhibition).

FIG. 21B shows IC₅₀ ELISA neutralizations curve for Omicron-binding mAbs 4-N22-A, 8-H5-A, and 10-L24-A. 4-N22-A inhibits the binding of SARS-CoV-2 Omicron BA.2 spike trimer to huACE2. IC₅₀ potency values are listed in nanomolar.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

The following Tables 1-49 illustrate the heavy chain and light chain variable region sequences as well as the hcCDRs and IcCDRs. Although the IMGT numbering scheme was used to designate the complementarity determining regions of the variable domains, it is also contemplated that alternative numbering schemes-including Kabat, Chothia, Martin, Gelfand, or Honneger—can be used to identify complementarity determining regions. See Dondelinger et al., “Understanding the Significance and Implications of Antibody Numbering and Antigen-Binding Surface/Residue Definition,” Frontiers in Immunol. 9:2278 (2018), which is hereby incorporated by reference in its entirety.

### LISTING OF SEQUENCES

TABLE 1
Sequences - Antibody Variable Region Sequences - 2-B16-B

SEQ ID NO: 1 Heavy Chain (HC), CDR1
GYTFTSYG
SEQ ID NO: 2 HC, CDR2
IYIGNGYT
SEQ ID NO: 3 HC, CDR3
ARDGTRDAMDY
SEQ ID NO: 4 Light Chain (LC), CDR1
ENIYSN
SEQ ID NO: 5 LC, CDR2
AAT
SEQ ID NO: 6 LC, CDR3
QHFYSSPWT
SEQ ID NO: 7 Heavy Chain Amino Acid Sequence
EVQLQQSGAELVRPGSSVKMSCKTSGYTFTSYGINWVKQRPGQGLEWIGYIYIGNGYTAYN
EKFKGKATLTSDTSSSTAYMQLSSLTSEDSAIYFCARDGTRDAMDYWGQGTSVTVSS
SEQ ID NO: 8 Heavy Chain Nucleotide Sequence
GAGGTCCAGCTTCAGCAGTCTGGAGCTGAGCTGGTGAGGCCTGGGTCCTCAGTGAAAA
TGTCCTGCAAGACTTCTGGATATACATTCACAAGCTACGGTATAAACTGGGTGAAGCAG
AGGCCTGGACAGGGCCTGGAATGGATTGGATATATTTATATTGGAAATGGTTATACTGC
GTACAATGAGAAGTTCAAGGGCAAGGCCACACTGACTTCAGACACATCCTCCAGCACA
GCCTACATGCAGCTCAGCAGTCTGACATCTGAGGACTCTGCAATCTATTTCTGTGCAAG
AGACGGTACTAGGGATGCTATGGACTACTGGGGTCAAGGAACCTCAGTCACCGTCTCC
TCA
SEQ ID NO: 9 Light Chain Amino Acid Sequence
DIQMTQSPASLSASVGETVTITCRASENIYSNLAWYQQKQGKSPQLLVYAATNLADGVPSRF
SGSGSGTQFSLKINSLQPEDFGSYYCQHFYSSPWTFGGGTKLEIK
SEQ ID NO: 10 Light Chain Nucleotide Sequence
GACATCCAAATGACTCAGTCTCCAGCCTCCCTATCTGCATCTGTGGGAGAAACTGTCAC
CATCACATGTCGAGCAAGTGAAAATATTTACAGTAATTTAGCATGGTATCAGCAGAAACA
GGGAAAATCTCCTCAACTCCTGGTCTATGCTGCAACAAATTTAGCAGATGGTGTGCCAT
CAAGGTTCAGTGGCAGTGGATCAGGCACACAGTTTTCTCTGAAGATCAACAGCCTGCAG
CCTGAAGATTTTGGGAGTTATTACTGTCAACATTTTTATAGTAGTCCGTGGACGTTCGGT
GGAGGCACCAAGCTGGAAATCAAA

TABLE 2
Sequences - Antibody Variable Region Sequences - 2-B20-A

SEQ ID NO: 11 Heavy Chain (HC), CDR1
GYTFTTYG
SEQ ID NO: 12 CDR2
INTFSGLP
SEQ ID NO: 13 CDR3
ARVKVHDYDGDFYGMDY
SEQ ID NO: 14 Light Chain (LC), CDR1
KSLLNSDGFTY
SEQ ID NO: 15 LC, CDR2
LVS
SEQ ID NO: 16 LC, CDR3
FQSNYLPLT
SEQ ID NO: 17 Heavy Chain Amino Acid Sequence
QIQLVQSGPELKKPGETVKISCKASGYTFTTYGMSWVKQAPGKGLKWMGWINTFSGLPTY
ADDFNGRFAFSLETSASTAYLQINNLKDEDTATYFCARVKVHDYDGDFYGMDYWGQGTSV
TVSS
SEQ ID NO: 18 Heavy Chain Nucleotide Sequence
CAGATCCAGTTGGTACAGTCTGGACCTGAGCTGAAGAAGCCTGGAGAGACAGTCAAAA
TCTCCTGCAAGGCTTCTGGATATACCTTCACAACCTATGGAATGAGCTGGGTGAAACAG
GCACCAGGAAAGGGTTTAAAGTGGATGGGCTGGATAAACACCTTCTCTGGACTGCCAA
CATATGCTGATGACTTCAACGGACGGTTTGCCTTCTCTTTGGAAACCTCTGCCAGCACT
GCCTATTTGCAGATCAACAACCTCAAAGATGAGGACACGGCGACATATTTCTGTGCAAG
AGTGAAGGTTCATGATTACGACGGGGATTTCTATGGTATGGACTACTGGGGTCAAGGAA
CCTCAGTCACCGTCTCCTCA
SEQ ID NO: 19 Light Chain Amino Acid Sequence
DVVLTQTPLSLPVNIGDQASISCKSTKSLLNSDGFTYLDWYLQKPGQSPQLLIYLVSNRFSGV
PDRFSGSGSGTDFTLKISRVEAEDLGVYYCFQSNYLPLTFGAGTKLELK
SEQ ID NO: 20 Light Chain Nucleotide Sequence
GATGTTGTTCTGACCCAAACTCCACTCTCTCTGCCTGTCAATATTGGAGATCAAGCCTCT
ATCTCTTGCAAGTCTACTAAGAGTCTTCTGAATAGTGATGGATTCACTTATTTGGACTGG
TACCTGCAGAAGCCAGGCCAGTCTCCACAGCTCCTAATATATTTGGTTTCTAATCGATTT
TCTGGAGTTCCAGACAGGTTCAGTGGCAGTGGGTCAGGAACAGATTTCACACTCAAGAT
CAGCAGAGTGGAGGCTGAGGATTTGGGAGTTTATTATTGCTTCCAGAGTAACTATCTTC
CTCTCACGTTCGGTGCTGGGACCAAGCTGGAGCTGAAA

TABLE 3
Sequences - Antibody Variable Region Sequences - 2-O15-A

SEQ ID NO: 31 Heavy Chain (HC), CDR1
GYTFTDCE
SEQ ID NO: 32 CDR2
IDPETGDT
SEQ ID NO: 33 CDR3
TRRRDYGNPYWYFDV
SEQ ID NO: 34 Light Chain (LC), CDR1
SSISSSN
SEQ ID NO: 35 LC, CDR2
GTS
SEQ ID NO: 36 LC, CDR3
QQWTSYPYT
SEQ ID NO: 37 Heavy Chain Amino Acid Sequence
QVQLQQSGAELVRPGASVKLSCKASGYTFTDCEMHWVKQTPVHGLEWIGVIDPETGDTAY
NQKFKGKATLTADKSSSTAYMELRSLTSEDSAVYYCTRRRDYGNPYWYFDVWGAGTTVTV
SS
SEQ ID NO: 38 Heavy Chain Nucleotide Sequence
CAGGTTCAACTGCAGCAGTCTGGGGCTGAGCTGGTGAGGCCTGGGGCTTCAGTGAAG
CTGTCCTGCAAGGCTTCGGGCTACACATTTACTGACTGTGAAATGCACTGGGTGAAGCA
GACCCCTGTGCATGGCCTGGAATGGATTGGAGTTATTGATCCTGAAACTGGTGATACTG
CCTACAATCAGAAGTTCAAGGGCAAGGCCACACTGACTGCAGACAAATCCTCCAGCACA
GCCTACATGGAGCTCCGCAGCCTGACATCTGAGGACTCTGCCGTCTATTACTGTACAAG
ACGCAGGGACTATGGTAACCCCTACTGGTACTTCGATGTCTGGGGCGCAGGGACCACG
GTCACCGTCTCCTCA
SEQ ID NO: 39 Light Chain Amino Acid Sequence
EIVLTQSPALMAASPGEKVTITCSVSSSISSSNLHWYQQKSGTSPKPWIYGTSNLASGVPVR
FSGSGSGTSYSLTISSMEAEDAATYYCQQWTSYPYTFGSGTKLEIK
SEQ ID NO: 40 Light Chain Nucleotide Sequence
GAAATTGTGCTCACCCAGTCTCCAGCACTCATGGCTGCATCTCCAGGGGAGAAGGTCA
CCATCACCTGCAGTGTCAGCTCAAGTATAAGTTCCAGCAACTTACACTGGTACCAGCAG
AAGTCAGGAACCTCCCCCAAACCCTGGATTTATGGCACATCCAACCTTGCTTCTGGAGT
CCCTGTTCGCTTCAGTGGCAGTGGATCTGGGACCTCTTATTCTCTCACAATCAGCAGCA
TGGAGGCTGAAGATGCTGCCACTTATTACTGTCAACAGTGGACTAGTTACCCATATACG
TTCGGATCGGGGACCAAGCTGGAAATAAAA

TABLE 4
Sequences - Antibody Variable Region Sequences - 3-K11-A

SEQ ID NO: 41 Heavy Chain (HC), CDR1
GYTFTSYV
SEQ ID NO: 42 HC, CDR2
INPYNDGT
SEQ ID NO: 43 HC, CDR3
AREAYRYFDV
SEQ ID NO: 44 Light Chain (LC), CDR1
ENIYSN
SEQ ID NO: 45 LC, CDR2
GAT
SEQ ID NO: 46 LC, CDR3
QHFWGIPWT
SEQ ID NO: 47 Heavy Chain Amino Acid Sequence
EVQLQQSGPELVKPGASVKMSCKASGYTFTSYVMHWVKQKPGQGLEWIGYINPYNDGTEY
NEKFKGKATLTSDKSSSTAYMELSSLTSEDSAVYHCAREAYRYFDVWGTGTTVTVSS
SEQ ID NO: 48 Heavy Chain Nucleotide Sequence
GAGGTCCAGCTGCAGCAGTCTGGACCTGAGCTGGTAAAGCCTGGGGCTTCAGTGAAGA
TGTCCTGCAAGGCTTCTGGATACACATTCACTAGCTATGTTATGCACTGGGTGAAGCAG
AAGCCTGGGCAGGGCCTTGAGTGGATTGGATATATTAATCCTTACAATGATGGTACTGA
GTACAATGAGAAATTCAAAGGCAAGGCCACACTGACTTCAGACAAATCCTCCAGCACAG
CCTACATGGAGCTCAGCAGCCTGACCTCTGAGGACTCTGCGGTCTATCACTGTGCAAG
AGAGGCCTACCGGTACTTCGATGTCTGGGGCACAGGGACCACGGTCACCGTCTCCTCA
SEQ ID NO: 49 Light Chain Amino Acid Sequence
DIQMTQSPASLSVSVGETVTITCRASENIYSNLAWYQQKQGKYPQLLVYGATNLADGVPSR
FSGSGSGTQYFLKINSLQSEDFGSYYCQHFWGIPWTFGGGTKLEIK
SEQ ID NO: 50 Light Chain Nucleotide Sequence
GACATCCAGATGACTCAGTCTCCAGCCTCCCTATCTGTATCTGTGGGAGAAACTGTCAC
CATCACATGTCGAGCAAGTGAGAATATTTACAGTAATTTAGCATGGTATCAGCAGAAACA
GGGAAAATATCCTCAGCTCCTGGTCTATGGTGCAACAAACTTAGCAGATGGTGTGCCAT
CAAGGTTCAGTGGCAGTGGATCAGGCACACAGTATTTCCTCAAGATCAACAGCCTGCAG
TCTGAAGATTTTGGGAGTTATTACTGTCAACATTTTTGGGGTATTCCGTGGACGTTCGGT
GGAGGCACCAAGCTGGAAATCAAA

TABLE 5
Sequences - Antibody Variable Region Sequences - 3-P17-A

SEQ ID NO: 51 Heavy Chain (HC), CDR1
GFSLTSYG
SEQ ID NO: 52 HC, CDR2
IWSGGIT
SEQ ID NO: 53 HC, CDR3
ARIITTVLDQFAD
SEQ ID NO: 54 Light Chain (LC), CDR1
QSVSTSTYSY
SEQ ID NO: 55 LC, CDR2
YAS
SEQ ID NO: 56 LC, CDR3
QHSWEIPPT
SEQ ID NO: 57 Heavy Chain Amino Acid Sequence
QVQLKQSGPGLVQPSQSLSITCTVSGFSLTSYGVHWVRQSPGKGLEWLGVIWSGGITDYN
AGFISRLSISKDNSKSQVFFTMNSLQADDTAIYYCARIITTVLDQFADWGQGTLVTVSA
SEQ ID NO: 58 Heavy Chain Nucleotide Sequence
CAGGTGCAGCTGAAGCAGTCAGGACCTGGCCTAGTGCAGCCCTCACAGAGCCTGTCCA
TCACCTGCACAGTCTCTGGTTTCTCATTAACTAGCTATGGTGTACACTGGGTTCGCCAGT
CTCCAGGAAAGGGTCTGGAGTGGCTGGGAGTGATATGGAGTGGTGGAATCACAGACTA
TAATGCAGGTTTCATATCCAGACTGAGCATCAGCAAGGACAATTCCAAGAGCCAAGTTT
TCTTTACAATGAACAGTCTGCAAGCTGATGACACAGCCATATATTACTGTGCCAGGATTA
TTACTACGGTTCTAGATCAGTTTGCTGACTGGGGCCAAGGGACTCTGGTCACTGTCTCT
GCA
SEQ ID NO: 59 Light Chain Amino Acid Sequence
DIVLTQSPASLAVSLGQRATISCRASQSVSTSTYSYIHWYQQKPGQPPKLLIKYASNLESGVP
ARFSGSGSGTDFTLNIHPVEEEDTATYYCQHSWEIPPTFGAGTKLELK
SEQ ID NO: 60 Light Chain Nucleotide Sequence
GACATTGTGCTGACACAGTCTCCTGCTTCCTTAGCTGTATCTCTGGGGCAGAGGGCCAC
CATCTCATGCAGGGCCAGCCAAAGTGTCAGTACATCTACCTATAGTTATATTCACTGGTA
CCAACAGAAACCAGGACAGCCACCCAAACTCCTCATCAAGTATGCATCCAACCTAGAAT
CTGGGGTCCCTGCCAGGTTCAGTGGCAGTGGGTCTGGGACAGACTTCACCCTCAACAT
CCATCCTGTGGAGGAGGAGGATACTGCAACATATTACTGTCAGCACAGTTGGGAGATTC
CTCCCACGTTCGGTGCTGGGACCAAGCTGGAGCTGAAA

TABLE 6
Sequences - Antibody Variable Region Sequences - 4-A22-A

SEQ ID NO: 61 Heavy Chain (HC), CDR1
GYTFTSYV
SEQ ID NO: 62 HC, CDR2
INPYNDGT
SEQ ID NO: 63 HC, CDR3
AREAYRYFDV
SEQ ID NO: 64 Light Chain (LC), CDR1
ENIYSN
SEQ ID NO: 65 LC, CDR2
AAT
SEQ ID NO: 66 LC, CDR3
QHFWGTPWT
SEQ ID NO: 67 Heavy Chain Amino Acid Sequence
EVQLQQSGPELVKPGASVKMSCKASGYTFTSYVMHWVKQKPGQGLEWIGYINPYNDGTEY
NEKFKGKATLTSDKSSSTAYMELSSLTSEDSAVYYCAREAYRYFDVWGTGTTVTVSS
SEQ ID NO: 68 Heavy Chain Nucleotide Sequence
GAGGTCCAGCTGCAGCAGTCTGGACCTGAGCTGGTAAAGCCTGGGGCTTCAGTGAAGA
TGTCCTGCAAGGCTTCTGGATACACATTCACTAGCTATGTTATGCACTGGGTGAAGCAG
AAGCCTGGGCAGGGCCTTGAGTGGATTGGATATATTAATCCTTACAATGATGGTACTGA
GTACAATGAGAAGTTCAAAGGCAAGGCCACACTGACTTCAGACAAATCCTCCAGCACAG
CCTACATGGAGCTCAGCAGCCTGACCTCTGAGGACTCTGCGGTCTATTACTGTGCAAGA
GAGGCCTACCGGTACTTCGATGTCTGGGGCACAGGGACCACGGTCACCGTCTCCTCA
SEQ ID NO: 69 Light Chain Amino Acid Sequence
DIQMTQSPASLSVSVGETVTITCRASENIYSNLAWYQQKQGKSPQLLVYAATNLAEGVPSRF
SGSGSGTQYSLKINSLQSEDFGSYYCQHFWGTPWTFGGGTKLEIK
SEQ ID NO: 70 Light Chain Nucleotide Sequence
GACATCCAGATGACTCAGTCTCCAGCCTCCCTATCTGTATCTGTGGGAGAAACTGTCAC
CATCACATGTCGAGCAAGTGAGAATATTTACAGTAATTTAGCATGGTATCAGCAGAAACA
GGGAAAATCTCCTCAGCTCCTGGTCTATGCTGCAACAAACTTAGCAGAAGGTGTGCCAT
CAAGGTTCAGTGGCAGTGGATCAGGCACACAGTATTCCCTCAAGATCAACAGCCTGCA
GTCTGAAGATTTTGGGAGTTATTACTGTCAACATTTTTGGGGTACTCCGTGGACGTTCG
GTGGAGGCACCAAACTGGAAATCAAA

TABLE 7
Sequences - Antibody Variable Region Sequences - 4-C22-A

SEQ ID NO: 71 Heavy Chain (HC), CDR1
GFTFSSFG
SEQ ID NO: 72 HC, CDR2
ISSGGGFT
SEQ ID NO: 73 HC, CDR3
ARRLYDYDWFAY
SEQ ID NO: 74 Light Chain (LC), CDR1
QDVVTA
SEQ ID NO: 75 LC, CDR2
WAS
SEQ ID NO: 76 LC, CDR3
QQYSSYPLT
SEQ ID NO: 77 Heavy Chain Amino Acid Sequence
EVQLVESGGDLVKPGGSLKLSCAASGFTFSSFGMSWVRQTPDKRLEWVATISSGGGFTYY
PDSVEGRFTISRDNAKNTLYLQMSSLKSEDTAMYYCARRLYDYDWFAYWGQGTLVTVSA
SEQ ID NO: 78 Heavy Chain Nucleotide Sequence
GAGGTGCAGCTGGTGGAGTCTGGGGGAGACTTAGTGAAGCCTGGAGGGTCCCTGAAA
CTCTCCTGTGCAGCCTCTGGATTCACTTTCAGTAGCTTTGGCATGTCTTGGGTTCGCCA
GACTCCAGACAAGAGGCTGGAGTGGGTCGCAACCATTAGTAGTGGTGGTGGTTTCACC
TACTATCCAGACAGTGTGGAGGGGCGATTCACCATCTCCAGAGACAATGCCAAGAACA
CCCTGTACCTGCAAATGAGCAGTCTGAAGTCTGAGGACACAGCCATGTATTACTGTGCA
AGACGGCTCTATGATTACGACTGGTTTGCTTACTGGGGCCAAGGGACTCTGGTCACTGT
CTCTGCA
SEQ ID NO: 79 Light Chain Amino Acid Sequence
DIVMTQSHKIMSTSVGDRVSITCKASQDVVTAVAWYQKKPGQSPKLLIHWASTRHTGVPDR
FTGSGSGTDFTLTISNVQSEDLADYFCQQYSSYPLTFGAGTKLELK
SEQ ID NO: 80 Light Chain Nucleotide Sequence
GACATTGTGATGACCCAGTCTCACAAAATCATGTCCACATCAGTAGGAGACAGGGTCAG
CATCACCTGCAAGGCCAGTCAGGATGTGGTTACTGCTGTAGCCTGGTATCAAAAGAAAC
CAGGGCAATCTCCTAAACTACTGATTCATTGGGCATCCACCCGGCACACTGGAGTCCCT
GATCGCTTCACAGGCAGTGGATCTGGGACAGATTTCACTCTCACCATTAGCAATGTGCA
GTCTGAAGACTTGGCAGATTATTTCTGTCAGCAATATAGCAGCTATCCTCTCACGTTCGG
TGCTGGGACCAAGCTGGAGCTGAAA

TABLE 8
Sequences - Antibody Variable Region Sequences - 4-O14-B

SEQ ID NO: 81 Heavy Chain (HC), CDR1
GYAFISHW
SEQ ID NO: 82 HC, CDR2
IDPNSSGS
SEQ ID NO: 83 HC, CDR3
ARSPYYSNPYWYFDV
SEQ ID NO: 84 Light Chain (LC), CDR1
SSISSSN
SEQ ID NO: 85 LC, CDR2
GTS
SEQ ID NO: 86 LC, CDR3
QQWSSYPFT
SEQ ID NO: 87 Heavy Chain Amino Acid Sequence
QVQLQQPGAELVKPGASVKLSCKASGYAFISHWMHWVRQRPGRGLEWIGRIDPNSSGSK
DNEKFRSKATLTVDKPSSTAYMQLSSLTSEDSAVYYCARSPYYSNPYWYFDVWGAGTTVT
VSS
SEQ ID NO: 88 Heavy Chain Nucleotide Sequence
CAGGTCCAACTGCAGCAGCCTGGGGCTGAGCTTGTGAAGCCTGGGGCTTCAGTGAAG
CTGTCCTGCAAGGCTTCTGGCTACGCCTTCATTAGCCACTGGATGCACTGGGTGAGGC
AGAGGCCTGGACGAGGCCTTGAGTGGATTGGAAGGATTGATCCTAATAGTAGTGGTTC
TAAAGATAATGAGAAGTTCAGGAGCAAGGCCACACTGACTGTAGACAAACCCTCCAGCA
CAGCCTACATGCAGCTCAGCAGCCTGACATCTGAGGACTCTGCGGTCTATTATTGTGCA
AGATCCCCCTACTATAGTAACCCTTACTGGTACTTCGATGTCTGGGGCGCAGGGACCAC
GGTCACCGTCTCCTCA
SEQ ID NO: 89 Light Chain Amino Acid Sequence
EIVLTQSPALMAASPGEKVTITCSVSSSISSSNLHWYQQKSETSPKPWIYGTSNLASGVPVR
FSGSGSGTSYSLTITNMEAEDAATYYCQQWSSYPFTFGSGTKLEIK
SEQ ID NO: 90 Light Chain Nucleotide Sequence
GAAATTGTGCTCACCCAGTCTCCAGCACTCATGGCTGCATCTCCAGGGGAGAAGGTCA
CCATCACCTGCAGTGTCAGTTCAAGTATAAGTTCCAGCAACTTGCACTGGTACCAGCAG
AAGTCAGAAACCTCCCCCAAACCCTGGATTTATGGCACATCCAACCTGGCTTCTGGAGT
CCCGGTTCGCTTCAGTGGCAGTGGATCTGGGACCTCTTATTCTCTCACAATCACCAACA
TGGAGGCTGAAGATGCTGCCACTTATTACTGTCAACAGTGGAGTAGTTATCCATTCACG
TTCGGCTCGGGGACAAAGTTGGAAATAAAA

TABLE 9
Sequences - Antibody Variable Region Sequences - 6-C19-A

SEQ ID NO: 91 Heavy Chain (HC), CDR1
GNTFTTAG
SEQ ID NO: 92 HC, CDR2
INTHSGEP
SEQ ID NO: 93 HC, CDR3
ARYGDYYYFDC
SEQ ID NO: 94 Light Chain (LC), CDR1
GNIHNY
SEQ ID NO: 95 LC, CDR2
NAK
SEQ ID NO: 96 LC, CDR3
QHFWTTPWS
SEQ ID NO: 97 Heavy Chain Amino Acid Sequence
QIQLVQSGPELKKPGETVKISCKASGNTFTTAGMQWVQKMPGKGFKWIGWINTHSGEPKY
AEDFKGRFAFSLETSASTAYLQISNLKNEDTATYFCARYGDYYYFDCWGQGTTLTVSS
SEQ ID NO: 98 Heavy Chain Nucleotide Sequence
CAGATCCAGTTGGTGCAGTCTGGACCTGAGCTGAAGAAGCCTGGAGAGACAGTCAAGA
TCTCCTGCAAGGCTTCTGGGAATACCTTCACAACTGCTGGAATGCAGTGGGTGCAAAAG
ATGCCAGGAAAGGGTTTTAAGTGGATTGGCTGGATAAACACCCACTCTGGAGAGCCAAA
ATATGCAGAAGACTTCAAGGGACGGTTTGCCTTCTCTTTGGAAACCTCTGCCAGCACTG
CCTATTTACAGATAAGCAACCTCAAAAATGAGGACACGGCTACGTATTTCTGTGCGAGA
TATGGTGATTACTACTACTTTGACTGCTGGGGCCAAGGCACCACTCTCACAGTCTCCTC
A
SEQ ID NO: 99 Light Chain Amino Acid Sequence
DIQMTQSPASLSASVGETVTITCRASGNIHNYLAWYQQKQGKSPQLLVYNAKTLADGVPSR
FSGSGSGTQYSLKINSLQPEDFGSFYCQHFWTTPWSFGGGTKLEIK
SEQ ID NO: 100 Light Chain Nucleotide Sequence
GACATCCAGATGACTCAGTCTCCAGCCTCCCTATCTGCATCTGTGGGAGAAACTGTCAC
CATCACATGTCGAGCAAGTGGGAATATTCACAATTATTTAGCATGGTATCAGCAGAAACA
GGGAAAATCTCCTCAGCTCCTGGTCTATAATGCAAAAACCTTAGCAGATGGTGTGCCAT
CAAGGTTCAGTGGCAGTGGATCAGGAACACAATATTCTCTCAAGATCAACAGCCTGCAG
CCTGAAGATTTTGGGAGTTTTTACTGTCAACATTTTTGGACGACTCCGTGGTCGTTCGGT
GGAGGCACCAAGCTGGAAATCAAA

TABLE 10
Sequences - Antibody Variable Region Sequences - 6-N1-A

SEQ ID NO: 101 Heavy Chain (HC), CDR1
GYTFTDYY
SEQ ID NO: 102 HC, CDR2
INPNNGGI
SEQ ID NO: 103 HC, CDR3
ARRQVFYDGYYDYAMDY
SEQ ID NO: 104 Light Chain (LC), CDR1
QSVSTSSYSY
SEQ ID NO: 105 LC, CDR2
YAS
SEQ ID NO: 106 LC, CDR3
QHNWEIPPT
SEQ ID NO: 107 Heavy Chain Amino Acid Sequence
EVQLQQSGPELVKPGASVKISCKASGYTFTDYVMNWVQQSHGKSLEWIGDINPNNGGIRY
NQNFKGKATLTVEKSSSTAYMELRSLTSEDSAVYYCARRQVFYDGYVDYAMDYWGQGTS
VTVSS
SEQ ID NO: 108 Heavy Chain Nucleotide Sequence
GAGGTCCAGCTGCAACAATCTGGACCTGAGCTGGTGAAGCCTGGGGCTTCAGTGAAGA
TATCCTGTAAGGCTTCTGGATACACGTTCACTGACTACTACATGAACTGGGTGCAGCAG
AGCCATGGAAAGAGCCTTGAGTGGATTGGTGATATTAATCCTAACAATGGTGGTATTCG
CTACAACCAGAACTTCAAGGGCAAGGCCACATTGACTGTAGAGAAGTCCTCCAGCACA
GCCTACATGGAGCTCCGCAGCCTGACATCTGAGGACTCTGCAGTCTATTACTGTGCAAG
AAGGCAGGTATTCTATGATGGTTACTACGACTATGCTATGGACTATTGGGGTCAAGGAA
CCTCAGTCACCGTCTCCTCA
SEQ ID NO: 109 Light Chain Amino Acid Sequence
DIVLTQSPAS LAVSLGQRATISCRASQSVSTSSYSYM HWYQQKPGQ PPKLLI KYASN
LESGVPARFSGSGSGTDFTLNIHPVEEEDTATYFCQHNWEIPPTFGGGTKLEIK
SEQ ID NO: 110 Light Chain Nucleotide Sequence
GACATTGTGCTGACACAGTCTCCTGCTTCCTTAGCTGTATCTCTGGGGCAGAGGGCCAC
CATCTCATGCAGGGCCAGCCAAAGTGTCAGTACATCTAGCTATAGTTATATGCACTGGT
ACCAACAGAAACCAGGACAGCCACCCAAACTCCTCATCAAGTATGCATCCAACCTAGAA
TCTGGGGTCCCTGCCAGGTTCAGTGGCAGTGGGTCTGGGACAGACTTCACCCTCAACA
TCCATCCTGTGGAGGAGGAAGATACTGCAACATATTTCTGTCAGCACAATTGGGAGATT
CCTCCCACGTTCGGAGGGGGGACCAAGCTGGAAATAAAA

TABLE 11
Sequences - Antibody Variable Region Sequences - 9-G24-A

SEQ ID NO: 131 Heavy Chain (HC), CDR1
GYTFTDYE
SEQ ID NO: 132 HC, CDR2
IDPETGDT
SEQ ID NO: 133 HC, CDR3
ARRRDYGNPYWYFDV
SEQ ID NO: 134 Light Chain (LC), CDR1
SSISSSN
SEQ ID NO: 135 LC, CDR2
GTS
SEQ ID NO: 136 LC, CDR3
QQWSNYPYT
SEQ ID NO: 137 Heavy Chain Amino Acid Sequence
QVQLQQSGAELVRPGASVKLSCKASGYTFTDYEMHWVKQTPVHGLEWIGVIDPETGDTAY
NQKFKDKATLTADKSSSTAYMELRSLTSEDSAVYYCARRRDYGNPYWYFDVWGAGTAVTV
SS
SEQ ID NO: 138 Heavy Chain Nucleotide Sequence
CAGGTTCAACTGCAGCAGTCTGGGGCTGAGCTGGTGAGGCCTGGGGCTTCAGTGAAG
CTGTCCTGCAAGGCTTCGGGCTACACATTTACTGACTATGAAATGCACTGGGTGAAGCA
GACACCTGTGCATGGCCTGGAATGGATTGGAGTTATTGATCCTGAAACTGGTGATACTG
CCTACAATCAGAAGTTCAAGGACAAGGCCACACTGACTGCAGACAAATCCTCCAGCACA
GCCTACATGGAGCTCCGCAGCCTGACATCTGAGGACTCTGCCGTCTATTACTGTGCAAG
ACGCAGGGACTATGGTAACCCCTACTGGTACTTCGATGTCTGGGGCGCAGGGACCGCG
GTCACCGTCTCCTCA
SEQ ID NO: 139 Light Chain Amino Acid Sequence
EIVL TQSPALMAASPG E KVTITCSASSSISSSN LHWYQQKSGTS P KPWIYGTSN
LASGVPVRFSGSGSGTSYSLTISSMEAEDAATYVCQQWSNYPYTFGSGTKLEIK
SEQ ID NO: 140 Light Chain Nucleotide Sequence
GAAATTGTGCTCACCCAGTCTCCAGCACTCATGGCTGCATCTCCAGGGGAGAAGGTCA
CCATCACCTGCAGTGCCAGCTCAAGTATAAGTTCCAGCAACTTACACTGGTACCAGCAG
AAGTCAGGAACCTCCCCCAAACCCTGGATTTATGGCACATCCAACCTTGCTTCTGGAGT
CCCTGTTCGCTTCAGTGGCAGTGGATCTGGGACCTCTTATTCTCTCACAATCAGCAGCA
TGGAGGCTGAAGATGCTGCCACTTATTACTGTCAACAGTGGAGTAATTACCCATATACG
TTCGGATCGGGGACCAAGTTGGAGATTAAA

TABLE 12
Sequences - Antibody Variable Region Sequences - 9-M12-A

SEQ ID NO: 141 Heavy Chain (HC), CDR1
GFTFSSYG
SEQ ID NO: 142 HC, CDR2
ISSGGGYT
SEQ ID NO: 143 HC, CDR3
ARPLIPTMVEAFDY
SEQ ID NO: 144 Light Chain (LC), CDR1
QSVGNN
SEQ ID NO: 145 LC, CDR2
YAS
SEQ ID NO: 146 LC, CDR3
QQHYSSPFT
SEQ ID NO: 147 Heavy Chain Amino Acid Sequence
EVQLVESGGDLVKPGGSLKLSCTASGFTFSSYGMSWVRQTPDKRLEWVTTISSGGGYTYV
PDSVKGRFTISRDNAKNTLYLHMSSLKSEDTAMYFCARPLIPTMVEAFDYWGQGTSLTVSS
SEQ ID NO: 148 Heavy Chain Nucleotide Sequence
GAGGTGCAGCTGGTGGAGTCTGGGGGAGACTTAGTGAAGCCTGGAGGGTCCCTGAAA
CTCTCCTGTACAGCCTCTGGATTCACTTTCAGTAGCTATGGCATGTCTTGGGTTCGCCA
GACTCCAGACAAGAGGCTGGAGTGGGTCACAACCATTAGTAGTGGTGGTGGTTA
CACCTACTATCCAGACAGTGTGAAGGGGCGATTCACCATCTCCAGAGACAATGCCAAGA
ACACCCTGTACCTGCACATGAGCAGTCTGAAGTCTGAGGACACAGCCATGTATTTCTGT
GCAAGACCCCTTATTCCTACGATGGTAGAAGCATTTGACTACTGGGGCCAAGGC
ACCTCTCTCACAGTCTCCTCA
SEQ ID NO: 149 Light Chain Amino Acid Sequence
SIVMTQTPKFLPVSAGD RVTMTCKASQSVGNNVAWYQQKPGQSPKLLIYYASN
RYTGVPD RFTGSGSGADFTFTISSVQVEDLA VYFCQQHYSSPFTFGTGTKLEIK
SEQ ID NO: 150 Light Chain Nucleotide Sequence
AGTATTGTGATGACCCAGACTCCCAAATTCCTGCCTGTATCAGCAGGAGACAGGGTTAC
CATGACCTGCAAGGCCAGTCAGAGTGTGGGTAATAATGTAGCCTGGTACCAACAGAAG
CCAGGACAGTCTCCTAAACTGCTGATATACTATGCATCCAATCGCTACACTGGAGT
CCCTGATCGCTTCACTGGCAGTGGATCTGGGGCAGATTTCACTTTCACCATCAGCAGTG
TGCAGGTTGAAGACCTGGCAGTTTATTTCTGTCAGCAGCATTATAGCTCTCCATTCACGT
TCGGCACGGGGACAAAATTGGAAATAAAA

TABLE 13
Sequences - Antibody Variable Region Sequences - 10-J24-A

SEQ ID NO: 151 Heavy Chain (HC), CDR1
GFTFSSYA
SEQ ID NO: 152 HC, CDR2
ISGGGGFT
SEQ ID NO: 153 HC, CDR3
ARDEDGKHDYFDY
SEQ ID NO: 154 Light Chain (LC), CDR1
QDVGTA
SEQ ID NO: 155 LC, CDR2
WAS
SEQ ID NO: 156 LC, CDR3
QQYASYPLT
SEQ ID NO: 157 Heavy Chain Amino Acid Sequence
EVQLVESGGGLVKPGGSLKLSCAASGFTFSSYAMSWVRQNPAKRLEWVATISGGGGFTYY
PDSVKGRFTISRDNAKNTLYLQMNSLRSEDTAMYYCARDEDGKH DYFDYWGQGTIL TVSS
SEQ ID NO: 158 Heavy Chain Nucleotide Sequence
GAAGTGCAGCTGGTGGAGTCTGGGGGAGGCTTAGTGAAGCCTGGAGGGTCCCTGAAA
CTCTCCTGTGCAGCCTCTGGATTCACTTTCAGTAGCTATGCCATGTCTTGGGTTCGCCA
GAATCCGGCGAAGAGGCTGGAGTGGGTCGCAACCATTAGCGGTGGTGGTGGTTTCAC
CTACTATCCAGACAGTGTGAAGGGCCGATTCACCATCTCCAGAGACAATGCCAAGAACA
CCCTATACCTGCAAATGAACAGTCTGAGGTCTGAGGACACAGCCATGTATTACTGTGCA
AGGGATGAAGATGGTAAACACGACTACTTTGACTACTGGGGCCAAGGCACCACTCTCA
CAGTCTCCTCA
SEQ ID NO: 159 Light Chain Amino Acid Sequence
DIVMTQSHKFMSTSVGDRVSITCKASQDVGTAVAWYQQKPGQSPKLLIYWASTRHTGVPD
RFTGSGSGTDFTLTISNVQSE D LADYFCQQYASYPLTFGGGTKLVIK
SEQ ID NO: 160 Light Chain Nucleotide Sequence
GACATTGTGATGACCCAGTCTCACAAATTCATGTCCACATCAGTAGGAGACAGGGTCAG
CATCACCTGCAAGGCCAGTCAGGATGTGGGTACTGCTGTAGCCTGGTATCAACAGAAA
CCAGGGCAATCTCCTAAACTACTGATTTACTGGGCATCCACCCGGCACACTGGAG
TCCCTGATCGCTTCACAGGCAGTGGATCTGGGACAGATTTCACTCTCACCATHAGCAAT
GTGCAGTCTGAAGACTTGGCAGATTATTTCTGTCAGCAATATGCTAGCTATCCTCTGAC
GTTCGGTGGAGGCACCAAGCTGGTAATCAAA

TABLE 14
Sequences - Antibody Variable Region Sequences - 1-B11-A

SEQ ID NO: 161 Heavy Chain (HC), CDR1
GYTFTDHY
SEQ ID NO: 162 HC, CDR2
IFPGSGST
SEQ ID NO: 163 HC, CDR3
ARSGGLRLGFAY
SEQ ID NO: 164 Light Chain (LC), CDR1
QDISNY
SEQ ID NO: 165 LC, CDR2
YTS
SEQ ID NO: 166 LC, CDR3
QQVNTLPYT
SEQ ID NO: 167 Heavy Chain Amino Acid Sequence
QVQLQQSGPELVKPGASVKISCKASGYTFTDHYINWVKQRPGQGLEWIGWIFPGSGSTYYNEQFKG
KATLTVDKSSNTAYMLLSSLTSEDSAVYFCARSGGLRLGFAYWGQGTLVTVSA
SEQ ID NO: 168 Heavy Chain Nucleotide Sequence
CAGGTCCAGCTACAGCAGTCTGGACCTGAACTGGTGAAGCCTGGGGCTTCAGTGAAGATATCCT
GCAAGGCTTCTGGCTACACCTTCACTGACCACTATATAAATTGGGTGAAGCAGAGGCCTGGACA
GGGACTTGAGTGGATTGGATGGATTTTTCCTGGAAGTGGTAGTACTTACTACAATGAGCAGTTCA
AGGGCAAGGCCACGCTTACTGTAGACAAATCCTCCAACACAGCCTACATGTTGCTCAGCAGCCT
GACCTCTGAGGACTCTGCGGTCTATTTCTGTGGAAGATCAGGGGGATTACGACTGGGGTTTGCT
TACTGGGGCCAAGGGACTCTGGTCACTGTCTCTGCA
SEQ ID NO: 169 Light Chain Amino Acid Sequence
DIQMTQTTSSLSASLGDRVTISCRASQDISNYLNWYQQKPDGTVKLLIYYTSRLHSGVPSRFSGSGS
GTDYSLTISNLEQEDISTYFCQQVNTLPYTFGGGTKLEIK
SEQ ID NO: 170 Light Chain Nucleotide Sequence
GATATCCAGATGACACAGACTACATCCTCCCTGTCTGCCTCTCTGGGAGACAGAGTCACCATCA
GTTGCAGGGCAAGTCAGGACATTAGCAATTATTTAAACTGGTATCAGCAGAAACCAGATGGAAC
TGTTAAACTCCTGATCTACTACACATCAAGATTACACTCAGGAGTCCCATCAAGGTTCAGTGGCA
GTGGGTCTGGAACAGATTATTCTCTCACCATTAGCAACCTGGAGCAAGAAGATATTTCCACTTAC
TTTTGCCAACAGGTTAATACGCTTCCGTACACGTTCGGAGGGGGGACCAAGCTGGAAATAAAA

TABLE 15
Sequences - Antibody Variable Region Sequences - 1-L10-A

SEQ ID NO: 171 Heavy Chain (HC), CDR1
GFTFNNYW
SEQ ID NO: 172 HC, CDR2
MRLKSDNYAT
SEQ ID NO: 173 HC, CDR3
IEGYWTY
SEQ ID NO: 174 Light Chain (LC), CDR1
SSVSSSY
SEQ ID NO: 175 LC, CDR2
STS
SEQ ID NO: 176 LC, CDR3
LQVHRSPPYT
SEQ ID NO: 177 Heavy Chain Amino Acid Sequence
EVKLEESGGGLVQPGGSMKLSCVASGFTFNNYWMNWVRQSPEKGLEWVAQMRLKSDNYATHYA
ESVKGRFTISRDDSKSSVYLQMNNLRAEDTGIYYCIEGYWTYWGQGTLVTVSA
SEQ ID NO: 178 Heavy Chain Nucleotide Sequence
GAAGTGAAGCTTGAGGAGTCTGGAGGAGGCTTGGTGCAACCTGGAGGATCCATGAAACTCTCC
TGTGTTGCCTCTGGATTCACTTTCAATAACTACTGGATGAACTGGGTCCGCCAGTCTCCAGAGA
AGGGGCTTGAGTGGGTTGCTCAAATGAGATTGAAATCTGATAATTATGCAACACATTATGCGGA
GTCTGTGAAAGGGAGGTTCACCATCTCAAGAGATGATTCCAAAAGTAGTGTCTATCTGCAAATG
AACAACTTAAGGGCTGAAGACACTGGAATTTATTACTGTATTGAAGGTTACTGGACTTACTGGGG
CCAAGGGACTCTGGTCACTGTCTCTGCA
SEQ ID NO: 179 Light Chain Amino Acid Sequence
QIVLTQSPAIMSASLGERVTMTCTASSSVSSSYLHWYQQKPGSSPKLWIYSTSNLASGVPARFSGNG
SGTSYSLTISSMEAEDAATYYCLQYHRSPPYTFGGGTKLEIK
SEQ ID NO: 180 Light Chain Nucleotide Sequence
CAAATTGTTCTCACCCAGTCTCCAGCAATCATGTCTGCATCTCTAGGGGAACGGGTCAC
CATGACCTGCACTGCCAGCTCAAGTGTAAGTTCCAGTTACTTGCACTGGTACCAGCAGA
AGCCAGGATCCTCCCCCAAACTCTGGATTTATAGCACATCCAACCTGGCTTCTGGAGTC
CCAGCTCGCTTCAGTGGCAATGGGTCTGGGACCTCTTACTCTCTCACAATCAGCAGCAT
GGAGGCTGAAGATGCTGCCACTTATTACTGCCTCCAGTATCATCGCTCCCCACCGTACA
CGTTCGGAGGGGGGACCAAGCTGGAAATAAAA

TABLE 16
Sequences - Antibody Variable Region Sequences - 2-H7-A

SEQ ID NO: 181 Heavy Chain (HC), CDR1
GYTFTGYT
SEQ ID NO: 182 HC, CDR2
IYPENGDT
SEQ ID NO: 183 HC, CDR3
ARDGYDVLYGMDY
SEQ ID NO: 184 Light Chain (LC), CDR1
KSVSTSGYSY
SEQ ID NO: 185 LC, CDR2
LAS
SEQ ID NO: 186 LC, CDR3
QHSRELPWT
SEQ ID NO: 187 Heavy Chain Amino Acid Sequence
QAYLQQSGAELVRPGASVKMSCKASGYTFTGYTMHWVKQTPRQGLEWIGAIYPENGDTSYNQKFK
GKATLTVDKSSSTAYMQLSSLTSEDSAVYFCARDGYDVLYGMDYWGQGTSVTVSS
SEQ ID NO: 188 Heavy Chain Nucleotide Sequence
CAGGCTTATCTACAGCAGTCTGGGGCTGAGCTGGTGAGGCCTGGGGCCTCAGTGAAGATGTCC
TGCAAGGCTTCTGGCTACACATTTACCGGTTACACTATGCACTGGGTAAAGCAGACACCTAGAC
AGGGCCTGGAATGGATTGGAGCTATTTATCCAGAAAATGGTGATACTTCCTACAATCAGAAGTTC
AAGGGCAAGGCCACACTGACTGTCGACAAATCCTCCAGCACAGCCTACATGCAGCTCAGCAGC
CTGACATCTGAAGACTCTGCGGTCTATTTCTGTGCAAGAGATGGTTACGACGTTCTCTATGGTAT
GGACTACTGGGGTCAAGGAACCTCAGTCACCGTCTCCTCA
SEQ ID NO: 189 Light Chain Amino Acid Sequence
DIVLTQSPASLAVSLGQRATISCRASKSVSTSGYSYMHWYQQKPGQPPKLLIYLASNLESGVPARFS
GSGSGTDFTLNIHPVEEEDAATYYCQHSRELPWTFGGGTKLDIK
SEQ ID NO: 190 Light Chain Nucleotide Sequence
GACATTGTGCTGACACAGTCTCCTGCTTCCTTAGCTGTATCTCTGGGGCAGAGGGCCACCATCT
CATGCAGGGCCAGCAAAAGTGTCAGTACATCTGGCTATAGTTATATGCACTGGTACCAACAGAAA
CCAGGACAGCCACCCAAACTCCTCATCTATCTTGCATCCAACCTAGAATCTGGGGTCCCTGCCA
GGTTCAGTGGCAGTGGGTCTGGGACAGACTTCACCCTCAACATCCATCCTGTGGAGGAGGAGG
ATGCTGCAACCTATTACTGTCAGCACAGTAGGGAGCTTCCGTGGACGTTCGGTGGAGGCACCA
AGCTGGATATCAAA

TABLE 17
Sequences - Antibody Variable Region Sequences - 2-J9-A

SEQ ID NO: 191 Heavy Chain (HC), CDR1
GYTFTDYN
SEQ ID NO: 192 HC, CDR2
INPNNGGT
SEQ ID NO: 193 HC, CDR3
ARGGFYGNYGAYYGMDF
SEQ ID NO: 194 Light Chain (LC), CDR1
ENIYSH
SEQ ID NO: 195 LC, CDR2
NTK
SEQ ID NO: 196 LC, CDR3
QHHYGIPPE
SEQ ID NO: 197 Heavy Chain Amino Acid Sequence
EVQLQQSGPELVKPGASVKMSCKASGYTFTDYNIHWVKQSHGKSLEWIGYINPNNGGTDYNQKFQ
GKATLTVNKSSSTAYMELRSLTSEDSAVYYCARGGFYGNYGAYYGMDFWGQGTSVTVSS
SEQ ID NO: 198 Heavy Chain Nucleotide Sequence
GAGGTCCAGCTGCAACAGTCTGGACCTGAGTTGGTGAAGCCTGGGGCTTCAGTGAAGATGTCC
TGCAAGGCTTCTGGATACACATTCACTGACTACAACATACACTGGGTGAAGCAGAGCCATGGAA
AGAGCCTTGAGTGGATTGGATATATTAACCCTAACAATGGTGGTACTGACTACAACCAGAAGTT
CCAGGGCAAGGCCACATTGACTGTAAACAAGTCCTCCAGCACAGCCTACATGGAGCTCCGCAG
CCTGACATCGGAAGATTCTGCAGTCTATTACTGTGCAAGAGGGGGGTTTTATGGCAATTACGGG
GCTTACTATGGTATGGACTTCTGGGGTCAAGGAACCTCAGTCACCGTCTCCTCA
SEQ ID NO: 199 Light Chain Amino Acid Sequence
DIQMTQSPASLSASVGETVTITCRTSENIYSHLAWYQQTQGKSPQLLVYNTKTLAEGVPSRFTGSGS
GTQFSLKINSLQPEDFGSYYCQHHYGIPPEFGGGTKLEIK
SEQ ID NO: 200 Light Chain Nucleotide Sequence
GACATCCAGATGACTCAGTCTCGAGCCTCCGTATCTGCATCTGTGGGAGAAACTGTCAGCATCA
CATGTGGAAGAAGTGAGAATATTTATAGTCATTTAGCATGGTATGAGCAGACACAGGGAAAATCT
CCTCAGCTCCTGGTCTATAATACAAAAACCTTAGCAGAAGGTGTGCCATCAAGGTTCACTGGCA
GTGGATCAGGCACACAGTTTTCTCTGAAGATCAACAGCCTGCAGCCTGAAGATTTTGGGAGTTA
TTACTGTCAACATCATTATGGTATTCCTCCGGAGTTCGGTGGAGGCACCAAGCTGGAAATCAAA

TABLE 18
Sequences - Antibody Variable Region Sequences - 2-O12-A

SEQ ID NO: 201 Heavy Chain (HC), CDR1
GYTFTDYY
SEQ ID NO: 202 HC, CDR2
INSNNGGT
SEQ ID NO: 203 HC, CDR3
ARRGDGHFGGFTY
SEQ ID NO: 204 Light Chain (LC), CDR1
ESVDNSGISF
SEQ ID NO: 205 LC, CDR2
AAS
SEQ ID NO: 206 LC, CDR3
QQSKEVPWT
SEQ ID NO: 207 Heavy Chain Amino Acid Sequence
EVQLQQSGPELVKPGASVKISCKASGYTFTDYYMNWVKQSHGKSLEWIGDINSNNGGTTYDQKFK
GKATLTVDRSSSTAYMELRSLTSEDSAVYYCARRGDGHFGGFTYWGQGTLVTVSA
SEQ ID NO: 208 Heavy Chain Nucleotide Sequence
GAGGTCCAGCTGCAACAATCTGGACCTGAACTGGTGAAGCCTGGGGCCTCAGTGAAGATATCC
TGTAAGGCTTCTGGATACACGTTCACTGACTACTACATGAACTGGGTGAAGCAGAGCCATGGAA
AGAGCCTTGAGTGGATTGGAGATATTAATTCTAACAATGGTGGTACTACCTACGACCAGAAGTT
CAAGGGCAAGGCCACTTTGACTGTAGACAGGTCCTCCAGCACAGCCTACATGGAGCTCCGCAG
CCTGACATCTGAGGACTCTGCAGTCTATTACTGTGCAAGGAGAGGGGATGGTCACTTTGGGGG
GTTTACTTATTGGGGCCAAGGGACTCTGGTCACTGTCTCTGCA
SEQ ID NO: 209 Light Chain Amino Acid Sequence
DIVLTQSPASLAVSLGQRATISCRASESVDNSGISFMNWFQQKPGQPPKLLIYAASNQGSGVPARFS
GSGSGTDFSLNIHPMEEDDTAMYFCQQSKEVPWTFGGGTKLEK
SEQ ID NO: 210 Light Chain Nucleotide Sequence
GACATTGTGCTGACCCAATCTCCAGCTTCTTTGGCTGTGTCTCTAGGGCAGAGGGCCACCATCT
CCTGCAGAGCCAGCGAAAGTGTTGATAATTCTGGCATTAGTTTTATGAACTGGTTCCAACAGAAA
CCAGGACAGCCACCCAAACTCCTCATCTATGCTGCATCCAACCAAGGATCCGGGGTCCCTGCCA
GGTTTAGTGGCAGTGGGTCTGGGACAGACTTCAGCCTCAACATCCATCCTATGGAGGAGGATG
ATACTGCAATGTATTTCTGTCAGCAAAGTAAGGAGGTTCCGTGGACGTTCGGTGGAGGCACCAA
GCTGGAAATCAAA

TABLE 19
Sequences - Antibody Variable Region Sequences - 2-P2-A

SEQ ID NO: 211 Heavy Chain (HC), CDR1
GYTFTSYG
SEQ ID NO: 212 HC, CDR2
IYIGNGYT
SEQ ID NO: 213 HC, CDR3
ARGGVDY
SEQ ID NO: 214 Light Chain (LC), CDR1
QSIVHSNGNTY
SEQ ID NO: 215 LC, CDR2
KVS
SEQ ID NO: 216 LC, CDR3
FQGSHVPYT
SEQ ID NO: 217 Heavy Chain Amino Acid Sequence
EVQLQOSGAELVRPGSSVKMSCKTSGYTFTSYGINWVKQRPGQGLEWIGYIYIGNGYTEYNEKFKG
KATLTSDTSSSTAYMQLSSLTSEDSAIYFCARGGVDYWGQGTTLIVSS
SEQ ID NO: 218 Heavy Chain Nucleotide Sequence
GAGGTCCAGCTTCAGCAGTCTGGAGCTGAGCTGGTGAGGCCTGGGTCCTCAGTGAAGATGTCC
TGCAAGACTTCTGGATATACATTCACAAGCTACGGTATAAACTGGGTGAAGCAGAGGCCTGGAC
AGGGCCTGGAATGGATTGGATATATTTATATTGGAAATGGTTATACTGAATACAATGAGAAGTTC
AAGGGTAAGGCCACACTGACTTCAGACACATCCTCCAGCACAGCCTACATGCAGCTCAGCAGC
CTGACATCTGAGGACTCTGCAATCTATTTCTGTGCAAGAGGAGGGGTTGACTACTGGGGCCAAG
GCACCACTCTCACAGTCTCCTCA
SEQ ID NO: 219 Light Chain Amino Acid Sequence
DVLMTQTPLSLPVSLGDQASISCRSSQSIVHSNGNTYLEWYLQKPGQSPKLLIYKVSNRFSGVPDRF
SGSGSGTDFTLKISRVEAEDLGVYYCFQGSHVPYTFGGGTKLEIK
SEQ ID NO: 220 Light Chain Nucleotide Sequence
GATGTTTTGATGACCCAAACTCCACTCTCCCTGGCTGTCAGTCTTGGAGATCAAGCCTCCATCTC
TTGCAGATCTAGTCAGAGTATTGTACATAGTAATGGAAACACCTATTTAGAATGGTACCTGCAGA
AACCAGGCCAGTCTCCAAAGCTCCTGATCTACAAAGTTTCCAACCGATTTTCTGGGGTCCCAGA
CAGGTTCAGTGGCAGTGGATCAGGGACAGATTTCACACTCAAGATCAGCAGAGTGGAGGCTGA
GGATCTGGGAGTTTATTACTGCTTTCAAGGTTCACATGTTCCGTACACGTTCGGAGGGGGGACC
AAGCTGGAAATAAAA

TABLE 20
Sequences - Antibody Variable Region Sequences - 3-E13-A

SEQ ID NO: 221 Heavy Chain (HC), CDR1
GYTFTDYN
SEQ ID NO: 222 HC, CDR2
INPNNGGT
SEQ ID NO: 223 HC, CDR3
ARGGFYGNYGAYYGMDF
SEQ ID NO: 224 Light Chain (LC), CDR1
ENIYSH
SEQ ID NO: 225 LC, CDR2
NAK
SEQ ID NO: 226 LC, CDR3
QHHYGIPPE
SEQ ID NO: 227 Heavy Chain Amino Acid Sequence
EVQLQQSGPELVKPGASVKMSCKASGYTFTDYNIHWVKQSHGKSLEWIGYINPNNGGTDYNQKFQ
GKATLTVNKSSSTAYMELRSLTSEDFAVYYCARGGFYGNYGAYYGMDFWGQGTSVTVSS
SEQ ID NO: 228 Heavy Chain Nucleotide Sequence
GAGGTCCAGCTGCAACAGTCTGGACCTGAGCTGGTGAAGCCTGGGGCTTCAGTGAAGATGTCC
TGCAAGGCTTCTGGATACACATTCACTGACTACAACATACACTGGGTGAAACAGAGCCATGGAA
AGAGCCTTGAGTGGATTGGATATATTAACCCTAACAATGGTGGTACTGACTACAACCAGAAGTT
CCAGGGCAAGGCCACATTGACTGTAAACAAGTCCTCCAGCACAGCCTACATGGAGCTCCGCAG
CCTGACATCGGAAGATTTTGCAGTCTATTACTGTGCAAGAGGGGGGTTTTATGGCAATTACGGG
GCTTACTATGGTATGGACTTCTGGGGTCAAGGAACCTCAGTCACCGTCTCCTCA
SEQ ID NO: 229 Light Chain Amino Acid Sequence
DIQMTQSPASLSASVGETVTITCRTSENIYSHLAWYQQTQGKSPQLLVYNAKTLAEGVPSRFSGSGS
GTHFSLKINSLQPEDFGSYYCQHHYGPPEFGGGTKLEIK
SEQ ID NO: 230 Light Chain Nucleotide Sequence
GACATCCAGATGACTCAGTCTCGAGCCTCCGTATCTGCATCTGTGGGAGAAACTGTCAGCATCA
CATGTGGAAGAAGTGAGAATATTTATAGTCATTTAGCATGGTATGAGCAGACACAGGGAAAATCT
CCTCAGCTCCTGGTCTATAATGCAAAAACCTTAGCAGAAGGTGTGCCATCAAGGTTCAGTGGCA
GTGGATCAGGCACACATTTTTCTCTGAAGATCAACAGCCTGCAGCCTGAAGATTTTGGGAGTTA
TTACTGTCAACATCATTATGGTATTCCTCCGGAGTTCGGTGGAGGCACCAAGCTGGAAATCAAA

TABLE 21
Sequences - Antibody Variable Region Sequences - 4-A15-A

SEQ ID NO: 231 Heavy Chain (HC), CDR1
GNAFSSYW
SEQ ID NO: 232 HC, CDR2
IYPGDGDT
SEQ ID NO: 233 HC, CDR3
ASGGYGSSYERDFDY
SEQ ID NO: 234 Light Chain (LC), CDR1
QNINVW
SEQ ID NO: 235 LC, CDR2
KAS
SEQ ID NO: 236 LC, CDR3
QQGHSYPLT
SEQ ID NO: 237 Heavy Chain Amino Acid Sequence
QVQLQQSGAELVKPGASVKISCKASGNAFSSYWMNWVKQRPGKGLEWIGQIYPGDGDTNFNGRF
KGKATLTADISSTTAYMLLSSLTSEDSAVYFCASGGYGSSYERDFDYWGQGTTLTVSS
SEQ ID NO: 238 Heavy Chain Nucleotide Sequence
CAGGTTCAGCTGCAGCAGTCTGGGGCTGAGCTGGTGAAGCCTGGGGCCTCAGTGAAGATTTCC
TGCAAAGCTTCAGGCAACGCATTCAGTAGTTACTGGATGAACTGGGTGAAGCAGAGGCCTGGA
AAGGGTCTTGAGTGGATTGGACAGATTTATCCTGGAGATGGTGATACTAACTTCAACGGAAGGT
TCAAGGGCAAGGCCACACTGACTGCAGACATATCCTCCACCACAGCCTACATGCTGCTCAGCAG
CCTGACCTCTGAGGACTCTGCGGTCTATTTCTGTGCAAGCGGGGGCTACGGTAGTAGCTACGA
GAGGGACTTTGACTACTGGGGCCAAGGCACCACTCTCACAGTCTCCTCA
SEQ ID NO: 239 Light Chain Amino Acid Sequence
DIQMNQSPSSLSASLGDTITITCHASQNINVWLSWYQQKPGNPKLLIYKASNLHTGVPSRFSGSGS
GTGFTLTISSLQPEDIATYYCQQGHSYPLTFGGGTKLEIK
SEQ ID NO: 240 Light Chain Nucleotide Sequence
GACATCCAGATGAACCAGTCTCCATCCAGTCTGTCTGCATCCCTTGGAGACACAATTACCATCAC
TTGCCATGCCAGTCAGAACATTAATGTTTGGTTAAGCTGGTACCAGCAGAAACCAGGAAATATTC
CTAAACTATTGATCTATAAGGCTTCCAACTTGCACACAGGCGTCCCATCAAGGTTTAGTGGCAGT
GGATCTGGAACAGGTTTCACATTAACCATCAGCAGCCTGCAGCCTGAAGACATTGCCACTTACT
ACTGTCAACAGGGTCACAGTTATCCTCTCACGTTCGGAGGGGGGACCAAGCTGGAAATAAAA

TABLE 22
Sequences - Antibody Variable Region Sequences - 4-C3-A

SEQ ID NO: 241 Heavy Chain (HC), CDR1
GYSFTGYY
SEQ ID NO: 242 HC, CDR2
IYPYNGVS
SEQ ID NO: 243 HC, CDR3
ARAYGSSYDYYYGLDY
SEQ ID NO: 244 Light Chain (LC), CDR1
QSVDYDGAGY
SEQ ID NO: 245 LC, CDR2
SAS
SEQ ID NO: 246 LC, CDR3
QQSNDDPYT
SEQ ID NO: 247 Heavy Chain Amino Acid Sequence
EVQLQQSGPELVKPGASVKISCKASGYSFTGYYMHWVKQSHGNILDWIGYIYPYNGVSSYNQKFKG
KATLTVDKSSSTAYMELRSLTSEASAVYYCARAYGSSYDYYYGLDYWGQGTSVTVSS
SEQ ID NO: 248 Heavy Chain Nucleotide Sequence
GAGGTTCAGCTGCAGCAGTCTGGACCTGAGCTGGTGAAGCCTGGGGCTTCAGTGAAGATATCC
TGCAAGGCCTCTGGTTACTCATTCACTGGCTACTACATGCACTGGGTGAAGCAGAGCCATGGAA
ATATCCTCGATTGGATTGGATATATTTATCCTTACAATGGTGTTTCTAGCTACAACCAGAAATTCA
AGGGCAAGGCCACATTGACTGTAGACAAGTCCTCTAGCACAGCCTACATGGAGCTCCGCAGCC
TGACATCTGAGGCCTCTGCAGTCTATTACTGTGCAAGAGCCTACGGCAGTAGCTACGATTATTA
CTATGGTTTGGACTACTGGGGTCAAGGAACCTCAGTCACCGTCTCCTCA
SEQ ID NO: 249 Light Chain Amino Acid Sequence
DIVLTQSPASLAVSLGQRATISCKASQSVDYDGAGYMNWYQQKPGQPPKLLISSASNLQSGIPARFS
GSGSGTDFTLNIHPVEEEDAATYYCQQSNDDPYTFGGGTKLEIK
SEQ ID NO: 250 Light Chain Nucleotide Sequence
GACATTGTACTGACCCAATCTCCAGCTTCTTTGGCTGTGTCTCTGGGGCAGAGGGCCACCATCT
CCTGCAAGGCCAGCCAAAGTGTTGATTATGATGGTGCTGGTTATATGAACTGGTACCAACAGAAA
CCAGGACAGCCACCCAAACTCCTCATCTCTTCTGCATCCAATCTACAATCTGGGATCCCAGCCA
GGTTTAGTGGCAGTGGGTCTGGGACAGACTTCACCCTCAACATCCATCCTGTGGAGGAGGAGG
ATGCTGCAACCTATTACTGTCAGCAAAGTAATGACGATCCGTACACGTTCGGCGGGGGGACCA
AGCTGGAAATAAAA

TABLE 23
Sequences - Antibody Variable Region Sequences - 4-K13-A

SEQ ID NO: 251 Heavy Chain (HC), CDR1
GYTFTSYT
SEQ ID NO: 252 HC, CDR2
IYPGNVDT
SEQ ID NO: 253 HC, CDR3
ARDGYYAMDY
SEQ ID NO: 254 Light Chain (LC), CDR1
QSLLYSSNQKNY
SEQ ID NO: 255 LC, CDR2
WAS
SEQ ID NO: 256 LC, CDR3
QQYYSYPWT
SEQ ID NO: 257 Heavy Chain Amino Acid Sequence
QAYLQQSGAELVRPGASVKMSCKASGYTFTSYTVHWVKQTPRQGLEWIGAIYPGNVDTSYNQNFK
GKATLTVDKSSSTAYMQLSSLTSEDSAVYFCARDGYYAMDYWGQGTSVTVSS
SEQ ID NO: 258 Heavy Chain Nucleotide Sequence
CAGGCTTATCTACAGCAGTCTGGGGCTGAGCTGGTGAGGCCTGGGGCCTCAGTGAAGATGTCC
TGCAAGGCTTCTGGCTACACATTTACCAGTTACACTGTGCACTGGGTAAAGCAGACACCTAGAC
AGGGCCTGGAATGGATTGGAGCTATTTATCCAGGAAATGTTGATACTTCCTACAATCAGAATTTC
AAGGGCAAGGCCACACTGACTGTAGACAAATCCTCCAGCACAGCCTACATGCAGCTCAGCAGCC
TGACATCTGAAGACTCTGCGGTCTATTTCTGTGCAAGAGATGGTTACTATGCTATGGACTACTGG
GGTCAAGGAACCTCAGTCACCGTCTCCTCA
SEQ ID NO: 259 Light Chain Amino Acid Sequence
DIVLSQSPSSLPVSVGEKVTMSCKSSQSLLYSSNQKNYLAWYQQKPGQSPKLLIYWASTRESGVPDR
FTGSGSGTDFTLTISSVKAEDLAVYYCQQYYSYPWTFGGGTKLEIK
SEQ ID NO: 260 Light Chain Nucleotide Sequence
GACATTGTGTTGTCACAGTCTCCATCCTCCCTACCTGTGTCAGTTGGAGAGAAGGTTACTATGA
GCTGCAAGTCCAGTCAGAGCCTTTTATATAGTAGCAATCAAAAGAACTACTTGGCCTGGTACCA
GCAGAAACCAGGGCAGTCTCCTAAACTGCTGATTTACTGGGCATCCACTAGGGAATCTGGGGTC
CCTGATCGCTTCACAGGCAGTGGATCTGGGACAGATTTCACTCTCACCATCAGCAGTGTGAAGG
CTGAAGACCTGGCAGTTTATTACTGTCAGCAATATTATAGCTATCCGTGGACGTTCGGTGGAGG
CACCAAGCTGGAAATCAAA

TABLE 24
Sequences - Antibody Variable Region Sequences - 4-L4-A

SEQ ID NO: 261 Heavy Chain (HC), CDR1
GYTFTSYT
SEQ ID NO: 262 HC, CDR2
IYPGNVDT
SEQ ID NO: 263 HC, CDR3
ARDGYYAMDY
SEQ ID NO: 264 Light Chain (LC), CDR1
QSLLYSSNQKNY
SEQ ID NO: 265 LC, CDR2
WAS
SEQ ID NO: 266 LC, CDR3
QQYYSYPWT
SEQ ID NO: 267 Heavy Chain Amino Acid Sequence
QAYLQQSGAELVRPGASVKMSCKASGYTFTSYTVHWVKQTPRQGLEWIGGIYPGNVDTSYNQNFK
GKATLTVDKSSSTAYMQLSSLTSEDSAVYFCARDGYYAMDYWGQGTSVTVSS
SEQ ID NO: 268 Heavy Chain Nucleotide Sequence
CAGGCTTATCTACAGCAGTCTGGGGCTGAGCTGGTGAGGCCTGGGGCCTCAGTGAAGATGTCC
TGCAAGGCTTCTGGCTACACATTTACCAGTTACACTGTGCACTGGGTAAAGCAGACACCTAGAC
AGGGCCTGGAATGGATTGGAGGTATTTATCGAGGAAATGTTGATACTTCGTACAATCAGAATTTC
AAGGGCAAGGCCACACTGACTGTAGACAAATCCTCCAGCACAGCCTACATGCAGCTCAGCAGCC
TGACATCTGAAGACTCTGCGGTCTATTTCTGTGCAAGAGATGGTTACTATGCTATGGACTACTGG
GGTCAAGGAACCTCAGTCACCGTCTCCTCA
SEQ ID NO: 269 Light Chain Amino Acid Sequence
DIVLSQSPSSLPVSVGEKVTIVISCKSSQSLLYSSNQKNYLGWYQQKPGQSPKLLIYWASTRESGVPD
RFTGSGSGTDFTLTISSVKAEDLAVYYCQQYYSYPWTFGGGTKLEIK
SEQ ID NO: 270 Light Chain Nucleotide Sequence
GACATTGTGTTGTCACAGTCTCGATCCTCCCTACCTGTGTGAGTTGGAGAGAAGGTTACTATGA
GCTGCAAGTCCAGTCAGAGCCTTTTATATAGTAGCAATCAAAAGAACTACTTGGGCTGGTACCA
GCAGAAACCAGGGCAGTCTCCTAAACTGCTGATTTACTGGGCATCCACTAGGGAATCTGGGGT
CCCTGATCGCTTCACAGGCAGTGGATCTGGGACAGATTTTACTCTCACCATCAGTAGTGTGAAG
GCTGAAGACCTGGCAGTTTATTACTGTCAGCAATATTATAGCTATCCGTGGACGTTCGGTGGAG
GCACCAAGCTGGAAATCAAA

TABLE 25
Sequences - Antibody Variable Region Sequences - 5-H22-A

SEQ ID NO: 271 Heavy Chain (HC), CDR1
GYTFTDYE
SEQ ID NO: 272 HC, CDR2
IDLETGGT
SEQ ID NO: 273 HC, CDR3
TREFPHYYGSRYGFPY
SEQ ID NO: 274 Light Chain (LC), CDR1
SSVSY
SEQ ID NO: 275 LC, CDR2
STS
SEQ ID NO: 276 LC, CDR3
QQRSSYPYT
SEQ ID NO: 277 Heavy Chain Amino Acid Sequence
QVQLQQSGAELVRPGASVTLSCKASGYTFTDYEMHWMKQTPVHGLEWIGAIDLETGGTVYNQKF
KGKAILTADKSSNTAYMALRSLTSEDSAVYYCTREFPHYYGSRYGFPYWGQGTLVTVSA
SEQ ID NO: 278 Heavy Chain Nucleotide Sequence
CAGGTTCAACTGCAGCAGTCTGGGGCTGAGCTGGTGAGGCCTGGGGCTTCAGTGACGCTGTCC
TGCAAGGCTTCGGGCTACACATTTACTGACTATGAAATGCACTGGATGAAACAGACACCTGTGC
ATGGCCTGGAATGGATTGGAGCTATTGATCTTGAAACTGGTGGTACTGTCTACAATCAGAAGTTC
AAGGGCAAGGCCATACTGACTGCAGACAAATCCTCCAACACAGCCTACATGGCGCTCCGCAGTC
TGACATCTGAGGACTCTGCCGTCTATTACTGTACAAGAGAGTTCCCCCATTACTACGGTAGTAGG
TACGGTTTTCCTTACTGGGGCCAGGGGACTCTGGTCACTGTCTCTGCA
SEQ ID NO: 279 Light Chain Amino Acid Sequence
QIVLTQSPASMSASPGEKVTITCSASSSVSYMHWLQQKPGTSPKLWIYSTSNLASGVPARFSGSGSGT
SYSLTISRMEAEDAATYYCQQRSSYPYTFGGGTKLEIK
SEQ ID NO: 280 Light Chain Nucleotide Sequence
CAAATTGTTCTCACCCAGTCTCCAGCAATCATGTCTGCATCTCCAGGGGAGAAGGTCACCATAAC
CTGTAGTGCCAGCTCAAGTGTAAGTTACATGCACTGGTTACAGCAGAAGCCAGGCACTTCTCCC
AAACTCTGGATTTATAGCACATCXAACCTGGCTTCTGGAGTCCCTGCTCGCTTCAGTGGCAGTG
GATCTGGGACCTCTTACTCTCTCACAATCAGCCGAATGGAGGCTGAAGATGCTGCCACTTATTAC
TGCCAGCAAAGGAGTAGTTACCCGTACACGTTCGGAGGGGGGACCAAGCTGGAAATAAAA

TABLE 26
Sequences - Antibody Variable Region Sequences - 6-O12-A

SEQ ID NO: 281 Heavy Chain (HC), CDR1
GYTFTSYW
SEQ ID NO: 282 HC, CDR2
INPSNGGT
SEQ ID NO: 283 HC, CDR3
ARLGWFLPLGS
SEQ ID NO: 284 Light Chain (LC), CDR1
SSVSY
SEQ ID NO: 285 LC, CDR2
STS
SEQ ID NO: 286 LC, CDR3
HQWSGYMYT
SEQ ID NO: 287 Heavy Chain Amino Acid Sequence
QVQLQQPGTELVKPGASVKLSCKASGYTFTSYWMHWVKQRPGQGLEWIGNINPSNGGTNYNEKF
RSKATLTVDKSSSTAYMQLSSLTSEDSAVYYCARLGWFLPLGSWGQGTTLTVSS
SEQ ID NO: 288 Heavy Chain Nucleotide Sequence
CAGGTCCAACTGCAGCAGCCTGGGACTGAACTGGTGAAGCCTGGGGCTTCAGTGAAGCTGTCC
TGCAAGGCTTCTGGCTACACCTTCACCAGCTACTGGATGCACTGGGTGAAGCAGAGGCCTGGA
CAAGGCCTTGAGTGGATTGGAAATATTAATCCTAGCAATGGTGGTACTAACTACAATGAGAAGTT
CAGGAGCAAGGCCACACTGACTGTAGACAAATCCTCCAGCACAGCCTACATGCAGCTCAGCAGC
CTGACATCTGAGGACTCTGCGGTCTATTATTGTGCAAGACTGGGATGGTTTCTACCCCTTGGGTC
CTGGGGCCAAGGCACCACTCTCACAGTCTCCTCA
SEQ ID NO: 289 Light Chain Amino Acid Sequence
QIVLTQSPAIMSASLGEVITLTCSASSSVSYMHWYQQKSGTSPKLLYSTSNLASGVPSRFSGSGSGTF
YSLTISSVEAEDAADYYCHQWSGYMYTFGGGTKLEKK
SEQ ID NO: 290 Light Chain Nucleotide Sequence
CAAATTGTTCTCACCCAGTCTCCAGCAATCATGTCTGCATCTCTAGGGGAGGTGATCACCCTAAC
CTGCAGTGCCAGCTCGAGTGTAAGTTACATGCACTGGTACCAGCAGAAGTCAGGCACTTCTCCC
AAACTCTTGATTTATAGCACATCCAACCTGGCTTCTGGAGTCCCTTCTCGCTTCAGTGGCAGTGG
GTGTGGGACCTTTTATTCTCTCAGAATCAGCAGTGTGGAGGCTGAAGATGGTGGCGATTATTACT
GCCATCAGTGGAGTGGTTATATGTACACATTCGGAGGGGGGACCAAACTGGAAATAAAA

TABLE 27
Sequences - Antibody Variable Region Sequences - 8-N24-A

SEQ ID NO: 291 Heavy Chain (HC), CDR1
GYTFTSYW
SEQ ID NO: 292 HC, CDR2
IHPSDSDT
SEQ ID NO: 293 HC, CDR3
AIPLYSYDNSGGYYPMDY
SEQ ID NO: 294 Light Chain (LC), CDR1
QNVRTA
SEQ ID NO: 295 LC, CDR2
LAS
SEQ ID NO: 296 LC, CDR3
LQHWNYPFT
SEQ ID NO: 297 Heavy Chain Amino Acid Sequence
QVQLQQPGAELVKPGASVKVSCKASGYTFTSYWMHWVKQRPGQGLEWIGRIHPSDSDTNYYQKF
KGKATLSVDKSSSTAYMQLSSLTSEDSAVYYCAIPLYSYDNSGGYYPMDYWGQGTSVTVSS
SEQ ID NO: 298 Heavy Chain Nucleotide Sequence
CAGGTCCAACTGCAGCAGCCTGGGGCTGAACTGGTGAAGCCTGGGGCTTCAGTGAAGGTGTCC
TGCAAGGCTTCTGGCTACACCTTCACCAGCTACTGGATGCACTGGGTGAAGCAGAGGCCTGGC
CAAGGCCTTGAGTGGATTGGAAGGATTCATCCTTCTGATAGTGATACTAACTACTATCAAAAGTT
CAAGGGCAAGGCCACATTGTCTGTAGACAAATCCTCCAGCACAGCCTACATGCAGCTCAGCAG
CCTGACATCTGAGGACTCTGCGGTCTATTACTGTGCAATTCCCCTTTATTCCTACGATAATAGCG
GAGGTTACTATCCTATGGACTACTGGGGTCAAGGAACCTCAGTCACCGTCTCCTCA
SEQ ID NO: 299 Light Chain Amino Acid Sequence
DSVMTQSQKFMSTSVGDRVTITCKASQNVRTAVAWYQQKPGQSPKKLIYLASNRHTGVPDRFTGS
GSGTDFTLAISNVQSEDLADYFCLQHWNYPFTFGSGTRLEIK
SEQ ID NO: 300 Light Chain Nucleotide Sequence
GACATTGTGATGACCCAGTCTCAAAAATTCATGTCCACATCAGTAGGAGACAGGGTCACCATCA
CCTGCAAGGCCAGTCAGAATGTCCGTACTGCTGTAGCCTGGTATCAACAGAAACCAGGGCAGTC
TCCTAAAAAACTGATTTACTTGGCATCCAACCGGCACACTGGAGTCCCTGATCGCTTCACAGGCA
GTGGATCTGGGACAGATTTCACTCTCGCCATTAGCAATGTGCAATCTGAAGACCTGGCAGATTA
TTTCTGTCTGCAACATTGGAATTATCCATTCACGTTCGGCTCGGGGACAAGGTTGGAAATAAAA

TABLE 28
Sequences - Antibody Variable Region Sequences - 9-J11-A

SEQ ID NO: 301 Heavy Chain (HC), CDR1
GYTFTTYP
SEQ ID NO: 302 HC, CDR2
FHPYNDDS
SEQ ID NO: 303 HC, CDR3
ARGSYGPYWYFDV
SEQ ID NO: 304 Light Chain (LC), CDR1
GDIHNY
SEQ ID NO: 305 LC, CDR2
NAK
SEQ ID NO: 306 LC, CDR3
QHFWSIPYT
SEQ ID NO: 307 Heavy Chain Amino Acid Sequence
QVQLQQSGAELVKPGASVKMSCMASGYTFTTYPIEWMKQIHGKSLEWIGNFHPYNDDSKYNEKFK
GKATLSVEKSSSTVYLELSRSTSDDSAVYYCARGSYGPYWYFDVWGTGTTVTVSS
SEQ ID NO: 308 Heavy Chain Nucleotide Sequence
CAGGTTCAGCTGCAGCAGTCTGGGGCTGAACTAGTGAAGCCTGGAGCCTCAGTGAAGATGTCC
TGCATGGCTTCTGGCTACACCTTCACTACCTATCCTATAGAGTGGATGAAGCAGATTCATGGAA
AGAGCCTAGAGTGGATTGGAAATTTTCATCCTTACAATGATGATTCTAAGTAGAATGAAAAGTTC
AAGGGCAAGGCCACATTGAGTGTAGAAAAATCCTCTAGCACAGTCTACTTGGAGCTCAGCCGAT
CAACATCTGATGACTCTGCTGTTTATTACTGTGCAAGGGGGTCTTATGGCCCCTACTGGTACTTC
GATGTCTGGGGCACAGGGACCACGGTCACCGTCTCCTCA
SEQ ID NO: 309 Light Chain Amino Acid Sequence
DIQMTQSPASLSASVGETVTITCRASGDIHNYLVWYQQKQGKSPQLLVYNAKALADGVPSRFSGSG
SGTQYSLKINSLQPDDFGSYYCQHFWSIPYTFGGGTKLEIK
SEQ ID NO: 310 Light Chain Nucleotide Sequence
GACATCCAGATGACTCAGTCTCCAGCCTCCCTATCTGCATCTGTGGGAGAAACTGTCACCATCAC
ATGTCGAGCAAGTGGGGATATTCACAATTATTTAGTATGGTATCAGCAGAAACAGGGAAAATCTC
CTCAGCTCCTGGTCTATAATGCAAAAGCCTTAGCAGATGGTGTGCCATCAAGGTTCAGTGGCAG
TGGATCAGGAACACAATATTCTCTCAAGATCAACAGCCTGCAGCCTGATGATTTTGGGAGTTATT
ACTGTCAACATTTTTGGAGTATTCCGTACACGTTCGGAGGGGGGACCAAGCTGGAAATAAAA

TABLE 29
Sequences - Antibody Variable Region Sequences - 9-L13-A

SEQ ID NO: 311 Heavy Chain (HC), CDR1
GFSLTSYA
SEQ ID NO: 312 HC, CDR2
IWTGGGT
SEQ ID NO: 313 HC, CDR3
ARGPPLIYYGDLYYFDY
SEQ ID NO: 314 Light Chain (LC), CDR1
QDINKY
SEQ ID NO: 315 LC, CDR2
YTS
SEQ ID NO: 316 LC, CDR3
LQYDNLWT
SEQ ID NO: 317 Heavy Chain Amino Acid Sequence
QVQLKESGPGLVAPSQSLSITCTVSGFSLTSYAISWVRQPPGKGLEWLGLIWTGGGTNYNSALKSR
LSISKDNSKSQIFLKMNSLQTDDTARYYCARGPPLNYYGDLYYFDYWGQGTTLTVSS
SEQ ID NO: 318 Heavy Chain Nucleotide Sequence
CAGGTGCAGCTGAAGGAGTCAGGACCTGGCCTGGTGGCGCCCTCACAGAGCCTGTCCATCACA
TGCACTGTCTCTGGGTTCTCATTAACCAGCTATGCTATAAGCTGGGTTCGCCAGCCACCAGGGA
AGGGTCTGGAGTGGCTTGGACTAATATGGAGTGGTGGAGGCACAAATTATAATTGAGCTCTCAA
ATCCAGACTGAGCATCAGCAAAGACAACTCCAAGAGTCAAATTTTCTTAAAAATGAACAGTCTG
CAACTGATGACACAGCCAGGTACTACTGTGCCAGAGGGCCTCCTCTCATCTATTATGGTGACTT
GTACTACTTTGACTACTGGGGCCAAGGCACCACTCTCACAGTCTCCTCA
SEQ ID NO: 319 Light Chain Amino Acid Sequence
DIQMTQSPSSLSASLGGKVTITCKASQDINKYIAWYQHKPGKGPRLLIHYTSTLQPGIPSRFSGSGSG
RDYSFSISNLEPEDIATYYCLQYDNLWTFGGGTKLEIK
SEQ ID NO: 320 Light Chain Nucleotide Sequence
GACATCCAGATGACACAGTGTGCATCGTGACTGTCTGCATGTGTGGGAGGGAAAGTCACCATGA
CTTGCAAGGCAAGCCAAGACATTAACAAGTATATAGCTTGGTACCAACACAAGCCTGGAAAAGG
TCCTAGGCTGCTCATACATTACACATCTACATTACAGCCAGGCATCCCATCAAGGTTCAGTGGAA
GTGGGTCTGGGAGAGATTATTCCTTCAGCATCAGCAACCTGGAGCCTGAAGATATTGCAACTTA
TTATTGTCTACAGTATGATAATCTGTGGACGTTCGGTGGAGGCACCAAGCTGGAAATCAAA

TABLE 30
Sequences - Antibody Variable Region Sequences - 9-P9-A

SEQ ID NO: 321 Heavy Chain (HC), CDR1
GFSFSNFG
SEQ ID NO: 322 HC, CDR2
ISSGGDYI
SEQ ID NO: 323 HC, CDR3
TRGYFDY
SEQ ID NO: 324 Light Chain (LC), CDR1
QSIVHSNGNTY
SEQ ID NO: 325 LC, CDR2
TVS
SEQ ID NO: 326 LC, CDR3
FQGSHVPYT
SEQ ID NO: 327 Heavy Chain Amino Acid Sequence
DVKLVESGEGLVKPGGSLKLSCAASGFSFSNFGMSWVRQTPEKRLEWVAYISSGGDYIYYADTVK
GRFTISRDNARNTLYLQMSSLKSEDTANYYCTRGYFDYWGQGTTLTVSS
SEQ ID NO: 328 Heavy Chain Nucleotide Sequence
GACGTGAAGCTGGTGGAGTCTGGGGAAGGCTTAGTGAAGCCTGGAGGGTCCCTGAAACTCT
CCTGTGCAGCCTCCGGATTCAGTTTCAGTAACTTTGGCATGTCTTGGGTTCGCCAGACTCCAG
AGAAGAGGCTGGAGTGGGTGGGATAGATTAGTAGTGGTGGTGATTACATCTACTATGCAGACA
CTGTGAAGGGCCGATTCACCATCTCCAGAGACAATGCCAGGAACACCCTGTACCTGCAAATG
AGCAGTCTGAAGTCTGAGGACACAGCCATATATTACTGTACAAGGGGATACTTTGACTACT
GGCCAAGGCACCACTCTCACAGTCTCCTCA
SEQ ID NO: 329 Light Chain Amino Acid Sequence
DVLMTQTPLSLPVSLGDQASISCRSSQSIVHSNGNTYLEWYLQKPGQSPKLLIYTVSNRFSGVPDR
FSGSGSGTDFTLKISRVEAEDLGVYYCFQGSHVPYTFGGGTKLEMR
SEQ ID NO: 330 Light Chain Nucleotide Sequence
GATGTTTTGATGACCCAAACTCCACTCTCCCTGCCTGTCAGTCTTGGAGATCAAGCCTCCATCT
CTTGCAGATCTAGTCAGAGCATTGTACATAGTAATGGAAACACCTATTTAGAATGGTACCTGCA
GAAACCAGGCCAGTCTCCAAAGCTCCTGATCTACACAGTTTCCAACCGATTTTCTGGGGTCCC
AGACAGGTTCAGTGGCAGTGGATCGGGGACAGATTTCACACTCAAGATCAGCAGAGTGGAGG
CTGAGGATCTGGGAGTTTATTACTGCTTTCAAGGTTCACATGTTCCGTACACGTTCGGAGGGG
GGACCAAGCTGGAAATGAGA

TABLE 31
Sequences - Antibody Variable Region Sequences - 10-B11-A

SEQ ID NO: 331 Heavy Chain (HC), CDR1
GYTFTSYW
SEQ ID NO: 332 HC, CDR2
IHPSDSDT
SEQ ID NO: 333 HC, CDR3
AIPLYSYDNSGGYYPMDY
SEQ ID NO: 334 Light Chain (LC), CDR1
QNVRTA
SEQ ID NO: 335 LC, CDR2
LAS
SEQ ID NO: 336 LC, CDR3
LQHWNYPFT
SEQ ID NO: 337 Heavy Chain Amino Acid Sequence
QVQLQQPGAELVKPGASVKVSCKASGYTFTSYWMHWVKQRPGQGLEWIGRIHPSDSD
TNYYQKFKGKATLSVDKSSSTAYMQLSSLTSEDSAVYYCAIPLYSYDNSGGYYPMDYWG
QGTSVTVSS
SEQ ID NO: 338 Heavy Chain Nucleotide Sequence
CAGGTCCAACTGCAGCAGCCTGGGGCTGAACTGGTGAAGCCTGGGGCTTCAGTGAA
GGTGTCCTGCAAGGCTTCTGGCTACACCTTCACCAGCTACTGGATGCACTGGGTGAA
GCAGAGGCCTGGCCAAGGCCTTGAGTGGATTGGAAGGATTCATCCTTCTGATAGTG
ATACTAACTACTATCAAAAGTTCAAGGGCAAGGCCACATTGTCTGTAGACAAATCCTC
CAGCACAGCCTACATGCAGCTCAGCAGCCTGACATCTGAGGACTCTGCGGTCTATTA
CTGTGCAATTCCCCTTTATTCCTACGATAATAGCGGAGGTTACTATCCTATGGACTACT
GGGGTCAAGGAACCTCAGTCACCGTCTCCTCA
SEQ ID NO: 339 Light Chain Amino Acid Sequence
DIVMTQSQKFMSTSVGDRVTITCKASQNVRTAVAWYQQKPGQSPKKLIYLASNRHTGVP
DRFTGSGSGTDFTLAISNVQSEDLADYFCLQHWNYPFTFGSGTRLEIK
SEQ ID NO: 340 Light Chain Nucleotide Sequence
GACATTGTGATGACCCAGTCTCAAAAATTCATGTCCACATCAGTAGGAGACAGGGTC
ACCATCACCTGCAAGGCCAGTCAGAATGTCCGTACTGCTGTAGCCTGGTATCAACAG
AAACCAGGGCAGTCTCCTAAAAAACTGATTTACTTGGCATCCAACCGGCACACTGGA
GTCCCTGATCGCTTCACAGGCAGTGGATCTGGGACAGATTTCACTCTCGCCATTAGC
AATGTGCAATCTGAAGACCTGGCAGATTATTTCTGTCTGCAACATTGGAATTATCCAT
TCACGTTCGGCTCGGGGACAAGGTTGGAAATAAAA

TABLE 32
Sequences - Antibody Variable Region Sequences - 10-L24-A

SEQ ID NO: 341 Heavy Chain (HC), CDR1
GYTFTDYY
SEQ ID NO: 342 HC, CDR2
INSNNGGT
SEQ ID NO: 343 HC, CDR3
ARRGDGHFGGFTY
SEQ ID NO: 344 Light Chain (LC), CDR1
ESVDNSGISF
SEQ ID NO: 345 LC, CDR2
AAS
SEQ ID NO: 346 LC, CDR3
QQSKEVPWT
SEQ ID NO: 347 Heavy Chain Amino Acid Sequence
EVQLQQSGPELVKPGASVKISCKASGYTFTDYYMNWVKQSHGKSLEWIGDINSNNGGTTYDQKF
KGKATLTVDRSSSTAYMELRSLTSEDSAVYYCARRGDGHFGGFTYWGQGTLVTVSA
SEQ ID NO: 348 Heavy Chain Nucleotide Sequence
GAGGTCCAGCTGCAACAATCTGGACCTGAACTGGTGAAGCCTGGGGCCTCAGTGAAGATATC
CTGTAAGGCTTCTGGATACACGTTCACTGACTACTACATGAACTGGGTGAAGCAGAGCCATG
GAAAGAGCCTTGAGTGGATTGGAGATATTAATTCTAACAATGGTGGTACTACCTACGACCAGA
AGTTCAAGGGCAAGGCCACTTTGACTGTAGACAGGTCCTCCAGTACAGCCTACATGGAGCTCC
GCAGCCTGACATCTGAGGACTCTGCAGTCTATTACTGTGCMGGAGAGGGGATGGTCACTTT
GGGGGGTTTACTTATTGGGGCCAAGGGACTCTGGTCACTGTCTCTGCA
SEQ ID NO: 349 Light Chain Amino Acid Sequence
DIVLTQSPASLAVSLGQRATISCRASESVDNSGISFMNWFQQKPGQPPKLLIYAASNQGSGVPAR
FSGSGSGTDFSLNIHPMEEDDTAMYFCQQSKEVPWTFGGGTKLEIK
SEQ ID NO: 350 Light Chain Nucleotide Sequence
GACATTGTGCTGACCCAATCTCCAGCTTCTTTGGCTGTGTCTCTAGGGCAGAGGGCCACCATC
TCCTGCAGAGCCAGCGAAAGTGTTGATAATTCTGGCATTAGTTTTATGAACTGGTTCCAACAGA
AACCAGGACAGCCACCCAAACTCCTCATCTATGCTGCATCCAACCAAGGATCCGGGGTCCCT
GCCAGGTTTAGTGGCAGTGGGTCTGGGACAGACTTCAGCCTCAACATCCATCCTATGGAGGA
GGATGATACTGCAATGTATTTCTGTCAGCAAAGTAAGGAGGTTCCGTGGAGGTTGGGTGGAG
GCACCAAGCTGGAAATCAAA

TABLE 33
Sequences - Antibody Variable Region Sequences - 10-O3-A

SEQ ID NO: 351 Heavy Chain (HC), CDR1
GFSLTNYA
SEQ ID NO: 352 HC, CDR2
IWSDGRT
SEQ ID NO: 353 HC, CDR3
VRIEGGSYVGWYFDV
SEQ ID NO: 354 Light Chain (LC), CDR1
QDINKF
SEQ ID NO: 355 LC, CDR2
YTS
SEQ ID NO: 356 LC, CDR3
LQYDNLLT
SEQ ID NO: 357 Heavy Chain Amino Acid Sequence
QVQLKESGPGLVAPSQSLSITCTVSGFSLTNYAVHWVRQSPGKGLEWLGVIWSDGRTDYNAAFI
SRLSISKDNSKSQVFFKMNSLQADDTANYYCVRIEGGSYVGWYFDVWGAGTTVTVSS
SEQ ID NO: 358 Heavy Chain Nucleotide Sequence
CAGGTGCAGCTGAAGGAGTCAGGACCTGGCCTGGTGGCGCCCTCACAGAGCCTGTCCATCA
CCTGGACAGTCTCTGGTTTCTCATTAACCAACTATGCTGTACACTGGGTTCGCCAGTCTCCAG
GAAAGGGTCTGGAGTGGCTGGGAGTGATATGGAGTGATGGAAGGACAGACTATAATGCAGCT
TTCATATCTAGACTGAGCATCAGCAAGGACAACTCCAAGAGCCAAGTTTTCTTTAAAATGAACA
GTCTGCAAGCTGATGACACAGCCATATACTACTGTGTCAGAATCGAGGGTGGTAGCTACGTTG
GCTGGTACTTCGATGTCTGGGGCGCAGGGACCACGGTCACCGTCTCCTCA
SEQ ID NO: 359 Light Chain Amino Acid Sequence
DIQMTQSPSSLSASLGGTVTITCKASQDINKFIAWYQHKPGKGPRLLIHYTSTLQPGIPSRFSGSGS
GRDYSFSISNLEPEDIATYYCLQYDNLLTFGAGTKLELK
SEQ ID NO: 360 Light Chain Nucleotide Sequence
GACATCCAGATGACACAGTCTCCATCCTCACTGTCTGCATCTCTGGGAGGCACAGTCACCATC
ACTTGCAAGGCAAGCCAAGACATTAACAAGTTTATAGCTTGGTACCAACACAAGCCTGGAAAA
GGTCCTAGGCTGCTCATTCATTACACATCTACATTACAGCCAGGCATCCCATCAAGGTTCAGT
GGAAGTGGGTCTGGGAGAGATTATTCCTTCAGCATCAGCAACCTGGAGCCTGAAGATATTGC
AACTTATTATTGTCTACAGTATGATAATCTGCTCACGTTCGGTGCTGGGACCAAGCTGGAGCT
GAAA

TABLE 34
Sequences - Antibody Variable Region Sequences - 4-M3-A

SEQ ID NO: 361 Heavy Chain (HC), CDR1
GYSFTGYF
SEQ ID NO: 362 HC, CDR2
INPNNGET
SEQ ID NO: 363 HC, CDR3
ARGGTGTG
SEQ ID NO: 364 Light Chain (LC), CDR1
QGISSN
SEQ ID NO: 365 LC, CDR2
YGT
SEQ ID NO: 366 LC, CDR3
IQYAQFPYT
SEQ ID NO: 367 Heavy Chain Amino Acid Sequence
EVQLQQSGPELVKPGDSVKISCKASGYSFTGYFMNWVMQSHGKSLEWIGRINPNNGET
FYNQKFKGKATLTVDKSSDTAHMELRSLTSEDSAVYYCARGGTGTGWGQGTTLTVSS
SEQ ID NO: 368 Heavy Chain Nucleotide Sequence
GAGGTTCAGCTGCAGCAGTCTGGACCTGAGCTGGTGAAGCCTGGGGATTCAGTGAA
GATATCCTGCAAGGCTTCTGGTTACTCATTTACTGGCTACTTTATGAACTGGGTGATG
CAGAGCCATGGAAAGAGCCTTGAGTGGATTGGACGTATTAATCCTAACAATGGTGAA
ACTTTCTACAACCAGAAGTTCAAGGGCAAGGCCACATTGACTGTAGACAAATCCTCT
GACACAGCCCACATGGAGCTCCGGAGCCTGACATCTGAGGACTCTGCAGTCTATTAT
TGTGCAAGAGGGGGGACTGGGACGGGGTGGGGCCAAGGCACCACTCTCACAGTCT
CCTCA
SEQ ID NO: 369 Light Chain Amino Acid Sequence
DILMTQSPSSMSVSLGDTVSITCHASQGISSNIGWLQQKPGKSFKGLIYYGTNLEDGVPS
RFSGSGSGADYSLTISSLESEDLADYYCIQYAQFPYTFGGGTKLEIK
SEQ ID NO: 370 Light Chain Nucleotide Sequence
GACATCCTGATGACCCAATCTCCATCCTCCATGTCTGTATCTCTGGGAGACACAGTCA
GCATCACTTGCCATGCAAGTCAGGGCATTAGCAGTAATATAGGGTGGTTGCAGCAGA
AACCAGGGAAATCATTTAAGGGCCTGATCTATTATGGAACCAACTTGGAAGATGGAG
TTCCATCAAGGTTCAGTGGCAGTGGATCTGGAGCAGATTATTCTCTCACCATCAGCA
GCCTGGAATCTGAAGATCTTGCAGACTATTACTGTATACAGTATGCTCAGTTTCCGTA
CACGTTCGGAGGGGGGACCAAACTGGAAATAAAA

TABLE 35
Sequences - Antibody Variable Region Sequences - 4-N22-A

SEQ ID NO: 371 Heavy Chain (HC), CDR1
GYTFTNYW
SEQ ID NO: 372 HC, CDR2
INPSNGGT
SEQ ID NO: 373 HC, CDR3
ARETGNY
SEQ ID NO: 374 Light Chain (LC), CDR1
QGISSN
SEQ ID NO: 375 LC, CDR2
HGT
SEQ ID NO: 376 LC, CDR3
VQYAQFPYT
SEQ ID NO: 377 Heavy Chain Amino Acid Sequence
QVQLQQPGTELVKPGASVKLSCKASGYTFTNYWMYWVKQRPGQGLDWIGNINPSNGG
TNYNEKFKSKATLTVDKSSSTAYMQLSSLTSEDSAVYYCARETGNYWGQGTTLTVSS
SEQ ID NO: 378 Heavy Chain Nucleotide Sequence
CAGGTCCAACTGCAGCAGCCTGGGACTGAACTGGTGAAGCCTGGGGCTTCAGTGAA
GCTGTCCTGCAAGGCTTCTGGCTACACCTTCACCAATTACTGGATGTACTGGGTGAA
GCAGAGGCCTGGACAAGGCCTTGATTGGATTGGAAATATTAATCCTAGCAATGGTGG
TACTAACTACAATGAGAAGTTCAAGAGCAAGGCCACACTGACTGTAGACAAATCCTC
CAGCACAGCCTACATGCAGCTCAGCAGCCTGACATCTGAGGACTCTGCGGTCTATTA
TTGTGCAAGAGAGACTGGGAACTACTGGGGCCAAGGCACCACTCTCACAGTCTCCT
CA
SEQ ID NO: 379 Light Chain Amino Acid Sequence
DILMTQSPSSMSVSLGDTVSISCHASQGISSNIGWLQQKPGKSFKGLIYHGTNLEDGVPS
RFSGSGSGADYSLTISSLESEDFADYYCVQYAQFPYTFGGGTKLEK
SEQ ID NO: 380 Light Chain Nucleotide Sequence
GACATCCTGATGACCCAATCTCCATCCTCCATGTCTGTATCTCTGGGAGACACAGTCA
GCATCTCTTGCCATGCGAGTCAGGGCATTAGCAGTAATATAGGGTGGTTGCAGCAGA
AACCAGGGAAATCATTTAAGGGCCTGATCTATCATGGAACCAACTTGGAAGATGGAG
TTCCATCAAGGTTCAGTGGCAGTGGATCTGGAGCAGATTATTCTCTCACCATCAGCA
GCCTGGAATCTGAAGATTTTGCAGACTATTACTGTGTACAGTATGCTCAGTTTCCGTA
CACGTTCGGAGGGGGGACCAAGCTGGAAATAAAA

TABLE 36
Sequences - Antibody Variable Region Sequences - 7-B10-A

SEQ ID NO: 381 Heavy Chain (HC), CDR1
GFTFNTYA
SEQ ID NO: 382 HC, CDR2
IRSKSSNYAR
SEQ ID NO: 383 HC, CDR3
VRDHDYYGSSYWFAY
SEQ ID NO: 384 Light Chain (LC), CDR1
QSLVHNNGNTY
SEQ ID NO: 385 LC, CDR2
KVS
SEQ ID NO: 386 LC, CDR3
SQSTHVPWT
SEQ ID NO: 387 Heavy Chain Amino Acid Sequence
EVQLVESGGGLVQPKGSLKLSCAASGFTFNTYAMHWVRQAPGKGLEWVARIRSKSSNY
ARYYADSVKDRFTISRDESQSMLYLQMNNLKTEDTAMYYCVRDHDYYGSSYWFAYWG
QGTLVTVSA
SEQ ID NO: 388 Heavy Chain Nucleotide Sequence
GAGGTGCAGCTTGTTGAGTCTGGTGGAGGATTGGTGCAGCCTAAAGGATCATTGAA
ACTCTCATGTGCCGCCTCTGGTTTCACCTTCAATACCTATGCCATGCACTGGGTCCG
CCAGGCTCCAGGAAAGGGTTTGGAATGGGTTGCTCGCATAAGAAGTAAAAGTAGTAA
TTATGCAAGATATTATGCCGATTCAGTGAAAGACAGATTCACCATTTCCAGAGATGAG
TCACAAAGCATGCTCTATCTGCAAATGAACAACCTGAAAACTGAGGACACAGCCATG
TATTACTGTGTGAGAGATCACGATTACTACGGTAGTAGTTACTGGTTTGCTTACTGGG
GCCAAGGGACTCTGGTCACTGTCTCTGCA
SEQ ID NO: 389 Light Chain Amino Acid Sequence
DVVMTQTPLSLPVSLGDQASISCSSSQSLVHNNGNTYLHWYLQKPGQSPKLLIYKVSNRF
SGVPDRFSGSGSGTDFTLKISRVEAEDLGVYFCSQSTHVPWTFGGGTKLEIK
SEQ ID NO: 390 Light Chain Nucleotide Sequence
GATGTTGTGATGACCCAAACTCCACTCTCCCTGCCTGTCAGTCTTGGAGATCAAGCCT
CCATCTCTTGCAGCTCTAGTCAGAGCCTTGTACACAATAATGGAAACACCTATTTACA
TTGGTACCTGCAGAAGCCGGGCCAGTCTCCAAAGCTCCTGATCTACAAAGTTTCCAA
CCGATTTTCTGGGGTCCCAGACAGGTTCAGTGGCAGTGGATCAGGGACAGATTTCA
CACTCAAGATCAGCAGAGTGGAGGCTGAGGATCTGGGAGTTTATTTCTGCTCTCAAA
GTACACATGTTCCGTGGACGTTCGGTGGAGGCACCAAGCTGGAAATCAAA

TABLE 37
Sequences - Antibody Variable Region Sequences - 8-H5-A

SEQ ID NO: 391 Heavy Chain (HC), CDR1
GFSLSTSGMG
SEQ ID NO: 392 HC, CDR2
IYWDDDK
SEQ ID NO: 393 HC, CDR3
ARRDLHDYDGWFAY
SEQ ID NO: 394 Light Chain (LC), CDR1
QNVGTN
SEQ ID NO: 395 LC, CDR2
SAS
SEQ ID NO: 396 LC, CDR3
QQYNSYPLT
SEQ ID NO: 397 Heavy Chain Amino Acid Sequence
QVTLKESGPGILQSSQTLSLTCSLSGFSLSTSGMGVSWVRQPSGKGLEWLAHIYWDDDK
RYTPSLKSRLTISTDSSRKQVFLKITSVDTADTATYYCARRDLHDYDGWFAYWGQGTLVT
VSA
SEQ ID NO: 398 Heavy Chain Nucleotide Sequence
CAGGTTACTCTGAAAGAGTCTGGCCCTGGGATATTGCAGTCCTCCCAGACCCTCAGT
CTGACTTGTTCTTTGTCTGGGTTTTCACTGAGCACTTCTGGTATGGGTGTGAGCTGGG
TTCGTCAGCCTTCAGGAAAGGGTCTGGAGTGGCTGGCACACATTTACTGGGATGAT
GACAAGCGCTATACCCCTTCCCTGAAGAGCCGGCTCACAATCTCCACGGATTCCTCC
AGAAAGCAGGTATTCCTCAAGATCACCAGTGTGGACACTGCAGATACTGCCACATAC
TACTGTGCTCGAAGAGATCTTCATGATTACGACGGCTGGTTTGCTTACTGGGGCCAA
GGG ACTCTGGTCACTGTCTCTGCA
SEQ ID NO: 399 Light Chain Amino Acid Sequence
DIVMTQSQKFMSTAVGDRVSVTCKASQNVGTNVAWYQQKPGQSPKALIYSASFRYSGV
PDRFSGSGSGTDFTLTISNVQSEDLAEYFCQQYNSYPLTFGSGTKLEIK
SEQ ID NO: 400 Light Chain Nucleotide Sequence
GACATTGTGATGACCCAGTCTCAAAAATTCATGTCCACAGCAGTAGGAGACAGGGTC
AGCGTCACCTGCAAGGCCAGTCAAAATGTGGGTACTAATGTCGCCTGGTATCAACAG
AAACCAGGGCAATCTCCTAAAGCACTGATTTACTCGGCATCCTTCCGCTACAGTGGA
GTCCCTGATCGCTTCTCAGGCAGTGGATCTGGGACAGATTTCACTCTCACCATCAGC
AATGTGCAGTCTGAAGACTTGGCAGAGTATTTCTGTCAGCAATATAACAGCTATCCTC
TCACGTTCGGCTCGGGGACAAAGTTGGAAATAAAA

TABLE 38
Sequences - Antibody Variable Region Sequences - 2-G20-A

SEQ ID NO: 401 Heavy Chain (HC), CDR1
GYTFPSYV
SEQ ID NO: 402 HC, CDR2
IYPYNDGT
SEQ ID NO: 403 HC, CDR3
ARGDYGDSAWFPY
SEQ ID NO: 404 Light Chain (LC), CDR1
KSVSTSGYSY
SEQ ID NO: 405 LC, CDR2
LAS
SEQ ID NO: 406 LC, CDR3
QLSRELPWT
SEQ ID NO: 407 Heavy Chain Amino Acid Sequence
EVQLQQSGPELVKPGASVKMSCKASGYTFPSYVLHWVKQKPGQGLEWIGYIYPYNDGT
NYNEKFKGKATLTSDKSSSTAYMELSSLTSEDSAVYYCARGDYGDSAWFPYWGQGTLV
TVSA
SEQ ID NO: 408 Heavy Chain Nucleotide Sequence
GAGGTCCAGCTGCAGCAGTCTGGACCTGAGCTGGTAAAGCCTGGGGCTTCAGTGAA
GATGTCCTGCAAGGCTTCTGGATACACATTCCCTAGTTATGTTTTGCACTGGGTGAA
GCAGAAGCCTGGGCAGGGCCTTGAGTGGATTGGATATATTTATCCTTACAATGATGG
TACTAACTACAATGAGAAGTTCAAAGGCAAGGCCACACTGACTTCAGACAAATCCTC
CAGCACAGCCTACATGGAGCTCAGCAGCCTGACCTCTGAGGACTCTGCGGTCTATTA
CTGTGCAAGGGGAGACTATGGTGACTCCGCCTGGTTTCCTTACTGGGGGCCAAGGGA
CTCT GGTCACTGTCTCTGCA
SEQ ID NO: 409 Light Chain Amino Acid Sequence
DIVLTQSPASLAVSLGQRATISCRASKSVSTSGYSYMHWYQQKPGQPPKLLIYLASNLKS
GVPARFSGGGSGTDFTLNIHPVEEEDAATYYCQLSRELPWTFGGGTKLEIK
SEQ ID NO: 410 Light Chain Nucleotide Sequence
GACATTGTGCTGACACAGTCTCCTGCTTCCTTAGCTGTATCTCTGGGGCAGAGGGCC
ACCATCTCATGCAGGGCCAGCAAAAGTGTCAGTACATCTGGCTATAGTTATATGCACT
GGTACCAACAGAAACCAGGACAGCCACCCAAACTCCTCATCTATCTTGCATCCAACCT
AAAATCTGGGGTCCCTGCCAGGTTCAGTGGCGGTGGGTCTGGGACAGACTTCACCC
TCAACATCCATCCTGTGGAGGAGGAGGATGCTGCAACCTATTACTGTCAGCTCAGTA
GGGAGCTTCCGTGGACGTTCGGTGGAGGCACCAAGCTGGAAATCAAA

TABLE 39
Sequences - Antibody Variable Region Sequences - 3-E2-A

SEQ ID NO: 411 Heavy Chain (HC), CDR1
GYTFTDYY
SEQ ID NO: 412 HC, CDR2
INPNNGGT
SEQ ID NO: 413 HC, CDR3
ARRGDGHYGGFTY
SEQ ID NO: 414 Light Chain (LC), CDR1
ESVDNSGISF
SEQ ID NO: 415 LC, CDR2
AAS
SEQ ID NO: 416 LC, CDR3
QQSKEVPWT
SEQ ID NO: 417 Heavy Chain Amino Acid Sequence
EVQLQQSGPELVKPGASVKISCKASGYTFTDYYMNWVKQSHGKSLEWIGDINPNNGGT
SYTQKFKGKATLTVDKSSSKAYMELRSLTSEDSAVYYCARRGDGHYGGFTYWGQGTLV
TVSA
SEQ ID NO: 418 Heavy Chain Nucleotide Sequence
GAGGTCCAGCTGCAACAATCTGGACCTGAACTGGTGAAGCCTGGGGCTTCAGTGAA
GATATCCTGTAAGGCTTCTGGATACACGTTCACTGACTACTACATGAACTGGGTGAA
GCAGAGCCATGGAAAGAGCCTTGAGTGGATTGGAGATATTAATCCTAACAATGGTG
GTACTAGCTACACCCAGAAGTTCAAGGGCAAGGCCACATTGACTGTAGACAAGTCCT
CCAGCAAAGCCTACATGGAGCTCCGCAGCCTGACATCTGAGGACTCTGCAGTCTATT
ACTGTGCAAGAAGAGGGGATGGTCACTACGGGGGATTTACTTACTGGGGCCAAGGG
ACTC TGGTCACTGTCTCTGCA
SEQ ID NO: 419 Light Chain Amino Acid Sequence
DIVLTQSPASLVVSLGQRATISCRASESVDNSGISFMNWFQQKPGQPPKLLIYAASNQGS
GVPARFSGSGSGTDFSLNIHPMEEDDTAMYFCQQSKEVPWTFGGGTKLEIK
SEQ ID NO: 420 Light Chain Nucleotide Sequence
GACATTGTGCTGACCCAATCTCCAGCTTCTTTGGTTGTGTCTCTAGGGCAGAGGGCC
ACCATCTCCTGCAGAGCCAGCGAAAGTGTTGATAATTCTGGCATTAGTTTTATGAACT
GGTTCCAACAGAAACCAGGACAGCCACCCAAACTCCTCATCTATGCTGCATCCAACC
AAGGATCCGGGGTCCCTGCCAGGTTTAGTGGCAGTGGGTCTGGGACAGACTTCAGC
CTCAACATCCATCCTATGGAGGAGGATGATACTGCAATGTATTTCTGTCAGCAAAGTA
AGGAGGTTCCGTGGACGTTCGGTGGAGGCACCAAGCTGGAAATCAAA

TABLE 40
Sequences - Antibody Variable Region Sequences - 4-K16-A

SEQ ID NO: 421 Heavy Chain (HC), CDR1
GYTFTTYG
SEQ ID NO: 422 HC, CDR2
MSTYSGVP
SEQ ID NO: 423 HC, CDR3
ASGDVGY
SEQ ID NO: 424 Light Chain (LC), CDR1
QNVGTN
SEQ ID NO: 425 LC, CDR2
SAS
SEQ ID NO: 426 LC, CDR3
QQYNIYPST
SEQ ID NO: 427 Heavy Chain Amino Acid Sequence
QIQLVQSGPELKKPGETVKISCKASGYTFTTYGMSWVKQAPGKGLKWMGWMSTYSGV
PTYADDFKGRFAFSLETSASTAYLQINNLKNEDTATYFCASGDVGYWGQGTTLTVSS
SEQ ID NO: 428 Heavy Chain Nucleotide Sequence
CAGATCCAGTTGGTACAGTCTGGACCTGAGCTGAAGAAGCCTGGAGAGACAGTCAA
GATCTCCTGCAAGGCTTCTGGGTATACCTTCACAACCTATGGAATGAGCTGGGTGAA
ACAGGCTCCAGGAAAGGGTTTAAAGTGGATGGGCTGGATGAGCACCTACTCTGGAG
TGCCAACATATGCTGATGACTTCAAGGGACGGTTTGCCTTCTCTTTGGAAACCTCTGC
CAGCACTGCCTATTTGCAGATCAACAACCTCAAAAATGAGGACACGGCTACATATTTC
TGTGCAAGCGGGGACGTCGGCTACTGGGGCCAAGGCACCACTCTCACAGTCTCCTCA
SEQ ID NO: 429 Light Chain Amino Acid Sequence
DIVMTQSQKFMSTSVGDRVSVTCKASQNVGTNVAWYQQKPGQSPKALIYSASYRYSGVPD
RFTGSGSGTDFTLTISTVQSEDLAEYFCQQYNIYPSTFGGGTKLEIK
SEQ ID NO: 430 Light Chain Nucleotide Sequence
GACATTGTGATGACCCAGTCTCAAAAATTCATGTCCACATCAGTAGGAGACAGGGTC
AGCGTCACCTGCAAGGCCAGTCAGAATGTGGGTACTAATGTAGCCTGGTATCAACAG
AAACCAGGGCAATCTCCTAAAGCACTGATTTACTCGGCATCCTACCGGTACAGTGGA
GTCCCTGATCGCTTCACAGGCAGTGGATCTGGGACAGATTTCACTCTCACCATCAGC
ACTGTGCAGTCTGAAGACTTGGCAGAGTATTTCTGTCAGCAATATAACATCTATCCGT
CCACGTTCGGAGGGGGGACCAAGCTGGAAATAAAA

TABLE 41
Sequences - Antibody Variable Region Sequences - 6-C19-A-WT

SEQ ID NO: 431 Heavy Chain (HC), CDR1
GYTFTDYN
SEQ ID NO: 432 HC, CDR2
INPNNGGT
SEQ ID NO: 433 HC, CDR3
ARGGFYGNYGSYYGMDF
SEQ ID NO: 434 Light Chain (LC), CDR1
ENIYSH
SEQ ID NO: 435 LC, CDR2
NAK
SEQ ID NO: 436 LC, CDR3
QHHYGIPPE
SEQ ID NO: 437 Heavy Chain Amino Acid Sequence
EVQLQQSGPELVKPGASVKMSCKASGYTFTDYNIHWVKQSHGKSLEWIGYINPNNGGT
DYNQKFQGKATLTVDKSSSTAYMELRSLTSEDSAVYYCARGGFYGNYGSYYGMDFWG
QGTSVTVSS
SEQ ID NO: 438 Heavy Chain Nucleotide Sequence
GAGGTCCAGCTGCAACAGTCTGGACCTGAGCTGGTGAAGCCTGGGGCTTCAGTGAA
GATGTCCTGCAAGGCTTCTGGATACACATTCACTGACTACAACATACACTGGGTGAA
GCAGAGCCATGGAAAGAGCCTTGAGTGGATTGGATATATTAACCCTAACAATGGTG
GTACTGACTACAACCAGAAGTTCCAGGGCAAGGCCACATTGACTGTAGACAAGTCCT
CCAGCACAGCCTACATGGAGCTCCGCAGCCTGACATCGGAAGATTCTGCAGTCTATT
ACTGTGCAAGAGGGGGGTTTTATGGCAATTACGGGTCTTACTATGGTATGGACTTCT
GGG GTCAAGGAACCTCAGTCACCGTCTCCTCA
SEQ ID NO: 439 Light Chain Amino Acid Sequence
DIQMTQSPASLSASVGETVTITCRTSENIYSHLAWYQQTQGKSPHLLVYNAKTLAEGVPS
RFSGSGSGTQFSLKINSLQPEDFGSYFCQHHYGIPPEFGGGTKLEIK
SEQ ID NO: 440 Light Chain Nucleotide Sequence
GACATCCAGATGACTCAGTCTCCAGCCTCCCTATCTGCATCTGTGGGAGAAACTGTC
ACCATCACATGTCGAACAAGTGAGAATATTTATAGTCATTTAGCATGGTATCAGCAGA
CACAGGGAAAATCTCCTCACCTCCTGGTCTATAATGCAAAAACCTTAGCAGAAGGTG
TGCCATCAAGGTTCAGTGGCAGTGGATCAGGCACACAGTTTTCTCTGAAGATCAACA
GCCTGCAGCCTGAAGATTTTGGGAGTTATTTCTGTCAACATCATTATGGTATTCCTCC
GGAGTTCGGTGGAGGCACCAAGCTGGAAATCAAA

TABLE 42
Sequences - Antibody Variable Region Sequences - 6-L8-A

SEQ ID NO: 441 Heavy Chain (HC), CDR1
GFNIKNTY
SEQ ID NO: 442 HC, CDR2
INPANDDT
SEQ ID NO: 443 HC, CDR3
VYDVRWYFDV
SEQ ID NO: 444 Light Chain (LC), CDR1
QGLVHSNGNTY
SEQ ID NO: 445 LC, CDR2
KVS
SEQ ID NO: 446 LC, CDR3
SQSTHVPPT
SEQ ID NO: 447 Heavy Chain Amino Acid Sequence
EVQLQQSVAELVRPGASVKLSCTPSGFNIKNTYMHWMKQRPEQGLEWIGRINPANDDT
KYAPKFQDKATITADTSSNTAYLQLSSLTSEDTAIYYCVYDVRWYFDVWGTGTTVTVSS
SEQ ID NO: 448 Heavy Chain Nucleotide Sequence
GAGGTTCAGCTGCAGCAGTCTGTGGCAGAGCTTGTGAGGCCAGGGGCCTCAGTCAA
GTTGTCCTGCACACCTTCTGGCTTCAACATTAAAAACACCTATATGCACTGGATGAAG
CAGAGGCCTGAACAGGGCCTGGAGTGGATTGGAAGGATTAATCCTGCGAATGATGA
TACTAAATATGCCCCGAAGTTCCAGGACAAGGCCACTATAACTGCAGACACATCCTCC
AACACAGCCTACCTGCAGCTCAGCAGCCTGACATCTGAGGACACTGCCATCTATTAC
TGTGTCTACGACGTACGGTGGTATTTCGATGTCTGGGGCACAGGGACCACGGTCACC
G TCTCCTCA
SEQ ID NO: 449 Light Chain Amino Acid Sequence
DVVMTQTPLSLPVSLGDQASISCRSSQGLVHSNGNTYLHWYLQKPGQSPKLLIYKVSNR
FSGVPDRFSGSGSGTDFTLKISRVEAEDLGVYFCSQSTHVPPTFGAGTKLELK
SEQ ID NO: 450 Light Chain Nucleotide Sequence
GATGTTGTGATGACCCAAACTCCACTCTCCCTGCCTGTCAGTCTTGGAGATCAAGCCT
CCATCTCTTGCAGATCTAGTCAGGGCCTTGTACACAGTAATGGGAACACCTATTTACA
TTGGTACCTGCAGAAGCCAGGCCAGTCTCCAAAGCTCCTGATCTACAAAGTTTCCAA
CCGATTTTCTGGGGTCCCAGACAGGTTCAGTGGCAGTGGATCAGGGACAGATTTCA
CACTCAAGATCAGCAGAGTGGAGGCTGAGGATCTGGGAGTTTATTTCTGCTCTCAAA
GTACACATGTTCCTCCTACGTTCGGTGCTGGGACCAAGCTGGAGCTGAAA

TABLE 43
Sequences - Antibody Variable Region Sequences - 7-D7-A

SEQ ID NO: 451 Heavy Chain (HC), CDR1
GFSLTSYA
SEQ ID NO: 452 HC, CDR2
IWPGGGT
SEQ ID NO: 453 HC, CDR3
ARTGYYFDY
SEQ ID NO: 454 Light Chain (LC), CDR1
LSVNY
SEQ ID NO: 455 LC, CDR2
DTS
SEQ ID NO: 456 LC, CDR3
QQWSSNPPMYT
SEQ ID NO: 457 Heavy Chain Amino Acid Sequence
QVQLKESGPGLVAPSQSLSITCTVSGFSLTSYAISWVRQPPGKGLEWLGVIWPGGGTNY
NSALKSRLSISKDNSKSQVFLKMNSLQTDDTAKYYCARTGYYFDYWGQGTTLTVSS
SEQ ID NO: 458 Heavy Chain Nucleotide Sequence
CAGGTGCAGCTGAAGGAGTCAGGACCTGGCCTGGTGGCGCCCTCACAGAGCCTGTC
CATCACATGCACTGTCTCTGGGTTCTCATTAACCAGCTATGCTATAAGCTGGGTTCGC
CAGCCACCAGGAAAGGGTCTGGAGTGGCTTGGAGTAATATGGCCTGGTGGAGGCAC
AAATTATAATTCAGCTCTCAAATCCAGACTGAGCATCAGCAAAGACAACTCCAAGAGT
CAAGTTTTCTTAAAAATGAACAGTCTGCAAACTGATGACACAGCCAAATACTACTGTG
CCAGAACTGGGTACTACTTTGACTACTGGGGCCAAGGCACCACTCTCACAGTCTCCT
CA
SEQ ID NO: 459 Light Chain Amino Acid Sequence
QLILTQSPAIMSASPGEKVTMTCSASLSVNYMYWFQQKPGTSPKRWIHDTSKLASGVPA
RFSGSGSGTSYSLTISSMEAEDAATYYCQQWSSNPPMYTFGGGTKLEIK
SEQ ID NO: 460 Light Chain Nucleotide Sequence
CAACTTATTCTCACCCAGTCTCCAGCAATCATGTCTGCATCTCCAGGGGAGAAGGTCA
CCATGACCTGCAGTGCCAGCTTAAGTGTAAATTACATGTACTGGTTCCAGCAGAAGCC
AGGCACCTCCCCCAAAAGATGGATTCATGACACATCCAAACTGGCTTCTGGAGTCCC
TGCTCGCTTCAGTGGCAGTGGGTCTGGGACCTCTTACTCTCTCACAATCAGCAGCAT
GGAGGCTGAAGATGCTGCCACTTATTACTGCCAGCAGTGGAGTAGTAACCCACCCAT
GTACACGTTCGGAGGGGGGACCAAGCTGGAAATAAAA

TABLE 44
Sequences - Antibody Variable Region Sequences - 7-N20-A

SEQ ID NO: 461 Heavy Chain (HC), CDR1
GFTFSNYR
SEQ ID NO: 462 HC, CDR2
ITDKSDNYGA
SEQ ID NO: 463 HC, CDR3
SRDGYDWYFDL
SEQ ID NO: 464 Light Chain (LC), CDR1
QDVGIV
SEQ ID NO: 465 LC, CDR2
WAS
SEQ ID NO: 466 LC, CDR3
QQYSSYPT
SEQ ID NO: 467 Heavy Chain Amino Acid Sequence
QVQLVETGGGLVRPGNSLKLSCVTSGFTFSNYRMHWLRQPPGKRLEWIGIITDKSDNYG
ANYAESVKGRFAISRDDSKSSVYLEMKRLREEDTATYFCSRDGYDWYFDLWGTGTTVTV
SS
SEQ ID NO: 468 Heavy Chain Nucleotide Sequence
CAGGTGCAGCTTGTAGAGACCGGGGGAGGCTTGGTGAGGCCTGGAAATTCTCTGAA
ACTCTCCTGTGTTACGTCGGGATTCACTTTCAGTAACTACCGGATGCACTGGCTTCG
CCAGCCTCCAGGGAAGAGGCTGGAGTGGATTGGTATAATTACAGACAAATCTGATAA
TTATGGAGCAAATTATGCAGAGTCTGTGAAAGGCAGATTCGCCATTTCAAGAGATGA
TTCAAAAAGTAGTGTCTATCTAGAGATGAAGAGATTAAGAGAGGAAGACACTGCCAC
TTATTTTTGTAGTAGAGATGGTTACGACTGGTACTTCGATCTCTGGGGCACAGGGAC
CACGGTCACCGTCTCCTCA
SEQ ID NO: 469 Light Chain Amino Acid Sequence
DIVMTQSHKFMSTSVGDRVRITCKASQDVGIVVAWYQQKPGQSPKLLIYWASTRHTGVP
DRFTGSGSGTDFTLTISNVQSEDLADYFCQQYSSYPTFGGGTKLEIK
SEQ ID NO: 470 Light Chain Nucleotide Sequence
GACATTGTGATGACCCAGTCTCACAAATTCATGTCCACATCAGTAGGAGACAGGGTC
AGAATCACCTGTAAGGCCAGTCAGGATGTGGGAATTGTTGTAGCCTGGTATCAACAG
AAACCAGGGCAATCTCCTAAACTACTGATTTACTGGGCATCCACCCGGCACACTGGA
GTCCCTGATCGCTTCACAGGCAGTGGATCTGGGACAGATTTCACTCTCACCATTAGC
AATGTGCAGTCTGAAGACTTGGCAGATTATTTCTGTCAGCAATATAGCAGCTATCCCA
CGTTCGGAGGGGGGACCAAGCTGGAAATAAAA

TABLE 45
Sequences - Antibody Variable Region Sequences - 8-A17-A

SEQ ID NO: 471 Heavy Chain (HC), CDR1
GFSLTSYA
SEQ ID NO: 472 HC, CDR2
VWPGGGT
SEQ ID NO: 473 HC, CDR3
ARTGYHFDY
SEQ ID NO: 474 Light Chain (LC), CDR1
SSVSY
SEQ ID NO: 475 LC, CDR2
DTS
SEQ ID NO: 476 LC, CDR3
QQWSSNPPMYT
SEQ ID NO: 477 Heavy Chain Amino Acid Sequence
QVQLKESGPGLVAPSQSLSITCTVSGFSLTSYAISWVRQPPGKGLEWLGVVWPGGGTN
YNSALKSRLSINKDNSKSQVFLKMNSLQTDDTGRYYCARTGYHFDYWGQGTTLTVSS
SEQ ID NO: 478 Heavy Chain Nucleotide Sequence
CAGGTGCAGCTGAAGGAGTCAGGACCTGGCCTGGTGGCGCCCTCACAGAGCCTGTC
CATCACATGCACTGTCTCTGGGTTCTCATTAACCAGCTATGCTATAAGCTGGGTTCGC
CAGCCACCAGGAAAGGGTCTGGAGTGGCTTGGAGTAGTATGGCCTGGTGGAGGCA
CAAATTATAATTCAGCTCTCAAATCCAGACTGAGCATCAACAAAGACAACTCCAAGAG
TCAAGTTTTCTTAAAGATGAACAGTCTGCAAACTGATGACACAGGCAGGTACTACTGT
GCCAGAACTGGGTACCATTTTGACTACTGGGGCCAAGGCACCACTCTCACAGTCTCC
TCA
SEQ ID NO: 479 Light Chain Amino Acid Sequence
QIVLTQSPAIMSASPGEKVTMTCSVSSSVSYMHWYQQKSGTSPKRWIYDTSKLASGVPA
RFSGSGSGTSYSLTISSMEAEDAATYYCQQWSSNPPMYTFGGGTKLEIK
SEQ ID NO: 480 Light Chain Nucleotide Sequence
CAAATTGTTCTCACCCAGTCTCCAGCAATCATGTCTGCATCTCCAGGGGAGAAGGTCA
CCATGACCTGCAGTGTCAGCTCAAGTGTAAGTTACATGCACTGGTACCAGCAGAAGT
CAGGCACCTCCCCCAAAAGATGGATTTATGACACATCCAAACTGGCTTCTGGAGTCC
CTGCTCGTTTCAGTGGCAGTGGGTCTGGGACCTCTTACTCTCTCACAATCAGCAGCA
TGGAGGCTGAAGATGCTGCCACTTATTACTGCCAGCAGTGGAGTAGTAACCCACCCA
TGTACACGTTOGGAGGGGGGACCAAGCTGGAAATAAAA

TABLE 46
Sequences - Antibody Variable Region Sequences - 8-H3-A

SEQ ID NO: 481 Heavy Chain (HC), CDR1
GFTFNTYA
SEQ ID NO: 482 HC, CDR2
IRSKSSNYAT
SEQ ID NO: 483 HC, CDR3
VRDHDYYGSSYWFTY
SEQ ID NO: 484 Light Chain (LC), CDR1
QSLVHDNGNTY
SEQ ID NO: 485 LC, CDR2
KVS
SEQ ID NO: 486 LC, CDR3
SQSTHVPWT
SEQ ID NO: 487 Heavy Chain Amino Acid Sequence
EVQLVESGGGLVQPKGSLKLSCAASGFTFNTYAMHWVRQAPGKGLEWVARIRSKSSN
YATYYADSVKDRFTISRDDSQSMLYLQMNNLKTEDTAMYYCVRDHDYYGSSYWFTYWG
QGTLVTVSA
SEQ ID NO: 488 Heavy Chain Nucleotide Sequence
GAGGTGCAGCTTGTTGAGTCTGGTGGAGGATTGGTGCAGCCTAAAGGATCATTGAA
ACTCTCATGTGCCGCCTCTGGTTTCACCTTCAATACCTATGCCATGCACTGGGTCCG
CCAGGCTCCAGGAAAGGGTTTGGAATGGGTTGCTCGCATAAGAAGTAAAAGTAGTAA
TTATGCAACATATTATGCCGATTCAGTGAAAGACAGATTCACCATCTCCAGAGATGAT
TCACAAAGCATGCTCTATCTGCAAATGAACAACCTGAAAACTGAGGACACGGCCATG
TATTACTGTGTGAGAGATCACGATTACTACGGTAGTAGCTACTGGTTTACTTACTGGG
GCCAAGGGACTCTGGTCACTGTCTCTGCA
SEQ ID NO: 489 Light Chain Amino Acid Sequence
DVVMTQTPLSLPVSLGDQASISCRSSQSLVHDNGNTYLHWFLQKPGQSPKLLIYKVSNRF
SGVPDRFSGSGSGTDFTLKISRVETEDLGVYFCSQSTHVPWTFGGGTKLEIK
SEQ ID NO: 490 Light Chain Nucleotide Sequence
GATGTTGTGATGACCCAAACTCCACTCTCCCTGCCTGTCAGTCTTGGAGATCAAGCCT
CCATCTCTTGCAGATCTAGTCAGAGCCTTGTACACGATAATGGAAACACCTATTTACA
TTGGTTCCTGCAGAAGCCAGGCCAGTCTCCAAAGCTCCTGATCTACAAAGTTTCCAA
CCGATTTTCTGGGGTCCCAGACAGGTTCAGTGGCAGTGGATCAGGGACAGATTTCA
CACTCAAGATCAGCAGAGTGGAGACTGAGGATCTGGGAGTTTATTTCTGCTCTCAAA
GTACACATGTTCCGTGGACGTTCGGTGGAGGCACCAAGCTGGAAATCAAA

TABLE 47
Sequences - Antibody Variable Region Sequences - 8-L17-A

SEQ ID NO: 491 Heavy Chain (HC), CDR1
GFTFNTYA
SEQ ID NO: 492 HC, CDR2
IRSKSSNYAR
SEQ ID NO: 493 HC, CDR3
VRDHDYYGSSYWFAY
SEQ ID NO: 494 Light Chain (LC), CDR1
QSLVHNNGNTY
SEQ ID NO: 495 LC, CDR2
KVS
SEQ ID NO: 496 LC, CDR3
SQSTHVPWT
SEQ ID NO: 497 Heavy Chain Amino Acid Sequence
EVQLVESGGGLVQPKGSLKLSCAASGFTFNTYAMHWVRQAPGKGLEWVARIRSKSSNY
ARYYADSVKDRFTISRDDSQSMLYLQMNNLKTEDTAMYYCVRDHDYYGSSYWFAYWG
QGTLVTVSA
SEQ ID NO: 498 Heavy Chain Nucleotide Sequence
GAGGTGCAGCTTGTTGAGTCTGGTGGAGGATTGGTGCAGCCTAAAGGATCATTGAA
ACTCTCATGTGCCGCCTCTGGTTTCACCTTCAATACCTATGCCATGCACTGGGTCCG
CCAGGCTCCAGGAAAGGGTTTGGAATGGGTTGCTCGCATAAGAAGTAAAAGTAGTAA
TTATGCAAGATATTATGCCGATTCAGTGAAAGACAGATTCACCATCTCCAGAGATGAT
TCACAAAGCATGCTCTATCTGCAAATGAACAACCTGAAAACTGAGGACACAGCCATG
TATTACTGTGTGAGAGATCACGATTACTACGGTAGTAGTTACTGGTTTGCTTACTGGG
GCCAAGGGACTCTGGTCACTGTCTCTGCA
SEQ ID NO: 499 Light Chain Amino Acid Sequence
DVVMTQTPLSLPVSLGDQASISCRSSQSLVHNNGNTYLHWYLQKPGQSPKLLIYKVSNR
FSGVPDRFSGSGSGTDFTLKISRVEAEDLGVYFCSQSTHVPWTFGGGTKLEIK
SEQ ID NO: 500 Light Chain Nucleotide Sequence
GATGTTGTGATGACCCAAACTCCACTCTCCCTGCCTGTCAGTCTTGGAGATCAAGCCT
CCATCTCTTGCAGATCTAGTCAGAGCCTTGTACACAATAATGGAAACACCTATTTACA
TTGGTACCTGCAGAAGCCAGGCCAGTCTCCAAAGCTCCTGATCTACAAAGTTTCCAA
CCGATTTTCTGGGGTCCCAGACAGGTTCAGTGGCAGTGGATCAGGGACAGATTTCA
CACTCAAGATCAGCAGAGTGGAGGCTGAGGATCTGGGAGTTTATTTCTGCTCTCAAA
GTACACATGTTCCGTGGACGTTCGGTGGAGGCACCAAGCTGGAAATCAAA

TABLE 48
Sequences - Antibody Variable Region Sequences - 9-F6-A

SEQ ID NO: 501 Heavy Chain (HC), CDR1
GYTFTSYI
SEQ ID NO: 502 HC, CDR2
ILPYNDGT
SEQ ID NO: 503 HC, CDR3
ARWGYSNFLDS
SEQ ID NO: 504 Light Chain (LC), CDR1
QDINNY
SEQ ID NO: 505 LC, CDR2
YTS
SEQ ID NO: 506 LC, CDR3
QQGNTLPPT
SEQ ID NO: 507 Heavy Chain Amino Acid Sequence
EVQLQQSGPELVKPGASVKMSCKASGYTFTSYIMHWVKQKPGQGLEWIGYILPYNDGTK
YNEKFKGKATLTSDKSSSTAYMELRSLTSEDSAVYYCARWGYSNFLDSWGQGTTLTVSS
SEQ ID NO: 508 Heavy Chain Nucleotide Sequence
GAGGTCCAGCTGCAGCAGTCTGGACCTGAGCTGGTAAAGCCTGGGGCTTCAGTGAA
GATGTCCTGCAAGGCTTCTGGATACACATTCACTAGCTATATTATGCACTGGGTGAA
GCAGAAGCCTGGACAGGGCCTTGAGTGGATTGGATATATTCTTCCTTACAATGATGG
TACTAAGTACAATGAGAAGTTCAAAGGCAAGGCCACACTGACTTCAGACAAATCCTC
CAGCACAGCCTATATGGAGCTCAGAAGCCTGACCTCTGAGGACTCTGCGGTCTATTA
CTGTGCAAGATGGGGCTATAGTAACTTTCTTGACTCCTGGGGCCAAGGCACCACTCT
CACAGTCTCCTCA
SEQ ID NO: 509 Light Chain Amino Acid Sequence
DIQMTQTTSSLSASLGDRVTISCRASQDINNYLNWYQQKPDGTVKLLIYYTSRLHSGVPS
RFSGSGSGTDYSLTIRNLEQEDIATYFCQQGNTLPPTFGGGTKLEIK
SEQ ID NO: 510 Light Chain Nucleotide Sequence
GATATCCAGATGACACAGACTACATCCTCCCTGTCTGCCTCTCTGGGAGACAGAGTC
ACCATCAGTTGCAGGGCAAGTCAGGACATTAACAATTATTTAAACTGGTATCAGCAGA
AACCAGATGGAACTGTTAAACTCCTGATCTACTACACATCAAGATTACACTCAGGAGT
CCCATCAAGGTTCAGTGGCAGTGGGTCTGGAACAGATTATTCTCTCACCATTAGGAA
CCTGGAGCAAGAAGACATTGCCACTTACTTTTGCCAACAGGGTAATACGCTTCCTCC
GACGTTCGGTGGAGGCACCAAGCTGGAAATCAAA

TABLE 49
Sequences - Antibody Variable Region Sequences - 10-I12-A

SEQ ID NO: 511 Heavy Chain (HC), CDR1
GYTFTDSY
SEQ ID NO: 512 HC, CDR2
IFPGNDNT
SEQ ID NO: 513 HC, CDR3
ARSGGLRLGFAY
SEQ ID NO: 514 Light Chain (LC), CDR1
ESVDSYGHSF
SEQ ID NO: 515 LC, CDR2
LAS
SEQ ID NO: 516 LC, CDR3
QQNNEDPWT
SEQ ID NO: 517 Heavy Chain Amino Acid Sequence
RVQLQQSGPELVRPGTSVKISCKASGYTFTDSYINWVTQRPGQGLDWIGWIFPGNDNTY
YNEKFEGKATLTLDKSSSTAYMLLSSLTSEDSAVYFCARSGGLRLGFAYWGQGTLVTVSA
SEQ ID NO: 518 Heavy Chain Nucleotide Sequence
CGGGTCCAGCTACAGCAGTCTGGACCTGAGCTGGTGAGGCCTGGGACTTCAGTGAA
GATATCCTGCAAGGCTTCTGGCTACACCTTCACTGACTCCTATATAAACTGGGTGACG
CAGAGGCCTGGACAGGGACTTGATTGGATTGGATGGATTTTTCCTGGAAATGATAAT
ACTTACTACAATGAGAAGTTCGAGGGCAAGGCCACACTTACTCTTGACAAATCTTCCA
GTACAGCCTACATGTTGCTCAGCAGCCTGACCTCTGAGGACTCTGCGGTCTATTTCT
GTGCAAGATCAGGGGGATTACGACTGGGGTTTGCTTACTGGGGCCAAGGGACTCTG
GTC ACTGTCTCTGCA
SEQ ID NO: 519 Light Chain Amino Acid Sequence
NIVLTQSPASLAVSRGQRATISCRASESVDSYGHSFIHWYQQKPGQPPKILIYLASKLESG
VPARFSGSGSRTDFTLTIDPVEADDAATYYCQQNNEDPWTFGGGTKLEIK
SEQ ID NO: 520 Light Chain Nucleotide Sequence
AACATTGTGCTGACCCAATCTCCAGCTTCTTTGGCTGTGTCTCGAGGGCAGAGGGCC
ACCATATCCTGCAGAGCCAGTGAAAGTGTTGATAGTTATGGCCATAGTTTTATTCACT
GGTACCAGCAGAAACCAGGACAGCCACCCAAAATCCTCATCTATCTTGCATCCAAGT
TAGAATCTGGGGTCCCTGCCAGGTTCAGTGGCAGTGGGTCTAGGACAGACTTCACCC
TCACCATTGATCCTGTGGAGGCTGATGATGCTGCAACCTATTACTGTCAGCAAAATAA
TGAGGATCCGTGGACGTTCGGTGGAGGCACCAAGCTGGAAATCAAA

1. A monoclonal antibody, or antigen binding portion thereof, that binds to the SARS-Cov-2 spike protein (CoV-S), wherein the antibody comprises the vhCDR1, vhCDR2, vhCDR3, vlCDR1, vlCDR2 and vlCDR3 from an antibody selected from the group consisting of clone IDs: 4-N22-A (SEQ ID NOS: 371-376), 8-H5-A (SEQ ID NOS: 391-396), 10-L24-A (SEQ ID NOS: 341-346), 1-B11-A (SEQ ID NOS: 161-166), 1-L10-A (SEQ ID NOS: 171-176), 2-H7-A (SEQ ID NOS: 181-186), 2-J9-A (SEQ ID NOS: 191-196), 2-O12-A (SEQ ID NOS: 201-206), 2-P2-A (SEQ ID NOS: 211-216), 3-E13-A (SEQ ID NOS: 221-226), 4-A15-A (SEQ ID NOS: 231-236), 4-C3-A (SEQ ID NOS: 241-246), 4-K13-A (SEQ ID NOS: 251-256), 4-L4-A (SEQ ID NOS: 261-266), 5-H22-A (SEQ ID NOS: 271-276), 6-O12-A (SEQ ID NOS: 281-286), 8-N24-A (SEQ ID NOS: 291-296), 9-J11-A (SEQ ID NOS: 301-306), 9-L13-A (SEQ ID NOS: 311-316), 9-P9-A (SEQ ID NOS: 321-326), 10-B11-A (SEQ ID NOS:331-336), 10-O3-A (SEQ ID NOS: 351-356), 4-M3-A (SEQ ID NOS:361-366), 7-B10-A (SEQ ID NOS:381-386), 2-G20-A (SEQ ID NOS:401-406), 3-E2-A (SEQ ID NOS:411-416), 4-K16-A (SEQ ID NOS: 421-426), 6-C19-A-WT (SEQ ID NOS: 431-436), 6-C19-A (SEQ ID NOS: 91-96), 6-L8-A (SEQ ID NOS: 441-446), 7-D7-A (SEQ ID NOS: 451-456), 7-N20-A (SEQ ID NOS: 461-466), 8-A17-A (SEQ ID NOS: 471-476), 8-H3-A (SEQ ID NOS: 481-486), 8-L17-A (SEQ ID NOS:491-496), 9-F6-A (SEQ ID NOS:501-506), 10-I12-A (SEQ ID NOS: 511-516), 9-M12-A (SEQ ID NOS: 141-146), 3-P17-A (SEQ ID NOS: 51-56), 9-G24-A (SEQ ID NOS:131-136), 2-B16-B (SEQ ID NOS:1-6), 2-B20-A (SEQ ID NOS:11-16), 2-O15-A (SEQ ID NOS: 31-36), 3-K11-A (SEQ ID NOS:41-46), 4-A22-A (SEQ ID NOS: 61-66), 4-O14-B (SEQ ID NOS:81-86), 6-N1-A (SEQ ID NOS: 101-106), 4-C22-A (SEQ ID NOS: 71-76), and 10-J24-A (SEQ ID NOS: 151-156).

2. A monoclonal antibody, or antigen binding portion thereof, that binds to the SARS-Cov-2 spike protein (CoV-S), wherein the antibody comprises the variable heavy domain (VH) and variable light domain (VL) from an antibody selected from the group consisting of clone IDs: 4-N22-A, 8-H5-A, 10-L24-A, 1-B11-A, 1-L10-A, 2-H7-A, 2-J9-A, 2-O12-A, 2-P2-A, 3-E13-A, 4-A15-A, 4-C3-A, 4-K13-A, 4-L4-A, 5-H22-A, 6-O12-A, 8-N24-A, 9-J11-A, 9-L13-A, 9-P9-A, 10-B11-A, 10-O3-A, 4-M3-A, 7-B10-A, 2-G20-A, 3-E2-A, 4- K16-A, 6-C19-A-WT, 6-C19-A, 6-L8-A, 7-D7-A, 7-N20-A, 8-A17-A, 8-H3-A, 8-L17-A, 9-F6-A, 10-I12-A, 9-M12-A, 3-P17-A, 9-G24-A, 2-B16-B, 2-B20-A, 2-O15-A, 3-K11-A, 4-A22-A, 4-O14-B, 6-N1-A, 4-C22-A, and 10-J24-A (as depicted in Tables 1-49).

3. (canceled)

4. The monoclonal antibody according to claim 1, wherein the monoclonal antibody is humanized.

5. The monoclonal antibody according to claim 1, wherein the monoclonal antibody is IgG1, IgG2, IgG3, or IgG4 class.

6. The antigen binding portion of the monoclonal antibody according to claim 1.

7. The antigen binding portion according to claim 6, wherein the antigen binding portion comprises a Fab fragment, Fv fragment, or single-chain Fv antibody.

8. (canceled)

9. The monoclonal antibody, or antigen binding portion thereof, according to claim 1, wherein the monoclonal antibody, or antigen binding portion thereof, binds to one or more SARS-Cov-2 variants.

10. The monoclonal antibody, or antigen binding portion thereof, according to claim 1, wherein the monoclonal antibody, or antigen binding portion thereof, binds to the CoV-S of one or more SARS-Cov-2 Omicron variants.

11. The monoclonal antibody, or antigen binding portion thereof, according to claim 1, wherein the monoclonal antibody, or antigen binding portion thereof, neutralizes one or more SARS-Cov-2 variants.

12. The monoclonal antibody, or antigen binding portion thereof, according to claim 1, wherein the monoclonal antibody, or antigen binding portion thereof, neutralizes one or more SARS-Cov-2 Omicron variants.

13. A composition comprising a monoclonal antibody, or antigen binding portion thereof, according to claim 1.

14. A cell line that expresses the monoclonal antibody, or antigen binding portion thereof, according to claim 1.

15. A composition comprising:
a first nucleic acid encoding the variable heavy domain (VH) of the antibody of claim 1, and
a second nucleic acid encoding the variable light domain (VL) of the same antibody.

16. An expression vector comprising the first and second nucleic acids of claim 15.

17. A host cell comprising the expression vector of claim 16.

18. A method of making an antibody, or antigen binding portion thereof, comprising:
culturing the host cell of claim 17 under conditions wherein the antibody, or antigen binding portion thereof, is produced; and
recovering the antibody or antigen binding portion thereof.

19. A method of treating or preventing SARS-CoV-2 infection in a patient in need comprising administering to the patient the monoclonal antibody, or antigen binding portion thereof, according to claim 1.

20. A method of detecting SARS-CoV-2 in a human sample comprising:
contacting the human sample with the monoclonal antibody, or antigen binding portion thereof, according to claim 1, and
detecting binding of the antibody, or antigen binding portions thereof, to SARS-CoV-2 spike protein (CoV-S) as an indication of presence of SARS-CoV-2 in the sample.

21. The method of claim 20, further comprising a step of identifying one or more variants of SARS-CoV-2 spike protein in the human sample.

22. (canceled)

23. The method of claim 20, wherein the method uses a lateral flow assay.

24. A method of verifying the presence of a SARS-CoV-2 spike protein (CoV-S) of a specific SARS-CoV-2 variant or multiple variants in a vaccine comprising:
contacting the vaccine with one or more of the monoclonal antibodies, or antigen binding portion(s) thereof, according to claim 1, and
detecting binding of the one or more antibodies, or antigen binding portions thereof, to a CoV-S as an indication of the presence of the CoV-S of a specific SARS-CoV-2 variant or multiple variants in the vaccine.
